SEQUENCE LISTING (239 sequences)

SEQ ID NO: 1 - DNA, 30 bp, synthetic construct
  source 1..30: mol_type = other DNA
atttttgtagtgagaccagtcagcatcatc

SEQ ID NO: 2 - DNA, 30 bp, synthetic construct
  source 1..30: mol_type = other DNA
gtttctcatctaagcatatatgtccaaagt

SEQ ID NO: 3 - DNA, 60 bp, synthetic construct
  source 1..60: mol_type = other DNA
tgtatgaaaaattatatttttgtagtgagaccagtcagcatcatcacaccaaaagttagg

SEQ ID NO: 4 - DNA, 60 bp, synthetic construct
  source 1..60: mol_type = other DNA
cgcatcgatcgtgaagtttctcatctaagcatatatgtccaaagtcacgtaactaaggtt

SEQ ID NO: 5 - DNA, 100 bp, synthetic construct
  source 1..100: mol_type = other DNA
tagcattttcgtggggttaatgtatgaaaaattatatttttgtagtgagaccagtcagcatcatcacaccaaaagttaggcccgaatagtttgaaattag

SEQ ID NO: 6 - DNA, 100 bp, synthetic construct
  source 1..100: mol_type = other DNA
agttatcgtacgcctagggccgcatcgatcgtgaagtttctcatctaagcatatatgtccaaagtcacgtaactaaggtttcagccatttaacttttcaa

SEQ ID NO: 7 - DNA, 1050 bp, synthetic construct
  source 1..1050: mol_type = other DNA
gttgcttcttcttctgcattttgaacctatcattagaaggaggtcaaaaatttgttaatgggaaattttgaaactaaattcaccctctcttaaattattgagaccacttatccaacagtacatgtggtggtcgttggtggtggtggttgtcaccatttagaagttgcatgatgattgtcggtgttactgatagttgtcatccaaaagaggattacgcacaatctatggtggttgttggtgttggtggtcattgttgttcttattcgaagtattggtggtggttgtcgatcaaacactagttgcactccaactaacattgccaaaagatccaacccttaattgagacatttgttcaaataccttcaaagcaacaaaacaacattaaaaatctcttcacaaaaaaaaataaccttcttgagttgggaaagatagcacaacatcacaacccaacaacacctaacaatacccaatgaaaaaaaattaactcaacaaagaaacacatgtgaagaaataataatcgcaccacatgaaaaaaattaaatgcacatgaagaaaaaattagcacagatggacatgcttattcatgatggagatgtttacacatgaacttcaacccaaaatacttcaacatgagcttctactttcaatgtctttaactttttcattttcaaaaactaagaaaggtcttagcttttaagagattgtccttaaattaaagaagcatgtgcaatacacatgacaaattttcattaacgaaaatcaaattgtgataagaaagagagttcaattgtaatggagttgcaaaactaataagatttattagagggataatttatggaggaggtcaccttataataagtaggaggggatcgtcgtataatatgtattaaatgaaaggaaagagagtgtaatttattcttaaacctaatatgaaagtgattactatgatatagcattttcgtggggttaatgtatgaaaaattatatttttgtagtgagaccagtcagcatcatcacaccaaaagttaggcccgaatagtttgaaattag

SEQ ID NO: 8 - DNA, 1050 bp, synthetic construct
  source 1..1050: mol_type = other DNA
agttatcgtacgcctagggccgcatcgatcgtgaagtttctcatctaagcatatatgtccaaagtcacgtaactaaggtttcagccatttaacttttcaaatgaacctaatctttcaactttattctaatcttttcttctataataggaaagccattgcttgaagaaagaaccacgttgaaaccaaatttcctcaggaagtaaactttgacaagttaccttgccttcgacccccgtggcttcatcacacaagacaagtcctgattagatagatatattcagtttgtgtagttgaaaaaaatgaaaagtggaaatgagatttatttttccccttcagaaaacagaagttgtgacttgtgacacatatatatagcgagcgaaaacaaagttcatttaatttttcttatattaattatgtattcattttatttttcttactgaataaattacacatgatctaaattaagaaaaataaaaatataattaatgctgtaaaaaaatataattaaatctttcaattttgatcaaagctaaagcataagcatcgcgattcaaaaaagaaaagataagaaaatgcacgggggttgattttatgttcaatttaaaagtatattttatatagatatcttctttttgtaaaatcacggacatctttttataaagtaatcttcgtcgagccgaaaacatttacaacatagtgcagttaattctcgaagtagatttttttcctatactcaaattattgtttaaaaagaaatcaagcatacactactttcacttttaaattattatatatatacctttagttacatacaataatagtacatgaaaacttttctcataagacataaatacataatgattcatatgatttctttttctatttatatctcagttaattagtattgtcccttatttttaagtttagaccgcaagaattttctacaagaaataatcttcttcgatattggaacattcacaatattatgaaaataatttttatggtgtttttttttcatacagaaagcttgcagctgaaaatcttagttaa

SEQ ID NO: 9 - DNA, 3201 bp, synthetic construct
  source 1..3201: mol_type = other DNA
ccagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccttgctaccttaagagagcgccggcgcgtttgttgtgcggccgcctggtgtttgctccaaacagtagcaaccaaaaaaacaaacacaagccgtttttattatataagacgagagattcaccaaacaaatttcttgtggggaaaatataaccaaaatttcaagcagctagcttattgtctcccaaacagacaagtcgcaaaccaaaccaaagcaacaaaaccgtgcgtgcagatatctccagggtatgacaaaataaaatttattaattattcaaatatattacagtttacaactgtgcagcagatacaattcttgatcacacgaagcaaattacagaaactaggttcaacaaagcaagctccctttgctggcaccagtacacaaaacagacagaaaaacagatatagtacaaaaccaaaacaccgccggcggcctccagaaggattaggccggcctactagagggccttcttgtacgatagctttgcaaaatcctccgcgaacttcttaacctgctcccaatccgtgtactcgacctccttgctcgtgtctgtctctcccccagtcatccgcattattagctggatcatcaccttgtcgatccaccggtatcgcgggtaccgaagggcccctgcgaacactccgcacaacgcgggctgccagggggtagcaagcaagaattttcggacataagggtttgtctggggagtacgcttctcgggcttccttgctgtgaggtttacgcagaagaacgcgcttggcatgttgttcagctgatccacgtttctcttgatgaacttgtcaagcacggcgttgaagtggccgtaacgaatactcgcaccgattagcacctgatcgtattgggtgaggttcacgtgctccccgtgggtgaggtcgatcacgtcgcattcggccttctccttcatgcaggaggcgatgtatgaagcaattgcgtgggtctggccgtcccgcgtcgagtacagtaccaaggccttcgacgcgtcgcacctcaccacgaagctccgactggaggtacgagagaacttctcaaagagtccgttcttcttcagctggagctgcttgaaggacttcaaactattgggcttactggggaacgacaccgaaatgcgcgggtgggcggagaatagataggtcttgttggagatcgagtgagtactgcacaacgagatgcccgagaagctcacgacggacgaagatgacgccgccatggtggtactcgagtaccctgtataatccaaaacaaagaaaaacaaatgaatcaatcaccaatttttcattgtgtgtacaacaaggttaaaagaatatgggttatgaaaactggaagcagcagatcagcaattataatcaattcaatttacaacagacatcaaaaaaacttgctcatcaaattctccctgacaatcaataaatcgggtaaaaaatatccatgtactaatctaattaaaggacgtattttttgaatcatacaatacaaaaatccaaatctaaaaatttatatgaacccctaatcacaatctaaagactgtctttgaaaaacaaaggattaaatcatacaataaacagtaaaatcaagtaaacggttcaattcagaaggatttcaatcagatgaaagaaaagaacaaaaccctaattgaatttaaatttgacggatcaaaagaaaagatcgattaaacgatagtagctaataataataaaataatcgaagaatagaaagtagattataccttgaaacggaattgggtgctaggtttgaatgaatctcttttccaaaggaaccggttttgtagcgatggggaagaaaagaatgaagaagtttagtttacctgatttgcttttatagcaaaaatcgattggctgttatattacgccaacggcagctcttccaattaaaacgtgacacgtggacaaactgtgacttgttacctaaccctttgtgatacgacaacgtttggtataagagagtccacgtctattatatgctgtcttgcaccgcctcgagacaattcttctaactaagatcgattgtgatacaggtgcgggtacaatacaatacgggtacgtgaattagcggtttttaaaaaaaaattaagatacgatacggtaatgacttatgatacgttaacaatgatttataataaaacttatatttgaagaaaaattaacactgattactctttattattgtattattataaaagatacaatttttaaaaaatagtacatgtgattattaaaataaattttttaaaaattttcttataaatcatattacattttcatatgatgtgatattgagagaaacatcattaaaagaacttaacattaattttatttaatgttgtctaactgaatcacctctctcgctacgttcttccacaagaaatataattttaagtgtttcatcgagaatcaattatactgatgcatatctaaaattgaatcatatacatttataatgaatttttacaacattgtctcacttttttactcttcatgtttttctaggatacgtatctataaagtatcttagaaatatccttgtgtatatgtagagtatcacataagtatccgataaaatatttattaaaaattaaaataattaatatgatacttcgtgcgtacgtatccgatatgagtacgtctctgtattgaagtatacaggtttcatagctgatacatctctttcacggttttgtctaatgtacccaaatcataagtaaaatagtattgcgtcataaactagtgtgtgttttttattttcacgtgtccgtcttttcgtttttttagtgcgctgacgtttaaaaattattaacagtataacttcgtatagcatacattatacgaagttatcgtacgcctagggccgcatcgatcgtgaagtttctcatctaagc

SEQ ID NO: 10 - DNA, 5201 bp, synthetic construct
  source 1..5201: mol_type = other DNA
gttgcttcttcttctgcattttgaacctatcattagaaggaggtcaaaaatttgttaatgggaaattttgaaactaaattcaccctctcttaaattattgagaccacttatccaacagtacatgtggtggtcgttggtggtggtggttgtcaccatttagaagttgcatgatgattgtcggtgttactgatagttgtcatccaaaagaggattacgcacaatctatggtggttgttggtgttggtggtcattgttgttcttattcgaagtattggtggtggttgtcgatcaaacactagttgcactccaactaacattgccaaaagatccaacccttaattgagacatttgttcaaataccttcaaagcaacaaaacaacattaaaaatctcttcacaaaaaaaaataaccttcttgagttgggaaagatagcacaacatcacaacccaacaacacctaacaatacccaatgaaaaaaaattaactcaacaaagaaacacatgtgaagaaataataatcgcaccacatgaaaaaaattaaatgcacatgaagaaaaaattagcacagatggacatgcttattcatgatggagatgtttacacatgaacttcaacccaaaatacttcaacatgagcttctactttcaatgtctttaactttttcattttcaaaaactaagaaaggtcttagcttttaagagattgtccttaaattaaagaagcatgtgcaatacacatgacaaattttcattaacgaaaatcaaattgtgataagaaagagagttcaattgtaatggagttgcaaaactaataagatttattagagggataatttatggaggaggtcaccttataataagtaggaggggatcgtcgtataatatgtattaaatgaaaggaaagagagtgtaatttattcttaaacctaatatgaaagtgattactatgatatagcattttcgtggggttaatgtatgaaaaattatatttttgtagtgagaccagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatccttgctaccttaagagagcgccggcgcgtttgttgtgcggccgcctggtgtttgctccaaacagtagcaaccaaaaaaacaaacacaagccgtttttattatataagacgagagattcaccaaacaaatttcttgtggggaaaatataaccaaaatttcaagcagctagcttattgtctcccaaacagacaagtcgcaaaccaaaccaaagcaacaaaaccgtgcgtgcagatatctccagggtatgacaaaataaaatttattaattattcaaatatattacagtttacaactgtgcagcagatacaattcttgatcacacgaagcaaattacagaaactaggttcaacaaagcaagctccctttgctggcaccagtacacaaaacagacagaaaaacagatatagtacaaaaccaaaacaccgccggcggcctccagaaggattaggccggcctactagagggccttcttgtacgatagctttgcaaaatcctccgcgaacttcttaacctgctcccaatccgtgtactcgacctccttgctcgtgtctgtctctcccccagtcatccgcattattagctggatcatcaccttgtcgatccaccggtatcgcgggtaccgaagggcccctgcgaacactccgcacaacgcgggctgccagggggtagcaagcaagaattttcggacataagggtttgtctggggagtacgcttctcgggcttccttgctgtgaggtttacgcagaagaacgcgcttggcatgttgttcagctgatccacgtttctcttgatgaacttgtcaagcacggcgttgaagtggccgtaacgaatactcgcaccgattagcacctgatcgtattgggtgaggttcacgtgctccccgtgggtgaggtcgatcacgtcgcattcggccttctccttcatgcaggaggcgatgtatgaagcaattgcgtgggtctggccgtcccgcgtcgagtacagtaccaaggccttcgacgcgtcgcacctcaccacgaagctccgactggaggtacgagagaacttctcaaagagtccgttcttcttcagctggagctgcttgaaggacttcaaactattgggcttactggggaacgacaccgaaatgcgcgggtgggcggagaatagataggtcttgttggagatcgagtgagtactgcacaacgagatgcccgagaagctcacgacggacgaagatgacgccgccatggtggtactcgagtaccctgtataatccaaaacaaagaaaaacaaatgaatcaatcaccaatttttcattgtgtgtacaacaaggttaaaagaatatgggttatgaaaactggaagcagcagatcagcaattataatcaattcaatttacaacagacatcaaaaaaacttgctcatcaaattctccctgacaatcaataaatcgggtaaaaaatatccatgtactaatctaattaaaggacgtattttttgaatcatacaatacaaaaatccaaatctaaaaatttatatgaacccctaatcacaatctaaagactgtctttgaaaaacaaaggattaaatcatacaataaacagtaaaatcaagtaaacggttcaattcagaaggatttcaatcagatgaaagaaaagaacaaaaccctaattgaatttaaatttgacggatcaaaagaaaagatcgattaaacgatagtagctaataataataaaataatcgaagaatagaaagtagattataccttgaaacggaattgggtgctaggtttgaatgaatctcttttccaaaggaaccggttttgtagcgatggggaagaaaagaatgaagaagtttagtttacctgatttgcttttatagcaaaaatcgattggctgttatattacgccaacggcagctcttccaattaaaacgtgacacgtggacaaactgtgacttgttacctaaccctttgtgatacgacaacgtttggtataagagagtccacgtctattatatgctgtcttgcaccgcctcgagacaattcttctaactaagatcgattgtgatacaggtgcgggtacaatacaatacgggtacgtgaattagcggtttttaaaaaaaaattaagatacgatacggtaatgacttatgatacgttaacaatgatttataataaaacttatatttgaagaaaaattaacactgattactctttattattgtattattataaaagatacaatttttaaaaaatagtacatgtgattattaaaataaattttttaaaaattttcttataaatcatattacattttcatatgatgtgatattgagagaaacatcattaaaagaacttaacattaattttatttaatgttgtctaactgaatcacctctctcgctacgttcttccacaagaaatataattttaagtgtttcatcgagaatcaattatactgatgcatatctaaaattgaatcatatacatttataatgaatttttacaacattgtctcacttttttactcttcatgtttttctaggatacgtatctataaagtatcttagaaatatccttgtgtatatgtagagtatcacataagtatccgataaaatatttattaaaaattaaaataattaatatgatacttcgtgcgtacgtatccgatatgagtacgtctctgtattgaagtatacaggtttcatagctgatacatctctttcacggttttgtctaatgtacccaaatcataagtaaaatagtattgcgtcataaactagtgtgtgttttttattttcacgtgtccgtcttttcgtttttttagtgcgctgacgtttaaaaattattaacagtataacttcgtatagcatacattatacgaagttatcgtacgcctagggccgcatcgatcgtgaagtttctcatctaagcatatatgtccaaagtcacgtaactaaggtttcagccatttaacttttcaaatgaacctaatctttcaactttattctaatcttttcttctataataggaaagccattgcttgaagaaagaaccacgttgaaaccaaatttcctcaggaagtaaactttgacaagttaccttgccttcgacccccgtggcttcatcacacaagacaagtcctgattagatagatatattcagtttgtgtagttgaaaaaaatgaaaagtggaaatgagatttatttttccccttcagaaaacagaagttgtgacttgtgacacatatatatagcgagcgaaaacaaagttcatttaatttttcttatattaattatgtattcattttatttttcttactgaataaattacacatgatctaaattaagaaaaataaaaatataattaatgctgtaaaaaaatataattaaatctttcaattttgatcaaagctaaagcataagcatcgcgattcaaaaaagaaaagataagaaaatgcacgggggttgattttatgttcaatttaaaagtatattttatatagatatcttctttttgtaaaatcacggacatctttttataaagtaatcttcgtcgagccgaaaacatttacaacatagtgcagttaattctcgaagtagatttttttcctatactcaaattattgtttaaaaagaaatcaagcatacactactttcacttttaaattattatatatatacctttagttacatacaataatagtacatgaaaacttttctcataagacataaatacataatgattcatatgatttctttttctatttatatctcagttaattagtattgtcccttatttttaagtttagaccgcaagaattttctacaagaaataatcttcttcgatattggaacattcacaatattatgaaaataatttttatggtgtttttttttcatacagaaagcttgcagctgaaaatcttagttaa

SEQ ID NO: 11 - DNA, 1000 bp, Glycine max
  source 1..1000: mol_type = genomic DNA
gttgcttcttcttctgcattttgaacctatcattagaaggaggtcaaaaatttgttaatgggaaattttgaaactaaattcaccctctcttaaattattgagaccacttatccaacagtacatgtggtggtcgttggtggtggtggttgtcaccatttagaagttgcatgatgattgtcggtgttactgatagttgtcatccaaaagaggattacgcacaatctatggtggttgttggtgttggtggtcattgttgttcttattcgaagtattggtggtggttgtcgatcaaacactagttgcactccaactaacattgccaaaagatccaacccttaattgagacatttgttcaaataccttcaaagcaacaaaacaacattaaaaatctcttcacaaaaaaaaataaccttcttgagttgggaaagatagcacaacatcacaacccaacaacacctaacaatacccaatgaaaaaaaattaactcaacaaagaaacacatgtgaagaaataataatcgcaccacatgaaaaaaattaaatgcacatgaagaaaaaattagcacagatggacatgcttattcatgatggagatgtttacacatgaacttcaacccaaaatacttcaacatgagcttctactttcaatgtctttaactttttcattttcaaaaactaagaaaggtcttagcttttaagagattgtccttaaattaaagaagcatgtgcaatacacatgacaaattttcattaacgaaaatcaaattgtgataagaaagagagttcaattgtaatggagttgcaaaactaataagatttattagagggataatttatggaggaggtcaccttataataagtaggaggggatcgtcgtataatatgtattaaatgaaaggaaagagagtgtaatttattcttaaacctaatatgaaagtgattactatgatatagcattttcgtggggttaatgtatgaaaaattatatttttgtagtgaga

SEQ ID NO: 12 - DNA, 1000 bp, Glycine max
  source 1..1000: mol_type = genomic DNA
atatatgtccaaagtcacgtaactaaggtttcagccatttaacttttcaaatgaacctaatctttcaactttattctaatcttttcttctataataggaaagccattgcttgaagaaagaaccacgttgaaaccaaatttcctcaggaagtaaactttgacaagttaccttgccttcgacccccgtggcttcatcacacaagacaagtcctgattagatagatatattcagtttgtgtagttgaaaaaaatgaaaagtggaaatgagatttatttttccccttcagaaaacagaagttgtgacttgtgacacatatatatagcgagcgaaaacaaagttcatttaatttttcttatattaattatgtattcattttatttttcttactgaataaattacacatgatctaaattaagaaaaataaaaatataattaatgctgtaaaaaaatataattaaatctttcaattttgatcaaagctaaagcataagcatcgcgattcaaaaaagaaaagataagaaaatgcacgggggttgattttatgttcaatttaaaagtatattttatatagatatcttctttttgtaaaatcacggacatctttttataaagtaatcttcgtcgagccgaaaacatttacaacatagtgcagttaattctcgaagtagatttttttcctatactcaaattattgtttaaaaagaaatcaagcatacactactttcacttttaaattattatatatatacctttagttacatacaataatagtacatgaaaacttttctcataagacataaatacataatgattcatatgatttctttttctatttatatctcagttaattagtattgtcccttatttttaagtttagaccgcaagaattttctacaagaaataatcttcttcgatattggaacattcacaatattatgaaaataatttttatggtgtttttttttcatacagaaagcttgcagctgaaaatcttagttaa

SEQ ID NO: 13 - DNA, 2010 bp, Glycine max
  source 1..2010: mol_type = genomic DNA
gttgcttcttcttctgcattttgaacctatcattagaaggaggtcaaaaatttgttaatgggaaattttgaaactaaattcaccctctcttaaattattgagaccacttatccaacagtacatgtggtggtcgttggtggtggtggttgtcaccatttagaagttgcatgatgattgtcggtgttactgatagttgtcatccaaaagaggattacgcacaatctatggtggttgttggtgttggtggtcattgttgttcttattcgaagtattggtggtggttgtcgatcaaacactagttgcactccaactaacattgccaaaagatccaacccttaattgagacatttgttcaaataccttcaaagcaacaaaacaacattaaaaatctcttcacaaaaaaaaataaccttcttgagttgggaaagatagcacaacatcacaacccaacaacacctaacaatacccaatgaaaaaaaattaactcaacaaagaaacacatgtgaagaaataataatcgcaccacatgaaaaaaattaaatgcacatgaagaaaaaattagcacagatggacatgcttattcatgatggagatgtttacacatgaacttcaacccaaaatacttcaacatgagcttctactttcaatgtctttaactttttcattttcaaaaactaagaaaggtcttagcttttaagagattgtccttaaattaaagaagcatgtgcaatacacatgacaaattttcattaacgaaaatcaaattgtgataagaaagagagttcaattgtaatggagttgcaaaactaataagatttattagagggataatttatggaggaggtcaccttataataagtaggaggggatcgtcgtataatatgtattaaatgaaaggaaagagagtgtaatttattcttaaacctaatatgaaagtgattactatgatatagcattttcgtggggttaatgtatgaaaaattatatttttgtagtgagaatgatatagcatatatgtccaaagtcacgtaactaaggtttcagccatttaacttttcaaatgaacctaatctttcaactttattctaatcttttcttctataataggaaagccattgcttgaagaaagaaccacgttgaaaccaaatttcctcaggaagtaaactttgacaagttaccttgccttcgacccccgtggcttcatcacacaagacaagtcctgattagatagatatattcagtttgtgtagttgaaaaaaatgaaaagtggaaatgagatttatttttccccttcagaaaacagaagttgtgacttgtgacacatatatatagcgagcgaaaacaaagttcatttaatttttcttatattaattatgtattcattttatttttcttactgaataaattacacatgatctaaattaagaaaaataaaaatataattaatgctgtaaaaaaatataattaaatctttcaattttgatcaaagctaaagcataagcatcgcgattcaaaaaagaaaagataagaaaatgcacgggggttgattttatgttcaatttaaaagtatattttatatagatatcttctttttgtaaaatcacggacatctttttataaagtaatcttcgtcgagccgaaaacatttacaacatagtgcagttaattctcgaagtagatttttttcctatactcaaattattgtttaaaaagaaatcaagcatacactactttcacttttaaattattatatatatacctttagttacatacaataatagtacatgaaaacttttctcataagacataaatacataatgattcatatgatttctttttctatttatatctcagttaattagtattgtcccttatttttaagtttagaccgcaagaattttctacaagaaataatcttcttcgatattggaacattcacaatattatgaaaataatttttatggtgtttttttttcatacagaaagcttgcagctgaaaatcttagttaa

SEQ ID NO: 14 - DNA, 5000 bp, Glycine max
  source 1..5000: mol_type = genomic DNA
ttttttttataaaaaaaaagggaaaaacttaggtagagttagtaacctgtttgtggcgatttaaaacatctgggttaagagatccatctaagagcctagacagaatctggaagaaagaaaaagagaaatgtaaaaagaaaaagaagtgaatttgcgtgaatagaacgcgtgagagaagaaaaagaagggacctcgatctggccattggctgcagcaatatgaagaggagagtggcgatcgtagagagttgtttggtgcaagagagaagggtcggaatccaagagagttgtgacaatttgaaggtcgccgtgcagcacggcggtgaagaggccgtggtcatggttgccactgcaactcaggctctgacccattattacgatgggagagaggtgaagaatgggttttgttattctacttgttcttaacctcttgtgggtttggtttggctgcgccacctgagctggtggtggttataaataaatgagtcttgactcagacattgaagttgtaagttgtaaggtgttgtgggagacaatggcaaaggttttagagagagaaggggtagcttagcttagctgaccgtgtcagtgattagcagttactgagaatgacttcgggcagcagtcagattccgcgtaagcctgtttggctccgctacccagataaatgaaatgagataaatacattgcactgcctcttcatcttattatccaatccaacaagctagagggtccccccttcccttcttttttttttccaacaccatcaatatattttaaattcattaaaaatgataggtttcagcctcattagtcattgaaaaaaaataaatagatctccgcataacatacatatggaaatgattcaattttaacttaggaatatttacttataacactattgaaattaattatattaaaaaaattggataatgcgggactaagccaacaaaaaggaagttacacatataacaaattaagttctctaaaatatcagaaaacttttacaatttaattgatgcataattttattttatctatagataaaattttataataatttacatatattatttaactaattaaactagatctcttcgattgcttgtgcatttgaacttatgaacaagctcatcttattttttcttgtataaatcttttctgataaatttatccaaacaagttcaccaataaaaaaaatctttaaattcagggttgctttttactatttgagatttattggcgctataaattgtagtgtaagtagctgtaatattccaatcttccaacgcagcagcctgtgggcggggagcatttagcgattattggggcaattttacagcacctttctcttaataatttgggcacctgctctatcatgcagaccaatttatgagacatgcatggatacctttcatggtatgtagcagataataggtggctggagatttcaacaccacaatattccttcaccatggctgcctaaatgctcatcctcatgtaccacttgatcaagtcattattatattgacttgatgattgaccttaattagccatattcttgggagaaggcaatttcttagcaaatgacgacttagctaaacatttggcatctgatctatagatccctgtattcatgcaattctgtcttttgtttcctttctaatcgcgcgcatgttctgattataacaagtgtttagatcgtagatttgttgtctttagtaccattcattcacaaaaacaaatgggtacgtgctagattgacaaaacaagtgtgacttagagatatatatgcattgataattgcaagattcgatcgatttcatttcagtatatactaataataataactatacgttcccccatcctaaatcttaaatttattattaattcaaaactattattcacaattgtacaattgattagtagtcaatgcccatacaaaataagagtaataaaataaaatcatttatatttatattagattctagctattgttgtggaaaaattataaaaaatactcattttattagatttattatttatattaggcaaataagaataactagtgtttaaattgacttaatttatcaagaaagtttagtttacttatttgagtagaatgtgtgatcttcttattatgtttaatttttatgaatataaaaaattgatctaatttttttaagatcgccgtgaataaaataaaggaaaatacattacataaaacttaaaataaaatctgaagcgggaaatatataactagttatcaacaacgacctttcttagactcccataagatgaaataaaactgaattccatggagtatataaaatggatcataatttggctgctgcagacaccgttaagtttaattttctttctttttttataaaaaataaaattatgagtttgatttttttttctacacgctataatatcagttgctctaaaagtgtcatttttttggttaaaagtattcaaaaagtttctgataatactggcgaggatataattttgccaaagaccggagatatataaccctgctattttttcataatattcctaacttaagtcactagaattgtctcccatgaaatatatgttgtagtaaataatcagcaaacatacggagatataactttgattggttgggcaaatgttgttaaaataaacgaaataatcagctacaattgacgatcatatattatttctaccacaattaattgatgtacatatatgttatactaaataatcagctaacatacgggagtataattccagttaattgagcatgtattgtgtgagtggttataaataccttatgcttattttatttttacgcataaaaaatttaatcagctaacatattgcacgcttaccttttactcttcacaaatcgcaccatattatgctaatacacgcctgtttttcatttttatttgtgttccccaaaggtacgtaattaaatctagagcaactatattcactttccactatttagtatttattacacgtgaatctcctacttaaaattgcatttctcttgctttctctaacccgaaattgttagtgacgatgttagttttttttttaaatacaaaaataagccttactaatttagccacatacgatgttagttgttttgacatgattttgatgatgctaaagaagaatcaaataatgttgtttcaaaggataaatatttcttcaagattaatataaggttgtttcaacaaacaaaaccttacttcaagattaactcaagatcaagtcttgcctcaaaacaaagtatttccaagacatttaagactttggtaatcgattaccagacaatgtaatcgattacaagaagacaattttgcaaaatatctgttaatcgattaccagatatttgtaatcgattactagtaatggaactcttgaaattcaaattcaaaagtcacgacccttcaaaatataactgtgtaatcgattactagtgaaaaaaatttagaaaaaactttttgaaaagacacatctcttcaaatcattttgaaaaggcacgaagagtctatatatatgtgtgtctgacttcaaaaagtaatagggagatattccaagataacttcattatcaaatgttctctcaacaactcttaggtaaatacttgcaaatctatgaatagttaatccaagaacttcaaattgtactatccactctaaaggagagaaatccttttgttcttcttagaaagtcaattataatcaagagactggttgtctcttgaattgtgagtttcctgaacacaagaaaaagagattccttgagtgttcagaagttataaaaatgatttttacaaagatagtggaaaatctaaagtgggttgtttgaggactgaacgtaggcacggaaagtggttgaactagtataaatcgagtttgcatttctctcttttcttatcttatttattttattacagtcaattttgtcttgcacgtttaaagaacattattaaattgattgttgcttcttcttctgcattttgaacctatcattagaaggaggtcaaaaatttgttaatgggaaattttgaaactaaattcaccctctcttaaattattgagaccacttatccaacagtacatgtggtggtcgttggtggtggtggttgtcaccatttagaagttgcatgatgattgtcggtgttactgatagttgtcatccaaaagaggattacgcacaatctatggtggttgttggtgttggtggtcattgttgttcttattcgaagtattggtggtggttgtcgttcaaacactagttgcactccaactaacattgccaaaagatccaacccttaattgagacatttgttcaaataccttcaaagcaacaaaacaacgttaaaaatctcttcacaaaaaaaaataaccttcttgagttgggaaagatagcacaacatcacaacccaacaacacctaacaatacccaatgaaaaaaaattaactcaacaaagaaacacatgtgaagaaataataatcgcaccacatgaaaaaaattaaatgcacatgaagaaaaaattagcacagatggacatgcttattcatgatggagatgtttacacatgaacttcaacccaaaatacttcaacatgagcttctactttcaatgtctttaactttttcattttcaaaaactaagaaaggtcttagcttttaagagattgtccttaaattaaagaagcatgtgcaatacacatgacaaattttcattaacgaaaatcaaattgtgataagaaagagagttcaattgtaatggagttgcaaaactaataagatttattagagggataatttatggaggaggtcaccttataataagtaggaggggatcgtcgtataatatgtattaaatgaaaggaaagagagtgtaatttattcttaaacctaatatgaaagtgattactatgatatagcattttcgtggggttaatgtatgaaaaattatatttttgtagtgaga

SEQ ID NO: 15 - DNA, 5000 bp, Glycine max
  source 1..5000: mol_type = genomic DNA
atatatgtccaaagtcacgtaactaaggtttcagccatttaacttttcaaatgaacctaatctttcaactttattctaatcttttcttctataataggaaagccattgcttgaagaaagaaccacgttgaaaccaaatttcctcaggaagtaaactttgacaagttaccttgccttcgacccccgtggcttcatcacacaagacaagtcctgattagatagatatattcagtttgtgtagttgaaaaaaatgaaaagtggaaatgagatttatttttccccttcagaaaacagaagttgtgacttgtgacacatatatatagcgagcgaaaacaaagttcatttaatttttcttatattaattatgtattcattttatttttcttactgaataaattacacatgatctaaattaagaaaaataaaaatataattaatgctgtaaaaaaatataattaaatctttcaattttgatcaaagctaaagcataagcatcgcgattcaaaaaagaaaagataagaaaatgcacgggggttgattttatgttcaatttaaaagtatattttatatagatatcttctttttgtaaaatcacggacatctttttataaagtaatcttcgtcgagccgaaaacatttacaacatagtgcagttaattctcgaagtagatttttttcctatactcaaattattgtttaaaaagaaatcaagcatacactactttcacttttaaattattatatatatacctttagttacatacaataatagtacatgaaaacttttctcataagacataaatacataatgattcatatgatttctttttctatttatatctcagttaattagtattgtcccttatttttaagtttagaccgcaagaattttctacaagaaataatcttcttcgatattggaacattcacaatattatgaaaataatttttatggtgtttttttttcatacagaaagcttgcagctgaaaatcttagttaagagaaagcatgtatcgcttaaattaatcactcactcattattttatattaatgtcaaccatcaacttttttttttacaatatttatgcatttttaattgacaccttttaagcttgtcacgcttgtttctttcatcatcaagtggcaactgtccacaagaggatacccctcttatctctagcgatttacaagcacacgcaccaataaaataaaaaataaaaaaataaaaaatactctcttactcgttggtctatgctttcacaatttcataagcatgtataattccaaccattggtcactgtcatcggtcattgctgggcctgtgtagggcaggtataatttcatataatttcattcgataactgtagtttttttattgagatatttgataagttcacttagatgttattattagcaacaagtcacatatcatattcaattaactaagttattcctccttagtattaaaatgtcattatttagtgtcaaaagtctgattatatttttgtgatgatatggaatttacgaatatttctactctttttttcggttcaactagtagtactcaactttattgcataaagctaagaaaccttcattgcataattgataaatacaacttgacaggatttaaggcccatgtaagtaacgtcattgaattaaggatccctaagtagtttctgtatgagcctaataagcaagctccatctaggtaaaagatttagacttttttcatatccattcccctattttattaagtacactaccctttagtttttcttttgattttttttatcataaatgctccctataccaaaaatacccttatatcattattccaaaaaatatttttttttaaaatgatgtttaaattttttcttccaaaaagtacattccacaatacaaattttaaacatcattctagaaagtattttctataatacaaaataatagattccgaaaacgtactttccagagtactatttaaaatttttgtattccacaaagtaatttttagaagtaaaagaatctaggtatttgtgaaatttaataaaaaaaatacaatcatcttagtaaagtgacacacaaagaaaacattctaatgtgtttatatctcatctcttaaacctctaattaaattacacaacatgggcaaatcatgtatgaagatttgaagttggtaaacacttttcctagtttatggcaagagaataaagctattaggtcagtaaaatccagaaagtgaggaagaccaaagtaagatatcaacttggagtaataaagagtgaaagagaggaatgattctaatgaaaaagaggggaatgtttcattgccatggaaagagtacattgttgggggaggggggagagtgtgagattgtgtgaaggaaaaaacaaagtttaaaataaaaagattatttaagtcttttgagttcaaaatagaagaagggaagtgtagttagaaaaaagggagcgaatatagcaagtgtccaaaaattttaaatgtcatctggctcaatttgaggattattggggcaatccgacccaatcagaaccatcttttacacccttgccggtatcctccaagacagaagagctaaaatccaccaagacagaagagcttttatttgctattagttattaaattgtttttagaaggataatactttaaaaaataacttatatttatgatcactaaattgattttttaaatgactaataatgacattaaaaaaagtcatttataaacagaataaaaagcaccaagctctaaaataacccccgtcactaatagttaaaagtttgagtagttttccctgaatactaatattgtagctgcatagaaaaaaatactaatattgaagtcaccatttcctttaaaaaaatatattgtagtcgtcaccattgtataacaaaaacaaatacctagcctccgttaatatgatatttcttaaattgttaatataaatatcgagtctaaactaacagatatattgtactaatattttatatattgaaaaataactaatttccaatattggaaaattttctaattataatatttatatatactaacattctaacaatgcaatgctcaagataaatatttattttaaataactaaaatttgtaaaaataaatttttttaataaaaaaagttatattttgaatttaggataaataatttatattgtgatatattttttaattattaatatttacaaaaatattaaattttaatttagaaattaaaaaaggatagattaaaagaagatataagctgtataaaaattaaatattagaaaataatataaataaactaaaattaaaattaaattcatatatgatccattttattaactggtaatgccatttctctactttgttatttgtttggattagtgttttttataagcatgattgtattaaacatattttaaattttttaatattattcttacaattttttcataattaacaaaatttagggaaatcagattgttttccagttcaaaactgagaaagtctttgagtttaaagaatgtttataaaagtatttatatttataagttcagtaactaatattgataagtttttggtaataaaaattatacttttatgatttatctctcttataagcaatatttatattggttgtgtagattgggcctaggaatcaaaatggaattactttgtatattttgttttatcctggtagctagagtcctgagaaaaagataacatcggacagggttactcgattcatcagattgcttggtcactgactcagtcttgactcttgactgttaccgcagttcctttaaccgaattaaccgaaccttacatcatcaacaatagccgaagcccagtcatccaccccaacagcagttacagttaatataggcgagagattattgtgtttataacggttcttgacctaattaatttatttagtatatagtatacttgtttgttcacttcacactgcattgattttaacgcaaacgcttagtgatttaacgtggaacacgggttttcgatctcattccattccagctagctctatgttactgttattttaataaccagaaattaatattaaattaatctctagaaaacgcaattttgatccccacatattacaacatctttttagtgttacctttgcaaatttggagtaagcaatgtgcaggtcaagcgtgaatctactcgatattaatttgaaaatagtttatcaagacatagtttcaactactacaggtatacgatgcttttcaataatacattaacacacattaagatataaatactccaaatgctacgtattaattatatacattacatttattataatccatgcttatcctgaattggctttcatgacatgtccttagaaacattttttggtataggtaatgtatatataggaaacaattgggcttaactttttgtatagtgcctcagcctctccgtacctcttgtgcagtagattgcttttttcagagtattaatacatatatctttgatgttcaaagaaaaaaaaaatccatgcttatcctgtttaattcaaagtaaattagttctagagattgaagaatatttcataaaaaggcttgttactccctaacctcagtaaattcataaaaatagagacaaaataatatagcagtgataatggtacagaaaaatgtacaacgcttaattagacggacctcacaaccgtaagcatggtcacctttgcttttttttttttcctgcctccctgtctctcttttatttctttccatcttcatataacaatgttactgtgtaatgtccggtttagaatgtgtgaaggtaacctttcccattaaaaaacatgacgtgcgtgccatgtagaatagtaactagcagtaactttttcccattgattcccttttagttctctcttactttgtcgatttgaacaaatatatagcattgcttttacatacaaatatattttcaccaatgcacattact

SEQ ID NO: 16 - DNA, 442 bp, synthetic construct
  source 1..442: mol_type = other DNA
ctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt

SEQ ID NO: 17 - DNA, 34 bp, synthetic construct
  source 1..34: mol_type = other DNA
ataacttcgtatagcatacattatacgaagttat

SEQ ID NO: 18 - DNA, 1010 bp, Medicago truncatula
  source 1..1010: mol_type = genomic DNA
actgttaataatttttaaacgtcagcgcactaaaaaaacgaaaagacggacacgtgaaaataaaaaacacacactagtttatgacgcaatactattttacttatgatttgggtacattagacaaaaccgtgaaagagatgtatcagctatgaaacctgtatacttcaatacagagacgtactcatatcggatacgtacgcacgaagtatcatattaattattttaatttttaataaatattttatcggatacttatgtgatactctacatatacacaaggatatttctaagatactttatagatacgtatcctagaaaaacatgaagagtaaaaaagtgagacaatgttgtaaaaattcattataaatgtatatgattcaattttagatatgcatcagtataattgattctcgatgaaacacttaaaattatatttcttgtggaagaacgtagcgagagaggtgattcagttagacaacattaaataaaattaatgttaagttcttttaatgatgtttctctcaatatcacatcatatgaaaatgtaatatgatttataagaaaatttttaaaaaatttattttaataatcacatgtactattttttaaaaattgtatcttttataataatacaataataaagagtaatcagtgttaatttttcttcaaatataagttttattataaatcattgttaacgtatcataagtcattaccgtatcgtatcttaatttttttttaaaaaccgctaattcacgtacccgtattgtattgtacccgcacctgtatcacaatcgatcttagttagaagaattgtctcgaggcggtgcaagacagcatataatagacgtggactctcttataccaaacgttgtcgtatcacaaagggttaggtaacaagtcacagtttgtccacgtgtcacgttttaattggaagagctgccgttggcgtaatataacagccaatcgatttttgctataaaagcaaatcaggtaaactaaacttcttc

SEQ ID NO: 19 - DNA, 82 bp, Medicago truncatula
  source 1..82: mol_type = genomic DNA
attcttttcttccccatcgctacaaaaccggttcctttggaaaagagattcattcaaacctagcacccaattccgtttcaag

SEQ ID NO: 20 - DNA, 502 bp, Medicago truncatula
  source 1..502: mol_type = genomic DNA
gtataatctactttctattcttcgattattttattattattagctactatcgtttaatcgatcttttcttttgatccgtcaaatttaaattcaattagggttttgttcttttctttcatctgattgaaatccttctgaattgaaccgtttacttgattttactgtttattgtatgatttaatcctttgtttttcaaagacagtctttagattgtgattaggggttcatataaatttttagatttggatttttgtattgtatgattcaaaaaatacgtcctttaattagattagtacatggatattttttacccgatttattgattgtcagggagaatttgatgagcaagtttttttgatgtctgttgtaaattgaattgattataattgctgatctgctgcttccagttttcataacccatattcttttaaccttgttgtacacacaatgaaaaattggtgattgattcatttgtttttctttgttttggattatacagggt

SEQ ID NO: 21 - DNA, 234 bp, synthetic construct
  source 1..234: mol_type = other DNA
atggcggcgtcatcttcgtccgtcgtgagcttctcgggcatctcgttgtgcagtactcactcgatctccaacaagacctatctattctccgcccacccgcgcatttcggtgtcgttccccagtaagcccaatagtttgaagtccttcaagcagctccagctgaagaagaacggactctttgagaagttctctcgtacctccagtcggagcttcgtggtgaggtgcgacgcgtcg

SEQ ID NO: 22 - DNA, 537 bp, synthetic construct
  source 1..537: mol_type = other DNA
aaggccttggtactgtactcgacgcgggacggccagacccacgcaattgcttcatacatcgcctcctgcatgaaggagaaggccgaatgcgacgtgatcgacctcacccacggggagcacgtgaacctcacccaatacgatcaggtgctaatcggtgcgagtattcgttacggccacttcaacgccgtgcttgacaagttcatcaagagaaacgtggatcagctgaacaacatgccaagcgcgttcttctgcgtaaacctcacagcaaggaagcccgagaagcgtactccccagacaaacccttatgtccgaaaattcttgcttgctaccccctggcagcccgcgttgtgcggagtgttcgcaggggcccttcggtacccgcgataccggtggatcgacaaggtgatgatccagctaataatgcggatgactgggggagagacagacacgagcaaggaggtcgagtacacggattgggagcaggttaagaagttcgcggaggattttgcaaagctatcgtacaagaaggccctctag

SEQ ID NO: 23 - DNA, 400 bp, synthetic construct
  source 1..400: mol_type = other DNA
tccttctggaggccgccggcggtgttttggttttgtactatatctgtttttctgtctgttttgtgtactggtgccagcaaagggagcttgctttgttgaacctagtttctgtaatttgcttcgtgtgatcaagaattgtatctgctgcacagttgtaaactgtaatatatttgaataattaataaattttattttgtcataccctggagatatctgcacgcacggttttgttgctttggtttggtttgcgacttgtctgtttgggagacaataagctagctgcttgaaattttggttatattttccccacaagaaatttgtttggtgaatctctcgtcttatataataaaaacggcttgtgtttgtttttttggttgctactgtttggagcaaacaccag

SEQ ID NO: 24 - DNA, 505 bp, synthetic construct
  source 1..505: mol_type = other DNA
cgaagctcggtcccgtgggtgttctgtcgtctcgttgtacaacgaaatccattcccattccgcgctcaagatggcttcccctcggcagttcatcagggctaaatcaatctagccgacttgtccggtgaaatgggctgcactccaacagaaacaatcaaacaaacatacacagcgacttattcacacgagctcaaattacaacggtatatatcctgccagtcagcatcatcacaccaaaagttaggcccgaatagtttgaaattagaaagctcgcaattgaggtctacaggccaaattcgctcttagccgtacaatattactcaccggtgcgatgccccccatcgtaggtgaaggtggaaattaatgatccatcttgagaccacaggcccacaacagctaccagtttcctcaagggtccaccaaaaacgtaagcgcttacgtacatggtcgataagaaaaggcaatttgtagatgttaacatccaacgtcgctttcagggatcctt

SEQ ID NO: 25 - protein, 178 aa, Enterobacter cloacae
KALVLYSTRDGQTHAIASYIASCMKEKAECDVIDLTHGEHVNLTQYDQVLIGASIRYGHFNAVLDKFIKRNVDQLNNMPSAFFCVNLTARKPEKRTPQTNPYVRKFLLATPWQPALCGVFAGALRYPRYRWIDKVMIQLIMRMTGGETDTSKEVEYTDWEQVKKFAEDFAKLSYKKAL

SEQ ID NO: 26 - DNA, 552 bp, synthetic construct
  source 1..552: mol_type = other DNA
atgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttga

SEQ ID NO: 27 - protein, 183 aa, Streptomyces viridochromogenes
MSPERRPVEIRPATAADMAAVCDIVNHYIETSTVNFRTEPQTPQEWIDDLERLQDRYPWLVAEVEGVVAGIAYAGPWKARNAYDWTVESTVYVSHRHQRLGLGSTLYTHLLKSMEAQGFKSVVAVIGLPNDPSVRLHEALGYTARGTLRAAGYKHGGWHDVGFWQRDFELPAPPRPVRPVTQI

SEQ ID NO: 28 - DNA, 1023 bp, synthetic construct
  source 1..1023: mol_type = other DNA
atgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtga

SEQ ID NO: 29 - protein, 340 aa, Stenotrophomonas maltophilia
MLTFVRNAWYVAALPEELSEKPLGRTILDTPLALYRQPDGVVAALLDICPHRFAPLSDGILVNGHLQCPYHGLEFDGGGQCVHNPHGNGARPASLNVRSFPVVERDALIWIWPGDPALADPGAIPDFGCRVDPAYRTVGGYGHVDCNYKLLVDNLMDLGHAQYVHRANAQTDAFDRLEREVIVGDGEIQALMKIPGGTPSVLMAKFLRGANTPVDAWNDIRWNKVSAMLNFIAVAPEGTPKEQSIHSRGTHILTPETEASCHYFFGSSRNFGIDDPEMDGVLRSWQAQALVKEDKVVVEAIERRRAYVEANGIRPAMLSCDEAAVRVSREIEKLEQLEAA

SEQ ID NO: 30 - DNA, 888 bp, synthetic construct
  source 1..888: mol_type = other DNA
atgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctga

SEQ ID NO: 31 - protein, 295 aa, Sphingobium herbicidovorans
MHAALTPLTNKYRFIDVQPLTGVLGAEITGVDLREPLDDSTWNEILDAFHTYQVIYFPGQAITNEQHIAFSRRFGPVDPVPILKSIEGYPEVQMIRREANESSRYIGDDWHADSTFLDAPPAAVVMRAIEVPEYGGDTGFLSMYSAWETLSPTMQATIEGLNVVHSATKVFGSLYQATNWRFSNTSVKVMDVDAGDRETVHPLVVTHPVTGRRALYCNQVYCQKIQGMTDAESKSLLQFLYEHATQFDFTCRVRWKKDQVLVWDNLCTMHRAVPDYAGKFRYLTRTTVAGDKPSR

SEQ ID NO: 32 - DNA, 1368 bp, synthetic construct
  source 1..1368: mol_type = other DNA
atgcttcatggagcttcatctaggccagctactgccaggaagtctagcgggctcagtggcaccgtgcgcatccctggcgataaaagtatttcacacaggagcttcatgttcggaggacttgctagtggagagacgagaatcactggtttgcttgagggcgaagatgttatcaacaccggtaaggcgatgcaagcaatgggtgccagaatccgaaaagagggcgatacgtggatcatcgacggtgttggtaacggaggattgctcgctcccgaagcgccacttgactttgggaacgcagctacggggtgccgtcttactatgggactggtaggcgtgtatgactttgactctaccttcatcggtgacgcgagcctcactaagagaccaatgggacgagtgctgaatcccctgagggagatgggtgtccaggtgaaatctgaggatggtgatcgtcttccggttactctgcgaggccccaagacccccacgccaatcacgtacagggttccgatggcgtcagcacaggtcaagtcagcggtactcctggcgggcctcaacacacctggaatcacaaccgtgattgaacccatcatgactagagaccacacggagaagatgttgcagggtttcggcgctaatctaacggtcgaaaccgacgccgacggcgtgaggacaatccgcttggagggcagaggtaaactgactggccaagtcatcgatgtgcctggagatccctcgtccacagcgtttcccctcgtagctgcgttgctcgtccctggatctgatgtgacgatcctgaatgtcctcatgaatccaactagaaccggcctcatcctcacattgcaggagatgggtgctgacatcgaggttatcaatcctaggttggcaggtggagaggatgtggccgatctgcgcgtgcgttctagtacactcaaaggcgtgaccgtccctgaggatcgcgctccatccatgatcgacgagtaccccattctcgccgttgctgctgcgtttgccgagggcgcaactgtaatgaacggccttgaggagttgagggttaaggagagtgacaggctgtccgcggtggcgaatggcctgaagctaaacggcgtggactgcgacgaaggtgaaacgtcccttgtagtccgtggtcgcccagacgggaaggggttggggaatgcttcgggagctgctgtggcgacgcaccttgatcatagaatcgccatgtcatttctggtgatgggacttgtctccgagaatccggtgaccgttgacgatgctaccatgatcgccacctcctttcctgagttcatggacctcatggcaggcttgggggccaagatcgagctgtctgatactaaggccgcttga

SEQ ID NO: 33 - protein, 454 aa, Oryza sativa
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDATMIATSFPEFMDLMAGLGAKIELSDTKAA

SEQ ID NO: 34 - DNA, 1368 bp, synthetic construct
  source 1..1368: mol_type = other DNA
atgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttga

SEQ ID NO: 35 - protein, 455 aa, synthetic construct
MLHGASSRPATARKSSGLSGTVRIPGDKSISHRSFMFGGLASGETRITGLLEGEDVINTGKAMQAMGARIRKEGDTWIIDGVGNGGLLAPEAPLDFGNAATGCRLTMGLVGVYDFDSTFIGDASLTKRPMGRVLNPLREMGVQVKSEDGDRLPVTLRGPKTPTPITYRVPMASAQVKSAVLLAGLNTPGITTVIEPIMTRDHTEKMLQGFGANLTVETDADGVRTIRLEGRGKLTGQVIDVPGDPSSTAFPLVAALLVPGSDVTILNVLMNPTRTGLILTLQEMGADIEVINPRLAGGEDVADLRVRSSTLKGVTVPEDRAPSMIDEYPILAVAAAFAEGATVMNGLEELRVKESDRLSAVANGLKLNGVDCDEGETSLVVRGRPDGKGLGNASGAAVATHLDHRIAMSFLVMGLVSENPVTVDDATMIATSFPEFMDLMAGLGAKIELSDTKAA

SEQ ID NO: 36 - DNA, 50 bp, Glycine max
  source 1..50: mol_type = genomic DNA
gttgcttcttcttctgcattttgaacctatcattagaaggaggtcaaaaa

SEQ ID NO: 37 - DNA, 50 bp, Glycine max
  source 1..50: mol_type = genomic DNA
tttgttaatgggaaattttgaaactaaattcaccctctcttaaattattg

SEQ ID NO: 38 - DNA, 50 bp, Glycine max
  source 1..50: mol_type = genomic DNA
agaccacttatccaacagtacatgtggtggtcgttggtggtggtggttgt

SEQ ID NO: 39 - DNA, 50 bp, Glycine max
  source 1..50: mol_type = genomic DNA
caccatttagaagttgcatgatgattgtcggtgttactgatagttgtcat

SEQ ID NO: 40 - DNA, 50 bp, Glycine max
  source 1..50: mol_type = genomic DNA
ccaaaagaggattacgcacaatctatggtggttgttggtgttggtggtca

SEQ ID NO: 41 - DNA, 50 bp, Glycine max
  source 1..50: mol_type = genomic DNA
ttgttgttcttattcgaagtattggtggtggttgtcgatcaaacactagt

SEQ ID NO: 42 - DNA, 50 bp, Glycine max
  source 1..50: mol_type = genomic DNA
tgcactccaactaacattgccaaaagatccaacccttaattgagacattt

SEQ ID NO: 43 - DNA, 50 bp, Glycine max
  source 1..50: mol_type = genomic DNA
gttcaaataccttcaaagcaacaaaacaacattaaaaatctcttcacaaa

SEQ ID NO: 44 - DNA, 50 bp, Glycine max
  source 1..50: mol_type = genomic DNA
aaaaaataaccttcttgagttgggaaagatagcacaacatcacaacccaa

SEQ ID NO: 45 - DNA, 50 bp, Glycine max
  source 1..50: mol_type = genomic DNA
caacacctaacaatacccaatgaaaaaaaattaactcaacaaagaaacac

SEQ ID NO: 46 - DNA, 50 bp, Glycine max
  source 1..50: mol_type = genomic DNA
atgtgaagaaataataatcgcaccacatgaaaaaaattaaatgcacatga

SEQ ID NO: 47 - DNA, 50 bp, Glycine max
  source 1..50: mol_type = genomic DNA
agaaaaaattagcacagatggacatgcttattcatgatggagatgtttac

SEQ ID NO: 48 - DNA, 50 bp, Glycine max
  source 1..50: mol_type = genomic DNA
acatgaacttcaacccaaaatacttcaacatgagcttctactttcaatgt

SEQ ID NO: 49 - DNA, 50 bp, Glycine max
  source 1..50: mol_type = genomic DNA
ctttaactttttcattttcaaaaactaagaaaggtcttagcttttaagag

SEQ ID NO: 50 - DNA, 50 bp, Glycine max
  source 1..50: mol_type = genomic DNA
attgtccttaaattaaagaagcatgtgcaatacacatgacaaattttcat

SEQ ID NO: 51 - DNA, 50 bp, Glycine max
  source 1..50: mol_type = genomic DNA
taacgaaaatcaaattgtgataagaaagagagttcaattgtaatggagtt

SEQ ID NO: 52 - DNA, 50 bp, Glycine max
  source 1..50: mol_type = genomic DNA
gcaaaactaataagatttattagagggataatttatggaggaggtcacct

SEQ ID NO: 53 - DNA, 50 bp, Glycine max
  source 1..50: mol_type = genomic DNA
tataataagtaggaggggatcgtcgtataatatgtattaaatgaaaggaa

SEQ ID NO: 54 - DNA, 50 bp, Glycine max
  source 1..50: mol_type = genomic DNA
agagagtgtaatttattcttaaacctaatatgaaagtgattactatgata

SEQ ID NO: 55 - DNA, 50 bp, Glycine max
  source 1..50: mol_type = genomic DNA
tagcattttcgtggggttaatgtatgaaaaattatatttttgtagtgaga

SEQ ID NO: 56 - DNA, 50 bp, Glycine max
  source 1..50: mol_type = genomic DNA
ttttttttataaaaaaaaagggaaaaacttaggtagagttagtaacctgt

SEQ ID NO: 57 - DNA, 50 bp, Glycine max
  source 1..50: mol_type = genomic DNA
cagaatctggaagaaagaaaaagagaaatgtaaaaagaaaaagaagtgaa

SEQ ID NO: 58 - DNA, 50 bp, Glycine max
  source 1..50: mol_type = genomic DNA
gccattggctgcagcaatatgaagaggagagtggcgatcgtagagagttg

SEQ ID NO: 59 - DNA, 50 bp, Glycine max
  source 1..50: mol_type = genomic DNA
aggtcgccgtgcagcacggcggtgaagaggccgtggtcatggttgccact

SEQ ID NO: 60 - DNA, 50 bp, Glycine max
  source 1..50: mol_type = genomic DNA
gttttgttattctacttgttcttaacctcttgtgggtttggtttggctgc

SEQ ID NO: 61 - DNA, 50 bp, Glycine max
  source 1..50: mol_type = genomic DNA
tgaagttgtaagttgtaaggtgttgtgggagacaatggcaaaggttttag

SEQ ID NO: 62 - DNA, 50 bp, Glycine max
  source 1..50: mol_type = genomic DNA
actgagaatgacttcgggcagcagtcagattccgcgtaagcctgtttggc

SEQ ID NO: 63 - DNA, 50 bp, Glycine max
  source 1..50: mol_type = genomic DNA
atcttattatccaatccaacaagctagagggtccccccttcccttctttt

SEQ ID NO: 64 - DNA, 50 bp, Glycine max
  source 1..50: mol_type = genomic DNA
tcagcctcattagtcattgaaaaaaaataaatagatctccgcataacata

SEQ ID NO: 65 - DNA, 50 bp, Glycine max
  source 1..50: mol_type = genomic DNA
ttgaaattaattatattaaaaaaattggataatgcgggactaagccaaca

SEQ ID NO: 66 - DNA, 50 bp, Glycine max
  source 1..50: mol_type = genomic DNA
cttttacaatttaattgatgcataattttattttatctatagataaaatt

SEQ ID NO: 67 - DNA, 50 bp, Glycine max
  source 1..50: mol_type = genomic DNA
ttgcttgtgcatttgaacttatgaacaagctcatcttattttttcttgta

SEQ ID NO: 68 - DNA, 50 bp, Glycine max
  source 1..50: mol_type = genomic DNA
ctttaaattcagggttgctttttactatttgagatttattggcgctataa

SEQ ID NO: 69 - DNA, 50 bp, Glycine max
  source 1..50: mol_type = genomic DNA
tgggcggggagcatttagcgattattggggcaattttacagcacctttct

SEQ ID NO: 70 - DNA, 50 bp, Glycine max
  source 1..50: mol_type = genomic DNA
gcatggatacctttcatggtatgtagcagataataggtggctggagattt

SEQ ID NO: 71 - DNA, 50 bp, Glycine max
  source 1..50: mol_type = genomic DNA
taccacttgatcaagtcattattatattgacttgatgattgaccttaatt

SEQ ID NO: 72 - DNA, 50 bp, Glycine max
  source 1..50: mol_type = genomic DNA
aacatttggcatctgatctatagatccctgtattcatgcaattctgtctt

SEQ ID NO: 73 - DNA, 50 bp, Glycine max
  source 1..50: mol_type = genomic DNA
tcgtagatttgttgtctttagtaccattcattcacaaaaacaaatgggta

SEQ ID NO: 74 - DNA, 50 bp, Glycine max
  source 1..50: mol_type = genomic DNA
aattgcaagattcgatcgatttcatttcagtatatactaataataataac

SEQ ID NO: 75 - DNA, 50 bp, Glycine max
  source 1..50: mol_type = genomic DNA
tattcacaattgtacaattgattagtagtcaatgcccatacaaaataaga

SEQ ID NO: 76 - DNA, 50 bp, Glycine max
  source 1..50: mol_type = genomic DNA
ggaaaaattataaaaaatactcattttattagatttattatttatattag

SEQ ID NO: 77 - DNA, 50 bp, Glycine max
  source 1..50: mol_type = genomic DNA
tagtttacttatttgagtagaatgtgtgatcttcttattatgtttaattt

SEQ ID NO: 78 - DNA, 50 bp, Glycine max
  source 1..50: mol_type = genomic DNA
ataaaggaaaatacattacataaaacttaaaataaaatctgaagcgggaa

SEQ ID NO: 79 - DNA, 50 bp, Glycine max
  source 1..50: mol_type = genomic DNA
aataaaactgaattccatggagtatataaaatggatcataatttggctgc

SEQ ID NO: 80 - DNA, 50 bp, Glycine max
  source 1..50: mol_type = genomic DNA
ttatgagtttgatttttttttctacacgctataatatcagttgctctaaa

SEQ ID NO: 81 - DNA, 50 bp, Glycine max
  source 1..50: mol_type = genomic DNA
cgaggatataattttgccaaagaccggagatatataaccctgctattttt

SEQ ID NO: 82 - DNA, 50 bp, Glycine max
  source 1..50: mol_type = genomic DNA
gttgtagtaaataatcagcaaacatacggagatataactttgattggttg

SEQ ID NO: 83 - DNA, 50 bp, Glycine max
  source 1..50: mol_type = genomic DNA
atattatttctaccacaattaattgatgtacatatatgttatactaaata

SEQ ID NO: 84 - DNA, 50 bp, Glycine max
  source 1..50: mol_type = genomic DNA
tgagtggttataaataccttatgcttattttatttttacgcataaaaaat

SEQ ID NO: 85 - DNA, 50 bp, Glycine max
  source 1..50: mol_type = genomic DNA
ccatattatgctaatacacgcctgtttttcatttttatttgtgttcccca

SEQ ID NO: 86 - DNA, 50 bp, Glycine max
  source 1..50: mol_type = genomic DNA
tatttattacacgtgaatctcctacttaaaattgcatttctcttgctttc

SEQ ID NO: 87 - DNA, 50 bp, Glycine max
  source 1..50: mol_type = genomic DNA
aataagccttactaatttagccacatacgatgttagttgttttgacatga

SEQ ID NO: 88 - DNA, 50 bp, Glycine max
  source 1..50: mol_type = genomic DNA
atttcttcaagattaatataaggttgtttcaacaaacaaaaccttacttc

SEQ ID NO: 89 - DNA, 50 bp, Glycine max
  source 1..50: mol_type = genomic DNA
atttaagactttggtaatcgattaccagacaatgtaatcgattacaagaa

SEQ ID NO: 90 - DNA, 50 bp, Glycine max
  source 1..50: mol_type = genomic DNA
ttactagtaatggaactcttgaaattcaaattcaaaagtcacgacccttc

SEQ ID NO: 91 - DNA, 50 bp, Glycine max
  source 1..50: mol_type = genomic DNA
ttttgaaaagacacatctcttcaaatcattttgaaaaggcacgaagagtc

SEQ ID NO: 92 - DNA, 50 bp, Glycine max
  source 1..50: mol_type = genomic DNA
taacttcattatcaaatgttctctcaacaactcttaggtaaatacttgca

SEQ ID NO: 93 - DNA, 50 bp, Glycine max
  source 1..50: mol_type = genomic DNA
aggagagaaatccttttgttcttcttagaaagtcaattataatcaagaga

SEQ ID NO: 94 - DNA, 50 bp, Glycine max
  source 1..50: mol_type = genomic DNA
cttgagtgttcagaagttataaaaatgatttttacaaagatagtggaaaa

SEQ ID NO: 95 - DNA, 50 bp, Glycine max
  source 1..50: mol_type = genomic DNA
ctagtataaatcgagtttgcatttctctcttttcttatcttatttatttt

SEQ ID NO: 96 - DNA, 50 bp, Glycine max; identical to SEQ ID NO: 36
  source 1..50: mol_type = genomic DNA

SEQ ID NO: 97 - DNA, 50 bp, Glycine max; identical to SEQ ID NO: 38
  source 1..50: mol_type = genomic DNA

SEQ ID NO: 98 - DNA, 50 bp, Glycine max; identical to SEQ ID NO: 40
  source 1..50: mol_type = genomic DNA

SEQ ID NO: 99 - DNA, 50 bp, Glycine max; identical to SEQ ID NO: 42
  source 1..50: mol_type = genomic DNA

SEQ ID NO: 100 - DNA, 50 bp, Glycine max; identical to SEQ ID NO: 44
  source 1..50: mol_type = genomic DNA

SEQ ID NO: 101 - DNA, 50 bp, Glycine max; identical to SEQ ID NO: 46
  source 1..50: mol_type = genomic DNA

SEQ ID NO: 102 - DNA, 50 bp, Glycine max; identical to SEQ ID NO: 48
  source 1..50: mol_type = genomic DNA

SEQ ID NO: 103 - DNA, 50 bp, Glycine max; identical to SEQ ID NO: 50
  source 1..50: mol_type = genomic DNA

SEQ ID NO: 104 - DNA, 50 bp, Glycine max; identical to SEQ ID NO: 52
  source 1..50: mol_type = genomic DNA

SEQ ID NO: 105 - DNA, 50 bp, Glycine max; identical to SEQ ID NO: 54
  source 1..50: mol_type = genomic DNA

SEQ ID NO: 106 - DNA, 50 bp, Glycine max
  source 1..50: mol_type = genomic DNA
atatatgtccaaagtcacgtaactaaggtttcagccatttaacttttcaa

SEQ ID NO: 107 - DNA, 50 bp, Glycine max
  source 1..50: mol_type = genomic DNA
atgaacctaatctttcaactttattctaatcttttcttctataataggaa

SEQ ID NO: 108 - DNA, 50 bp, Glycine max
  source 1..50: mol_type = genomic DNA
agccattgcttgaagaaagaaccacgttgaaaccaaatttcctcaggaag

SEQ ID NO: 109 - DNA, 50 bp, Glycine max
  source 1..50: mol_type = genomic DNA
taaactttgacaagttaccttgccttcgacccccgtggcttcatcacaca

SEQ ID NO: 110 - DNA, 50 bp, Glycine max
  source 1..50: mol_type = genomic DNA
agacaagtcctgattagatagatatattcagtttgtgtagttgaaaaaaa

SEQ ID NO: 111 - DNA, 50 bp, Glycine max
  source 1..50: mol_type = genomic DNA
tgaaaagtggaaatgagatttatttttccccttcagaaaacagaagttgt

SEQ ID NO: 112 - DNA, 50 bp, Glycine max
  source 1..50: mol_type = genomic DNA
gacttgtgacacatatatatagcgagcgaaaacaaagttcatttaatttt

SEQ ID NO: 113 - DNA, 50 bp, Glycine max
  source 1..50: mol_type = genomic DNA
tcttatattaattatgtattcattttatttttcttactgaataaattaca

SEQ ID NO: 114 - DNA, 50 bp, Glycine max
  source 1..50: mol_type = genomic DNA
catgatctaaattaagaaaaataaaaatataattaatgctgtaaaaaaat

SEQ ID NO: 115 - DNA, 50 bp, Glycine max
  source 1..50: mol_type = genomic DNA
ataattaaatctttcaattttgatcaaagctaaagcataagcatcgcgat

SEQ ID NO: 116 - DNA, 50 bp, Glycine max
  source 1..50: mol_type = genomic DNA
tcaaaaaagaaaagataagaaaatgcacgggggttgattttatgttcaat

SEQ ID NO: 117 - DNA, 50 bp, Glycine max
  source 1..50: mol_type = genomic DNA
ttaaaagtatattttatatagatatcttctttttgtaaaatcacggacat

SEQ ID NO: 118 - DNA, 50 bp, Glycine max
  source 1..50: mol_type = genomic DNA
ctttttataaagtaatcttcgtcgagccgaaaacatttacaacatagtgc

SEQ ID NO: 119 - DNA, 50 bp, Glycine max
  source 1..50: mol_type = genomic DNA
agttaattctcgaagtagatttttttcctatactcaaattattgtttaaa

SEQ ID NO: 120 - DNA, 50 bp, Glycine max
  source 1..50: mol_type = genomic DNA
aagaaatcaagcatacactactttcacttttaaattattatatatatacc

SEQ ID NO: 121 - DNA, 50 bp, Glycine max
  source 1..50: mol_type = genomic DNA
tttagttacatacaataatagtacatgaaaacttttctcataagacataa

SEQ ID NO: 122 - DNA, 50 bp, Glycine max
  source 1..50: mol_type = genomic DNA
atacataatgattcatatgatttctttttctatttatatctcagttaatt

SEQ ID NO: 123 - DNA, 50 bp, Glycine max
  source 1..50: mol_type = genomic DNA
agtattgtcccttatttttaagtttagaccgcaagaattttctacaagaa

SEQ ID NO: 124 - DNA, 50 bp, Glycine max
  source 1..50: mol_type = genomic DNA
ataatcttcttcgatattggaacattcacaatattatgaaaataattttt

SEQ ID NO: 125 - DNA, 50 bp, Glycine max
  source 1..50: mol_type = genomic DNA
atggtgtttttttttcatacagaaagcttgcagctgaaaatcttagttaa

SEQ ID NO: 126 - DNA, 50 bp, Glycine max; identical to SEQ ID NO: 107
  source 1..50: mol_type = genomic DNA

SEQ ID NO: 127 - DNA, 50 bp, Glycine max; identical to SEQ ID NO: 109
  source 1..50: mol_type = genomic DNA

SEQ ID NO: 128 - DNA, 50 bp, Glycine max; identical to SEQ ID NO: 111
  source 1..50: mol_type = genomic DNA

SEQ ID NO: 129 - DNA, 50 bp, Glycine max; identical to SEQ ID NO: 113
  source 1..50: mol_type = genomic DNA

SEQ ID NO: 130 - DNA, 50 bp, Glycine max; identical to SEQ ID NO: 115
  source 1..50: mol_type = genomic DNA

SEQ ID NO: 131 - DNA, 50 bp, Glycine max; identical to SEQ ID NO: 117
  source 1..50: mol_type = genomic DNA

SEQ ID NO: 132 - DNA, 50 bp, Glycine max; identical to SEQ ID NO: 119
  source 1..50: mol_type = genomic DNA

SEQ ID NO: 133 - DNA, 50 bp, Glycine max; identical to SEQ ID NO: 121
  source 1..50: mol_type = genomic DNA

SEQ ID NO: 134 - DNA, 50 bp, Glycine max; identical to SEQ ID NO: 123
  source 1..50: mol_type = genomic DNA

SEQ ID NO: 135 - DNA, 50 bp, Glycine max; identical to SEQ ID NO: 125
  source 1..50: mol_type = genomic DNA

SEQ ID NO: 136 - DNA, 50 bp, Glycine max
  source 1..50: mol_type = genomic DNA
atgtcaaccatcaacttttttttttacaatatttatgcatttttaattga

SEQ ID NO: 137 - DNA, 50 bp, Glycine max
  source 1..50: mol_type = genomic DNA
tccacaagaggatacccctcttatctctagcgatttacaagcacacgcac

SEQ ID NO: 138 - DNA, 50 bp, Glycine max
  source 1..50: mol_type = genomic DNA
tatgctttcacaatttcataagcatgtataattccaaccattggtcactg

SEQ ID NO: 139 - DNA, 50 bp, Glycine max
  source 1..50: mol_type = genomic DNA
tcattcgataactgtagtttttttattgagatatttgataagttcactta

SEQ ID NO: 140 - DNA, 50 bp, Glycine max
  source 1..50: mol_type = genomic DNA
ttcctccttagtattaaaatgtcattatttagtgtcaaaagtctgattat

SEQ ID NO: 141 - DNA, 50 bp, Glycine max
  source 1..50: mol_type = genomic DNA
tcaactagtagtactcaactttattgcataaagctaagaaaccttcattg

SEQ ID NO: 142 - DNA, 50 bp, Glycine max
  source 1..50: mol_type = genomic DNA
tcattgaattaaggatccctaagtagtttctgtatgagcctaataagcaa

SEQ ID NO: 143 - DNA, 50 bp, Glycine max
  source 1..50: mol_type = genomic DNA
ttattaagtacactaccctttagtttttcttttgattttttttatcataa

SEQ ID NO: 144 - DNA, 50 bp, Glycine max
  source 1..50: mol_type = genomic DNA
ttttttaaaatgatgtttaaattttttcttccaaaaagtacattccacaa

SEQ ID NO: 145 - DNA, 50 bp, Glycine max
  source 1..50: mol_type = genomic DNA
tagattccgaaaacgtactttccagagtactatttaaaatttttgtattc

SEQ ID NO: 146 - DNA, 50 bp, Glycine max
  source 1..50: mol_type = genomic DNA
taaaaaaaatacaatcatcttagtaaagtgacacacaaagaaaacattct

SEQ ID NO: 147 - DNA, 50 bp, Glycine max
  source 1..50: mol_type = genomic DNA
ggcaaatcatgtatgaagatttgaagttggtaaacacttttcctagttta

SEQ ID NO: 148 - DNA, 50 bp, Glycine max
  source 1..50: mol_type = genomic DNA
accaaagtaagatatcaacttggagtaataaagagtgaaagagaggaatg

SEQ ID NO: 149 - DNA, 50 bp, Glycine max
  source 1..50: mol_type = genomic DNA
gttgggggaggggggagagtgtgagattgtgtgaaggaaaaaacaaagtt

SEQ ID NO: 150 - DNA, 50 bp, Glycine max
  source 1..50: mol_type = genomic DNA
agtgtagttagaaaaaagggagcgaatatagcaagtgtccaaaaatttta

SEQ ID NO: 151 - DNA, 50 bp, Glycine max
  source 1..50: mol_type = genomic DNA
gaaccatcttttacacccttgccggtatcctccaagacagaagagctaaa

SEQ ID NO: 152 - DNA, 50 bp, Glycine max
  source 1..50: mol_type = genomic DNA
ttagaaggataatactttaaaaaataacttatatttatgatcactaaatt

SEQ ID NO: 153 - DNA, 50 bp, Glycine max
  source 1..50: mol_type = genomic DNA
aataaaaagcaccaagctctaaaataacccccgtcactaatagttaaaag

SEQ ID NO: 154 - DNA, 50 bp, Glycine max
  source 1..50: mol_type = genomic DNA
actaatattgaagtcaccatttcctttaaaaaaatatattgtagtcgtca

SEQ ID NO: 155 - DNA, 50 bp, Glycine max
  source 1..50: mol_type = genomic DNA
taaattgttaatataaatatcgagtctaaactaacagatatattgtacta

SEQ ID NO: 156 - DNA, 50 bp, Glycine max
  source 1..50: mol_type = genomic DNA
aattataatatttatatatactaacattctaacaatgcaatgctcaagat

SEQ ID NO: 157 - DNA, 50 bp, Glycine max
  source 1..50: mol_type = genomic DNA
aaaaaagttatattttgaatttaggataaataatttatattgtgatatat

SEQ ID NO: 158 - DNA, 50 bp, Glycine max
  source 1..50: mol_type = genomic DNA
aaaaaggatagattaaaagaagatataagctgtataaaaattaaatatta

SEQ ID NO: 159 - DNA, 50 bp, Glycine max
  source 1..50: mol_type = genomic DNA
ttttattaactggtaatgccatttctctactttgttatttgtttggatta

SEQ ID NO: 160 - DNA, 50 bp, Glycine max
  source 1..50: mol_type = genomic DNA
ttattcttacaattttttcataattaacaaaatttagggaaatcagattg

SEQ ID NO: 161 - DNA, 50 bp, Glycine max
  source 1..50: mol_type = genomic DNA
aagtatttatatttataagttcagtaactaatattgataagtttttggta

SEQ ID NO: 162 - DNA, 50 bp, Glycine max
  source 1..50: mol_type = genomic DNA
tggttgtgtagattgggcctaggaatcaaaatggaattactttgtatatt

SEQ ID NO: 163 - DNA, 50 bp, Glycine max
  source 1..50: mol_type = genomic DNA
ggttactcgattcatcagattgcttggtcactgactcagtcttgactctt

SEQ ID NO: 164 - DNA, 50 bp, Glycine max
  source 1..50: mol_type = genomic DNA
acaatagccgaagcccagtcatccaccccaacagcagttacagttaatat

SEQ ID NO: 165 - DNA, 50 bp, Glycine max
  source 1..50: mol_type = genomic DNA
tagtatatagtatacttgtttgttcacttcacactgcattgattttaacg

SEQ ID NO: 166 - DNA, 50 bp, Glycine max
  source 1..50: mol_type = genomic DNA
tccagctagctctatgttactgttattttaataaccagaaattaatatta

SEQ ID NO: 167 - DNA, 50 bp, Glycine max
  source 1..50: mol_type = genomic DNA
tttagtgttacctttgcaaatttggagtaagcaatgtgcaggtcaagcgt

SEQ ID NO: 168 - DNA, 50 bp, Glycine max
  source 1..50: mol_type = genomic DNA
ctactacaggtatacgatgcttttcaataatacattaacacacattaaga

SEQ ID NO: 169 - DNA, 50 bp, Glycine max
  source 1..50: mol_type = genomic DNA
aatccatgcttatcctgaattggctttcatgacatgtccttagaaacatt

SEQ ID NO: 170 - DNA, 50 bp, Glycine max
  source 1..50: mol_type = genomic DNA
tatagtgcctcagcctctccgtacctcttgtgcagtagattgcttttttc

SEQ ID NO: 171 - DNA, 50 bp, Glycine max
  source 1..50: mol_type = genomic DNA
cttatcctgtttaattcaaagtaaattagttctagagattgaagaatatt

SEQ ID NO: 172 - DNA, 50 bp, Glycine max
  source 1..50: mol_type = genomic DNA
gacaaaataatatagcagtgataatggtacagaaaaatgtacaacgctta

SEQ ID NO: 173 - DNA, 50 bp, Glycine max
  source 1..50: mol_type = genomic DNA
ttcctgcctccctgtctctcttttatttctttccatcttcatataacaat

SEQ ID NO: 174 - DNA, 50 bp, Glycine max
  source 1..50: mol_type = genomic DNA
aaaaaacatgacgtgcgtgccatgtagaatagtaactagcagtaactttt

SEQ ID NO: 175 - DNA, 50 bp, Glycine max
  source 1..50: mol_type = genomic DNA
atatagcattgcttttacatacaaatatattttcaccaatgcacattact

SEQ ID NO: 176 - DNA, 22 bp, synthetic construct
  source 1..22: mol_type = other DNA
aagagcgaatttggcctgtaga

SEQ ID NO: 177 - DNA, 32 bp, synthetic construct
  source 1..32: mol_type = other DNA
tgaaagtgattactatgatatagcattttcgt

SEQ ID NO: 178 - DNA, 18 bp, synthetic construct
  source 1..18: mol_type = other DNA
attgcgagctttctaatt

SEQ ID NO: 179 - DNA, 20 bp, synthetic construct
  source 1..20: mol_type = other DNA
ccctgtgatcgcagactcaa

SEQ ID NO: 180 - DNA, 20 bp, synthetic construct
  source 1..20: mol_type = other DNA
cggagatgctgtcgtgagaa

SEQ ID NO: 181 - DNA, 18 bp, synthetic construct
  source 1..18: mol_type = other DNA
actctcgctagacacttc

SEQ ID NO: 182 - DNA, 12196 bp, synthetic construct
  source 1..12196: mol_type = other DNA
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgtagccagtcagcatcatcacaccaaaagttaggcccgaatagtttgaaattagaaagctcgcaattgaggtctgtcgaccctgcactaactataacggtcctaaggtagcgatttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgaggcctagcaggccggtaccgagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttatgacggccacgagcgaactcctgcaggtgtttaaactagggataatagctgtttgccaatcgttcttcttggcgcgcccatagagcagtcttgtctcaaaaaaatcgaattaagcagttgaaagtgttcgtcttatcaagatgaaaatctgacaaaaattttgttaaaatttggtagtatttgataagatatgtaccttaaaattgatgttaataatcttcttatccaaaaaaagaaccaatccattgcagtcttaaatgaaatatttcaagaaaggattgagccaaaaaccgtgtttataaaattttcaaatccacaatttccacattcacttggatattacaagtgtggcatctcataaaaaaaaagaaaaagaaaaaccacgtggactattatatccagccacgtggctttaaaatcttatccaaaatcacctctcatccaacggataaggtaaccacacagccttatccaaccacatcacacgaatccactctatatatactccatataaccatcactcacacgtctttcatcacacaagtaaaacagcatcactaaaagaaaaaaaacaagaatttcaggtttagctttctctccgatctcggctttcgatctgatctcgtctgatctccgatttcgatccccgtccgatctcgatctgatcccatccgatctgtgattgattcgttgagattcagatctgatcttaggatctctctgatagatcatagattcaatctctcgagatagagatgattgatgttgttcagatcggttttgatctctgatctgatcagctcgattgattcgatttgattctcgattcgatttgatcgacaattgatctgattctctaattgatgttctgtttttgttacaggttttggtaccatgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttgattaattaaacacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaaggccgtgacggccacgagcgaactcctgcaggtgtttaaactagggataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccatgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctgaggccggccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgggccgtgacggccacgagcgaactcctgcaggtgtttaaactagggataacagggtaataggtctcacgcggcaaatcctaccacctcatttaaactagggataatagctgtttgccaatcgttcttcttggcgcgccaattaaattcaacacgtttgttatatattttttattgaaattattcttcattcgtcttttaatggataaaaaggtataatcaagtatattttatacacatctttctatttgtgtgtaccaaatgttaaaatggccaattttgaccaaaaaaccgcataattttcttaatttcttaaatatgattaattcatcaataacttggaatttcacaatacacaaaagtgggtgtagttaccgttattatatttatacacaacaactcatctcctcatagaaagaaaagaaaaataaaataagaaatcaaaaaacgacaagataaccaatctccacatcatccacgtggcgtaaggataaggtcacaaccaccactcagccacgtggcagaatcttatccaatcactctcaccacacaaaccttatccacttctatatataatctcttcttctcattatcactcaccacacatccttgcaaaagtaaagagaaaaaacaaacaagacaggtaaacccagatctctttcttctcttctcttcatctcgatctctccattttcataaacccaattttttctctgatttgtttgatttggtttggatctttctgtgtttccatggttttaggaattttaggatagatttttgtttgttcatgttattcatcggatatatagatttcaaattcttttgcaatttttctctctctttagttttgctcaattttggttgttgttgtgatgagtgttctctttatgggtttatctgagcttggtgagagttttttgatattgattttgcaggtggtaccctcagcgctgtgcctgttgcgatcgcaccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgagcgatcgcttaattaattaattaagagctcatgtctgctgcggcggccttcacagtttgtttatttcctactgtttgctgcggcgattgttgttgttttctgttttataaataataaaggaggaggagatttgttttggtttgtgtttgtttccatccttgctgctccatcacactatctgtaatttgtaaacagcgacaataaataaattaataaatttggtttctcatacctatatgtgtctgtttggaggcttgtttgtttgagacatctgtctggttgtttttttgctgccagccggtagtataaattttgtttttggacgacgaaaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctaattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 183 - DNA, 10196 bp, synthetic construct
  source 1..10196: mol_type = other DNA
gccagtcagcatcatcacaccaaaagttaggcccgaatagtttgaaattagaaagctcgcaattgaggtctgtcgaccctgcactaactataacggtcctaaggtagcgatttattagagtagattagaatcttttatgccaagtattgataaattaaatcaagaagataaactatcataatcaacatgaaattaaaagaaaaatctcatatatagtattagtattctctatatatattatgattgcttattcttaatgggttgggttaaccaagacatagtcttaatggaaagaatcttttttgaactttttccttattgattaaattcttctatagaaaagaaagaaattatttgaggaaaagtatatacaaaaagaaaaatagaaaaatgtcagtgaagcagatgtaatggatgacctaatccaaccaccaccataggatgtttctacttgagtcggtcttttaaaaacgcacggtggaaaatatgacacgtatcatatgattccttcctttagtttcgtgataataatcctcaactgatatcttcctttttttgttttggctaaagatattttattctcattaatagaaaagacggttttgggcttttggtttgcgatataaagaagaccttcgtgtggaagataataattcatcctttcgtctttttctgactcttcaatctctcccaaagcctaaagcgatctctgcaaatctctcgcgactctctctttcaaggtatattttctgattctttttgtttttgattcgtatctgatctccaatttttgttatgtggattattgaatcttttgtataaattgcttttgacaatattgttcgtttcgtcaatccagcttctaaattttgtcctgattactaagatatcgattcgtagtgtttacatctgtgtaatttcttgcttgattgtgaaattaggattttcaaggacgatctattcaatttttgtgttttctttgttcgattctctctgttttaggtttcttatgtttagatccgtttctctttggtgttgttttgatttctcttacggcttttgatttggtatatgttcgctgattggtttctacttgttctattgttttatttcagggtatggcgacggctacgacgactgctacggcggcgtttagtggtgtagtcagtgtaggaacggagactcgaaggatttattcgttttctcatcttcaaccttctgcggcttttccggcgaagcctagttccttcaaatctctcaaattaaagcagagcgcgaggctcacacggcggcttgatcatcggccgttcgttgtccgatgtatgctcactttcgttagaaacgcttggtacgttgctgcacttcctgaggagttgagcgagaagcctctaggaagaactatcctcgatactccactagctctctatcgtcaacctgacggagttgtcgctgccctgcttgatatttgtccgcatcgcttcgctccgttgagtgacggtattctagtcaacggacatctccagtgtccatatcacggtctggaatttgacggaggtggccagtgtgtccacaacccgcacggcaacggagcccgccctgcttctctgaacgtgcgatcattccctgtcgtggaaagagacgcattgatctggatctggcctggagatccagcactcgcagatcccggtgctatccctgactttgggtgtcgtgttgatccagcttaccgtactgtcggaggttacggtcacgtggactgcaactacaagctccttgtggataacctcatggatcttggacacgctcagtacgtgcaccgcgctaacgcccaaacagacgccttcgatagacttgagcgtgaggtgatcgttggcgacggcgagatccaggcgctcatgaagatccctggtggcacaccctcagttctcatggctaagttcttgcgtggtgctaacacaccagttgacgcctggaacgacatccggtggaataaggtgtcggctatgctgaacttcatcgcggtcgcgccggaagggacgccgaaggagcagtcaatccactcccgaggaacccatatccttactcctgagaccgaggcaagctgccattacttcttcggtagttcccgcaacttcggtatagacgatccagagatggacggtgttctcaggagctggcaagctcaagccctggtgaaggaggacaaagtggtcgttgaagctatcgaaaggcggagggcttacgtcgaagcgaacgggatcagacccgccatgttgtcctgcgacgaggcagccgtcagggtatccagggagattgagaagctcgaacaactagaagcggcgtgaggcctagcaggccggtaccgagtactctccaacatggacacaccatgggattgtgtaacataaataatgtgtcgtttgtaatgaatgctcgcaacttttctagctaattaagctagagttgaacttgagctacttttatgtaccctaaagaggcacaatctttgctgttgatgtactatgatcatgttataatatgatgaaaatggagtgtgcctcattttataatttttattttcctgagtatatgtttttagggctaaacaccttataaaaaaaggtcacttagaatatgaaacatgaacttttgtaaaaaagtagagattaaaattgaaatcaaaaatttttataggatcaatattcgaagaatttttttagagggattaaaattaaatatagtttcggactgacccaaggcacaatccggctccgctcgggttcgacctgagtccaccatgcatctgtcaccttaccattgacacgccctaaaatacattagatcgcagtacaaattgagagttatgacggccacgagcgaactcctgcaggtgtttaaactagggataatagctgtttgccaatcgttcttcttggcgcgcccatagagcagtcttgtctcaaaaaaatcgaattaagcagttgaaagtgttcgtcttatcaagatgaaaatctgacaaaaattttgttaaaatttggtagtatttgataagatatgtaccttaaaattgatgttaataatcttcttatccaaaaaaagaaccaatccattgcagtcttaaatgaaatatttcaagaaaggattgagccaaaaaccgtgtttataaaattttcaaatccacaatttccacattcacttggatattacaagtgtggcatctcataaaaaaaaagaaaaagaaaaaccacgtggactattatatccagccacgtggctttaaaatcttatccaaaatcacctctcatccaacggataaggtaaccacacagccttatccaaccacatcacacgaatccactctatatatactccatataaccatcactcacacgtctttcatcacacaagtaaaacagcatcactaaaagaaaaaaaacaagaatttcaggtttagctttctctccgatctcggctttcgatctgatctcgtctgatctccgatttcgatccccgtccgatctcgatctgatcccatccgatctgtgattgattcgttgagattcagatctgatcttaggatctctctgatagatcatagattcaatctctcgagatagagatgattgatgttgttcagatcggttttgatctctgatctgatcagctcgattgattcgatttgattctcgattcgatttgatcgacaattgatctgattctctaattgatgttctgtttttgttacaggttttggtaccatgtctcccgagcgacggccagtagagattagacccgcaacagccgccgacatggcggcagtatgtgacatcgtaaatcactacatcgagacatccacagtaaactttcgtaccgaaccacaaactcctcaggaatggattgatgacttggagagactacaagaccgctatccgtggctcgtggcagaggtcgaaggcgtcgtcgcgggaatcgcctacgctggaccgtggaaggctcgaaatgcttacgactggactgtggaatccactgtttacgtctcgcatcgccatcagaggctgggtctgggcagcaccctgtacacccacttactcaaatctatggaggctcagggctttaagtcagtggtggccgtcatcggactacctaacgatccttctgtgcgcctacatgaagcactaggctacacggcacgcggaacattgagggccgccgggtacaaacacggaggatggcacgacgttggattctggcaacgggatttcgaacttccagcacctccaagacccgttagaccagttacccagatttgattaattaaacacatattcaagctctattactctgtttttaggttgagaatattgtgaagtgtgttgtgtgtgcgttctgttttatgtaaaagtaaattctgtgaattatattttgtaaggaataaattatgtaagaatggtttaattatctattgtctcacatcgttttttatgtctattttaagtattggtattgtattctatttatacacaagagttaataatagcataacataaattccacaaggatcaagtaaaataatatccacatgaaacatctaagaaaggaaaacaatatggctcaatttcctatctttctttatttatgttttagtcatctttctttccctatttcttgttgaaagctaataggagtatggtactttctacgccatttttttttaagtactctcttttacacaatattttatcaaattgtagtgacattttattttacgatgcagttaatgttcgttgtgttactgtcaatgattgtccaaaggtgaaaggccgtgacggccacgagcgaactcctgcaggtgtttaaactagggataatagctgtttgccaatcgttcttcttggcgcgccgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggtggtaccatgcacgcggctttgacacctttgaccaacaagtatcggttcatcgacgttcaaccactcacaggcgtgctcggcgcagagattaccggagtggacctgagggagcccttagacgactccacttggaacgagatcctcgacgcctttcacacctaccaagttatctactttcctggacaggcgatcaccaacgagcagcacattgccttctcaaggaggttcggaccggtagatccagttccaattctcaaatccattgagggttatcccgaggtgcagatgattagacgagaagccaacgagtcctcacggtacataggcgacgattggcacgcagacagcaccttccttgacgctcctccggctgccgtggttatgcgcgcaatagaggtgccggagtacggcggcgataccggtttcctatcaatgtactctgcatgggagacgctctcaccaacgatgcaagccaccattgaaggtctaaacgtggttcactcagctactaaggtcttcggaagtctttaccaggcgacgaattggaggttcagtaacaccagtgtgaaggtgatggatgtggacgctggagacagggagacggtgcatccactcgtagttacacaccctgtaactggacgcagagccctttactgcaaccaggtttactgccagaagatccagggaatgactgatgcggagtctaagtccctgcttcaattcctctacgaacacgccacccaattcgacttcacttgtcgtgttcggtggaagaaggaccaagtgctcgtgtgggataacctttgcaccatgcaccgagcagtaccagactacgccgggaaattccgctatctcacccgcactacagtggccggagacaagcctagccgctgaggccggccgcatgaataatcaagctcataaatttcatggctttgaatttgtactattttggttactagaaagtgtatttgtgtgtttatgcagtaataaatctctaagagatatatgtttgttattttttataattatccaaaaaatcgttaatgttgaaaattgattcaaaattgatattgaagttctgaaaaaatcgtggcgtgattaaaaatccaaacttttttataaaataatattgtgtctatatcttttataaatgacgataaatgggataaagtaaatgaaacaaaaccgttaatgcaatgttcatctgcacaatatatataattaaaaaacattataaaaccttgtttcttcactcatttacaatcttgaaattttagtctttaccatttgaaagtacaatcttttcatgaaagtttatagtacaaatcaagagtttggataagctgctctgctttttataatcactgggaaatgatttatgacttggaaaaacaacttgggccgtgacggccacgagcgaactcctgcaggtgtttaaactagggataacagggtaataggtctcacgcggcaaatcctaccacctcatttaaactagggataatagctgtttgccaatcgttcttcttggcgcgccaattaaattcaacacgtttgttatatattttttattgaaattattcttcattcgtcttttaatggataaaaaggtataatcaagtatattttatacacatctttctatttgtgtgtaccaaatgttaaaatggccaattttgaccaaaaaaccgcataattttcttaatttcttaaatatgattaattcatcaataacttggaatttcacaatacacaaaagtgggtgtagttaccgttattatatttatacacaacaactcatctcctcatagaaagaaaagaaaaataaaataagaaatcaaaaaacgacaagataaccaatctccacatcatccacgtggcgtaaggataaggtcacaaccaccactcagccacgtggcagaatcttatccaatcactctcaccacacaaaccttatccacttctatatataatctcttcttctcattatcactcaccacacatccttgcaaaagtaaagagaaaaaacaaacaagacaggtaaacccagatctctttcttctcttctcttcatctcgatctctccattttcataaacccaattttttctctgatttgtttgatttggtttggatctttctgtgtttccatggttttaggaattttaggatagatttttgtttgttcatgttattcatcggatatatagatttcaaattcttttgcaatttttctctctctttagttttgctcaattttggttgttgttgtgatgagtgttctctttatgggtttatctgagcttggtgagagttttttgatattgattttgcaggtggtaccctcagcgctgtgcctgttgcgatcgcaccatggctgacgagtcatggagggcgccggcgatagtgcaagagctggcggcagccggcgtcgaggagccgccgagccgatacctgctacgggagaaagaccgttctgacgtcaagctggtcgccgccgagctgccggagcctctcccagtcgttgatctcagccggctagatggtgccgaggaggccaccaagctcagggtggctctgcagaattggggcttcttcctgcttaccaaccatggagtagaagcctctctgatggacagcgtgatgaacttgtcgagagagttcttcaaccagccaatcgaacggaagcagaaattcagcaacttgatcgatggcaagaacttccagattcaagggtatggaactgaccgggtggttacccaagatcagatcctggactggtctgatcggttgcatctcagagttgaacccaaggaggagcaagatcttgccttctggcctgaccatcctgaatctttcagggatgttctgaacaagtatgcatcaggaactaaaagaattagagacgatatcattcaggctatggccaagcttcttgagcttgatgaggattacttcttggaccgactcaacgaagctcctgcattcgcaagattcaactactaccctccctgtccaaggcctgaccttgtgttcggcatcaggcctcactccgacggcaccctcttgacgattcttctcgtcgacaaagatgtcagtggcctgcaagttcagagggatggcaagtggtccaacgttgaggcaactcctcacacattgctgatcaacttaggtgacaccatggaggtaatgtgcaatggcatcttcaggagcccggtgcacagggtggtgacaaacgccgagaaggagaggatctccctggccatgttatacagcgtgaacgatgagaaagacattgagccggcggctggtttgctggatgagaatcggcctgcaagatacaggaaagtgagcgtcgaagagttcagggccgggatctttggaaaattctctcgaggagagaggtacatcgactccctgaggatctgagcgatcgcttaattaattaattaagagctcatgtctgctgcggcggccttcacagtttgtttatttcctactgtttgctgcggcgattgttgttgttttctgttttataaataataaaggaggaggagatttgttttggtttgtgtttgtttccatccttgctgctccatcacactatctgtaatttgtaaacagcgacaataaataaattaataaatttggtttctcatacctatatgtgtctgtttggaggcttgtttgtttgagacatctgtctggttgtttttttgctgccagccggtagtataaattttgtttttggacgacgaaaaatagagtgaggttgatttgcggccgcacaacaaacgccggcgctctcctctcttaaggtagccggtgacacccttgggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgct

SEQ ID NO: 184 - DNA, 30 bp, synthetic construct
  source 1..30: mol_type = other DNA
tctaatagtcttgtagccagtcagcatcat

SEQ ID NO: 185 - DNA, 60 bp, synthetic construct
  source 1..60: mol_type = other DNA
tccatcgtcccttagtctaatagtcttgtagccagtcagcatcatcacaccaaaagttag

SEQ ID NO: 186 - DNA, 100 bp, synthetic construct
  source 1..100: mol_type = other DNA
cagagtgttatttgctttaatccatcgtcccttagtctaatagtcttgtagccagtcagcatcatcacaccaaaagttaggcccgaatagtttgaaatta

SEQ ID NO: 187 - DNA, 1050 bp, synthetic construct
  source 1..1050: mol_type = other DNA
aattaaggtgaattgtgactgaacggaatcttcgacatcctaacccggtatcggggaaagtacctgtaaaaatacttcgatgatcaagttagttaagtttaaaatacttatgtatcagtatatgaaatgattgaaacgtatcttgctgtgttttctttggatgtgtgtgtgtgtcctactttattagagatccattgaaaagtgagaacttcaagctattaagacggattttttcagtgtgacacttaataatttgtaaatttgttctaagatttgtattaaattttaattcttattgtattggattttttttggaaattagcagatatggtattaagctttttgaaatttcacttattatttattttttattattttctctttatttttttagtctttttatctctcttactttcacctaggttattttatacaacctgaaaaaaatcaatgaaataaaataatatttttataaaattaactttattatcattatttttttatgattttgccacccatatcattaaattataaaggatataagtgaaaaaaattaatacttcattaaaaaattaaaataataattattttagtataatttttttttcttactaacgattattattaaatgaagggaatagtagtatcatggccatttactttcttaggagagacaaagttagttgagaatcgacacaattaaaatgttagttagaggtgatctgttagttaaataaataaagagggtaggagaaaggtagattcattccatcttgtatcattctatttataagcatttactctgtgaaaggaaaacctttgtgaaggatattcattcaaaacttttgcatctatcaatttcactcctctctttccctagttcgtaaataaaaattaaaaaaaaaaatcaacacggtgactggaagccacacctacagtctacagatatgcagagtgttatttgctttaatccatcgtcccttagtctaatagtcttgtagccagtcagcatcatcacaccaaaagttaggcccgaatagtttgaaatta

SEQ ID NO: 188 - DNA, 30 bp, synthetic construct
  source 1..30: mol_type = other DNA
atcatactcattgctaattgtcttctatca

SEQ ID NO: 189 - DNA, 60 bp, synthetic construct
  source 1..60: mol_type = other DNA
ttctccatattgaccatcatactcattgctaattgtcttctatcattctatgtgctaccc

SEQ ID NO: 190 - DNA, 100 bp, synthetic construct
  source 1..100: mol_type = other DNA
ttggtaattactctttctttttctccatattgaccatcatactcattgctaattgtcttctatcattctatgtgctacccgaaaagaaaaaaaaacagac

SEQ ID NO: 191 - DNA, 1050 bp, synthetic construct
  source 1..1050: mol_type = other DNA
ttggtaattactctttctttttctccatattgaccatcatactcattgctaattgtcttctatcattctatgtgctacccgaaaagaaaaaaaaacagacatctgttaaagagtagctgcagctttattctcttatgcatctgtcaaaccaaggagaaaaaaaatgtcaaactgaaaatttatacgtttttcctttatttcctttttaaacaattcttacattcgctattttaaacgaacatacaaatgtcttactgttttattatttcaattatcaccgttgaattgtgtcaaatttctagcaattctaggtctttaatattgactaagtaactttacccccctaaaaaaacaatagattctctctcatttccttgtgcttactaaaatcgaccctttgcaaaattatagttagaaacaccatacttgcagttgttggagtattacagtagaggctataaaaaaaaattacacagtaagaatggaagatactcgactcaaggtcaatttgacaagacaagaatacccgacctttccattaagtaacctaccaagcgtcacgactacagcaataggcataggtgaccgttgacaaaaaaaaaagagaaaaacattggtgtcctttcccatttctaattttaactattgtcttataaatagtgaaatatccctacacttcatagttttagctaatatacacagaataccacgatctcggatttggctgaactacaccgggcacactaaactcagttatcaaaagcagtatctatcattacgagaacttcctgattgcattgcaatacaacacaagaaaatactaacaacgttagaccaagtttgcataaaaattgatactttatacccaaatgtaagatctaaattgacagcaaattaatgaccctatgcacaaaacaaatgtaaacttcgtgcagcataaatagagctgttgcttcttgcacctcaaagttacttccagcataaacaaagggatgtaggaaacaacccataaatagttgtcaggaaaagtaacggggtattaccttagaa

SEQ ID NO: 192 - DNA, 6466 bp, synthetic construct
  source 1..6466: mol_type = other DNA
tgggggctgcctgtacttcccaaaacttcgcttccctgacccatcatatccaggactggacgattggcttgattgataccagatgggtgagtcgagtccacctcggtagcggcatttatggcaacgattgcagccacgttgacctccatcattttttctcatgctcatcatggcctccatcatggtggtcatttggtctttcatggcctccatgtcggccttcatctgctcttgaacttcatctatctcactcatgattctagccttggcacgtgtttggtaagggtaccgtaaagcgcgttcgttcttttttattactatgattacattttgacgatgatgatgattgtaggaaagaatgaaatgagtaatgaaacaactaaataaacgtgaatgcatgacaatgataagttgctgaagtattataaatttacataggacattcagtggaacgtagggtcgaatcaaatcctatttcattaaaaacaatattgttcatcttgacagagccaaagcataactagaaatacaacatggacacatcagcgattcctaattatgtgggtcattagttcgaccatgtgttggcagtaacttgaaagactatgaacttcatcgggagcagagtatgtgtcagtcaccgccttggctctggctaacaaccttgggatctcttggctctcatttagagtaagagcaaatttgtccatccatttcatggcttctttatgcaataactctatcaccccttctcttgcttccctttcaacctgcaaggtcgacacttttgcctgttcgtcttctagccttcgcccatgactagcagctaggttcaccttctcttcatattggtcaatgattatcaacatattttcttttgttttgctcaactgttctctcaaacttctcttcgatctctgacaactctttaacttatcctctaacatcaggttttccatacttgatttgtccctcttggcttttctaagtttgagctcgttactgctgccccacaaagcccctcgaaacttgttcctgctccactcttccttttgggcttttttgtttcccgctctagcgcttcaatcgtggttatcaagctccaaacactgatagtttaaactgaaggcgggaaacgacaatctgatccccatcaagctctagctagagcggccgcgttatcaagcttctgcaggtcctgctcgagtggaagctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgatgagctcaagaattcgagctcggtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctcagactctagtgactaccaccttcactctcctcaagcatttcagcctcttccccgctcagactccttagctttgggagccaaattatcccttacgttctcgacttcaaccatatgtgatagctgcctatgataccatggctacttccccttagttctttatctttcctttccgctttattccatgccttaccgatcctctgaagtgtctttgcattagcttcattgaaacctcacgcgatgaaaggtgtgatggtctcctccgatggcgcacttctcatagggtaacctaattgtcttacgaccaacataggattataattaatacaacccctcgtccctataaaagggacatttggaaatccttcacataagcataacactcctacccctctttctttccactgtgggaaccaactaatggacgctcctatcatgcctgccaagagttcttcccaatttgcctcgtcctttcctgagcacatgcgatgaccttgtatggggtagacagatctactttcatgattgaagacgtgggataccaaccacacataaagagcaggcgcacaacagaaaatcctcgtagtgctcttcttgcatcttaagtcaaatgtatcatacacttatgctaaaacaacaatgatcgggctttccttgctatggtgataagcaagaaaagcatcgattgctactagatccaccaactcgtctacattcgaaaatagtactatcccaaacactagcagtgctaatacgtcgatgaatgatgcccactctccttggctggccagagtttccgccttctcctccaatcacttccttggtattccccctaccctattcctactttgcttcactcagtctaattctcatttcgagatcttgacaactcctgctattctcgccatagaaggatagtacccagaaaaaaggtatggcttccttcctcctatcgggcatcctaagatcccttcgaactcctctatggttggtgctaactgaaagtccccaaaagtgaagcatctgagtgattggtcatagtattgggtgagagatgcgatg

SEQ ID NO: 193 - DNA, 4303 bp, synthetic construct
  source 1..4303: mol_type = other DNA
caaacactgatagtttaaactgaaggcgggaaacgacaatctgatccccatcaagctctagctagagcggccgcgttatcaagcttctgcaggtcctgctcgagtggaagctaattctcagtccaaagcctcaacaaggtcagggtacagagtctccaaaccattagccaaaagctacaggagatcaatgaagaatcttcaatcaaagtaaactactgttccagcacatgcatcatggtcagtaagtttcagaaaaagacatccaccgaagacttaaagttagtgggcatctttgaaagtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagaattagcttgagctcaggatttagcagcattccagattgggttcaatcaacaaggtacgagccatatcactttattcaaattggtatcgccaaaaccaagaaggaactcccatcctcaaaggtttgtaaggaagaattcgatatcaagcttgatatcggaagtttctctcttgagggaggttgctcgtggaatgggacacatatggttgttataataaaccatttccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagctcgcttagctcagatgatagagcaccacaatttttgtggtagaaatcggtttgactccgatagcggctttttactatgattgttttgtgttaaagatgattttcataatggttatatatgtctactgtttttattgattcaatatttgattgttcttttttttgcagatttgttgaccagagatctaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccgtccagcaactgctcgtaagtcctctggtctttctggaaccgtccgtattccaggtgacaagtctatctcccacaggtccttcatgtttggaggtctcgctagcggtgaaactcgtatcaccggtcttttggaaggtgaagatgttatcaacactggtaaggctatgcaagctatgggtgccagaatccgtaaggaaggtgatacttggatcattgatggtgttggtaacggtggactccttgctcctgaggctcctctcgatttcggtaacgctgcaactggttgccgtttgactatgggtcttgttggtgtttacgatttcgatagcactttcattggtgacgcttctctcactaagcgtccaatgggtcgtgtgttgaacccacttcgcgaaatgggtgtgcaggtgaagtctgaagacggtgatcgtcttccagttaccttgcgtggaccaaagactccaacgccaatcacctacagggtacctatggcttccgctcaagtgaagtccgctgttctgcttgctggtctcaacaccccaggtatcaccactgttatcgagccaatcatgactcgtgaccacactgaaaagatgcttcaaggttttggtgctaaccttaccgttgagactgatgctgacggtgtgcgtaccatccgtcttgaaggtcgtggtaagctcaccggtcaagtgattgatgttccaggtgatccatcctctactgctttcccattggttgctgccttgcttgttccaggttccgacgtcaccatccttaacgttttgatgaacccaacccgtactggtctcatcttgactctgcaggaaatgggtgccgacatcgaagtgatcaacccacgtcttgctggtggagaagacgtggctgacttgcgtgttcgttcttctactttgaagggtgttactgttccagaagaccgtgctccttctatgatcgacgagtatccaattctcgctgttgcagctgcattcgctgaaggtgctaccgttatgaacggtttggaagaactccgtgttaaggaaagcgaccgtctttctgctgtcgcaaacggtctcaagctcaacggtgttgattgcgatgaaggtgagacttctctcgtcgtgcgtggtcgtcctgacggtaagggtctcggtaacgcttctggagcagctgtcgctacccacctcgatcaccgtatcgctatgagcttcctcgttatgggtctcgtttctgaaaaccctgttactgttgatgatgctactatgatcgctactagcttcccagagttcatggatttgatggctggtcttggagctaagatcgaactctccgacactaaggctgcttgatgagctcaagaattcgagctcggtaccggatcctctagctagagctttcgttcgtatcatcggtttcgacaacgttcgtcaagttcaatgcatcagtttcattgcgcacacaccagaatcctactgagtttgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgcaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgacctgcagccactcgaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgct

SEQ ID NO: 194 - DNA, 20 bp, synthetic construct
  source 1..20: mol_type = other DNA
tatcaagctccaaacactga

SEQ ID NO: 195 - DNA, 1222 bp, synthetic construct
  source 1..1222: mol_type = other DNA
cctgtacttcccaaaacttcgcttccctgacccatcatatccaggactggacgattggcttgattgataccagatgggtgagtcgagtccacctcggtagcggcatttatggcaacgattgcagccacgttgacctccatcattttttctcatgctcatcatggcctccatcatggtggtcatttggtctttcatggcctccatgtcggccttcatctgctcttgaacttcatctatctcactcatgattctagccttggcacgtgtttggtaagggtaccgtaaagcgcgttcgttcttttttattactatgattacattttgacgatgatgatgattgtaggaaagaatgaaatgagtaatgaaacaactaaataaacgtgaatgcatgacaatgataagttgctgaagtattataaatttacataggacattcagtggaacgtagggtcgaatcaaatcctatttcattaaaaacaatattgttcatcttgacagagccaaagcataactagaaatacaacatggacacatcagcgattcctaattatgtgggtcattagttcgaccatgtgttggcagtaacttgaaagactatgaacttcatcgggagcagagtatgtgtcagtcaccgccttggctctggctaacaaccttgggatctcttggctctcatttagagtaagagcaaatttgtccatccatttcatggcttctttatgcaataactctatcaccccttctcttgcttccctttcaacctgcaaggtcgacacttttgcctgttcgtcttctagccttcgcccatgactagcagctaggttcaccttctcttcatattggtcaatgattatcaacatattttcttttgttttgctcaactgttctctcaaacttctcttcgatctctgacaactctttaacttatcctctaacatcaggttttccatacttgatttgtccctcttggcttttctaagtttgagctcgttactgctgccccacaaagcccctcgaaacttgttcctgctccactcttccttttgggcttttttgtttcccgctctagcgcttcaatcgtggttatcaagctccaaacactgatagtttaaactgaaggcgggaaacgacaatctgatccccatcaagctctagctagagcggccgcgttatcaagcttctgcaggtcctgctcgagtggaagctaattctcagtccaaag

SEQ ID NO: 196 - DNA, 20 bp, synthetic construct
  source 1..20: mol_type = other DNA
taataacgctcagactctag

SEQ ID NO: 197 - DNA, 675 bp, synthetic construct
  source 1..675: mol_type = other DNA
gccaattgattgacaacatgcatcaatcgacctgcagccactcgaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctcagactctagtgactaccaccttcactctcctcaagcatttcagcctcttccccgctcagactccttagctttgggagccaaattatcccttacgttctcgacttcaaccatatgtgatagctgcctatgataccatggctacttccccttagttctttatctttcctttccgctttattccatgccttaccgatcctctgaagtgtctttgcattagcttcattgaaacctcacgcgatgaaaggtgtgatggtctcctccgatggcgcacttctcatagggtaacctaattgtcttacgaccaacataggattataattaatacaacccctcgtccctataaaagggacatttggaaatccttcacataagcataacactcctacccctctttctttccactgtgggaaccaactaatggacgctcctatcatgcctgccaagagttcttccc

SEQ ID NO: 198 - DNA, 3684 bp, synthetic construct
  source 1..3684: mol_type = other DNA
atgtcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 199 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 200 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDKETDYRATILRYGSKYYLAIMDKKYAKCLQKIDKDDVNGNYEKINYKLLPGPNKMLPRVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 201 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMRGWDKDVETDRRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 202 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 203 - DNA, 21 bp, synthetic construct
  source 1..21: mol_type = other DNA
gaatttctactaagtgtagat

SEQ ID NO: 204 - DNA, 21 bp, synthetic construct
  source 1..21: mol_type = other DNA
gtaatttctactgttgtagat

SEQ ID NO: 205 - DNA, 51 bp, synthetic construct
  source 1..51: mol_type = other DNA
gtaatttctactgttgtagatttataaggtgacctcctccataattttttt

SEQ ID NO: 206 - DNA, 51 bp, synthetic construct
  source 1..51: mol_type = other DNA
gtaatttctactgttgtagataaagattaggttcatttgaaaagttttttt

SEQ ID NO: 207 - DNA, 21 bp, synthetic construct
  source 1..21: mol_type = other DNA
taatttctactaagtgtagat

SEQ ID NO: 208 - DNA, 30 bp, synthetic construct
  source 1..30: mol_type = other DNA
gaaaagttaaatggctgaaaccttagttac

SEQ ID NO: 209 - DNA, 27 bp, synthetic construct
  source 1..27: mol_type = other DNA
tactatgatatagcattttcgtggggt

SEQ ID NO: 210 - DNA, 18 bp, synthetic construct
  source 1..18: mol_type = other DNA
catatatgtccaaagtca

SEQ ID NO: 211 - DNA, 27 bp, Glycine max
  source 1..27: mol_type = genomic DNA
tttgcaaacatcacatggccggataag

SEQ ID NO: 212 - DNA, 27 bp, Glycine max
  source 1..27: mol_type = genomic DNA
tttacttcctgaggaaatttggtttca

SEQ ID NO: 213 - DNA, 27 bp, Glycine max
  source 1..27: mol_type = genomic DNA
tttggcaagatccgctgcctgcttgag

SEQ ID NO: 214 - DNA, 27 bp, Glycine max
  source 1..27: mol_type = genomic DNA
tttaccttgatttggattaacccgcca

SEQ ID NO: 215 - DNA, 27 bp, Glycine max
  source 1..27: mol_type = genomic DNA
tttactaaatcgcatagcttgtgtgtt

SEQ ID NO: 216 - DNA, 27 bp, Glycine max
  source 1..27: mol_type = genomic DNA
tttacatgctgtcatcatctgttcact

SEQ ID NO: 217 - DNA, 27 bp, Glycine max
  source 1..27: mol_type = genomic DNA
tttccctatatctgtttcagtgattgc

SEQ ID NO: 218 - DNA, 27 bp, Glycine max
  source 1..27: mol_type = genomic DNA
tttggtgtatacgtgtgtgagacaact

SEQ ID NO: 219 - DNA, 27 bp, Glycine max
  source 1..27: mol_type = genomic DNA
tttgcggagtagcagagaggagattcg

SEQ ID NO: 220 - DNA, 27 bp, Glycine max
  source 1..27: mol_type = genomic DNA
tttgatcaaagctaaagcataagcatc

SEQ ID NO: 221 - DNA, 27 bp, Glycine max
  source 1..27: mol_type = genomic DNA
tttcgctcgctatatatatgtgtcaca

SEQ ID NO: 222 - DNA, 27 bp, Glycine max
  source 1..27: mol_type = genomic DNA
tttggacatatatgctatatcattctc

SEQ ID NO: 223 - DNA, 23 bp, Glycine max
  source 1..23: mol_type = genomic DNA
caaacatcacatggccggataag

SEQ ID NO: 224 - DNA, 23 bp, Glycine max
  source 1..23: mol_type = genomic DNA
cttcctgaggaaatttggtttca

SEQ ID NO: 225 - DNA, 23 bp, Glycine max
  source 1..23: mol_type = genomic DNA
gcaagatccgctgcctgcttgag

SEQ ID NO: 226 - DNA, 23 bp, Glycine max
  source 1..23: mol_type = genomic DNA
ccttgatttggattaacccgcca

SEQ ID NO: 227 - DNA, 23 bp, Glycine max
  source 1..23: mol_type = genomic DNA
ctaaatcgcatagcttgtgtgtt

SEQ ID NO: 228 - DNA, 23 bp, Glycine max
  source 1..23: mol_type = genomic DNA
catgctgtcatcatctgttcact

SEQ ID NO: 229 - DNA, 23 bp, Glycine max
  source 1..23: mol_type = genomic DNA
cctatatctgtttcagtgattgc

SEQ ID NO: 230 - DNA, 23 bp, Glycine max
  source 1..23: mol_type = genomic DNA
gtgtatacgtgtgtgagacaact

SEQ ID NO: 231 - DNA, 23 bp, Glycine max
  source 1..23: mol_type = genomic DNA
cggagtagcagagaggagattcg

SEQ ID NO: 232 - DNA, 23 bp, Glycine max
  source 1..23: mol_type = genomic DNA
atcaaagctaaagcataagcatc

SEQ ID NO: 233 - DNA, 23 bp, Glycine max
  source 1..23: mol_type = genomic DNA
gctcgctatatatatgtgtcaca

SEQ ID NO: 234 - DNA, 23 bp, Glycine max
  source 1..23: mol_type = genomic DNA
gacatatatgctatatcattctc

SEQ ID NO: 235 - DNA, 30 bp, Solanum lycopersicum
  source 1..30: mol_type = genomic DNA
ggatctaagaagagaagaattaaacaagat

SEQ ID NO: 236 - DNA, 3681 bp, synthetic construct
  source 1..3681: mol_type = other DNA
tccaagctggagaagtttacaaactgttacagcctctccaaaaccctcaggtttaaagcgatcccggtgggcaagacccaggagaacatcgacaacaagaggctcctggtggaagacgagaagcgcgccgaagactacaagggcgtgaagaagctgctcgataggtactacctcagctttattaacgacgtgctgcacagcatcaaactcaagaatctcaacaactacatctccctcttccgcaaaaagacccgcaccgagaaggagaacaaggagctggagaacctggagatcaacctccgcaaggaaatcgccaaagcgttcaagggcaatgaagggtacaagagcctcttcaagaaagacatcatcgaaactatcctcccagagtttctcgatgacaaggacgagatcgcgctggtgaactcctttaacgggttcacaaccgcgtttaccggcttctttgataacagggaaaatatgttctccgaggaggccaagtccaccagcatcgccttcaggtgtatcaacgagaacctcacccgctacatttccaatatggacattttcgagaaggtggatgcgatcttcgataagcacgaggtgcaggagatcaaagagaagattctcaattccgattatgacgtcgaggatttcttcgaaggggagttctttaattttgtgctcacacaagagggcattgacgtgtacaacgcgattatcgggggcttcgtcacagagtccggggagaagattaaggggctgaatgagtacatcaatctgtacaatcagaagaccaagcagaaactgccgaaattcaagccgctctacaagcaagtcctgtccgatagggaaagcctctccttctacggcgagggctataccagcgacgaggaggtgctggaagtcttccgcaacacactgaataagaatagcgagattttctcctccatcaagaagctcgagaagctctttaagaactttgacgagtacagctccgccgggattttcgtgaagaacgggccggcgatcagcaccatctccaaggacatctttggcgagtggaacgtcatcagggacaagtggaacgccgagtacgacgacatccacctgaagaagaaggcggtggtgaccgagaagtatgaggacgatcgcaggaagtccttcaaaaaaatcggctccttcagcctcgaacagctccaggagtatgccgatgcggatctgtccgtcgtcgagaagctgaaggaaatcatcattcagaaggtcgacgagatctataaagtgtacgggtccagcgagaagctgttcgacgccgactttgtgctcgagaagtccctcaaaaagaatgacgccgtggtggccattatgaaagacctgctcgactccgtgaagtccttcgaaaattacattaaagcgttctttggggaggggaaggaaactaacagggatgagtccttctatggcgactttgtcctcgcgtacgacatcctgctgaaggtcgaccacatttacgacgcgatccgcaactacgtgacacagaagccgtactccaaagacaagttcaagctgtacttccagaacccgcaatttatggggggctgggacaaggataaagagacagactaccgcgcgacaattctccgctatggctccaaatactatctggccatcatggacaagaagtacgcgaagtgcctgcagaagatcgacaaagacgacgtcaatggcaactatgaaaagatcaactacaagctgctgccgggcccgaacaagatgctcccgaaggtgttcttcagcaagaagtggatggcctactacaatccaagcgaggatattcagaaaatctataaaaacgggaccttcaagaagggggacatgtttaacctcaacgactgccacaagctcattgatttcttcaaggatagcatttcccgctacccgaaatggtccaatgcgtacgattttaacttctccgagacagaaaagtacaaagacatcgcgggcttttacagggaggtggaggagcaagggtataaagtttcttttgaatccgcgagcaagaaggaagtcgacaagctcgtcgaggagggcaagctctacatgttccaaatttataacaaggacttttccgacaagagccatgggaccccaaacctccacaccatgtacttcaaactgctctttgacgagaacaaccacgggcaaatcaggctgagcggcggcgccgaattattcatgcgcagggcctccctcaagaaggaagagctggtcgtccatccagccaattccccgatcgcgaacaagaacccggacaatccgaaaaagaccaccaccctgtcctacgacgtctacaaggacaaacgcttcagcgaagaccagtacgaattacacatcccaattgcgattaataagtgcccaaagaatatcttcaaaattaatacagaggtcagggtgctgctcaaacacgacgacaatccgtatgtcatcggcattgacaggggcgagcgcaatctgctctatatcgtggtcgtggatgggaagggcaatattgtggagcagtactccctgaacgagattatcaacaacttcaatgggattaggattaagaccgactatcacagcctgctcgacaagaaagaaaaagagaggtttgaggcccgccaaaactggacctccattgagaatatcaaagaattaaaggccggctatatttcccaagtcgtccacaagatctgcgagctggtggagaaatatgacgccgtgattgcgctcgaagacttaaattctgggttcaagaactcccgcgtgaaggtggaaaaacaggtgtatcagaaattcgagaaaatgctgatcgacaaactcaattatatggtggataagaagtccaacccgtgtgccacagggggcgcgctgaagggctatcagatcaccaacaagttcgagagcttcaagagcatgagcacccagaacgggtttattttctacatcccggcgtggctcacctccaagattgacccgagcaccggcttcgtgaacctcctgaagacaaagtatacctccattgccgacagcaagaagtttatctcctccttcgaccgcattatgtatgtgccggaggaggacctcttcgagttcgccctcgactacaaaaacttcagccgcacagatgcggattacatcaagaagtggaagctgtactcctacgggaacaggatccgcatcttcaggaatccaaaaaaaaataacgtctttgactgggaggaagtgtgcctgacatccgcctacaaggaactgttcaataaatacggcatcaattaccagcagggcgacattcgcgccctcctctgtgagcagtccgacaaagcgttttactccagcttcatggccctcatgtccctgatgctccaaatgaggaatagcatcacagggcgcaccgacgtcgacttcctcatcagcccggtgaagaactccgacgggatcttttacgactcccgcaactatgaggcgcaagagaatgcgatcctcccgaagaacgccgatgcgaacggggcctataatatcgccaggaaagtgctctgggccatcgggcagttcaaaaaggcggaggatgagaagctcgacaaggtgaaaattgccatttccaacaaggagtggctggagtacgcgcagacctccgtgaagcac

SEQ ID NO: 237 - DNA, 500 bp, Medicago truncatula
  source 1..500: mol_type = genomic DNA
aagggctctctgtcatgatttcatactttcattattgagctctgtaattacaattatgaccatgagaacatctcttattgtgtggccttttaattgctgatgttagtactgaaccaaagcttatcgtgatgatgtaaaagcaataagtacttgtttgtagcttctttgtgtctccctttgggcttaatacatctgtttagtgttgtggctttggcatagacttctcttggtaataatgccttgcaatgcaaaatttcaattatcaaattctattatgttctcaccttatggtaacagcttaccctgtggaagatgagattcttgagttgagtcattgccaatttttggcattagcttttgaattagtgaattttgacaaaaattaccgtgacactgattttgttgaagctcttaagtgtagtttttacaaaatttcagtggctcgttgtgattatgtcaaactcacggcgaatgtagttcttacagaatttcagtggctc

SEQ ID NO: 238 - DNA, 822 bp, synthetic construct
  source 1..822: mol_type = other DNA
agacatcctggaccaatatgctgaagattatgctacctacaccaggataggacttgaagcacttaaccttgaagattggttcgaagaaccagaacccgatccacctaaccctgtggaccgccagaggatagaggacatcctggacctactgaacgtcagcaatgacgactgaaagattcccaggacaccggcggaagtggtggacccagtctaggtgcgatgcttagtcgcgcacgatgactatgtcggaaggcatctttgctttcggcaaactttagtaatactttaaggaaagtattgtacaagttaggtgcagagacaataatgcacccagctttagctttgtttatggaattattgtgtcggttgcattattggatgcctgcgtgcaccctaagcaatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtgcaggtggggcctccactagcaataatacaagctttacagcttgcagtgactcatcctccaataatggagaaaaagacgtcagcagtgacgaacaagggtcgaaagacttgcctatataagggcattctcccctcagttgaagatcatcgaaagttggagcaataaactctctcttcaacaaatctatcttttatcttttatc

SEQ ID NO: 239 - DNA, 20 bp, synthetic construct
  source 1..20: mol_type = other DNA
aatttctactaagtgtagat

(19) United States
(12) Patent Application Publication
(10) Pub. No.: US 2025/0197952 A1
(43) Pub. Date: Jun. 19, 2025

(54) TRANSGENIC SOYBEAN EVENT GM_CSM63717 AND COMPOSITIONS AND METHODS FOR DETECTION AND USES THEREOF

(71) Applicant: Monsanto Technology LLC, St. Louis, MO (US)

(72) Inventors: Sarah Brown, Weldon Spring, MO (US); Christine Ellis, Manchester, MO (US); Robert Gaeta, Eureka, MO (US); Zane Goodwin, St. Louis, MO (US); Diane Hohorst, Foristell, MO (US); Clayton Tyler Larue, Chesterfield, MO (US); Linda Rymarquis, High Ridge, MO (US); Xiaohong Shi, St. Charles, MO (US); Janice Weihe, New Baden, IL (US); Xudong Ye, Chesterfield, MO (US)

(21) Appl. No.: 18/971,656

(22) Filed: Dec. 6, 2024

Related U.S. Application Data
(60) Provisional application No. 63/609,784, filed on Dec. 13, 2023.

(51) Int. Cl. C12Q 1/689; A01H 1/04; C07K 14/415; C12N 9/02; C12N 15/82; G01N 33/00; G01N 33/573

(52) U.S. Cl. CPC C12Q 1/689; A01H 1/045; C07K 14/415; C12N 9/001; C12N 15/8274; G01N 33/0098; G01N 33/573; C12Q 2600/13; C12Y 103/03004; G01N 2333/90206

(57) ABSTRACT

A transgenic soybean event, Gm_CSM63717, is provided. Transgenic plant cells, plant parts, plants, seeds, progeny plants, and agricultural and commodity products containing event Gm_CSM63717 are also provided. Recombinant DNA molecules unique to the event Gm_CSM63717, and methods of using and detecting Gm_CSM63717 are also provided. Soybean plants containing the event Gm_CSM63717 exhibit tolerance to PPO inhibitors.

### CROSS-REFERENCE TO RELATED APPLICATIONS

This application claims the priority of U.S. Provisional Appl. Ser. No. 63/609,784, filed Dec. 13, 2023, the entire disclosure of which is incorporated herein by reference.

### INCORPORATION OF SEQUENCE LISTING

The sequence listing contained in the file named “MONS572US_ST26.xml”, which is 293 kilobytes (measured in MS-Windows) and was created on Oct. 1, 2024, is filed herewith by electronic submission, and is incorporated herein by reference in its entirety.

### FIELD

The present disclosure relates to compositions and methods for providing herbicide tolerance in transgenic soybean plants. Recombinant DNA molecules present in and/or isolated from soybean event Gm_CSM63717 are provided. Also provided are transgenic soybean plants, plant parts, seeds, cells, and agricultural products comprising the soybean event Gm_CSM63717, as well as methods of producing and using transgenic soybean plants, plant parts, seeds, cells, and agricultural products comprising soybean event Gm_CSM63717, methods of detecting soybean event Gm_CSM63717, and methods of controlling weeds. Transgenic soybean plants, plant parts, seeds and cells comprising soybean event Gm_CSM63717 exhibit tolerance to a variety of PPO herbicides.

### BACKGROUND

Increasing sustainable crop production is crucial to meet the need for food for the growing global population, feed for increased demand on animal-based diets in developing nations, and expanded use of crop products to produce biofuel, fiber, and other agricultural product-based commodities, while using limited natural resources. In agricultural systems, the effective management of weedy species in agricultural fields is essential for maintaining favorable crop growing conditions and yield. Weeds compete with crops for space, nutrients, water, and light and can contaminate harvests, and present one of the major challenges to sustainable crop production. Averaged across seven years from 2007 to 2013, weed interference in soybean caused an average of 52.1% yield loss in the US and Canada, which equates to a loss of about $17.1 billion annually if no weed management tactics were employed (Soltani et al., 2017). Moreover, yield losses due to weeds are influenced by weather. Under drought conditions, numerous weed species exhibit increased competitiveness (Patterson, 1995). Selective herbicides had significantly contributed to weed management before the deployment of herbicide tolerant crops. Application of herbicides provides an important tool to reduce weed pressure, improve productivity and increase security for global crop production.

Soybean (Glycine max) is an important crop in many areas of the world. The introduction of genetically modified crops containing herbicide tolerance traits has successfully provided additional tools available to farmers to better control weeds. Transgenic herbicide tolerance enables the use of an herbicide in a crop growing environment without crop injury or with minimal crop injury (e.g., less than about 10% injury). Transgenic soybean traits have been used to impart tolerance to glyphosate, glufosinate, and 2, 4-D and are used broadly in commercial soybean production for weed management. However, weeds have evolved resistance to herbicides and weed resistance continues to present a challenge in soybean production today. Therefore, there is a need for additional herbicide tolerance trait options to manage weeds effectively and to sustain crop productivity. One of the solutions is to employ herbicide(s) with new or different mode(s) of action, and/or employ multiple herbicides with different modes of action.

Protoporphyrinogen IX oxidase (PPO) catalyzes the oxidation of protoporphyrinogen IX to produce protoporphyrin IX. This enzymatic step is conserved across prokaryotes and eukaryotes for the synthesis of tetrapyrroles such as heme. In plants, the production of chlorophyll is dependent on this PPO catalyzed reaction because protoporphyrin IX is an important precursor for chlorophyll synthesis. Because of the important role of PPO in plant chlorophyll synthesis, diverse protoporphyrinogen oxidase (PPO)-inhibiting herbicides have been developed and used for weed control in agriculture since the 1960s. Application of the PPO-inhibiting herbicides to sensitive plants results in the blockage of heme and chlorophyll biosynthetic pathways in the plastids, leading to the accumulation of pathway intermediates which leak from the plastids and undergo non-specific oxidation to protoporphyrin IX in the cytosol. In the presence of oxygen and light, protoporphyrin IX rapidly generates singlet oxygen, resulting in uncontrolled membrane lipid peroxidation and plant death. Development of a PPO-inhibiting herbicide tolerance trait through biotechnology will provide additional tools and additional herbicide mode of action to the farmers for diversifying weed control system to control weed and to reduce/prevent development of herbicide resistance (Larue et al, 2019). Such a PPO herbicide tolerance trait could either be deployed alone or could be stacked with other herbicide tolerance traits.

Combinations of herbicide tolerance traits are desirable to provide weed control options that increase grower flexibility and enable the use of multiple herbicide modes of action for controlling challenging weeds. Combining multiple desired traits in the genome can be achieved by several approaches: 1) by making crosses between two parents each having a desired trait at a randomly inserted site, and identifying progeny plants that have combination of the desired traits; 2) by retransforming a transgenic plant comprising one or more desired trait(s) with one or more genes for additional desired traits, either through random integration or through targeted integration of the one or more genes for additional desired traits; 3) by inserting multiple genes as a single DNA molecule into one location, or locus, in the genome, which provides a useful tool in weed control that is much simpler and less expensive to maintain during subsequent breeding into a diverse pool of elite germplasms; and 4) by targeting one or more desired traits to a specific genomic location (site directed integration) carrying one or more desired traits, in a new transformation event, followed by crosses between the new event and another event carrying the one or more desired event at the specific genomic location, resulting in progeny plants that have combination of the desired traits at one location and segregate together.

The expression of transgenes in a transgenic plant, plant part, seed, cell or progeny, and thus their effectiveness, may be influenced by many factors, such as the regulatory elements used in the transgenes' expression cassettes, the combination and/or interaction of these regulatory elements, the chromosomal location of the transgene insertion site, the chromatin structure of the genome at or near the transgene insertion site, and the presence or proximity of any endogenous cis and/or trans regulatory elements or genes close to the transgene insertion site. In addition, the performance of the traits in the transgenic plant is further complicated when the transgenic insert comprises multiple expression cassettes, each having a different transgene conferring a distinct trait. These differences or factors may result in variation in the level of transgene expression or in the spatial or temporal pattern of transgene expression among different transgenic insertion events of the same expression cassettes. Furthermore, different transgenic events can also vary in terms of the molecular quality of the events. For example, a transgenic event may contain two or more copies of the transgene insertion at one or more chromosomal locations, or a transgenic insertion may be truncated relative to the intended insertion or contain vector backbone sequences, or a transgene may be inserted into an endogenous gene or in a repeated region. In the case of site directed integration of desired traits, the machinery for site directed integration, such as gRNA or nuclease, which has to be excised from the commercial events, may not be completely removed. Such characteristics may result in undesirable outcomes, such as gene silencing, altered pattern and/or expression of the transgene, and/or altered pattern and/or expression of endogenous genes. There may also be undesirable phenotypic or agronomic differences among different events.

Even in the case of targeted sequence insertion, variability in the level of transgene expression between independent but genetically identical targeted sequence insertion (TSI) events was observed in a subset of transgenic events (Verkest et al., 2019). This expression variability and silencing occurred independently of the transgene sequence and could be attributed to DNA methylation that was further linked to different DNA methylation mechanisms. Transgene integration into targeted loci through Cre-lox mediated recombination has also been reported to produce a large percentage of targeted integration events that showed a partial spatial pattern of transgene expression due to differential silencing (Day et al., 2000). The fact that a considerable variation in transgene expression was observed shows that even when integration events are targeted, selection remains necessary similarly to the practice for random integration events in order to identify targeted insertion events with stable and desirable gene of interest expression over generations.

A commercially useful transgenic event requires that the transgene(s) in the transgenic insert express in the manner necessary for that trait to be successful, and involves rigorous testing, evaluation, and selection. Such tests include in vitro and/or in planta testing different regulatory elements (e.g., promoters, introns, leaders, and 3′ UTRs) and combinations of different regulatory elements for desirable spatial and temporal expression of the transgene(s), as well as examining whether to target the product of the transgene(s) (protein(s)) to subcellular compartments such as chloroplasts to select for the best expression cassette(s). For site directed integration of a transgene, once a targeted insertion strategy/method is chosen, the target sites are identified, screened and selected. The selected combinations of expression cassette(s), targeting sites and gRNA are then used for transformation to produce transgenic plants.

For these reasons, the performance of different transformation events from the same transformation construct can vary widely, and the identification of transformation events conferring the most beneficial traits or characteristics without other potential off-types or concerns is needed to select a superior event for commercial use. Therefore, a large number of individual transgenic events must be produced and analyzed to select an event having superior commercial properties, which can be a significant undertaking that involves analysis and selection among many different transformation events.

To establish a transgenic event for commercial use requires rigorous molecular characterization, greenhouse testing, and field trials over multiple years, in multiple locations and under a variety of conditions, allowing extensive agronomic, phenotypic, and molecular data to be obtained. The resulting data are then analyzed to select an event that is suitable for commercial purposes. The commercial event, once identified as having the desired transgene expression, molecular characteristics, efficacy and field performance, can then be introgressed into other soybean genetic backgrounds using plant breeding methods. The resulting soybean varieties contain the new traits combined with other desirable qualities such as native traits, disease tolerance traits, insect control traits, high-yielding germplasms or traits, and/or one or more other transgenic herbicide tolerance traits.

### SUMMARY

Recombinant DNA molecules are provided herein. Examples of such recombinant DNA molecules include recombinant DNA molecules comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. In some embodiments, the recombinant DNA molecule is derived from a soybean plant, seed, plant part, plant cell, progeny plant, or commodity product comprising soybean event Gm_CSM63717, a representative sample of seed comprising the event having been deposited as ATCC Accession No. PTA-127604. In some embodiments, the recombinant DNA molecule is comprised in a soybean plant, seed, plant part, plant cell, or progeny plant comprising soybean event Gm_CSM63717, or a commodity product produced therefrom, a representative sample of seed comprising the event having been deposited as ATCC Accession No. PTA-127604. The recombinant DNA molecule can be formed by the insertion of a heterologous nucleic acid molecule into the genomic DNA of a soybean plant or soybean cell. The recombinant DNA molecule can comprise an amplicon diagnostic for the presence of soybean event Gm_CSM63717.

DNA molecules that function as DNA probes are provided. An example of such a DNA molecule is a DNA molecule comprising a polynucleotide segment of sufficient length to function as a DNA probe that hybridizes specifically under stringent hybridization conditions with soybean event Gm_CSM63717 DNA in a sample. Detecting hybridization of the DNA molecule under the stringent hybridization conditions is diagnostic for the presence of soybean event Gm_CSM63717 in the sample.

Also provided is a DNA molecule comprising a polynucleotide segment of sufficient length to function as a DNA probe specific for detecting in a sample at least one of: a 5′ junction sequence between flanking soybean genomic DNA and the transgenic insert of soybean event Gm_CSM63717; a 3′ junction sequence between the transgenic insert of soybean event Gm_CSM63717 and flanking soybean genomic DNA; SEQ ID NO:9; and a fragment of SEQ ID NO:9 comprising a sufficient length of contiguous nucleotides of SEQ ID NO:9 to identify the sequence as a fragment of the transgenic insert of Gm_CSM63717.

The DNA probe can comprise SEQ ID NO:178. Alternatively or in addition, the DNA probe can comprise a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing. The sample can be derived from a soybean plant, seed, plant part, plant cell, progeny plant, or commodity product.

A pair of DNA molecules is provided. The pair of DNA molecules comprises a first DNA molecule and a second DNA molecule. The first and the second DNA molecules are different from one another, and each comprise a fragment of SEQ ID NO:10 or a complement thereof and function as DNA primers when used together in an amplification reaction with DNA comprising soybean event Gm_CSM63717 to produce an amplicon diagnostic for soybean event Gm_CSM63717 in a sample. For example, the first and the second DNA molecules can comprise SEQ ID NO:176 and SEQ ID NO:177. The amplicon can comprise a nucleotide sequence selected from the group consisting of: SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 4,201-4,202 of SEQ ID NO:10.

Methods of detecting the presence of soybean event Gm_CSM63717 in a sample derived from a soybean seed, plant, plant part, plant cell, progeny plant, or commodity product are provided. In a first example of such a method, the method comprises: (a) contacting the sample with any of the the DNA molecules that functions as a DNA probe specific for soybean event Gm_CSM63717 described herein; (b) subjecting the sample and the DNA molecule that functions as a probe to stringent hybridization conditions; and (c) detecting the hybridization of the DNA molecule that functions as a probe to a DNA molecule in the sample. The hybridization of the DNA molecule that functions as a probe to the DNA molecule in the sample is diagnostic for the presence of soybean event Gm_CSM63717 in the sample.

Another method of detecting the presence of soybean event Gm_CSM63717 in a sample derived from a soybean seed, plant, plant part or plant cell, progeny plant or commodity product is provided. The method comprises: (a) contacting the sample with any of the pairs of DNA molecules that can be used to produce an amplicon diagnostic for soybean event Gm_CSM63717 described herein; (b) performing an amplification reaction sufficient to produce a DNA amplicon; and (c) detecting the presence of the DNA amplicon. The DNA amplicon comprises at least one of: a 5′ junction sequence between flanking soybean genomic DNA and the transgenic insert of soybean event Gm_CSM63717, a 3′ junction sequence between flanking soybean genomic DNA and the transgenic insert of soybean event Gm_CSM63717, SEQ ID NO: 9, and a fragment of SEQ ID NO: 9 comprising a sufficient length of contiguous nucleotides of SEQ ID NO: 9 to identify the sequence as a fragment of the transgenic insert of Gm_CSM63717. The presence of the DNA amplicon indicates the presence of soybean event Gm_CSM63717 in the sample. The DNA amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length. The DNA amplicon can comprise a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,201-4,202 of SEQ ID NO:10.

A further method of detecting the presence of soybean event Gm_CSM63717 in a sample of DNA derived from a soybean seed, plant, plant part, plant cell, progeny plant or commodity product is provided. The method comprises: (a) contacting the sample with any of the DNA molecules that function as a probe specific for soybean event Gm_CSM63717 described herein; and (b) performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,201-4,202 of SEQ ID NO:10.

Another method of detecting the presence of soybean event Gm_CSM63717 in a sample derived from a soybean seed, plant, plant part, cell, progeny plant or commodity product is provided. The method comprises: (a) contacting the sample with an antibody specific for the PPO (protoporphyrinogen oxidase) protein encoded by soybean event Gm_CSM63717; and (b) detecting binding of the antibody to the protein in the sample. The binding of the antibody indicates the presence of soybean event Gm_CSM63717 in the sample.

DNA detection kits for detecting the presence of soybean event Gm_CSM63717 in a sample are provided. One example of such a DNA detection kit is a kit comprising any of the the pairs of DNA primers that can be used as primers to produce an amplicon diagnostic for soybean event Gm_CSM63717 described herein. Another example of a DNA detection kit is a kit comprising any of the DNA molecules that function as probes specific for soybean event Gm_CSM63717 described herein.

Also provided are protein detection kits for detecting the presence of soybean event Gm_CSM63717 in a sample. One example of such a kit is a kit comprising an antibody specific for the PPO protein encoded by soybean event Gm_CSM63717. Detecting binding of the antibody to the protein encoded by soybean event Gm_CSM63717 in a sample is diagnostic for the presence of soybean event Gm_CSM63717 in the sample.

Methods of determining the zygosity of a soybean plant, plant part, plant seed, or plant cell comprising soybean event Gm_CSM63717 are provided. One example of such a method comprises: (a) contacting a sample comprising DNA derived from the soybean plant, plant part, plant seed, or plant cell with a first primer set capable of producing a first amplicon diagnostic for the presence of soybean event Gm_CSM63717, and a second primer set capable of producing a second amplicon diagnostic for wildtype soybean genomic DNA not comprising soybean event Gm_CSM63717; (b) performing a nucleic acid amplification reaction; and (c) detecting the first amplicon and the second amplicon. The presence of both amplicons indicates that the plant, plant part, seed or cell is heterozygous for soybean event Gm_CSM63717. The presence of only the first amplicon indicates that the plant, plant part, seed, or cell is homozygous for soybean event Gm_CSM63717. For example, the first primer set can comprise SEQ ID NO:176 and SEQ ID NO:177, and the second primer set can comprise SEQ ID NO:208 and SEQ ID NO:209.

Another method of determining the zygosity of a soybean plant, plant part, plant seed, or plant cell comprising soybean event Gm_CSM63717 is provided. The method comprises: (a) contacting a sample comprising DNA derived from the soybean plant, plant part, plant seed, or plant cell with a probe set comprising at least a first probe that specifically hybridizes to soybean event Gm_CSM63717, and at least a second probe that specifically hybridizes to soybean genomic DNA that was disrupted by insertion of the heterologous DNA of soybean event Gm_CSM63717 but does not hybridize to soybean event Gm_CSM63717; and (b) hybridizing the probe set with the sample under stringent hybridization conditions. Detecting hybridization of only the first probe under the hybridization conditions is diagnostic for a soybean plant, plant part, seed or plant cell homozygous for soybean event Gm_CSM63717. Detecting hybridization of both the first probe and the second probe under the hybridization conditions is diagnostic for a soybean plant, plant part, seed, or plant cell heterozygous for soybean event Gm_CSM63717. For example, the probe set can comprise SEQ ID NO:178 and SEQ ID NO:210.

DNA constructs are provided. One example of a DNA construct provided herein is a DNA construct comprising an expression cassette, wherein the expression cassette comprises in operable linkage i) a ubiquitin 2 (UBQ) promoter, a leader sequence, and an intron sequence from Medicago truncatula, ii) a codon optimized chloroplast transit peptide coding sequence of APG6 (Albino and Pale Green 6) from Adansonia digitata, iii) a codon-optimized protoporphyrinogen oxidase coding sequence from Enterobacter cloacae, and iv) a synthetic 3′ UTR sequence. For example, the DNA construct comprises SEQ ID NO:9. The DNA construct can further comprise at the 5′ or 3′ end of said construct: (a) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (b) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15.

Another DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9. The DNA construct comprises at the 5′ or 3′ end of said construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1.500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

Any of the DNA constructs can comprise at the 5′ end of said construct one or more nucleotide sequences selected from SEQ ID NOs:36-105. Any of the DNA constructs can comprise at the 3′ end of said construct one or more nucleotide sequences selected from SEQ ID NOs:106-175.

Methods for controlling or preventing weed growth in an area are provided. One example of such a method comprises planting soybean comprising event Gm_CSM63717 in the area and applying an effective amount of a PPO herbicide to control weeds in the area without injury to the soybean or with less than about 10% injury to the soybean.

Methods for controlling volunteer soybean comprising soybean event Gm_CSM63717 in an area are provided. One example of such a method comprises applying an herbicidally effective amount of at least one herbicide other than a PPO herbicide, wherein the herbicide application prevents growth of soybean comprising soybean event Gm_CSM63717. The herbicide other than a PPO herbicide can be selected from the group consisting of atrazine, topramezone, clopyralid, pyrithiobac, fluometuron, (3-(3,4-dichlorophenyl)-1,1-dimethylurea) (DCMU), trifloxysulfuron, paraquat, diquat, glyphosate, glufosinate, and combinations of any thereof.

Methods of obtaining a seed of a soybean plant or a soybean plant that is tolerant to PPO herbicides are provided. One example of such a method comprises: (a) obtaining a population of progeny seed or plants grown therefrom, at least one of which comprises soybean event Gm_CSM63717; and (b) identifying at least a first progeny seed or plant grown therefrom that comprises soybean event Gm_CSM63717. Identifying the progeny seed or plant grown therefrom that comprises soybean event Gm_CSM63717 can comprise: (a) growing the progeny seed or plant to produce progeny plants; (b) treating the progeny plants with an effective amount of a PPO herbicide; and (c) selecting a progeny plant that is tolerant to the PPO herbicide. Alternatively or in addition, identifying the progeny seed or plant grown therefrom that comprises soybean event Gm_CSM63717 can comprise detecting the presence of soybean event Gm_CSM63717 in a sample derived from the progeny seed or plant grown therefrom. Alternatively or in addition, identifying the progeny seed or plant grown therefrom that comprises soybean event Gm_CSM63717 comprises detecting the presence of the PPO protein encoded by soybean event Gm_CSM63717 in a sample derived from the progeny seed or plant grown therefrom.

Methods of improving tolerance to PPO herbicides in a soybean plant are provided. One example of such a method comprises: (a) inserting any of the DNA constructs described herein into the genome of a soybean cell; (b) generating a soybean plant from the soybean cell; and (c) selecting a soybean plant comprising the DNA construct. The selecting can comprise treating the soybean cell or plant with an effective amount of a PPO herbicide.

Soybean plants, plant seeds, plant parts, and plant cells comprising a recombinant DNA molecule. Are provided. The recombinant DNA molecule comprises a sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. The soybean plant, plant seed, plant part, or plant cell expresses a PPO herbicide tolerance gene. The soybean plant, plant seed, plant part, or plant cell is tolerant to one or more PPO herbicides.

Further soybean plants, plant seeds, plant parts, and plant cells are provided. The soybean plants, plant seeds, plant parts, or plant cells are tolerant to one or more PPO herbicides and comprise any of the DNA constructs described herein.

Any of the soybean plants, plant seeds, plant parts, or plant cells can further comprise at least one additional transgene for tolerance to at least one additional herbicide.

Any of the soybean plants, plant seeds, plant parts, or plant cells can comprise soybean event Gm_CSM63717, a representative sample of seed comprising the event having been deposited under ATCC Accession No. PTA-127604.

Any of the soybean plants, plant seeds, plant parts, or plant cells can be further defined as a progeny plant of any generation of a soybean plant comprising soybean event Gm_CSM63717, or a soybean plant part, plant seed, or plant cell derived therefrom.

A further soybean plant, plant part, plant seed, or plant cell is provided. The soybean plant, plant part, plant seed, or plant cell comprises soybean event Gm_CSM63717, a representative sample of seed comprising soybean event Gm_CSM63717 having been deposited under ATCC Accession No. PTA-127604.

The soybean plant part can comprise a microspore, pollen, an anther, an ovule, an ovary, a pod, a flower, an embryo, a stem, a bud, a node, a leaf, a root, or a callus.

Any of the soybean plants, plant seeds, plant parts, or plant cells can be obtained by any of the methods of obtaining a seed of a soybean plant or a soybean plant that is tolerant to PPO herbicides, or by any of the methods of improving tolerance to a PPO herbicide in a soybean plant described herein.

A further soybean plant, plant cell, plant part, or plant seed is provided. The soybean plant, plant cell, plant part, or plant seed comprises a recombinant DNA construct integrated in chromosome 13. The recombinant DNA construct confers tolerance to at least one PPO herbicide. The recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15. The at least 50 contiguous nucleotides of SEQ ID NO:11 or SEQ ID NO:14 can comprise one or more nucleotide sequences selected from SEQ ID NOs:36-105. The at least 50 contiguous nucleotides of SEQ ID NO:12 or SEQ ID NO:15 comprise one or more nucleotide sequences selected from SEQ ID NOs:106-175.

With respect to any of the soybean plants, plant cells, plant parts, or plant seeds tolerant to one or more PPO herbicides, any of the methods for controlling or preventing weed growth in an area comprising applying an effective amount of a PPO herbicide, any of the methods of obtaining a seed of a soybean plant or a soybean plant that is tolerant to PPO herbicides, and any of the methods of improving tolerance to PPO herbicides in a soybean plant, the PPO herbicide can be selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. The diphenylether can be selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. The N-phenylphthalimide can be selected from the group consisting of cinidon-ethyl, flumiclorac, flumiclorac-pentyl, and flumioxazin. The oxadiazole can be selected from the group consisting of oxadiargyl and oxadiazon. The oxazolidinedione can be pentoxazone. The phenylpyrazole can be selected from the group consisting of fluazolate, pyraflufen, and pyraflufen-ethyl. The pyrimidinedione can be selected from the group consisting of benzfendizone, butafenacil, epyrifencacil (S-3100), flupropacil, flufenoximacil, saflufenacil, and tiafenacil. The thiadiazole can be selected from the group consisting of fluthiacet-methyl and thidiazimin. The triazolinone can be selected from the group consisting of azafenidin, bencarbazone, carfentrazone, its salts and esters, and sulfentrazone. The benzoxazinone derivative can be 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)). The other PPO herbicide can be selected from the group consisting of chlorphthalim, flufenpyr, flufenpyr-ethyl, flumipropyn, pyraclonil, profluazol, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, and 2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate.

In any of the methods described herein involving use of an effective amount of a PPO herbicide, the effective amount of PPO herbicide can be about 0.0009 lb/acre to about 1.5 lb/acre over a growing season.

A method of producing a progeny soybean plant comprising soybean event Gm_CSM63717 is provided. The method comprises: (a) sexually crossing a first soybean plant that comprises soybean event Gm_CSM63717 with itself or a second soybean plant; (b) collecting one or more seeds produced from the cross; (c) growing one or more seeds to produce one or more progeny plants; and (d) selecting at least a first progeny plant or seed comprising soybean event Gm_CSM63717. Inbred or hybrid soybean plants or seed comprising soybean event Gm_CSM63717 that are produced by this method are also provided herein.

A nonliving or nonregenerable soybean plant material is provided. The nonliving or nonregenerable soybean plant material comprises any of the the recombinant DNA molecules provided herein or any of the DNA constructs provided herein.

Another nonliving or nonregenerable soybean plant material is provided. The nonliving or nonregenerable soybean plant material comprises soybean event Gm_CSM63717, a representative sample of seed comprising the soybean event soybean event Gm_CSM63717 having been deposited under ATCC Accession No. PTA-127604.

A commodity product is provided. The commodity product comprises any of the recombinant DNA molecules provided herein or any of the DNA constructs provided herein. The commodity product can be produced from a transgenic soybean plant, plant part, plant seed, or plant cell comprising soybean event Gm_CSM63717. The commodity product can comprise whole or processed seeds; viable or nonviable seeds; viable plant parts (such as roots, nodes, buds or leaves); viable plant cells; processed plant parts; processed plant tissues; dehydrated plant tissues; dehydrated plant parts; frozen plant tissues; frozen plant parts; food for human consumption such as soybean oil, soy milk, soy flour, soy grits, soy protein, soy protein concentrate, hydrolyzed vegetable protein, textured soy protein, lecithin, curd, tofu, vegetable soybean (edamame), soy sprouts, soy film (yuba), roasted soybeans, miso, tempeh, soy sauce, or natto; plant parts processed for animal feed such as soy meal; soy fiber; biodiesel; bio-composite building materials such as particleboard, laminated plywood, or lumber products; soy oil-based solvents; soy oil-based industrial lubricants; soy ink; soy candles; soy crayons; soy-based hydraulic fluid; or soy-based foams.

A method of producing a commodity product is provided. The method comprises: (a) obtaining a transgenic soybean plant, plant part, or plant seed comprising soybean event Gm_CSM63717; and (b) producing a commodity product from the transgenic soybean plant, plant part, or plant seed.

A method of controlling, preventing, or reducing the development of herbicide-tolerant weeds is provided. The method comprises cultivating in a crop growing environment a soybean plant comprising transgenes that provide tolerance to (i) a PPO herbicide and (ii) herbicides with at least three additional herbicide modes of action, the three additional herbicide modes of action each being different from one another.

Another method for controlling, preventing, or reducing the development of herbicide-tolerant weeds is provided. The method comprises: (a) cultivating in a crop growing environment a soybean plant comprising any of the DNA constructs described herein or event Gm_CSM63717, and at least three additional transgenes for providing tolerance to herbicides with at least three additional herbicide modes of action, the three additional herbicide modes of action each being different from one another, and (b) applying to the crop growing environment at least one herbicide selected from the group consisting of dicamba, glufosinate, 2,4-D, a PPO inhibitor, a P-triketone HPPD inhibitor, glyphosate, and any combination thereof, wherein the soybean plant is tolerant to the at least one herbicide.

In any of the methods for controlling, preventing, or reducing the development of herbicide-tolerant weeds, the transgenes that provide tolerance to the herbicides with the at least three additional herbicide modes of action can be present at a single genomic location in the soybean plant.

Also provided are methods of reducing loci for soybean breeding by site-directed insertion of a transgene that provides tolerance to a PPO herbicide at a genomic location in a soybean plant that is within about 1-6 cM of a locus in the genome of the soybean plant that comprises transgenes for tolerance to at least three additional herbicide modes of action, the three additional herbicide modes of action each being different from one another.

In any of the soybean plants, plant seeds, plant parts, or plant cells described herein that comprise at least one additional transgene for tolerance to at least one additional herbicide, and in any of the methods for controlling, preventing, or reducing the development of herbicide tolerant weeds or methods for reducing loci for soybean breeding, the additional transgenes can be selected from the group consisting of FT_Tv7, dicamba monooxygenase (DMO), phosphinothricin N-acetyltransferase (PAT), triketone dioxygenase (TDO), 5-enolpyruvylshikimate-3-phosphate synthase (EPSPS), and combinations of any thereof. For example, the FT_Tv7 transgene can comprise a polynucleotide sequence encoding a protein having the amino acid sequence of SEQ ID NO:31. The DMO transgene can comprise a polynucleotide sequence encoding a protein having the amino acid sequence of SEQ ID NO:29. The PAT transgene can comprise a polynucleotide sequence encoding a protein having the amino acid sequence of SEQ ID NO:27. The TDO transgene can comprise a polynucleotide sequence encoding a protein having the amino acid sequence of SEQ ID NO: 33. The EPSPS transgene can comprise a polynucleotide sequence encoding a protein having the amino acid sequence of SEQ ID NO:35. The additional transgenes can provide tolerance to herbicides having modes of action selected from the group consisting of inhibitors of glutamine synthetase, inhibitors of EPSPS, β-triketone HPPD inhibitors, synthetic auxins, and combinations of any thereof. The synthetic auxin can be selected from the group consisting of dicamba, 2,4-D, dichlorprop, mecoprop, 2,4,5-T (2,4,5-trichlorophenoxyacetic acid), and combinations of any thereof. The inhibitor of glutamine synthetase can comprise glufosinate. The β-triketone HPPD inhibitor can be selected from the group consisting of mesotrione, benzobicyclon (BBC), tembotrione, sulcotrione, tefuryltrione, and combinations of any thereof. The inhibitor of 5-enolpyruvylshikimate-3-phosphate synthase (EPSPS) can comprise glyphosate.

In any of the soybean plants, plant seeds, plant parts, plant cells described herein that comprise at least one additional transgene for tolerance to at least one additional herbicide, and in any of the methods for controlling, preventing, or reducing the development of herbicide-tolerant weeds or any of the methods for reducing loci for soybean breeding, the soybean plant, plant seed, plant part, or plant cell can further comprise soybean event MON89788 and/or soybean event Gm_CSM63714.

In any of the soybean plants, plant seeds, plant parts, plant cells described herein that comprise at least one additional transgene for tolerance to at least one additional herbicide, and in any of the methods for controlling, preventing, or reducing the development of herbicide-tolerant weeds or any of the methods for reducing loci for soybean breeding, the soybean plant, plant seed, plant part, or plant cell can further comprise a recombinant DNA molecule comprising a sequence selected from the group consisting of SEQ ID NO:182; SEQ ID NO:183; SEQ ID NO:184; SEQ ID NO:185; SEQ ID NO: 186; SEQ ID NO:187; SEQ ID NO:188; SEQ ID NO:189; SEQ ID NO:190; SEQ ID NO:191; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:182 or the full length of SEQ ID NO: 183; and a complete complement of any of the foregoing.

In any of the soybean plants, plant seeds, plant parts, plant cells described herein that comprise at least one additional transgene for tolerance to at least one additional herbicide, and in any of the methods for controlling, preventing, or reducing the development of herbicide-tolerant weeds or any of the methods for reducing loci for soybean breeding, the soybean plant, plant seed, plant part, or plant cell can further comprise a recombinant DNA molecule comprising a sequence selected from the group consisting of SEQ ID NO:192, SEQ ID NO:193, SEQ ID NO:194, SEQ ID NO:195, SEQ ID NO:196, SEQ ID NO:197; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:192 or the full length of SEQ ID NO:193; and a complete complement of any of the foregoing.

A further method for controlling or preventing weed growth in an area is provided. The method comprises planting soybean comprising event Gm_CSM63717, event Gm_CSM63714 and/or event MON89788 in the area and applying one or more herbicides selected from the group consisting of a PPO herbicide, dicamba, glufosinate, 2,4-D, β-triketone HPPD inhibitor, glyphosate, and combinations of any thereof to control weeds in the area without injury or with less than 10% injury to the soybean.

### BRIEF DESCRIPTION OF THE DRAWINGS

FIG. 1 illustrates the sequence of the soybean event Gm_CSM63717. Horizontal lines correspond to the positions of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:11, and SEQ ID NO:12 relative to SEQ ID NO:10. The horizontal arrows (SEQ ID NO:176, SEQ ID NO:177, SEQ ID NO:208, and SEQ ID NO:209) represent the approximate positions of illustrative primer pairs that can be used to detect soybean event Gm_CSM63717. The horizontal lines labeled SEQ ID NO:178 and SEQ ID NO:210 represent the approximate positions of illustrative DNA probes that can be used to detect soybean event Gm_CSM63717 or wildtype soybean sequence. “RB” refers to the Agrobacteriun T-DNA right border; “LB” refers to the Agrobacterium T-DNA left border. “Promoter” represents a promoter element; “Leader” represents a leader (5′ UTR) element; “Intron” represents an intron element; “CTP” represents a chloroplast transit peptide element; “3′ UTR” represents a 3′ UTR; “PPO” represents a protoporphyrinogen oxidase coding element; and “Lox” represents a lox recombination site. The horizontal line labeled SEQ ID NO:13 represents the relative location or position of the wildtype soybean genome where the transgene (SEQ ID NO:9) was inserted. The dashed line represents a 10-nucleotide deletion in the soybean genome at the site of transgene (SEQ ID NO:9) insertion.

FIG. 2 is a diagrammatic representation of the T-DNA cassettes in the Agrobacterium Ti plasmid used to transform and generate soybean event Gm_CSM63717 before and after T-DNA integration, and after Cre-mediated excision of the marker cassettes. “RB” refers to the Agrobacterium T-DNA right border; “LB” refers to the Agrobacterium T-DNA left border. “aadA”, “CRE”, “Cas12a”, “PPO”, and “gRNA” represent the aadA selectable marker cassette, the Cre recombinase cassette, the Cas12a nuclease cassette, the protoporphyrinogen oxidase cassette, and the gRNA cassette, respectively. “Lox” represents a lox recombination site. “5′ Flank” and “3′ Flank” represent the 5′ and 3′ flanking soybean genomic sequences at the site of T-DNA integration, respectively. A: “T-DNA Before Integration” represents T-DNA comprising the aadA, CRE. Cas12a, gRNA and PPO cassettes before transformation; B: “Inserted T-DNA After Integration” represents T-DNA comprising the aadA, CRE, Cas12a, gRNA and PPO cassettes integrated into the soybean genome after transformation; C: “Inserted T-DNA After Cre-Excision” represents the integrated T-DNA cassette after the aadA, CRE, Cas12a, and gRNA cassettes were excised, leaving behind one of the two Lox sites, the PPO cassette and the left and right borders.

FIG. 3 illustrates the approximate timelines for the research, testing, and development, leading to selection of the commercial event soybean event Gm_CSM63717. “POC” stands for Proof of Concept; “TFN” strands for Transformation; “GH” stands for Greenhouse; “SA” stands for South America; “NA” stands for North America.

FIG. 4 is a diagrammatic representation of the breeding process to produce marker-free soybean events. “R₀ Transformants” are the initial transgenic events generated by transformation with the binary transformation vector, which were hemizygous for the T-DNA allele comprising the aadA, Cre, Cas12a, gRNA and PPO cassettes. The R₀ transformants were self-pollinated to produce the _{R1} generation, wherein many of the progeny lost the aadA, Cre, Cas12a and gRNA cassettes flanked by the two Lox sites due to Cre-recombinase excision. Hemizygous T-DNA positive, aadA, Cre, Cas12a and gRNA negative (also referred to as “marker-free”) plants were selected and self-pollinated, resulting in a R₂ generation. R₂ plants homozygous for the inserted T-DNA allele without the aadA, CRE, Cas12a, gRNA cassettes were selected and self-pollinated giving rise to a R₃ generation. The R₃ generation plants were self-pollinated to produce a pure line of R₄ Gold Standard Seed. The R₃ generation plants which were marker-free and homozygous for the inserted T-DNA were used for inbred efficacy and agronomic field trials. Subsequent “R” generations (R₁, R₂, and R₃) represent successive generations produced through self-pollination of plants derived from the initial R₀ transformant that resulted in the soybean event Gm_CSM63717.

FIG. 5 shows the yield results of the seven targeted events of construct GmHT5-1 and GmHT5-2 in the fourth season agronomic field trials conducted in South America. The yield is presented as % Yield Change compared to the wild-type control.

FIG. 6 shows the yield results of the seven targeted events of construct GmHT5-1 and GmHT5-2 in the fourth season efficacy field trials conducted in South America. The yield is presented as % Yield Change compared to the untreated control. Herbicide treatments include 1) flumioxazin at 448 g/ha applied at pre-emergence (PRE), followed by V3 and R₁ stages (Upper Panel); and 2) epyrifenacil (rapidicil) at 80 g/ha applied at PRE, followed by V3 and R₁ stages (Lower Panel).

FIG. 7 shows the injury ratings of the seven targeted events of construct GmHT5-1 and GmHT5-2 in the fourth season efficacy field trials conducted in South America. The injury rating is presented as % Injury compared to the untreated control. Herbicide treatments include 1) flumioxazin at 448 g/ha applied at pre-emergence (PRE), followed by V3 and R₁ stages (Upper Panel); and 2) epyrifenacil (rapidicil) at 80 g/ha applied at PRE, followed by V3 and R1 stages (Lower Panel).

### BRIEF DESCRIPTION OF THE SEQUENCES

SEQ ID NO:1 is a 30-nucleotide sequence representing the 5′ junction region of the soybean genomic DNA and the integrated transgene insert. SEQ ID NO:1 corresponds to nucleotide positions 986-1,015 of SEQ ID NO:10.

SEQ ID NO:2 is a 30-nucleotide sequence representing the 3′ junction region of the integrated transgene insert and the soybean genomic DNA. SEQ ID NO:2 corresponds to nucleotide positions 4,187-4,216 of SEQ ID NO:10.

SEQ ID NO:3 is a 60-nucleotide sequence representing the 5′ junction region of the soybean genomic DNA and the integrated transgene insert. SEQ ID NO:3 corresponds to nucleotide positions 971-1,030 of SEQ ID NO:10.

SEQ ID NO:4 is a 60-nucleotide sequence representing the 3′ junction region of the integrated transgene insert and the soybean genomic DNA. SEQ ID NO:4 corresponds to nucleotide positions 4,172-4,231 of SEQ ID NO:10.

SEQ ID NO:5 is a 100-nucleotide sequence representing the 5′ junction region of the soybean genomic DNA and the integrated transgene insert. SEQ ID NO:5 corresponds to nucleotide positions 951-1,050 of SEQ ID NO:10.

SEQ ID NO:6 is a 100-nucleotide sequence representing the 3′ junction region of the integrated transgene insert and the soybean genomic DNA. SEQ ID NO:6 corresponds to nucleotide positions 4,152-4,251 of SEQ ID NO:10.

SEQ ID NO:7 is a 1,050-nucleotide sequence representing the 5′ genomic flank region of the soybean genomic DNA and 50 bp of the integrated transgene insert. SEQ ID NO:7 corresponds to nucleotide positions 1-1,050 of SEQ ID NO:10.

SEQ ID NO:8 is a 1,050-nucleotide sequence representing 50 bp of the 3′ junction region of the integrated transgene insert and the 3′ genomic flank region of the soybean genomic DNA. SEQ ID NO:8 corresponds to nucleotide positions 4,152-5,201 of SEQ ID NO:10.

SEQ ID NO:9 is a 3,201-nucleotide sequence corresponding to the transgene insert of soybean event Gm_CSM63717. SEQ ID NO:9 corresponds to nucleotide positions 1,001-4,201 of SEQ ID NO:10.

SEQ ID NO:10 is a 5,201-nucleotide sequence corresponding to the contig nucleotide sequence of the 5′ soybean genomic DNA sequence (SEQ ID NO: 11), the transgene insert in event Gm_CSM63717 (SEQ ID NO:9), and the 3′ soybean genomic DNA sequence (SEQ ID NO:12).

SEQ ID NO:11 is a 1,000-nucleotide sequence representing the 5′ flanking soybean genomic DNA up to the transgene insert (SEQ ID NO:9). SEQ ID NO:11 corresponds to nucleotide positions 1-1,000 of SEQ ID NO:10.

SEQ ID NO:12 is a 1,000-nucleotide sequence representing the 3′ flanking soybean genomic DNA after the transgene insert (SEQ ID NO:9). SEQ ID NO:12 corresponds to nucleotide positions 4,202-5,201 of SEQ ID NO:10.

SEQ ID NO:13 is a 2,010-nucleotide sequence representing wildtype soybean genomic DNA at the location where the transgenic sequence (SEQ ID NO:9) was inserted in event Gm_CSM63717. A 10-nucleotide fragment of SEQ ID NO:13 (nucleotides 1,001-1,010) was deleted in event Gm_CSM63717 due to insertion of the T-DNA.

SEQ ID NO:14 is a 5,000-nucleotide sequence representing soybean genomic DNA that flanks the transgenic insert at the 5′ end of the insert. The sequence was based on the genomic sequence of soybean Williams 82 germplasm.

SEQ ID NO:15 is a 5.000-nucleotide sequence representing soybean genomic DNA that flanks the transgenic insert at the 3′ end of the insert. The sequence was based on the genomic sequence of soybean Williams 82 germplasm.

SEQ ID NOs:16-24 are the nucleotide sequences for the genetic elements in the transgenic insert of soybean event Gm_CSM63717 and are further described in Table 1 hereinbelow.

SEQ ID NO:25 is the amino acid sequence of the protoporphyrinogen oxidase (PPO) from Enterobacter cloacae.

SEQ ID NOs:26 and 27 are the codon-optimized nucleotide and amino acid sequences of the phosphinothricin N-acetyltransferase gene (PAT), respectively.

SEQ ID NOs:28 and 29 are the codon-optimized nucleotide and amino acid sequences of the dicamba monooxygenase (DMO) gene, respectively.

SEQ ID NOs:30 and 31 are the codon-optimized nucleotide and amino acid sequences of the FT_Tv7 gene, respectively.

SEQ ID NOs:32 and 33 are the codon-optimized nucleotide and amino acid sequences of the triketone dioxygenase (TDO) gene, respectively.

SEQ ID NOs:34 and 35 are the codon-optimized nucleotide and amino acid sequences of the 5-enolpyruvylshikimate-3-phosphate synthase (EPSPS) gene, respectively.

SEQ ID NOs:36-105 are 50-nucleotide sequences in the 5′ flank genomic sequence of event Gm_CSM63717. SEQ ID NOs:36-55 are based on the genomic sequence of the transformation germplasm; SEQ ID NOs:56-105 are based on the genomic sequence of soybean Williams 82 germplasm.

SEQ ID NOs:106-175 are 50-nucleotide sequences in the 3′ flank genomic sequence of event Gm_CSM63717. SEQ ID NOs:106-125 are based on the genomic sequence of the transformation germplasm; SEQ ID NOs:126-175 are based on the genomic sequence of soybean Williams 82 germplasm.

SEQ ID NO:176 is a 22-nucleotide sequence corresponding to a thermal amplification primer referred to as SQ51702 used in event-specific assay and zygosity assay to detect soybean event Gm_CSM63717 DNA in a sample and is identical to the reverse complement of the nucleotide sequence corresponding to positions 1,068-1,089 of SEQ ID NO:10.

SEQ ID NO:177 is a 32-nucleotide sequence corresponding to a thermal amplification primer referred to as SQ52020 used in event-specific assay and zygosity assay to detect soybean event Gm_CSM63717 DNA in a sample and is identical to the nucleotide sequence corresponding to positions 931-962 of SEQ ID NO:10.

SEQ ID NO:178 is an 18-nucleotide sequence corresponding to a 6FAM-MGB probe referred to as PB50308 used in event-specific assay and zygosity assay to detect soybean event Gm_CSM63717 DNA in a sample and is identical to the reverse complement of the nucleotide sequence corresponding to positions 1,045-1,062 of SEQ ID NO:10.

SEQ ID NO:179 is a 20-nucleotide sequence corresponding to a thermal amplification primer referred to as SQ546 used as an internal control for the event assay for soybean event Gm_CSM63717 and hybridizes to a region of the soybean genome.

SEQ ID NO:180 is a 20-nucleotide sequence corresponding to a thermal amplification primer referred to as SQ549 used as an internal control for the event assay for soybean event Gm_CSM63717 and hybridizes to a region of the soybean genome.

SEQ ID NO:181 is an 18-nucleotide sequence corresponding to a VIC-MGB probe referred to as PB50207 used as an internal control for the event assay for soybean event Gm_CSM63717 and hybridizes to a region of the soybean genome.

SEQ ID NO:182 is the nucleotide sequence of soybean event Gm_CSM63714 corresponding to the contig nucleotide sequence of 5′ flanking soybean genomic sequence, transgenic insert, and 3′ flanking soybean genomic sequence.

SEQ ID NO:183 is the nucleotide sequence of the transgenic insert in soybean event Gm_CSM63714.

SEQ ID NOs:184-187 are 5′ junction sequences of soybean event Gm_CSM63714.

SEQ ID NOs:188-191 are 3′ junction sequences of soybean event Gm_CSM63714.

SEQ ID NO:192 is a 6466-nucleotide sequence of soybean event MON89788 corresponding to the contig nucleotide sequence of 5′ flanking soybean genomic sequence, transgenic insert, and 3′ flanking soybean genomic sequence.

SEQ ID NO:193 is the nucleotide sequence of the transgenic insert in soybean event MON89788 (nucleotides 1104-5409 of SEQ ID NO:9 of U.S. Pat. No. 7,632,985).

SEQ ID NOs:194-195 are 5′ junction sequences of soybean event MON89788.

SEQ ID NOs:196-197 are 3′ junction sequences of soybean event MON89788.

SEQ ID NOs:198 and 199 are the nucleotide and amino acid sequences of LbCas12a (also known as LbCpf1) of Lachnospiraceae bacterium ND2006, respectively.

SEQ ID NO:200 is the amino acid sequence for LbCas12a_V1 (G532R/K595R).

SEQ ID NO:201 is the amino acid sequence for LbCas12a_V2 (G532R/K538V/Y542R).

SEQ ID NO:202 is the amino acid sequence for Cas12a of Francisella novicida (FnCas12a).

SEQ ID NO:203 is the nucleotide sequence for the gRNA repeat for LbCas12a.

SEQ ID NO:204 is the nucleotide sequence for the gRNA repeat for FnCas12a.

SEQ ID NO:205 is the nucleotide sequence for the gRNA gRNA_5F-134.

SEQ ID NO:206 is the nucleotide sequence for the gRNA gRNA_3F-15.

SEQ ID NO:207 is a 21-nucleotide downstream mature crRNA scaffold sequence.

SEQ ID NO:208 is a 30-nucleotide sequence corresponding to a thermal amplification forward primer referred to as SQ52185 used in a zygosity assay for detection of the wildtype (WT) allele DNA in a sample and hybridizes to a region of the soybean genome. It is identical to the reverse complement of the nucleotide sequence corresponding to positions 4,220-4,249 of SEQ ID NO:10.

SEQ ID NO:209 is a 27-nucleotide sequence corresponding to a thermal amplification primer referred to as SQ52186 used in a zygosity assay for detection of the WT allele DNA in a sample and hybridizes to a region of the soybean genome. It corresponds to positions 941-967 of SEQ ID NO:10.

SEQ ID NO:210 is an 18-nucleotide sequence corresponding to a VIC-MGB probe referred to as PB50725 used in a zygosity assay for detection of the WT allele DNA in a sample and hybridizes to a region of the soybean genome. It corresponds to positions 4,201-4,218 of SEQ ID NO:10.

SEQ ID NOs:211-222 are the nucleotide sequences of gRNA target sites.

SEQ ID NOs:223-234 are the nucleotide sequences of the gRNA spacer sequences corresponding to SEQ ID NOs:211-222.

SEQ ID NO:235 is the nucleotide sequence of a nuclear localization signal (NLS) from tomato heat stress transcription factor HSFA1.

SEQ ID NO:236 is the nucleotide sequence of a plant codon optimized sequence encoding LbCas12a used for gRNA testing.

SEQ ID NO:237 is the 3′ UTR sequence derived from a Medicago truncatula gene.

SEQ ID NO:238 is the nucleotide sequence of an enhancer region from the Banana Streak Virus Strain Acuminata Vietnam plus the promoter and 5′ UTR region from the Dahlia mosaic virus.

SEQ ID NO:239 is a 20-nucleotide direct repeat or mature crRNA scaffold sequence.

### DETAILED DESCRIPTION

The following definitions, descriptions, and methods are provided to better define the invention and to guide those of ordinary skill in the art in the practice of the invention. Unless otherwise noted, terms are to be understood according to conventional usage by those of ordinary skill in the relevant art.

Herbicide tolerance is an important agronomic trait for effective weed control to maintain favorable crop growing conditions and crop yields and is achieved by engineering of herbicide tolerance transgenes in crop plants using modern plant biotechnology techniques. Soybean event Gm_CSM63717 confers tolerance to PPO herbicides and provides another mode of action for weed control and herbicide-resistant weed management.

Soybean event Gm_CSM63717 is provided. The event Gm_CSM63717 was produced by Agrobacterium-mediated transformation of soybean seed-derived embryo explants with a DNA construct comprising five transgene cassettes. The first cassette encoded a protoporphyrinogen oxidase (PPO) from Enterobacter cloacae for conferring tolerance to PPO herbicides. The second cassette encoded a Tn7 aminoglycoside-3′-adenylyltransferase (aadA) from E. coli as a selectable marker for selection of transgenic events. The third cassette encoded a codon optimized Cas12a nuclease from Lachnospiraceae bacterium ND2006 (LbCas12a or LbCpf1) for site directed integration of the transgenes. The fourth cassette encoded a gRNA for directing the Cas12a nuclease to its soybean genomic target region. The fifth cassette encoded a Cre recombinase from Escherichia virus P1. The aadA, Cas12a, Cre and gRNA cassettes were flanked by two lox sites and were removed upon expression of the Cre enzyme.

Plant transformation techniques, such as Agrobacterium-mediated or biolistic transformation, can be used to insert foreign DNA (also known as transgenic DNA) randomly or by site directed insertion (also known as targeted insertion) into a chromosome in a plant cell to produce a genetically engineered plant cell, also referred to as a “transgenic” or “recombinant” cell. Using these transformation techniques, many individual cells can be transformed, each resulting in a unique “transgenic event” or “event” due to insertion of the foreign DNA into the genome. A transgenic plant can then be regenerated from each individual transgenic cell. This results in every cell of the transgenic plant containing the uniquely inserted transgenic event as a stable part of its genome. The transgenic plant can then be used to produce progeny plants, each containing the unique transgenic event. The term “transgenic” refers to a plant, plant part, plant cell, seed, progeny plant, or DNA molecule, construct, or sequence comprising a transgene—e.g., a “transgenic cell” refers to a cell comprising a transgene.

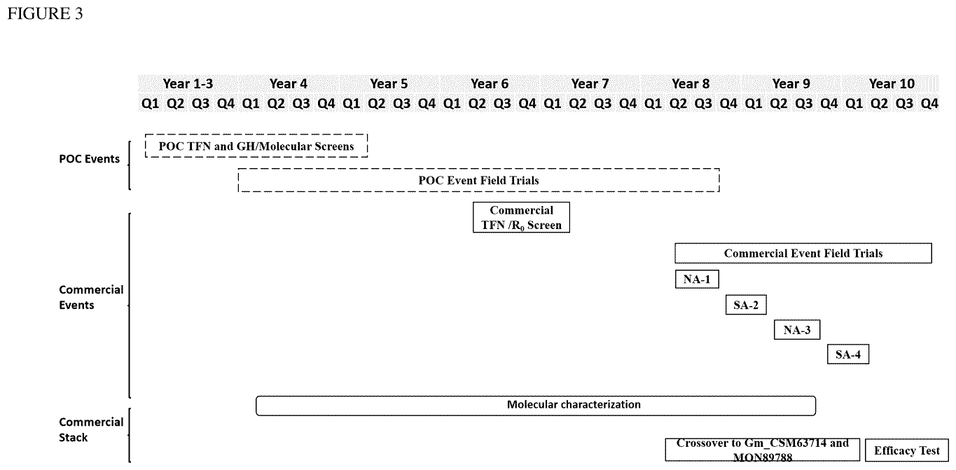

Soybean event Gm_CSM63717 was produced and identified by a complex research and development process. This process included: (i) design and selection of DNA constructs comprising the five transgene cassettes based on design and testing of individual transgene cassettes with combinations of different expression elements; (ii) identification and screening of different transgene target sites, followed by design and testing of different guide RNAs for efficient cutting at the target sites; (iii) transformation of thousands of soybean cells with the DNA constructs; (iv) regeneration of a large population of transgenic events; and (v) rigorous multi-year construct and event selection involving molecular characterization of the large number of transgenic events, greenhouse and field trials for herbicide tolerance efficacy and agronomic performance at different locations and in different geographies. Soybean event Gm_CSM63717 was thus produced and selected as a uniquely superior event useful for broad-scale agronomic commercial purposes. FIG. 3 illustrates the approximate timelines for the research, testing and development, leading to selection of the commercial event soybean event Gm_CSM63717.

Detailed molecular characterization was conducted on the transgenic events. Event Gm_CSM63717 was selected based on stringent molecular criteria, as well as other selection criteria such as herbicide tolerance efficacy and agronomic performance. The results from such molecular analyses confirmed that: (1) event Gm_CSM63717 contains a single T-DNA inserted at the targeted location, with one copy of the transgenic insert comprising only the PPO cassette; (2) no additional elements from the transformation construct were present other than the PPO expression cassette and one lox site between the left and right borders of the T-DNA, such as the transformation construct backbone sequence or the aadA, Cas12a, CRE and gRNA cassettes or fragment(s); (3) the transgenic DNA was inserted in an intergenic region, far away from any endogenous genes or repeat regions; and (4) the transgenic event produced the correct sized transcript and protein for the PPO transgene by Northern hybridization and Western hybridization analyses, respectively. Furthermore, DNA sequence analyses and protein expression assays were performed to: (1) determine the 5′ and 3′ transgenic insert-to-plant genome junctions; (2) confirm the organization of the elements within the insert; (3) verify the complete nucleotide sequence of the inserted transgenic DNA (SEQ ID NO:9); and (4) determine the PPO protein levels in different tissues such as leaf, root, and seed, and PPO protein levels in leaves over multiple generations. In addition, primers and probes were designed, and thermal amplification assays were developed and verified for producing specific amplicons diagnostic for the presence of event Gm_CSM63717 in a sample. As used herein, the 5′ and 3′ designations in reference to the junction, direction and site of the transgenic event insertion is relative to the right border to left border direction of the inserted T-DNA, with the 5′ junction and genomic sequence being upstream of the right border and transgene, and the 3′ junction and genomic sequence being downstream of the left border and transgene.

As used herein, an “expression cassette” or “cassette” or “transgene cassette” is a recombinant DNA molecule or sequence comprising a combination of distinct elements for expressing an RNA and/or protein encoded by the coding sequence of a transgene in a transformed plant cell or transformed plant comprising the transgene. As provided herein, an “expression cassette” or “cassette” or “transgene cassette” includes one or more regulatory element(s) operably linked to a coding or transcribable DNA sequence. The regulatory elements can include a promoter, a leader, a 5′ untranslated region (5′ UTR), an intron and/or a 3′ untranslated region (3′ UTR). The “expression cassette” or “cassette” or “transgene cassette” is recombinant and heterologous with respect to the transformed plant cell genome, as well as to the combination of the different genetic elements. For purposes of the present disclosure, such an “expression cassette” or “cassette” or “transgene cassette” is a recombinant DNA molecule or sequence that encodes a protein for conferring tolerance to at least one class of herbicides as described herein. Table 1 provides a list of the genetic elements contained in the transgene cassette in the transgenic insert (SEQ ID NO:9) of soybean event Gm_CSM63717.

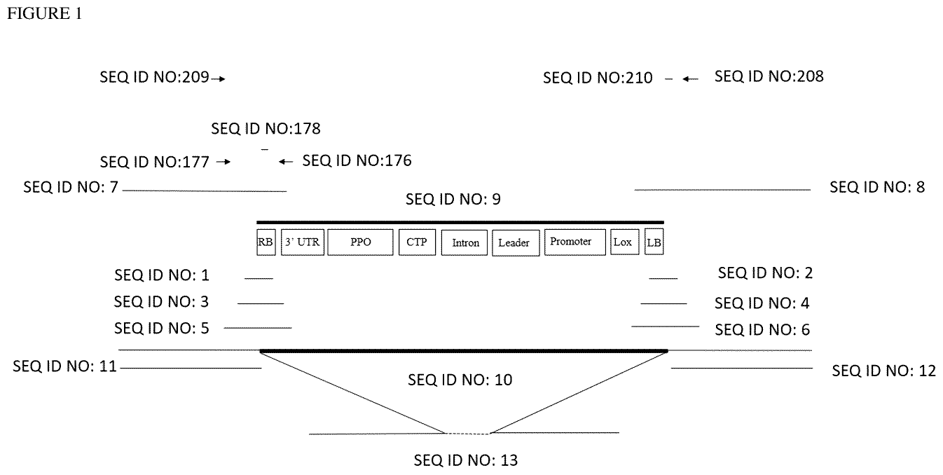

Insertion of the transgenic DNA into the genome of the soybean plant is accomplished by plant transformation methods known in the art and creates a new transgenic genomic DNA sequence, known as a “transgenic event” or an “event” or a “transgenic event locus.” The DNA sequence of the event consists of the inserted foreign DNA (referred to as the “transgenic insert”) and the genomic DNA adjacent to, or “flanking” the transgenic insert on either side of the insertion location. As used herein, the term “flanking” in reference to a transgenic event refers to the plant genomic sequence(s) adjacent to the transgenic DNA insertion in the genome of the transformed plant, plant part, plant tissue, or plant cell comprising the transgenic event on the 5′ and/or 3′ end(s) of the transgenic event insertion. Likewise. “flanking DNA” refers to a length of genomic DNA sequence adjacent to the transgenic DNA insertion in the genome of the transformed event on the 5′ and/or 3′ end(s) of the insertion. A “5′ flank”, therefore, means the soybean genomic DNA sequence adjacent to and upstream (or on the 5′ end) of the transgenic DNA insertion. For example, a “5′ flank” can include the soybean genomic DNA sequence immediately adjacent to and upstream (on the 5′ end) of the transgenic insertion, or any soybean genomic DNA sequence upstream (on the 5′ end) of the transgenic insertion that is not immediately adjacent to the transgenic insertion but is within about 5000 nucleotides, within about 3000 nucleotides, or within about 1000 nucleotides upstream of the transgenic insertion. Likewise, a “3′ flank” means the soybean genomic DNA sequence adjacent to and downstream (or on the 3′ end) of the transgenic insert. For example, a “3′ flank” can include the soybean genomic DNA sequence immediately adjacent to and downstream (on the 3′ end) of the transgenic insertion, or any soybean genomic DNA sequence downstream (on the 3′ end) of the transgenic insertion that is not immediately adjacent to the transgenic insertion but is within about 5000 nucleotides, within about 3000 nucleotides, or within about 1000 nucleotides downstream of the transgenic insertion. The DNA sequence of an event is unique to and specific for the event and can be readily identified when compared to other DNA sequences, such as that of other events or untransformed soybean genomic DNA. Soybean event Gm_CSM63717 has the new and unique DNA sequence provided as SEQ ID NO:10, which comprises a contiguous sequence comprising the 5′ soybean genomic flanking sequence provided as SEQ ID NO:11, the transgenic insert sequence provided as SEQ ID NO:9, and the 3′ soybean genomic flanking sequence provided as SEQ ID NO:12 (FIG. 1). Soybean event Gm_CSM63717 is thus a DNA molecule that is an integral part of the chromosome of transgenic soybean cells and plants comprising the event and as such is static and may be passed on to progeny cells and plants. As is described further in the Examples hereinbelow, various gene editing tools exist that would permit modification of the transgenic insert and/or the flanking genomic DNA of soybean event Gm_CSM63717, such as by deletion, insertion, transposition, or substitution of nucleic acid sequence(s); the event is still uniquely characterized by the presence of heterologous DNA at the particular position in the genome occupied by soybean event Gm_CSM63717 relative to flanking portions of the native soybean genome.

TABLE 1
Elements and Description of Soybean Event Gm_CSM63717

| | SEQ | Position in | |
|---|---|---|---|
| | ID | SEQ ID | |
| Element | NO | NO: 10 | Description |
| 5′ Flank sequence | 11 | 1-1000 | Soybean genomic DNA sequence flanking the 5′ end |
| | | | of the transgenic insert |
| Right border region | 24 | 1001-1290 | Right border sequence from Agrobacterium |
| | | | tumefaciens. |
| | | | SEQ ID NO: 24 is the full-length, 505-nucleotide T- |
| | | | DNA right border sequence. However, since a portion |
| | | | of this sequence was truncated at the time of the T- |
| | | | DNA insertion into the soybean genomic DNA, only |
| | | | nucleotides 216-505 of SEQ ID NO: 24 are present in |
| | | | SEQ ID NO: 10. |
| Intervening sequence 1 | — | 1291-1331 | Sequence used in DNA cloning, |
| T-Zm.GST314 | 23 | 1332-1731 | A synthetic 3′ UTR sequence. |
| Intervening sequence 2 | — | 1732-1744 | Sequence used in DNA cloning. |
| CR-ENTcl.PPO_H_N90 | 22 | 1745-2281 | Codon-optimized coding sequence of a |
| | | | protoporphyrinogen oxidase (PPO) from |
| | | | Enterobacter cloacae. |
| TS- ADAdi.APG6 | 21 | 2282-2515 | Codon-optimized chloroplast transit peptide (CTP) |
| | | | coding sequence of APG6 (Albino and Pale Green 6) |
| | | | from Adansonia digitata. |
| Intervening sequence 3 | — | 2516-2529 | Sequence used in DNA cloning. |
| I- Mt.Ubq2 | 20 | 2530-3031 | Intron sequence of a ubiquitin gene 2 from |
| | | | Medicago truncatula. |
| L- Mt.Ubq2 | 19 | 3032-3113 | 5′ UTR leader sequence of a ubiquitin gene 2 from |
| | | | Medicago truncatula. |
| P-Mt.Ubq2 | 18 | 3114-4123 | Promoter sequence of a ubiquitin 2 gene from |
| | | | Medicago truncatula. |
| RS-P1.lox | 17 | 4124-4157 | Lox recombination site. |
| Intervening sequence 4 | — | 4158-4190 | Sequence used in DNA cloning, |
| Left border region | 16 | 4191-4201 | Left border sequence from Agrobacterium |
| | | | tumefaciens. |
| | | | SEQ ID NO: 16 is the 442-nucleotide, full-length |
| | | | T-DNA left border sequence. A portion of this |
| | | | sequence was truncated during T-DNA insertion into |
| | | | the soybean genomic DNA. Only nucleotides 1-11 |
| | | | of SEQ ID NO: 16 are present in SEQ ID NO: 10. |
| 3′ Flank sequence | 12 | 4202-5201 | Soybean genomic DNA sequence flanking the 3′ end |
| | | | of the transgenic insert. |

Progeny of the original transformed cell and plant that comprise soybean event Gm_CSM63717 are provided. Such progeny may be produced by selfing of a soybean plant comprising the soybean event Gm_CSM63717, or by sexual cross or outcrossing between a soybean plant comprising soybean event Gm_CSM63717 and another plant that does or does not contain the event, or by any other method known in the art including any plant cell or tissue culture method, wherein the progeny includes the soybean event Gm_CSM63717. The other plant may be a transgenic plant comprising the same and/or different event(s) or may be a non-transgenic plant, and each parental plant in a cross or outcross may be the same or different germplasm or breeding line. Soybean event Gm_CSM63717 is passed from the original parent through each generation to the progeny. A “transgenic plant” or “plant”, therefore, can be the original transformant plant regenerated from the transformed plant cell and comprising the transgenic DNA and event, or a progeny plant of the original transformant plant, which may be separated from the transformant by one or more generations, that retains the transgenic DNA and event at the same specific location and sequence context in the plant's genome. The transformant or progeny plant may be homozygous or heterozygous for event Gm_CSM63717. In addition, a “transgenic plant” may comprise a plant having the transgenes stably inserted into the genome of at least one cell of the plant (i.e., soybean event Gm_CSM63717 in at least one cell of the plant), and the plant may be chimeric or non-chimeric with respect to the transgenes and/or event. A transgenic plant is chimeric with respect to a transgene if not all cells of the plant comprise the transgenes.

The present disclosure describes introduction of event Gm_CSM63717 into soybean, and thus the term “soybean event Gm_CSM63717” is used to refer to the event herein. However, those of skill in the art will understand that event Gm_CSM63717 could be introduced into other varieties or related soybean species by crosses, such as Glycine soja.

Soybean event Gm_CSM63717 provides to soybean cells, plants, plant parts, seeds and progeny that comprise the event tolerance to PPO herbicides. The terms “PPO herbicide”, “PPO inhibitor”, and “PPO-inhibiting herbicide” are used interchangeably herein and refer to chemical agents that target and inhibit the enzymatic activity of a protoporphyrinogen oxidase (PPO). Soybean event Gm_CSM63717 provides tolerance to various PPO herbicides, including, but not limited to, flumioxazin, epyrifenacil (also referred to as S-3100 or rapidicil; IU PAC name: ethyl [(3-{(2-chloro-5-[3,6-dihydro-3-methyl-2,6-dioxo-4-(trifluoromethyl)pyrimidin-1(2H)-yl]-4-fluorophenoxy}-2-pyridyl)oxy]acetate), lactofen, acifluorfen, pyraflufen, pyraflufen-ethyl, oxadiazon, butafenacil, pyridin-2-ylmethyl [(3-(2-chloro-4-fluoro-5-[3-methyl-2,6-dioxo-4-(trifluoromethyl)-3,6-dihydropyrimidin-1(2H)-yl]phenoxy)pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin.

Soybean event Gm_CSM63717 is characterized as a single copy insertion into a site-directed locus in the soybean genome, resulting in two new loci or junction sequences (e.g., the sequences set forth in SEQ ID NO:1, SEQ ID NO:2, SEQ ID NO:3, SEQ ID NO:4, SEQ ID NO:5, SEQ ID NO:6, SEQ ID NO:7 and SEQ ID NO:8) spanning portions of the inserted DNA and the soybean genomic DNA that are not known to appear or exist naturally in the soybean genome or other transgenic soybean events. i.e., they are unique to event Gm_CSM63717. SEQ ID NO:1, SEQ ID NO:3, SEQ ID NO:5 and SEQ ID NO:7 span the 5′ junction of the soybean genomic sequence and the transgenic DNA insert, and SEQ ID NO:2, SEQ ID NO:4, SEQ ID NO:6 and SEQ ID NO:8 span the 3′ junction. These junction sequences are useful in detecting the presence of the event Gm_CSM63717 in soybean cells, seed, plants, plant parts, progeny, and plant products, such as soybean commodity products. Polynucleotide or DNA molecular probes and/or primer pairs are described herein for use in identifying the presence of these various junction sequences in biological samples containing or derived from, or suspected of containing or being derived from, soybean cells, seeds, plants, plant parts, progeny, or commodity products that contain the event Gm_CSM63717.

As used herein, the term “derived” or “derived from” in reference to a particular DNA molecule, amplicon or sequence in relation to a soybean plant, plant part, seed, progeny, cell and/or soybean plant product, such as a commodity product, means that the DNA molecule, amplicon or sequence is taken, purified, isolated, or made, directly or indirectly, from such soybean plant, plant part, seed, progeny, cell and/or soybean plant product, such as a commodity product. Alternatively, the term “derived” or “derived from” in reference to a soybean plant product, such as a commodity product, in relation to soybean plant, plant part, seed, progeny, or cell, means that the soybean plant product is taken, purified, isolated, or made, directly or indirectly, from such soybean plant, plant part, seed, progeny, or cell.

“Capable of being detected” refers to the ability of a particular DNA molecule, segment or sequence to be detected in a sample, such as by amplification and determining its presence, size or sequence such as by DNA sequence analysis, and/or binding of a probe to the target DNA molecule, segment or sequence.

A “sample” is intended to refer to any composition comprising or derived from, either directly or indirectly, a biological sample, source, or material. The sample may generally comprise soybean DNA and/or substantially or completely pure, purified, or isolated soybean DNA. A “biological sample” contains biological materials, including but not limited to DNA obtained or derived from, either directly or indirectly, the genome of a soybean cell(s), tissue(s), seed(s), plant(s), plant part(s) and/or soybean plant product(s), such as a commodity product(s). Such soybean cell(s), tissue(s), seed(s), plant(s), plant part(s) and/or soybean plant product(s), such as a commodity product(s), may comprise soybean event Gm_CSM63717, or DNA molecule(s) and/or DNA segment(s) comprising soybean event Gm_CSM63717. In some embodiments, a sample or biological sample may comprise soybean cell(s), soybean tissue(s), soybean seed(s), soybean plant(s), soybean plant part(s) and/or soybean plant product(s), whose cells or cellular membranes have been fractured (e.g., disrupted or opened) to release the contents of the soybean cell(s) including genomic DNA or proteins and/or make the contents of the soybean cell(s) including genomic DNA or proteins accessible or usable for assays or testing. “Directly” refers to directly obtaining DNA by a skilled artisan from the soybean genome by fracturing soybean cells (or by obtaining samples of soybean that contain fractured soybean cells) and exposing or using the genomic DNA or protein from soybean cells for the purposes of detection. “Indirectly” refers to obtaining by a skilled artisan a target or specific reference DNA (e.g., a novel and unique junction segment(s) described herein as being diagnostic for the presence of the event Gm_CSM63717) in a particular sample, by means other than by obtaining directly via fracturing of soybean cells or obtaining a sample of soybean that contains fractured soybean cells. Such indirect means include, but are not limited to, amplification of a DNA segment that contains a DNA sequence targeted by a particular probe(s) and/or primer set(s) designed to bind with specificity to or near the target sequence, or amplification of a DNA segment comprising all or part of a target sequence that can be measured and characterized (e.g., measured by migration or separation from other segments of DNA and/or identification in an effective matrix, such as an agarose or acrylamide gel or the like, or characterized by direct sequence analysis of the amplicon(s), or cloning of the amplicon(s) into a vector(s) and direct sequencing of the inserted amplicon(s) present within such vector(s)).

As used herein, the term “recombinant” refers to a non-naturally occurring DNA, protein, combination, or organism that would not normally be found or exist in nature and is created by human intervention. As used herein, a “recombinant DNA molecule” is a DNA molecule comprising a combination of DNA molecules that would not naturally occur together and is the result of human intervention. Two or more elements of such combination of DNA sequences may be operably linked to one another. For example, a recombinant DNA molecule may comprise a combination of at least two DNA molecules heterologous with respect to each other, such as a DNA molecule that comprises a coding sequence operably linked to a heterologous promoter and/or other regulatory expression element(s), and/or a transgene and a heterologous plant genomic DNA adjacent to the transgene, and/or a DNA molecule that is artificially synthesized and comprises a polynucleotide sequence that deviates from any polynucleotide sequence that would normally exist in nature. A recombinant DNA molecule may comprise all or part of a junction sequence of the genome of the event and all or part of the transgenic insert of the genome of the event, and/or may comprise a recombinant or heterologous DNA fragment of soybean event Gm_CSM63717. Examples of recombinant DNA molecules include a DNA molecule comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing. Such recombinant DNA molecules can be derived from a soybean plant, seed, plant part, plant cell, progeny plant, or commodity product comprising soybean event Gm_CSM63717. Alternatively, such recombinant DNA molecules can be comprised in a soybean plant, seed, plant part, plant cell, or progeny plant comprising soybean event Gm_CSM63717, or a commodity product produced therefrom. A representative sample of seed comprising soybean event Gm_CSM63717 has been deposited as ATCC Accession No. PTA-127604. Such recombinant DNA molecules can be formed by the insertion of a heterologous nucleic acid molecule into the genomic DNA of a soybean plant or soybean cell. Such recombinant DNA molecules can be an amplicon diagnostic for the presence of soybean event Gm_CSM63717.

As used herein, a “recombinant” in reference to a plant, plant part, seed, plant cell, or progeny is a plant, plant part, seed, plant cell or progeny that would not normally exist in nature, is the result of human intervention, and contains a transgenic DNA molecule stably integrated into the genome of the plant, plant part, seed, plant cell, or progeny. As a result of such genomic insertion, the recombinant or transgenic plant, plant part, seed, plant cell, or progeny is something new and distinctly different from any related wildtype or naturally occurring plant, plant part, seed, plant cell or progeny. An example of a recombinant plant is a soybean plant containing the soybean event Gm_CSM63717.

As used herein, the term “transgene” refers to a DNA molecule artificially incorporated into an organism's genome as a result of human intervention, such as by plant transformation methods. A transgene may be heterologous to the organism. The term “transgenic insert” as used herein refers to the foreign or heterologous DNA inserted by plant transformation techniques into the soybean genome to produce soybean event Gm_CSM63717. The sequence for the transgenic insert of soybean event Gm_CSM63717 is provided as SEQ ID NO:9.

As used herein, the term “heterologous” in reference to a combination of two or more DNA sequences or elements means that the two or more DNA sequences or elements do not normally exist together as such combination in nature without human intervention. For example, a DNA molecule may be from a first species or a recombinant DNA molecule and inserted into the genome of a second species. The DNA molecule would thus be heterologous to the genome and the organism. As used herein, the term “heterologous” in reference to a DNA molecule, construct, sequence or protein in relation to a plant, microorganism, plant cell or plant genome means that the DNA molecule, construct, sequence or protein does not exist in nature as part of such a plant, microorganism, plant cell or plant genome, and/or does not exist in the same physical or genomic location, context or orientation as part of such a plant, microorganism, plant cell or plant genome in nature, without human intervention.

As used herein, the term “chimeric” refers to a single DNA molecule produced by fusing a first DNA molecule to a second DNA molecule, where neither first nor second DNA molecule would normally be found in that configuration fused to the other. The chimeric DNA molecule is thus a new DNA molecule not normally found in nature. An example of a chimeric DNA molecule is a DNA molecule comprising at least one sequence selected from SEQ ID NO:1-10.

As used herein, the term “isolated” in reference to a molecule means that the molecule is at least partially separated from other molecules that are normally associated with it in its native or natural state. In some embodiments, the term “isolated” refers to a DNA molecule that is at least partially separated from the nucleic acids or polynucleotide or DNA sequence(s) that normally flank and are covalently linked to the sequence of the DNA molecule in its native or natural state. An “isolated” DNA molecule may have a DNA sequence corresponding to a portion of the genome of a plant cell without other genomic DNA sequence(s) that normally flank and are covalently linked to the DNA sequence in nature. Such an “isolated” DNA molecule may comprise all or part of a transgene and/or transgenic event, which may comprise all or part of soybean event Gm_CSM63717 or the transgenes or expression cassettes described herein. Nucleic acid sequences or elements, such as a coding sequence, intron sequence, 5′ UTR, promoter sequence, 3′ UTR, and the like, that are naturally found within the DNA of the genome of an organism are not considered to be “isolated” so long as the element is within the genome of the organism and at the location within the genome in which it is naturally found. However, each of these elements, and subparts of these elements, would be “isolated” within the scope of this disclosure so long as the element or subpart is not within the genome of the organism, and at the location within the genome of the organism, in which it is naturally found. An “isolated” DNA molecule may be any recombinant DNA molecule or amplification product or amplicon, and/or may comprise any DNA sequence removed from its natural or biological state and covalently fused to another DNA molecule or sequence with which it is not associated in nature. Such an isolated DNA molecule could be created by the use of biotechnology techniques, such as by making a recombinant DNA or integrating a foreign or heterologous DNA molecule into the chromosome of a cell, plant, or seed. Thus, any DNA molecule comprising a transgenic, recombinant, chimeric or artificial nucleotide sequence, transgene or expression cassette would be considered to be an “isolated” DNA molecule since these sequences are not naturally occurring, regardless of whether the sequence, transgene or expression cassette is present within a plasmid, vector or construct used to transform plant cells, within the genome of a plant, plant part, plant tissue, plant cell or progeny, or is present in detectable amounts in tissues, progeny, biological samples or commodity products derived from a plant, plant part, plant tissue, progeny or plant cell. A recombinant DNA molecule or sequence, or any fragment derived therefrom, comprising all or part of a transgene or junction sequence of the soybean event Gm_CSM63717 would therefore also be considered to be “isolated.” An “isolated” DNA molecule may be extracted or purified from a transgenic plant(s), plant part(s), plant cell(s) and/or tissue(s), or may be present in a homogenate, extract or lysate from any such transgenic plant(s), plant part(s), plant cell(s) and/or tissue(s), or may be produced as an amplicon or amplification product from plant genomic DNA and/or extracted or purified DNA from transgenic plant(s), plant part(s), plant cell(s) and/or tissue(s), or a homogenate, extract or lysate from plant(s), plant part(s), plant cell(s) and/or tissue(s). For the purposes of this disclosure, any transgenic polynucleotide or DNA sequence, i.e., the nucleotide sequence of the DNA inserted into the genome of a plant or bacterium, or present in an extrachromosomal vector, would be considered to be an “isolated” nucleotide or DNA sequence whether it is present within the plasmid or similar structure used to transform the cells, within the genome of the plant or bacterium, or present in detectable amounts in tissues, progeny, biological samples or commodity products derived from the plant or bacterium. An “isolated” DNA molecule is a chemical or biochemical molecule, regardless of whether the molecule is referred to as a nucleic acid, a nucleic acid sequence, a polynucleotide sequence, a DNA sequence, a nucleic acid molecule, a polynucleotide molecule, a DNA molecule, or the like. An “isolated” molecule can provide industrial applicability when present in a plant cell or in a plant genome or when present outside of a plant cell, and therefore, provides and exhibits (and is intended to provide and exhibit) utility regardless of where the molecule is located.

As used herein, the term “correspond” or “corresponding”, or the like, when used in the context of a nucleotide position, mutation, insertion and/or substitution in any given polynucleotide (e.g., SEQ ID NO:9) with respect to a reference polynucleotide sequence (e.g., SEQ ID NO:10) refers to the position(s) of the polynucleotide residue(s) in the given sequence that has identity to the residue(s) in the reference nucleotide sequence when the given polynucleotide is aligned to the reference polynucleotide sequence using a global or local sequence alignment algorithm.

DNA molecules, fragments, and their corresponding DNA sequences, as well as methods of detection are provided. As used herein, the terms “DNA”, “DNA molecule” and “nucleic acid molecule” refer to a deoxyribonucleic acid (DNA) molecule. A DNA molecule may be of genomic or synthetic origin and/or comprise a recombinant or heterologous DNA molecule or sequence. A DNA molecule may be described by convention from the 5′ (upstream) end to the 3′ (downstream) end. As used herein, the term “DNA sequence” refers to the polynucleotide sequence of a DNA molecule, i.e. the sequence of consecutive nucleotides in the DNA molecule. As used herein in reference to nucleotides of a polynucleotide or DNA sequence or molecule, the terms “consecutive” and “contiguous” are interchangeable and synonymous and refer to the 5′ to 3′ order of nucleotides in a polynucleotide or DNA sequence, strand or molecule without any gap or interruption between them. The nomenclature used is that required by Title 37 of the United States Code of Federal Regulations § 1.822 and set forth in the tables in WIPO Standard ST.25 (1998), Appendix 2, Tables 1 and 3. By convention, DNA sequences and fragments thereof are disclosed with reference to the 5′ to 3′ direction of only one strand of the two complementary DNA sequence strands of a DNA molecule. By implication and intent, the complementary sequences of the sequences provided here (the sequences of the complementary strand), also referred to in the art as the reverse complementary or reverse complement sequences, are within the scope of the present disclosure and are expressly intended to be within the scope of the subject matter claimed. As used herein references to SEQ ID NOs:1-10 and fragments thereof include and refer to the sequence of the complementary strand and fragments thereof.

Also provided is a nucleic acid molecule comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of any one of SEQ ID NOs:1-10.

For example, a nucleic acid molecule is provided comprising a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to the full length of SEQ ID NO:10 or to the full length of SEQ ID NO: 9.

A DNA molecule, or a fragment derived therefrom, can also be extracted from plant(s), plant part(s), seed(s), progeny or plant cell(s), or a homogenate, extract or lysate from plant(s), plant part(s), plant cell(s) or seed(s) or progeny, or can be produced as an amplicon from extracted, purified or isolated DNA from plant part(s), plant cell(s) and/or tissue(s), progeny, or a homogenate, extract or lysate from plant(s), plant part(s), plant cell(s), progeny and/or seeds, which may further comprise soybean event Gm_CSM63717.

As used herein, the term “percent sequence identity” or “% sequence identity” refers to the percentage of identical nucleotides or amino acids in a linear polynucleotide or polypeptide sequence of a reference (“query”) sequence (or its complementary strand) as compared to a test (“subject”) sequence (or its complementary strand) when the two sequences are optimally aligned (with appropriate nucleotide or amino acid insertions, deletions, or gaps totaling less than 20 percent of the reference sequence over the window of comparison). Optimal alignment of sequences for aligning a comparison window are well known to those skilled in the art and may be conducted by tools such as the local homology algorithm of Smith and Waterman, the homology alignment algorithm of Needleman and Wunsch, the search for similarity method of Pearson and Lipman, and by computerized implementations of these algorithms such as GAP, BESTFIT, FASTA, and TFASTA available as part of the Sequence Analysis software package of the GCG® Wisconsin Package® (Accelrys Inc., San Diego, Calif.), MEGAlign (DNAStar Inc., 1228 S. Park St., Madison, Wis. 53715), and MUSCLE (version 3.6) (Edgar, “MUSCLE: multiple sequence alignment with high accuracy and high throughput” Nucleic Acids Research 32(5):1792-7 (2004)) for instance with default parameters. An “identity fraction” for aligned segments of a test sequence and a reference sequence is the number of identical components that are shared by the two aligned sequences divided by the total number of components in the portion of the reference sequence segment being aligned, that is, the entire reference sequence or a smaller defined part of the reference sequence. Percent sequence identity is represented as the identity fraction multiplied by 100. The comparison of one or more sequences may be to a full-length sequence or a portion thereof, or to a longer sequence. Soybean plants, progeny, seeds, cells, plant parts and commodity products comprising a detectable amount of a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9 are within the scope of the present disclosure.

As used herein, the term “fragment” refers to a smaller piece or sequence of a larger or whole DNA molecule or sequence. For example, a fragment of any one of SEQ ID NOs:1-12 and SEQ ID NOs:14-15 may include a sequence that is at least about 10 consecutive nucleotides, at least about 11 consecutive nucleotides, at least about 12 consecutive nucleotides, at least about 13 consecutive nucleotides, at least about 14 consecutive nucleotides, at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 consecutive nucleotides, at least about 40 consecutive nucleotides, at least about 45 consecutive nucleotides, at least about 50 consecutive nucleotides, at least about 60 consecutive nucleotides, at least about 70 consecutive nucleotides, at least about 80 consecutive nucleotides, at least about 90 consecutive nucleotides, at least about 100 consecutive nucleotides, at least about 150 consecutive nucleotides, at least about 200 consecutive nucleotides, at least about 250 consecutive nucleotides, at least about 300 consecutive nucleotides, at least about 400 consecutive nucleotides, or at least about 500 consecutive nucleotides of the larger, whole or complete DNA molecule or sequence.

For example, a “fragment” of the transgenic insert sequence (SEQ ID NO: 9) of soybean event Gm_CSM63717 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO: 9. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO: 9 or any fragment thereof.

Similarly, a fragment of the 5′ flank (SEQ ID NO:11 or SEQ ID NO:14) or 3′ flank (SEQ ID NO:12 or SEQ ID NO:15) of soybean event Gm_CSM63717 can comprise at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 60, at least about 70, at least about 80, at least about 90, at least about 100, at least about 150, at least about 200, at least about 250, at least about 300, at least about 400, or at least about 500 consecutive nucleotides of SEQ ID NO:11 or SEQ ID NO:14; or SEQ ID NO:12 or SEQ ID NO:15. In addition, the present disclosure encompasses nucleotide sequences that are at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to SEQ ID NO:11 or 12, or SEQ ID NO:14 or 15, or any fragment of either thereof.

As used herein, the term “about” indicates a value or a range of values which would be understood as an equivalent of a stated value and can be greater or lesser than the value or range of values stated. Each value or range of values preceded by the term “about” is also intended to encompass the embodiment of the stated absolute value or range of values.

The term “or” is used herein to mean “and/or” unless explicitly indicated to refer to alternatives only or the alternatives are mutually exclusive. Thus, the term “and/or” as used herein in a phrase such as “X and/or Y” is intended to include “X and Y”, “X or Y”, “X” (alone), and “Y” (alone). Likewise, the term “and/or” as used in a phrase such as “X, Y, and/or Z” is intended to encompass each of the following embodiments: X (alone); Y (alone); Z (alone); X and Y; X and Z; Y and Z; X, Y, and Z; X, Y, or Z; X or Z; Y or Z; Y or Z.

When used in conjunction with the word “comprising” or other open language, the words “a” and “an” denote “one or more,” unless specifically noted otherwise. The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

Soybean event Gm_CSM63717 is characterized as a transgenic insertion into a locus in the soybean genome, resulting in two new junctions (or joining or connection points). The DNA sequence of the region spanning the connection by phosphodiester bond linkage of one end of the transgenic insert to the flanking soybean genomic DNA is referred to herein as a “junction.” In other words, a junction is the connection point or covalent linkage of one end of the transgenic insert and the flanking genomic DNA as one contiguous molecule and is formed by the insertion of a heterologous nucleic acid molecule into the soybean genomic DNA. One junction is found at the 5′ end of the transgenic insert and the other is found at the 3′ end of the transgenic insert, referred to herein as the 5′ and 3′ junctions, respectively. A “junction sequence” refers to a DNA sequence of any length of consecutive nucleotides that spans the 5′ or 3′ junction of a transgenic event in the plant genome. For a “junction sequence” to be specific to a junction between a transgenic event and a flanking genomic sequence, the junction sequence will generally comprise a sufficient number of consecutive nucleotides at one end of the insertion and a sufficient number of consecutive nucleotides of the flanking genomic sequence. According to some embodiments, a “junction sequence” may comprise (i) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides at one end of the insertion and (ii) at least five (5) consecutive nucleotides, at least ten (10) consecutive nucleotides, at least fifteen (15) consecutive nucleotides, at least twenty (20) consecutive nucleotides, at least twenty five (25) consecutive nucleotides, at least thirty (30) consecutive nucleotides, at least thirty five (35) consecutive nucleotides, at least forty (40) consecutive nucleotides, at least forth five (45) consecutive nucleotides, or at least fifty (50) consecutive nucleotides of the flanking genomic DNA sequence, although it is understood that any length of consecutive nucleotides spanning a junction of a transgenic event in a plant genome may be a junction sequence. Junction sequences of soybean event Gm_CSM63717 are not known to appear or exist naturally in the soybean genome or other transgenic soybean events—they are unique to event Gm_CSM63717, and are apparent to, and a variety of junction sequences of soybean event Gm_CSM63717 can be determined by, one of skill in the art using SEQ ID NO:10. In SEQ ID NO:10, the 5′ junction is at nucleotides 1,000-1,001, and the 3′ junction is at nucleotides 4,201-4,202. Illustrative junction sequences of soybean event Gm_CSM63717 are provided as SEQ ID NOs:1-8. FIG. 1 illustrates the physical arrangement and locations of the illustrative junction sequences, arranged from 5′ to 3′ (left to right), relative to SEQ ID NO:10. The DNA sequence for the transgenic insert of soybean event Gm_CSM63717 is provided as SEQ ID NO:9. The DNA sequence of the transgenic insert and the soybean genomic DNA flanking each side of the transgenic insert is provided as SEQ ID NO:10. The 5′ junction sequences are provided as SEQ ID NO:1, SEQ ID NO:3, SEQ ID NO:5, and SEQ ID NO:7. The 3′ junction sequences are provided as SEQ ID NO:2, SEQ ID NO:4, SEQ ID NO:6, and SEQ ID NO:8. The junction sequences of soybean event Gm_CSM63717 may be present as part of the genome of a plant, seed, plant part, progeny or plant cell containing soybean event Gm_CSM63717, or a DNA molecule containing all or part of event Gm_CSM63717. The identification of any one or more of the junction sequences in a DNA molecule or a sample from a plant, plant part, seed, progeny, cell or commodity product indicates that the DNA molecule or plant, plant part, seed, progeny, cell or commodity product contains or comprises event Gm_CSM63717, or was obtained from a soybean plant, plant part, seed, progeny, cell or commodity product containing or comprising event Gm_CSM63717, and is diagnostic for the presence of soybean event Gm_CSM63717.

The junction sequences described herein are diagnostic for the presence of all or part of soybean event Gm_CSM63717. Thus, the identification or detection, directly or indirectly, of one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 in a sample or DNA molecule derived from a soybean plant, plant part, seed, progeny, cell, or a commodity product is diagnostic that the soybean plant, plant part, seed, progeny, cell, or a commodity product has or comprises all or part of soybean event Gm_CSM63717. The identification or detection, directly or indirectly, of a 5′ junction sequence and/or a 3′ junction sequence (each as provided or described herein) in a sample or DNA molecule derived from a soybean plant, plant part, seed, progeny, cell, or a commodity product is diagnostic that the soybean plant, plant part, seed, progeny, cell, or a commodity product has or comprises soybean event Gm_CSM63717. The present disclosure thus provides a DNA molecule that comprises at least one of the nucleotide sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10. Any segment of DNA derived from transgenic soybean event Gm_CSM63717 that is sufficient to include at least one of the sequences provided as SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10 is within the scope of the present disclosure. In addition, any DNA or polynucleotide molecule or sequence comprising a sequence complementary to any of the sequences described herein is also within the scope of the present disclosure.

Polynucleotide molecules are provided, which may be single or double stranded, that can be used either as primers or probes for detecting the presence of DNA comprising all or part of event Gm_CSM63717 in a sample derived from a soybean plant, plant part, seed, progeny, cell, or a commodity product. Such primers or probes are specific for a target polynucleotide sequence and, as such, are useful for the identification of soybean event Gm_CSM63717 polynucleotide by the methods described herein. A primer or probe can hybridize to a target polynucleotide sequence to allow for specific detection or amplification of a polynucleotide molecule that comprises, or is covalently linked and associated with, the target polynucleotide sequence. The primers and/or probes may be chosen to identify and distinguish detection of a particular transgenic event and not only the presence of a transgene in a plant genome. The target polynucleotide sequence may comprise all or part of soybean event Gm_CSM63717, a junction sequence and/or flanking genomic DNA. Probes and primers according to the present disclosure may have (i) complete or 100% sequence complementarity (i.e., 100% complementary) to a target polynucleotide sequence or (ii) incomplete sequence complementarity to a target polynucleotide, such as at least 60% complementary, at least 65% complementary, at least 70% complementary, at least 75% complementary, at least 80% complementary, at least 85% complementary, at least 90% complementary, at least 95% complementary, or at least 99% complementary to the target polynucleotide sequence as long as the probe or primer has sufficient complementarity to the target polynucleotide sequence to hybridize to the target polynucleotide sequence under stringent hybridization conditions that are suitable and necessary for use of the probe or primer in the relevant amplification or detection assay, reaction or method. As understood in the art, the percentage complementarity of a primer or probe may be lower if the length of the primer or probe is longer and depends on the stringency and use. Provided are illustrative polynucleotide molecules that can be used either as primers or probes for detecting the presence of soybean event Gm_CSM63717 in a sample. Detection of the presence of soybean event Gm_CSM63717 may be done by using methods known in the art, such as thermal or isothermal amplification of nucleic acids or nucleic acid hybridization techniques (such as Northern analysis and Southern analysis).

A “probe” is a nucleic acid molecule that is complementary to a strand of a target nucleic acid and is useful in hybridization detection methods. Probes include not only deoxyribonucleic or ribonucleic acids but also polyamides and other probe materials that bind specifically to a target DNA sequence and the detection of such binding can be useful in detecting the presence or absence of the target DNA sequence. A probe may be attached to a conventional detectable label or reporter molecule, such as a radioactive isotope, ligand, chemiluminescent agent, or enzyme. Such a probe is complementary to a strand of a target nucleic acid and, in the case of the present disclosure, to a strand of DNA from event Gm_CSM63717 whether from an event Gm_CSM63717-containing plant or from a sample that includes event Gm_CSM63717 DNA.

Provided herein is a DNA molecule comprising a polynucleotide segment of sufficient length to function as a DNA probe that hybridizes specifically under stringent hybridization conditions with soybean event Gm_CSM63717 DNA in a sample, wherein detecting hybridization of the DNA molecule under the stringent hybridization conditions is diagnostic for the presence of soybean event Gm_CSM63717 in the sample. Also provided is a DNA molecule comprising a polynucleotide segment of sufficient length to function as a DNA probe specific for detecting in a sample at least one of: (i) a 5′ junction sequence between flanking soybean genomic DNA and the transgenic insert of soybean event Gm_CSM63717; (ii) a 3′ junction sequence between the transgenic insert of soybean event Gm_CSM63717 and flanking soybean genomic DNA; (iii) SEQ ID NO:9; and (iv) a fragment of SEQ ID NO:9 comprising a sufficient length of contiguous nucleotides of SEQ ID NO:9 to identify the sequence as a fragment of the transgenic insert of Gm_CSM63717. An illustrative DNA sequence useful as a probe for detecting soybean event Gm_CSM63717 is provided as SEQ ID NO:178. Other DNA sequences useful as probes for detecting soybean event Gm_CSM63717 include SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing.

A “primer” is a DNA molecule or oligonucleotide that is designed for use in specific annealing or hybridization methods that involve an in vitro amplification reaction. A pair of primers may be used with template DNA (such as a sample of soybean event Gm_CSM63717 genomic DNA) in a thermal amplification reaction (such as polymerase chain reaction (PCR)) or any other suitable amplification method known in the art to produce an amplification product or amplicon, where the amplicon produced from such reaction would have a DNA sequence corresponding to sequence of the template DNA located between the two sites where the primers hybridized to the template DNA.

DNA amplification reactions, methods and techniques are known to those skilled in art. DNA amplification can be accomplished by any of the various nucleic acid amplification methods known in the art, including thermal and isothermal amplification methods such as polymerase chain reaction (PCR) and loop-mediated isothermal amplification (LAMP). Amplification methods are known in the art and are described, inter alia, in U.S. Pat. Nos. 4,683,195 and 4,683,202 and in PCR Protocols: A Guide to Methods and Applications, ed. Innis et al., Academic Press, San Diego, 1990. PCR amplification methods have been developed to amplify up to 22 kb (kilobase) of genomic DNA and up to 42 kb of bacteriophage DNA (Cheng et al., 1994). These methods as well as other methods known in the art of DNA amplification may be used in the practice of the present disclosure. Examples of DNA amplification methods include PCR, Recombinase Polymerase Amplification (RPA) (see for example U.S. Pat. No. 7,485,428), Strand Displacement Amplification (SDA) (see for example, U.S. Pat. Nos. 5,455,166 and 5,470,723), Transcription-Mediated Amplification (TMA) (see for example, Guatelli et al., 1990), Rolling Circle Amplification (RCA) (see for example, Fire and Xu, 1995; Liu, et al., 1996; Lizardi, et al., 1998; U.S. Pat. Nos. 5,714,320 and 6,235,502), Helicase Dependent Amplification (HDA) (see for example Vincent et al., 2004; U.S. Pat. No. 7,282,328), Multiple Displacement Amplification (MDA) (see for example Dean et al., 2002) and Loop-Mediated Isothermal Amplification (LAMP) (see for example Notomi et al., 2000). A sequence of the heterologous DNA insert and/or flanking genomic DNA sequence from soybean event Gm_CSM63717 can be verified or tested by amplifying such DNA molecules from soybean seed containing event Gm_CSM63717 DNA or soybean plants grown from the soybean seed containing event Gm_CSM63717 DNA, using primers derived from the sequences provided herein, followed by standard DNA sequencing of the PCR amplicon or a cloned DNA fragment thereof.

As used herein, an “amplification product” or “amplified DNA” or “amplicon” refers to the nucleic acid or DNA molecule or segment produced by a nucleic acid amplification reaction or method as further described herein, which is directed to a target nucleic acid or DNA molecule that is part of a template nucleic acid molecule. Amplification or amplifying refers to making multiple copies of a target DNA molecule or segment from a template DNA. For example, to determine whether a soybean plant, plant part, seed, progeny or plant cell, resulting from selfing or outcross of a parent comprising soybean event Gm_CSM63717 contains soybean event Gm_CSM63717, DNA may be extracted from the soybean plant tissue sample and subjected to an amplification reaction or method using a pair of primers that are specific for a target sequence that is uniquely associated or part of soybean event Gm_CSM63717, such as, for example, a first primer derived from a genomic DNA sequence in the region flanking the heterologous inserted DNA of soybean event Gm_CSM63717 that is elongated by polymerase 5′ to 3′ in the direction of the inserted DNA, and a second primer derived from the heterologous inserted DNA molecule that is elongated by the polymerase 5′ to 3′ in the direction of the flanking genomic DNA from which the first primer is derived. The amplicon may range in length depending on the length of the intervening polynucleotide or DNA sequence between the two primer target sequences in the template DNA molecule. Alternatively, a primer pair can be derived from the genomic sequence on both sides of the inserted heterologous DNA so as to produce an amplicon that includes the entire insert polynucleotide sequence (e.g., a forward primer targeted to the genomic portion on the 5′ end of SEQ ID NO:10 (i.e. upstream of SEQ ID NO:9) and a reverse primer targeted to the genomic portion on the 3′ end of SEQ ID NO:10 (i.e. downstream of SEQ ID NO:9) that amplifies a DNA molecule comprising the inserted DNA sequence (SEQ ID NO:9) identified herein in the soybean event Gm_CSM63717 genome. The use of the term “amplicon” specifically excludes primer dimers that may be formed in a DNA amplification reaction.

Provided herein is a pair of DNA molecules comprising a first DNA molecule and a second DNA molecule, wherein the first and the second DNA molecules are different from one another and comprise a fragment of SEQ ID NO:10 or a complement thereof and function as DNA primers when used together in an amplification reaction with DNA comprising soybean event Gm_CSM63717 to produce an amplicon diagnostic for soybean event Gm_CSM63717 in a sample. For example, the first and second DNA molecules can comprise SEQ ID NO:176 and SEQ ID NO:177. The amplicon described herein may comprise a DNA sequence comprising one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, or SEQ ID NO:10 wherein the fragment is at least 10 nucleotides in length and comprises nucleotides 1,000-1,001 or 4,201-4,202 of SEQ ID NO:10. According to present embodiments, the sequence of an amplicon comprises at least one junction sequence or two junction sequences, such as a 5′ junction sequence and/or a 3′ junction sequences for soybean event Gm_CSM63717. Amplification and detection of such an amplicon is indictive or diagnostic for soybean event Gm_CSM63717.

For practical purposes, one should design primers which produce amplicons of a limited size range, for example, between 100 to 1000 bases. Smaller (shorter polynucleotide length) sized amplicons in general are more reliably produced in thermal amplification reactions, allow for shorter cycle times, and can be easily separated and visualized on agarose gels or adapted for use in endpoint TaqMan®-like assays. Smaller amplicons can be produced and detected by methods known in the art of DNA amplicon detection. In addition, amplicons produced using the primer pairs can be cloned into vectors, propagated, isolated, and sequenced or can be sequenced directly with methods well established in the art. Any primer pair of forward and reverse primers, which may be identical or complementary to part of SEQ ID NO:10, such as SEQ ID NO: 176 and SEQ ID NO: 177, that is useful in a DNA amplification method to produce an amplicon diagnostic for soybean event Gm_CSM63717 or progeny thereof is an aspect of the disclosure. Any single isolated DNA polynucleotide primer molecule comprising at least 15 contiguous nucleotides of SEQ ID NO:10, or its complement that is useful in a DNA amplification method to produce an amplicon diagnostic for soybean event Gm_CSM63717 or progeny thereof is an aspect of the disclosure. Any single isolated DNA polynucleotide primer molecule comprising at least 15 contiguous nucleotides of SEQ ID NO:11 or SEQ ID NO:12, or its complement that is useful in a DNA amplification method to produce an amplicon diagnostic for plants comprising soybean event Gm_CSM63717 or progeny thereof is an aspect of the disclosure. Any single isolated DNA polynucleotide primer molecule comprising at least 15 contiguous nucleotides of SEQ ID NO:9, or its complement that is useful in a DNA amplification method to produce an amplicon diagnostic for soybean event Gm_CSM63717 or progeny thereof is an aspect of the disclosure.

A primer is typically designed to hybridize specifically to a complementary target DNA strand to form a hybrid between the primer and the target DNA strand. Hybridization or binding of a primer to the complementary target DNA strand is a point of recognition by a polymerase to begin extension of the primer (i.e., polymerization of additional nucleotides into a lengthening nucleotide molecule) using the target DNA strand as a template. Primer pairs refer to use of two primers binding opposite strands of a double stranded nucleotide segment for the purpose of amplifying the polynucleotide segment between the positions targeted for binding by the individual members of the primer pair, typically in a thermal amplification reaction or other conventional nucleic-acid amplification methods. Primer pairs are typically designed to hybridize to different nearby target positions of a template DNA molecule on opposing strands of the template DNA molecule such that the intervening region or sequence between the two primers can be specifically amplified for use or detection through multiple rounds of amplification.

To detect the presence or absence of soybean event Gm_CSM63717, the target positions and/or the intervening region or sequence of a template DNA molecule may comprise at least one junction sequence and/or at least a portion of the insert of soybean event Gm_CSM63717. To detect the absence of soybean event Gm_CSM63717, the target positions and/or the intervening region or sequence of a template DNA molecule may comprise soybean genomic DNA that does not include a junction sequence or any portion of the insert of soybean event Gm_CSM63717. Thus, the presence or absence of an amplicon with a primer pair may be diagnostic of the presence or absence, respectively, of soybean event Gm_CSM63717 in a DNA molecule or sample, or vice versa. This may also be possible with more than one primer pair. For example, a first primer pair may produce a first amplicon if soybean event Gm_CSM63717 is present, and a second primer pair may produce a second amplicon if soybean event Gm_CSM63717 is absent or not present. Alternatively, the size of an amplicon produced in an amplification reaction may also be diagnostic of the presence or absence of soybean event Gm_CSM63717 in a DNA molecule or sample—e.g., a primer pair may produce a first amplicon of a first size if soybean event Gm_CSM63717 is present or a second amplicon of a second size if soybean event Gm_CSM63717 is absent and not present; or a first primer pair may produce a first amplicon of a first size if soybean event Gm_CSM63717 is present, and a second primer pair may produce a second amplicon of a second size if soybean event Gm_CSM63717 is absent or not present. According to some of these embodiments, at least two primer pairs may be used wherein at least one of the primer pairs is used as an internal control and is not associated with soybean event Gm_CSM63717.

According to present embodiments, a primer pair to detect the presence or absence of all or part of soybean event Gm_CSM63717 in a DNA molecule or sample comprises a first primer and a second primer, wherein the first primer is complementary to a 5′ flanking genomic DNA sequence and the second primer is complementary to a sequence within the transgenic insert; or wherein the first primer is complementary to a 5′ flanking genomic DNA sequence and the second primer is complementary to a 3′ flanking genomic DNA sequence; or wherein the first primer is complementary to a sequence within the transgenic insert and the second primer is complementary to a 3′ flanking genomic DNA sequence. Each reference in this paragraph to a primer complementary to a 5′ flanking genomic DNA sequence, a 3′ flanking genomic DNA sequence, or a sequence within the transgenic insert of soybean event Gm_CSM63717 is also intended to potentially include a primer complementary to the reverse complement or opposing strand of the respective 5′ flanking genomic DNA sequence, 3′ flanking genomic DNA sequence, or sequence within the transgenic insert of soybean event Gm_CSM63717.

Illustrative DNA molecules useful as primers for detection of soybean event Gm_CSM63717 are provided as SEQ ID NO:176 and SEQ ID NO:177. The primer pair SEQ ID NO:176 and SEQ ID NO:177 can be useful as a first primer (complementary to a sequence within the transgenic insert) and a second primer (corresponding to a 5′ flanking genomic DNA sequence), wherein each primer has sufficient length of consecutive nucleotides of SEQ ID NO:10 or a sequence complementary to SEQ ID NO:10 to function as DNA primers that, when used together in an amplification reaction with template DNA derived from soybean event Gm_CSM63717, hybridize to opposite strands of the template DNA and produce an amplicon diagnostic for soybean event Gm_CSM63717 DNA in a sample. The primer pair SEQ ID NO:208 (complementary to a 3′ flanking genomic DNA sequence) and SEQ ID NO:209 (corresponding to a 5′ flanking genomic DNA sequence) are useful as a first primer and a second primer, wherein each primer has sufficient length of consecutive nucleotides of a locus within the soybean genome to function as DNA primers that, when used together in a thermal amplification reaction with template DNA, to produce an amplicon indictive or diagnostic for the wildtype DNA for the zygosity of Gm_CSM63717 event DNA in a sample. An amplicon diagnostic for event Gm_CSM63717 comprises a sequence not naturally found in the soybean genome.

A primer may further comprise an oligo tail sequence such as those used in the Kompetitive Allele-Specific PCR (KASP™) method. The allele-specific primers each harbor a unique tail sequence that corresponds with a universal FRET (fluorescence resonant energy transfer) cassette; one labelled with FAM™ dye and the other with HEX™ dye. During thermal cycling, the relevant allele-specific primer binds to the template and elongates, thus attaching the tail sequence to the newly synthesized strand. The complement of the allele-specific tail sequence is then generated during subsequent rounds of PCR, enabling the FRET cassette to bind to the DNA. The FRET cassette is no longer quenched and emits fluorescence.

Methods for designing and using primers and probes are well known in the art. DNA molecules comprising fragments of SEQ ID NOs:1-10 are useful as primers and probes for detecting soybean event Gm_CSM63717 and can readily be designed by one of skill in the art using the sequences provided herein. Such probes and primers are selected to be of sufficient length and sequence complementarity to a target sequence to hybridize specifically to a target sequence under stringency hybridization conditions. Probes and primers may have a complete sequence complementarity or identity with the target sequence, although probes and primers differing from the target sequence in terms of identity or complementarity but retaining the ability to form a stable double-stranded structure under particular hybridization conditions or reaction conditions and to hybridize to the target sequence may be designed by conventional methods.

Any conventional nucleic acid hybridization or amplification method can be used to identify or detect the presence of a target DNA from a transgenic plant, such as soybean event Gm_CSM63717, in a sample. Polynucleotide molecules or DNA molecules, also referred to as “polynucleotide segment or fragment of sufficient length” or “sufficient length of contiguous or consecutive nucleotides” therefore are capable of specifically hybridizing to a target DNA sequence under certain hybridization conditions or reaction conditions. As used herein, the term “of sufficient length” refers to any length that is sufficient to be useful in a detection method of choice. Probes and primers are generally at least about 8 nucleotides, at least about 10 nucleotides, at least about 12 nucleotides, at least about 14 nucleotides, at least about 16 nucleotides, at least about 18 nucleotides, at least about 20 nucleotides, at least about 22 nucleotides, at least about 24 nucleotides, at least about 26 nucleotides, at least about 28 nucleotides, or at least about 30 nucleotides or more in length. Such probes and primers hybridize specifically to a target DNA sequence under stringent hybridization conditions.

As used herein, two nucleic acid molecules are capable of specifically hybridizing to one another if the two molecules are capable of forming an anti-parallel, double-stranded nucleic acid structure. A nucleic acid molecule is the “complement” of another nucleic acid molecule if they exhibit complete complementarity. As used herein, two nucleic acid molecules exhibit “complete complementarity” and are “completely complementary” if every nucleotide of the first nucleic acid molecule is complementary to every nucleotide of the second nucleic acid molecule when they are aligned. Two molecules are “minimally complementary” if they can hybridize to one another with sufficient stability to permit them to remain annealed to one another under at least conventional “low stringency” conditions. Similarly, the molecules are “complementary” if they can hybridize to one another with sufficient stability to permit them to remain annealed to one another under conventional “high stringency” conditions. Conventional stringency conditions are described by Haymes et al., In: Nucleic Acid Hybridization, A Practical Approach, IRL Press, Washington, DC (1985), and by MR Green and J Sambrook, Molecular cloning: a laboratory manual, 4th Edition, Cold Spring Harbor Laboratory Press, Cold Spring Harbor, N.Y. (2012). Departures from complete complementarity are therefore permissible, as long as such departures do not completely preclude the capacity of the molecules to form a double-stranded structure. In order for a nucleic acid molecule to serve as a primer or probe, it need only be sufficiently complementary in sequence to be able to form a stable double-stranded structure under the particular solvent and salt concentrations and other conditions employed.

As used herein, a substantially homologous or complementary sequence in relation to a reference nucleic acid sequence is a nucleic acid sequence that will specifically hybridize to the reference nucleic acid sequence or its complement to which it is being compared under high stringency conditions. As used herein, “stringent hybridization conditions” refers to conditions under which a polynucleotide will hybridize to its target sequence, typically in a complex mixture of nucleic acids, but to essentially no other sequences. “Stringent conditions” or “stringent hybridization conditions” when referring to a polynucleotide probe, refer to conditions under which a probe will hybridize to its target sequence to a detectably greater degree than to other sequences (e.g., at least 2-fold over background). Stringent conditions are sequence-dependent and will be different in different circumstances. Longer sequences hybridize specifically at higher temperatures. Generally, stringent conditions are selected to be about 5-10° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of identity are detected (heterologous probing).

Appropriate stringency conditions which promote DNA hybridization, for example, 6× sodium chloride/sodium citrate (SSC) at about 45° C., followed by a wash of 2×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Regarding the amplification of a target polynucleotide (e.g., by PCR) using a particular amplification primer pair, “stringent conditions” or “stringent hybridization conditions” are conditions that permit the primer pair to hybridize to the target polynucleotide to which a primer having the corresponding sequence (or its complement) would bind and to produce an identifiable amplification product (the amplicon) having a soybean Gm_CSM63717 event specific region in a DNA thermal amplification reaction. The term “specific for” a target sequence indicates that a probe or primer hybridizes under stringent hybridization conditions only to the target sequence in a sample comprising the target sequence.

A polynucleotide molecule or DNA molecule of the present disclosure, such as a primer or a probe, will specifically hybridize to at least one of the nucleic acid molecule sequences selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to SEQ ID NO:10, or a complete complement of or fragment of any of the foregoing under stringent hybridization conditions, or under moderately stringent hybridization conditions if the sequence of the polynucleotide molecule is not identical to the at least one of the nucleic acid molecules. The hybridization of a nucleic acid molecule, such as a primer or probe, to the target DNA molecule can be detected by any number of methods known to those skilled in the art, which can include, but are not limited to, fluorescent tags, radioactive tags, antibody-based tags, and chemiluminescent tags.

An illustrative DNA molecule or polynucleotide useful as a probe for detecting soybean event Gm_CSM63717 is provided as SEQ ID NO:178. In some embodiments, a DNA molecule that functions as a probe comprises a nucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a complement of any of the foregoing or a fragment of any of the foregoing. In other embodiments, a DNA molecule comprises a polynucleotide segment of sufficient length to function as a DNA probe specific for at least one of: a) a 5′ junction sequence between flanking soybean genomic DNA and the transgenic insert of soybean event Gm_CSM63717; b) a 3′ junction sequence between the transgenic insert of soybean event Gm_CSM63717 and flanking soybean genomic DNA; c) SEQ ID NO:9; or d) a fragment of SEQ ID NO:9 comprising a sufficient length of contiguous nucleotides of SEQ ID NO:9 to identify the sequence as a fragment of the transgenic insert of Gm_CSM63717 in a sample of DNA.

A diagnostic amplicon produced by the methods described herein may be detected by a plurality of techniques known in the art, such as sequencing, restriction mapping, Southern analysis, or any other suitable polynucleotide or DNA hybridization, blotting, polymerization and/or amplification-based approach or technique. One method is Genetic Bit Analysis (Nikiforov et al., 1994) where a DNA oligonucleotide is designed that overlaps both the adjacent flanking genomic DNA sequence and the inserted DNA sequence—i.e., a junction sequence. The oligonucleotide is immobilized in wells of a microtiter plate. Following PCR of the region of interest (using, for example, one primer in the inserted sequence and one in the adjacent flanking genomic sequence), a single-stranded PCR product can be hybridized to the immobilized oligonucleotide and serve as a template for a single base extension reaction using a DNA polymerase and labeled dideoxynucleotide triphosphates (ddNTPs) specific for the expected next base. Readout may be fluorescent or ELISA-based. A signal indicates presence of the transgene/genomic junction sequence due to successful amplification, hybridization, and single base extension.

Another method is the pyrosequencing technique as described by Winge (2000). In this method, an oligonucleotide is designed that overlaps the adjacent genomic DNA and insert DNA junction. The oligonucleotide is hybridized to single-stranded PCR product from the region of interest (one primer in the inserted sequence and onein the flanking genomic sequence) and incubated in the presence of a DNA polymerase, ATP, sulfurylase, luciferase, apyrase, adenosine 5′ phosphosulfate and luciferin. DNTPs are added individually and the incorporation results in a light signal that is measured. A light signal indicates the presence of the transgene/genomic sequence due to successful amplification, hybridization, and single or multi-base extension.

Fluorescence Polarization as described by Chen et al. (1999) is a method that can be used to detect the amplicon of the present invention. Using this method an oligonucleotide is designed that overlaps the genomic flanking and inserted DNA junction. The oligonucleotide is hybridized to single-stranded PCR product from the region of interest (one primer in the inserted DNA and one in the flanking genomic DNA sequence) and incubated in the presence of a DNA polymerase and a fluorescent-labeled ddNTP. Single base extension results in incorporation of the ddNTP. Incorporation can be measured as a change in polarization using a fluorometer. A change in polarization indicates the presence of the transgene/genomic sequence due to successful amplification, hybridization, and single base extension.

Real-time polymerase chain reaction (PCR) has the ability to monitor the progress of the PCR as it occurs (i.e., in real time). Data are collected throughout the PCR process, rather than at the end of the PCR. In real-time PCR, reactions are characterized by the point in time during cycling when amplification of a target is first detected rather than the amount of target accumulated after a fixed number of cycles. In a real-time PCR assay, a positive reaction is detected by accumulation of a fluorescent signal. The higher the starting copy number of the nucleic acid target, the sooner a significant increase in fluorescence is observed. The cycle threshold (Ct value) is defined as the number of cycles required for the fluorescent signal to cross the threshold (i.e., exceeds background level). Ct levels are inversely proportional to the amount of target nucleic acid in the sample (i.e., the lower the Ct value, the greater the amount of target nucleic acid in the sample).

Taqman® (PE Applied Biosystems, Foster City, CA) is a method of detecting and quantifying the presence of a DNA sequence using real-time PCR and is fully described in the instructions provided by the manufacturer. Briefly, a FRET oligonucleotide probe is designed that overlaps the genomic flanking and insert DNA junction. The FRET probe and PCR primers (one primer in the insert DNA sequence and one in the flanking genomic sequence) are cycled in the presence of a thermal stable polymerase and dNTPs. Hybridization of the FRET probe results in cleavage and release of the fluorescent moiety away from the quenching moiety on the FRET probe. A fluorescent signal indicates the presence of the transgene/genomic sequence due to successful amplification and hybridization.

Molecular beacons have been described for use in sequence detection as described in Tyangi et al. (1996). Briefly, a FRET oligonucleotide probe is designed that overlaps the flanking genomic and insert DNA junction. The unique structure of the FRET probe results in it containing secondary structure that keeps the fluorescent and quenching moieties in close proximity. The FRET probe and PCR primers (one primer in the insert DNA sequence and one in the flanking genomic sequence) are cycled in the presence of a thermostable polymerase and dNTPs. Following successful PCR amplification, hybridization of the FRET probe to the target sequence results in the removal of the probe secondary structure and spatial separation of the fluorescent and quenching moieties. A fluorescent signal results and indicates the presence of the flanking/transgene insert sequence due to successful amplification and hybridization.

Other detection methods known in the art may be used. For example, microfluidics (see, e.g., U.S. Patent Publication No. 2006/068398; U.S. Pat. No. 6,544,734) provide methods and devices that can be used to separate and amplify DNA samples or molecules. Optical dyes can be used to detect and measure specific DNA molecules (see, e.g., WO/05017181). Nanotube devices (see, e.g., WO/06024023) that comprise an electronic sensor for the detection of DNA molecules or nanobeads that bind specific DNA molecules can then be detected. Nanopore sequencing technology, such as that described in Wang et al. (2021), Tayler et al. (2018), or Pearson et al. (2019), can also be used for event detection.

The DNA molecules and corresponding nucleotide sequences provided herein are therefore useful for, among other things, identifying soybean event Gm_CSM63717, detecting the presence of DNA derived from the transgenic soybean event Gm_CSM63717 in a sample, and monitoring samples for the presence and/or absence of soybean event Gm_CSM63717 or plant parts derived from soybean plants comprising event Gm_CSM63717.

Provided are proteins that can be used to produce antibodies for detecting the presence of soybean event Gm_CSM63717 in a sample. Such antibodies are specific for the PPO protein that is encoded by soybean event Gm_CSM63717. Methods for preparing a polyclonal antibody or a monoclonal antibody are well-known to those skilled in the art and can be used to make antibodies specific for the PPO protein encoded by soybean event Gm_CSM63717. For example, Lermontova et al (1997) describes antibodies to a PPO protein. The DNA sequence encoding the PPO protein is provided in SEQ ID NO:10 and the start positions and stop positions of the coding sequences are indicated in Table 1. The DNA sequence encoding the protein and the protein encoded by the sequence are useful to produce antibodies for detecting the presence of soybean event Gm_CSM63717 by the methods described herein. Detection for the presence of soybean event Gm_CSM63717 may be done by using any protein detection techniques known in the art, such as western blot analysis, immuno-precipitation, enzyme-linked immunosorbent assay (ELISA), antibody attachment to a detectable label or reporter molecule (such as a radioactive isotope, ligand, chemiluminescent agent, or enzyme), or enzymatic action on a reporter molecule. One method provides for contacting a sample with an antibody that binds to the PPO protein encoded by soybean event Gm_CSM63717 and then detecting the presence or absence of antibody binding. The binding of such antibody is diagnostic for the presence of one or more proteins encoded by soybean event Gm_CSM63717.

An alternative to antibody for protein detection is the aptamer-based detection method for detecting proteins or molecules of interest in a sample. As used herein, the term “aptamer(s)” or “aptamer sequences(s)” refers to short synthetic single-stranded oligonucleotide molecules with high-affinity and specificity binding to a target molecule such as a protein, polypeptide, lipid, glycoprotein, glycolipid, glycopeptide, saccharide, or polysaccharide by forming distinct tertiary structures (Ellington and Szostak, 1990; Robertson and Joyce, 1990; Tuerk and Gold, 1990; Wang et al., 2019). The single-stranded nucleic acid can be ssDNA, RNA or derivatives of either thereof. The aptamer comprises a three-dimensional structure held in certain conformation(s) that provide intermolecular contacts to specifically bind its given target. Although aptamers are nucleic acid-based molecules, the binding to the target molecule is not entirely dependent on a linear base sequence, but rather a particular secondary/tertiary/quaternary structure. The term aptamer also covers next generation aptamers such as X aptamers that cannot typically be amplified by PCR but can be adapted by adding a link primer. Such aptamers can specifically bind to proteins of interest but can also be easily amplified, sequenced etc. in a downstream process. The term aptamer also covers aptamers that include modified bases. It is envisaged that the aptamers may include traditional aptamers of 15 to 120 bases in length, as well as longer aptamers of approximately 200 bases in length (e.g., Ultramers® by Integrated DNA Technologies, Inc. Coralville, Iowa. USA). To detect the protein of interest in a sample, aptamers specific to the protein of interest are obtained and are then incubated with the sample. If the protein of interest is present in the sample, protein-aptamer conjugates are formed. Methods for detecting the aptamer/protein complex are known in the art, such as aptablotting or South-Western blot (Li et al., 2017; Sekhon et al., 2017), aptamer-based Western blot (Wang at al., 2020), aptamer sandwich assay (Svobodova et al., 2021). Chemical modifications, additional functional groups, and/or linkers can be added to the nucleic acid aptamer to provide increased binding affinity to a target protein, and to provide a convenient means to detect the target molecule. Such tags and or labels can comprise fluorescent, luminescent, absorbance, or radioactive-based chemical groups, or can comprise an enzyme or substrate that provides a detectable response such as a precipitate, either alone or in the presence of other factors.

Nucleic acid or protein detection kits for detecting the presence of soybean event Gm_CSM63717 are provided. Variations on such kits can also be developed using the compositions and methods disclosed herein and the methods well known in the art for protein and nucleic acid detection for identification of soybean event Gm_CSM63717. Protein and nucleic acid detection kits can be applied to methods for breeding with plants comprising soybean event Gm_CSM63717. Such kits contain primers and/or probes or antibodies or aptamers which are specific to soybean event Gm_CSM63717. Such DNA primers and/or probes may comprise fragments of one or more of SEQ ID NOs:1-10, or antibodies specific for a protein encoded by the soybean event Gm_CSM63717. The kits can also contain instructions for using the primers, probes, antibodies, or aptamers for detecting the presence of soybean event Gm_CSM63717. Kits may optionally also comprise reagents for performing the detection or diagnostic reactions described herein.

One example of a detection kit comprises at least one DNA molecule of sufficient length of contiguous nucleotides of SEQ ID NO:10 to function as a DNA probe useful for detecting the presence or absence of soybean event Gm_CSM63717 in a sample. The DNA derived from transgenic soybean plants comprising event Gm_CSM63717 would comprise a DNA molecule having at least one sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10, a complement of any of the foregoing, or a fragment of any of the foregoing. An illustrative DNA molecule sufficient for use as a probe is one comprising the sequence provided as SEQ ID NO:178. Other probes may be readily designed by one of skill in the art. The probe can include a junction sequence that spans the 5′ or 3′ junction between the soybean genomic DNA and the transgenic insert of soybean event Gm_CSM63717.

Another example of a detection kit comprises at least one primer pair that specifically hybridize to a target DNA and amplify a diagnostic amplicon under the appropriate reaction conditions useful for detecting the presence or absence of soybean event Gm_CSM63717 in a sample. A kit that contains DNA primers that are homologous or complementary to any portion of the soybean genomic region as set forth in SEQ ID NO:11 or 12 and to any portion of the inserted transgenic DNA as set forth in SEQ ID NO:9 is an object of the present disclosure. The kit may provide an agarose gel-based detection method or any number of methods of detecting the amplicon that are known in the art. Such a method may also include sequencing the amplicon or a fragment thereof. Illustrative DNA molecules sufficient for use as a primer pair are ones comprising the sequences provided as SEQ ID NO:176 and SEQ ID NO:177, and SEQ ID NO:208 and SEQ ID NO:209, respectively, wherein the primer pair SEQ ID NO:176 and SEQ ID NO:177 will produce an amplicon diagnostic for the presence of event Gm_CSM63717 in a sample; and the primer pair SEQ ID NO:208 and SEQ ID NO:209 will produce an amplicon indictive of wildtype DNA, therefore, diagnostic for the absence of event Gm_CSM63717 in a sample. Other primer pairs may be readily designed by one of skill in the art.

Another example of a detection kit comprise's an antibody specific for the PPO protein encoded by soybean event Gm_CSM63717. For example, such a kit may utilize a lateral flow strip comprising reagents activated when the tip of the strip is contacted with an aqueous solution. Illustrative protein sufficient for use in antibody production is the PPO protein encoded by the sequence provided as SEQ ID NO:10, or any fragment thereof. Detection of binding of the antibody to the PPO protein encoded by soybean event Gm_CSM63717 in a sample is diagnostic for the presence of soybean event Gm_CSM63717 in the sample.

The detection kits provided herein are useful for, among other things, identifying soybean event Gm_CSM63717, selecting plant varieties or hybrids comprising soybean event Gm_CSM63717, detecting the presence of DNA derived from the transgenic soybean plant comprising event Gm_CSM63717 in a sample, and monitoring samples for the presence and/or absence of soybean plants comprising event Gm_CSM63717, or plant parts derived from soybean plants comprising event Gm_CSM63717.

Soybean plants, progeny, seeds, cells, and plant parts comprising soybean event Gm_CSM63717 are provided, as well as commodity products produced using these. As used herein, the term “soybean” or “soy” means plant species within Glycine max and all plant varieties belonging to the genus Glycine that can be bred with Glycine max plants, including wild soybean species such as Glycine soja. The term “soybean” is intended to include soybean plants, plant parts, plant cells, plant tissue, seeds, progeny plants, and/or soybean commodity products. These soybean plants, plant parts, plant cells, plant tissues, seeds, progeny plants and commodity products contain or comprise soybean event Gm_CSM63717 or are derived from a transgenic soybean plant, plant part, plant cell, plant tissue, seed, progeny plant or commodity product containing or comprising event Gm_CSM63717. These soybean plants, plant parts, plant cells, plant tissues, seeds, progeny plants and commodity products contain a detectable amount of a polynucleotide or DNA molecule comprising at least one junction sequence and/or heterologous transgenic insert sequence of soybean event Gm_CSM63717, such as a polynucleotide or nucleic acid or DNA molecule having or comprising at least one of the sequences provided as SEQ ID NO:1. SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, a polynucleotide comprising a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9, and a complete complement of any of the foregoing. In some embodiments, the soybean plant, plant part, plant cell, plant tissue, or seed is further defined as a progeny plant of any generation of a soybean plant comprising soybean event Gm_CSM63717, or a soybean plant part, plant seed, or plant cell derived therefrom.

As used herein, “soybean event Gm_CSM63717” or “soybean event Gm_CSM63717 locus” refers to the genomic locus of the soybean event Gm_CSM63717 or a modified soybean event Gm_CSM63717 comprising the complete or partial flanking, junction and insertion sequences of the soybean event Gm_CSM63717 or the modified soybean event Gm_CSM63717. A modified soybean event Gm_CSM63717 comprises one or more mutations, edits and/or genetic modifications such as deletion, insertion, inversion or transposition in the soybean event Gm_CSM63717 locus relative to the soybean event Gm_CSM63717.

A modified soybean event Gm_CSM63717 and methods of making a modified soybean event Gm_CSM63717 are provided. As is described further herein, various mutagenesis or targeted genome editing techniques and related tools exist or could be made or engineered to permit genetic modification or mutation of the transgenic insert, junction and/or the flanking genomic DNA of soybean event Gm_CSM63717, such as by deletion, insertion, transposition, inversion, and/or substitution of nucleic acid sequence(s), or by insertion or introduction of a guide RNA target site or a cognate target site or CgRRS, and the transgenic event as modified may still be uniquely characterized by the presence of heterologous DNA and/or one or more sequences of the insertion, junction(s) and/or flanking sequence described herein at the particular position in the genome previously occupied by the unmodified soybean event Gm_CSM63717 relative to flanking portions or sequences of the native soybean genome. According to present embodiments, a modified transgenic event derived from soybean event Gm_CSM63717 may comprise all or part of the insertion sequence and/or transgene cassette of soybean event Gm_CSM63717 and/or one or more complete or partial flanking sequences described herein. As used herein, a “modified soybean event Gm_CSM63717” refers to the genomic DNA or sequence of the soybean event Gm_CSM63717 locus comprising one or more mutations, edits or genetic modifications relative to the genomic DNA or sequence of the soybean event Gm_CSM63717, wherein such mutations or edits or genetic modifications are introduced or made by a mutagenesis or targeted genome editing technique of a soybean plant, plant part, tissue or cell comprising the soybean event Gm_CSM63717. A “modified soybean event Gm_CSM63717” includes a “further modified soybean event Gm_CSM63717” made by first inserting a target site or cognate target site or CgRRS into the soybean event Gm_CSM63717 locus and then further modifying the soybean event Gm_CSM63717 locus as described herein. Methods and techniques of mutagenesis are known in the art and include, for example, chemical mutagenesis (i.e., treatment with a chemical mutagen, such as an azide, hydroxylamine, nitrous acid, acridine, nucleotide base analog, or alkylating agent—e.g., EMS (ethylmethane sulfonate), MNU (N-methyl-N-nitrosourea), etc.), physical mutagenesis (e.g., gamma rays, X-rays, UV, ion beam, other forms of radiation, etc.), and insertional mutagenesis (e.g., transposon or T-DNA insertion). Soybean plants, plant parts, plant seeds, plant tissues, and plant cells described herein include soybean plants, parts, seeds, tissues and cells comprising a modified soybean event Gm_CSM63717 or a further modified soybean event Gm_CSM63717.

The soybean plants, plant parts, plant cells, plant tissues, seeds, progeny plants and commodity products express or comprise a PPO herbicide tolerance gene, and are tolerant to one or more PPO herbicides including, for example, but not limited to flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, trifludimoxazin, and combinations of any thereof.

The present disclosure provides soybean plants, progeny, seeds, plant cells, and plant parts such as microspores, pollen, anthers, ovules, ovaries, flowers, pods, embryos, stems, buds, nodes, leaves, roots, and calli derived from a transgenic soybean plant comprising soybean event Gm_CSM63717. A representative sample of seed comprising soybean event Gm_CSM63717 has been deposited according to the Budapest Treaty for the purpose of enabling the present disclosure. The ATCC repository has assigned the Accession No. PTA-127604 to the seed comprising soybean event Gm_CSM63717.

Any of the soybean plants, plant seeds, plant parts, or plant cells can further comprise at least one additional transgene for tolerance to at least one additional herbicide. For example, the additional transgenes can be selected from the group consisting of phosphinothricin N-acetyltransferase (PAT), dicamba monooxygenase (DMO), FT_Tv7, triketone dioxygenase (TDO), 5-enolpyruvylshikimate-3-phosphate synthase (EPSPS), and combinations of any thereof. An illustrative PAT codon-optimized coding sequence and its corresponding amino acid sequence from Streptomyces viridochromogenes are provided as SEQ ID NO:26 and SEQ ID NO:27, respectively. An illustrative DMO codon-optimized coding sequence and its corresponding amino acid sequence from Stenotrophomonas maltophilia (aka Pseudomonas maltophilia) are provided as SEQ ID NO:28 and SEQ ID NO:29, respectively. An illustrative FT_Tv7 coding sequence and its corresponding amino acid sequence from Sphingobium herbicidovorans are provided as SEQ ID NO:30 and SEQ ID NO:31, respectively. An illustrative TDO codon-optimized coding sequence and its corresponding amino acid sequence from Oryza sativa are provided as SEQ ID NO:32 and SEQ ID NO:33, respectively. An illustrative EPSPS codon optimized coding sequence and its corresponding amino acid sequence from Agrobacterium CP4 strain are provided as SEQ ID NO:34 and SEQ ID NO:35, respectively. For example, the soybean plant, plant seed, plant part, or plant cell can further comprise soybean event Gm_CSM63714 and/or MON89788. The genetic elements in the transgenic inserts of soybean event Gm_CSM63714 and soybean event MON89788 are described hereinbelow.

The additional transgenes can provide tolerance to herbicides having different modes of action selected from the group consisting of inhibitors of glutamine synthetase (e.g., glufosinate), inhibitors of 4-hydroxyphenylpyruvate dioxygenase (HPPD) (e.g., mesotrione), synthetic auxins such as benzoic acid auxins (e.g., dicamba) and phenoxy auxins (e.g., 2,4-D), inhibitors of EPSPS (e.g., glyphosate), and combinations of any thereof.

A microorganism is provided. The microorganism comprises a polynucleotide molecule having the nucleotide sequence of SEQ ID NO:9, or a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:9. An example of such a microorganism is an Agrobacterium cell. Another example of such a microorganism is an E. coli cell.

A plant cell is provided comprising a polynucleotide molecule as described herein. For example, a plant cell is provided having a nucleotide sequence present in its genome, wherein the nucleotide sequence is selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, and a nucleic acid molecule comprising a polynucleotide having a nucleotide sequence that is at least 90%, at least 91%, at least 92%, at least 93%, at least 94%, at least 95%, at least 96%, at least 97%, at least 98%, at least 99%, at least 99.5%, at least 99.6%, at least 99.7%, at least 99.8%, or at least 99.9% identical to the full length of SEQ ID NO:10 or the full length of SEQ ID NO: 9.

Plant cells and microorganisms of the present disclosure are useful in many industrial applications, including but not limited to: (i) use as research tools for scientific inquiry or industrial research; (ii) use in culture for producing endogenous or recombinant carbohydrate, lipid, nucleic acid, enzymes or protein products or small molecules that may be used for subsequent scientific research or as industrial products; and (iii) for the plant cells of the present disclosure, use with modern plant tissue culture techniques to produce transgenic plants or plant tissue cultures that may then be used for agricultural research or production. The production and use of such transgenic plant cells utilize modern microbiological techniques and human intervention to produce a man-made, unique plant cell. In this process, a recombinant DNA is inserted into a plant cell's genome to create a transgenic plant cell that is separate and unique from naturally occurring plant cells. This transgenic plant cell can then be cultured much like bacteria and yeast cells using modern microbiology techniques and may exist in an undifferentiated, unicellular state. The new plant cell's genetic composition and phenotype is a technical effect created by the integration of a heterologous DNA into the genome of the cell.

Provided are methods of using a plant cell, such as transgenic plant cells. These include (i) methods of producing transgenic cells by integrating a recombinant DNA into the genome of the cell and then using this cell to derive additional cells possessing the same heterologous DNA; (ii) methods of culturing cells that contain recombinant DNA using modern microbiology techniques; (iii) methods of producing and purifying endogenous or recombinant carbohydrate, lipid, nucleic acid, enzymes or protein products from cultured cells; and (iv) methods of using modern plant tissue culture techniques with transgenic plant cells to produce transgenic plants or transgenic plant tissue cultures.

Plants, progeny, seeds, cells, and plant parts may contain one or more additional desirable trait(s). Such desirable traits may be transgenic traits, native traits, or traits produced by other methods such as genome editing, base editing, prime editing or other conventional mutagenesis methods. Desirable traits may be combined with soybean event Gm_CSM63717 by, for example, crossing a soybean plant comprising soybean event Gm_CSM63717 with another soybean plant containing the additional trait(s), or transgenic events. Such traits or transgenic events include, but are not limited to, increased insect resistance, increased water use efficiency, increased yield performance, increased drought resistance, increased disease resistance, increased seed quality, improved nutritional quality, hybrid seed production, and/or increase herbicide tolerance, in which the trait is measured with respect to a soybean plant lacking such transgenic trait. For example, the Gm_CSM63717 event could be stacked by breeding or by retransformation or by site directed integration/introgression with other events or combinations of events known in the art including, but not limited to:
A2704-12 (also known as EE-GM1, LL27, Liberty Link® Soybean for glufosinate herbicide tolerance, deposited as NCIMB41658 and described in PCT Publication No. WO2006/108674, U.S. Pat. Nos. 8,012,689 and 9,322,069, and USDA-APHIS Petition 96-068-0 1p, the entire contents and disclosure of each of which are incorporated herein by reference in their entirety),
A2704-21 (also known as Liberty Link® Soybean for glufosinate herbicide tolerance, described in USDA-APHIS Petition 96-068-0 1p for Determination of Nonregulated Status of Transgenic Glufosinate Resistance Soybean (GRS) Lines W62, W98, A2704-12, A2704-21, and A5547-35, the entire contents and disclosure of which are incorporated herein by reference in their entirety),
A5547-127 (also known as EE_GM2, LL55, Liberty Link® Soybean for glufosinate herbicide tolerance, deposited as NCIMB 41660, and described in PCT Publication No. WO2006/108675, U.S. Pat. Nos. 8,017,756 and 8,952,142, and USDA-APHIS Petition 98-014-01p, the entire contents and disclosure of each of which are incorporated herein by reference in their entirety),
EE-GM4 (for nematode resistance, deposited as ATCC PTA-123624, described in PCT Publication No. WO 2018/119361, U.S. Pat. No. 11,242,539, the entire contents and disclosure of each of which are incorporated herein by reference in their entirety),
A5547-35 (also known as Liberty Link® for glufosinate herbicide tolerance, described in USDA-APHIS Petition 96-068-01p for Determination of Nonregulated Status of Transgenic Glufosinate Resistance Soybean (GRS) Lines W62, W98, A2704-12, A2704-21, and A5547-35, the entire contents and disclosure of which are incorporated herein by reference in their entirety),
CV127 (also known as 127, Cultivance for sulfonylurea herbicide tolerance, deposited as NCIMB 41603, and described in PCT Publication No. WO2010/080829, U.S. Pat. Nos. 9,024,114 and 8,952,142 and 9,961,848, and USDA-APHIS Petition 09-015-01p, the entire contents and disclosure of each of which are incorporated herein by reference in their entirety),
DAS44406-6 (also known as Enlist E3 for glufosinate, glyphosate and 2,4-D herbicide tolerance, deposited as ATCC PTA-11336, and described in PCT Publication No. WO2012/075426, U.S. Pat. Nos. 9,540,655, 10,400,250, and 11,425,906, and USDA-APHIS Petition 11-234-01p, the entire contents and disclosure of each of which are incorporated herein by reference in their entirety),
DAS68416-4 (also known as Enlist Soybean for glufosinate and 2, 4-D herbicide tolerance, deposited as ATCC PTA-10442, and described in PCT Publication Nos. WO2011/066384, WO2011/066360, U.S. Pat. No. 9,944,944, and USDA-APHIS Petition 09-349-01p, the entire contents and disclosure of each of which are incorporated herein by reference in their entirety),
DAS81419 (for glufosinate herbicide tolerance and Lepidopteran insect resistance, deposited as ATCC PTA-12006, and described in PCT Publication No. WO2013/016520, U.S. Pat. No. 8,632,978, and USDA-APHIS Petition 12-272-01p, the entire contents and disclosure of each of which are incorporated herein by reference in their entirety),
DAS14536-7 (for glyphosate, aryloxyalkanoate, and glufosinate herbicide tolerance, deposited as ATCC PTA-11335, and described in PCT Publication No. WO2012/075429 and U.S. Pat. No. 9,540,656, the entire contents and disclosure of each of which are incorporated herein by reference in their entirety),
DAS21606-3 (also known as Event1606 for phenoxy auxinic and/or aryloxyalkanoate herbicide tolerance, deposited as ATCC PTA-11028, described in PCT Publication No. WO2012/033794 and U.S. Pat. No. 8,785,728, the entire contents and disclosure of which are incorporated herein by reference in their entirety),
DP356043 (also known as Optimum GAT™ for glyphosate and sulfonylurea herbicide tolerance, deposited as ATCC PTA-8287, and described in PCT Publication No. WO2008/002872, U.S. Pat. No. 7,951,995, USDA-APHIS Petition 06-271-01p, the entire contents and disclosure of each of which are incorporated herein by reference in their entirety),
DP305423 (also known as Treus™, Plenish™ for quality trait), described in PCT Publication No. WO2008/054747, U.S. Pat. Nos. 8,609,953, 9,816,098, 10,745,711, and 11,390,875, USDA-APHIS Petition 06-354-01p, the entire contents and disclosure of each of which are incorporated herein by reference in their entirety),
DBN9004 (for glyphosate and glufosinate herbicide tolerance), deposited as CGMCC No. 11171 (General Microbiology Center of the China Microbial Culture Collection Management Committee), described in PCT Publication No. WO2017215328, the entire contents and disclosure of which are incorporated herein by reference in their entirety),
FG72 (also known as EE-GM3 for glyphosate and isoxaflutole herbicide tolerance, deposited as NCIMB 41659, and described in PCT Publication No. WO2011/063411, U.S. Pat. Nos. 8,592,650, 3,642,748, 9,631,202 and 10,494,681, and USDA-APHIS Petition 09-328-01p, the entire contents and disclosure of each of which are incorporated herein by reference in their entirety),
GMB151 (also known as EE-GM5 for tolerance to HPPD inhibiting herbicides and nematode resistance, deposited as ATCC PTA-123625, and described in PCT Publication No. WO2018/119364, U.S. Pat. No. 11,555,202, and USDA-APHIS Petition 19-317-01p, the entire contents and disclosure of each of which are incorporated herein by reference in their entirety),
GTS 40-3-2 (also known as 40-3-2, Roundup Ready™ for glyphosate herbicide tolerance), described in USDA-APHIS Petition 93-258-01 for Determination of Nonregulated Status for Glyphosate-Tolerant Soybean Line 40-3-2, the entire contents and disclosure of which are incorporated herein by reference in their entirety),
GU262 (also known as Liberty Link™ for glufosinate herbicide tolerance), described in USDA-APHIS Petition 98-238-01p, the entire contents and disclosure of which are incorporated herein by reference in their entirety),
HB4 (also known as IND-00410-5, Verdeca HB4 Soybean for abiotic stress tolerance), deposited as ATCC PTA-125535, described in PCT Publication No. WO2020/197558, US Patent Publication No. 2022/0090114, and USDA-APHIS Petition 17-223-01p, and US FDA BNF No. 000155, the entire contents and disclosure of each of which are incorporated herein by reference in their entirety),
MON87701 (for Lepidopteran insect resistance), deposited as ATCC PTA-8194, and described in PCT Publication No. WO2009/064652, U.S. Pat. Nos. 8,049,071 and 8,455,198, and USDA-APHIS Petition 09-082-01p, the entire contents and disclosure of each of which are incorporated herein by reference in their entirety),
MON87705 (also known as Vistive Gold™ for modified product quality, deposited as ATCC PTA-9241, described in PCT Publication No. WO2010/037016, U.S. Pat. Nos. 8,692,080, 8,329,989, 9,572,311, and 10,344,292, and USDA-APHIS Petition 09-201-01p, the entire contents and disclosure of each of which are incorporated herein by reference in their entirety),
MON87708 (for dicamba herbicide tolerance in Genuity® Roundup Ready™ 2 Xtend™), deposited as ATCC PTA-9670, and described in PCT Publication No. WO2011/034704. U.S. Pat. Nos. 9,447,428, RE46292E1, and 11,130,961, and USDA-APHIS Petition 10-188-01r, the entire contents and disclosure of each of which are incorporated herein by reference in their entirety),
MON87712 (for increased yield, deposited as ATCC PTA-10296, described in PCT Publication No. WO2012051199, U.S. Pat. Nos. 9,493,786, 10,053,704, 10,604,765, and 10,696,976, and USDA-APHIS Petition 11-202-01p, the entire contents and disclosure of each of which are incorporated herein by reference in their entirety),
MON87751 (for Lepidopteran insect resistance), deposited as ATCC PTA-120166, and described in PCT Publication No. WO2014/201235, U.S. Pat. Nos. 9,719,145, 10,584,391 and 11,236,399, and USDA-APHIS Petition 13-337-01p, the entire contents and disclosure of each of which are incorporated herein by reference in their entirety),
MON87769 (for quality trait, deposited as ATCC PTA-8911, described in PCT Publication No. WO2009/102873, U.S. Pat. Nos. 8,692,076, and 8,999,411, and USDA-APHIS Petition 09-183-01p, the entire contents and disclosure of each of which are incorporated herein by reference in their entirety),

MON87754 (for quality trait, deposited as ATCC PTA-9385, described in PCT Patent Publication No. WO2010/024976, U.S. Pat. No. 9,078,406, the entire contents and disclosure of each of which are incorporated herein by reference in their entirety),

MON89788 (also known as Genuity® Roundup Ready 2 Yield™ for glyphosate herbicide tolerance), deposited as ATCC PTA-6708, and described in PCT Publication No. WO2006/130436, U.S. Pat. Nos. 7,608,761, 7,632,985, 8,053,184, 8,754,289, 9,017,947, 9,605,272, 9,944,945, 10,273,498, 11,390,881, and USDA-APHIS Petition 06-178-01p, the entire contents and disclosure of each of which are incorporated herein by reference in their entirety),
Gm_CSM63714 (for dicamba, glufosinate, 2,4-D and mesotrione herbicide tolerance), deposited as ATCC PTA-127099, described in US Patent Application Publication No. 2023/0348927, the entire contents and disclosure of which are incorporated herein by reference in their entirety),
SYHTOH2 (also known as 0H2, Herbicide-Tolerant Soybean Line for glufosinate and mesotrione herbicide tolerance), deposited as ATCC PTA-11226, and described in PCT Publication No. WO2012/082548, U.S. Pat. Nos. 10,184,134 and 11,053,514, and USDA APHIS Petition 12-215-01r, the entire contents and disclosure of each of which are incorporated herein by reference in their entirety),
W62 (also known as Liberty Link™ for glufosinate herbicide tolerance), described in Petition APHIS USDA Petition 96-068-01p for Determination of Nonregulated Status of Transgenic Glufosinate Resistance Soybean (GRS) Lines W62, W98, A2704-12, A2704-21, and A5547-35, the entire contents and disclosure of which are incorporated herein by reference in their entirety),
W98 (also known as Liberty Link™ for glufosinate herbicide tolerance), described in Petition APHIS USDA Petition 96-068-01p for Determination of Nonregulated Status of Transgenic Glufosinate Resistance Soybean (GRS) Lines W62, W98, A2704-12, A2704-21, and A5547-35, the entire contents and disclosure of which are incorporated herein by reference in their entirety),
260-05 (also known as G94-1, G94-19, G168 for modified product quality, described in USDA-APHIS Petition 97-008-01p, the entire contents and disclosure of which are incorporated herein by reference in their entirety),
DAS81419x DAS44406 (also known as Conkesta Enlist E3™ for glufosinate, glyphosate and 2,4-D herbicide tolerance, and Lepidopteran insect resistance),
DAS68416-4x MON89788 (also known as Enlist™ RoundUp Ready® 2 Soybean, for glufosinate, glyphosate and 2,4-D herbicide tolerance),
FG72x A5547-127 (also known as EE-GM3x EE-GM2, Liberty Link® GT27™ for glufosinate, glyphosate and isoxaflutole herbicide tolerance, deposited as ATCC PTA-11042, described in PCT Patent Publication No. WO2011/063413, the entire contents and disclosure of each of which are incorporated herein by reference in their entirety),
MON87701x MON89788 (also known as Intacta™ Roundup Ready™ 2 Pro for glyphosate herbicide tolerance and Lepidopteran insect resistance),
MON87751x MON87701x MON87708x MON89788 (for glyphosate and dicamba herbicide tolerance, and Lepidopteran insect resistance),
MON87705x MON87708 (for dicamba herbicide tolerance and modified product quality),
MON87705x MON89788 (also known as Vistive Gold for glyphosate herbicide tolerance, and modified product quality),
MON87708x MON89788 (also known as Roundup Ready 2 Xtend Soybean for glyphosate and dicamba herbicide tolerance),
MON87705x MON87708x MON89788 (for glyphosate and dicamba herbicide tolerance, and modified product quality),
MON87708x MON89788x A5547-127 (for glufosinate, glyphosate and dicamba herbicide tolerance),
MON87769x MON89788 (also known as Omega-3×Genuity Roundup Ready 2 Yield Soybeans for glyphosate herbicide tolerance, and modified product quality),
HB4x GTS 40-3-2 (for glyphosate herbicide tolerance, and abiotic stress tolerance),
DP305423x GTS 40-3-2 (for glyphosate sulfonylurea herbicide tolerance, and modified product quality),
DP305423x MON87708 (for dicamba herbicide tolerance, and modified product quality),
P305423x MON87708x MON89788 (for glyphosate and dicamba herbicide tolerance, and modified product quality),
DP305423x MON89788 (for glyphosate herbicide tolerance, and modified product quality).

As used herein, “Gm_CSM63714” refers to soybean event Gm_CSM63714. Soybean seed comprising event Gm_CSM63714 has been deposited under ATCC Accession No. PTA-127099 and is fully described and characterized in US Patent Application Publication No. 2023/0348927, the entire content and disclosure of which are incorporated herein by reference in their entirety. Transgenic soybean plants comprising soybean event Gm_CSM63714 comprise SEQ ID NO:182 (5′ soybean genomic flank sequence+transgenic insert+3′ soybean genomic flank sequence), SEQ ID NO:183 (transgenic insert), SEQ ID NOs:184-187 (5′ junction sequences), and SEQ ID NOs:188-191 (3′ junction sequences). The transgenic insert in soybean plants comprising event Gm_CSM63714 comprises four expression cassettes as shown in Table 2. The first expression cassette comprises in operable linkage (i) promoter, leader, and intron sequences of a ubiquitin (UB3) gene from Arabidopsis thaliana, (ii) an APG6 (Albino and Pale Green 6) chloroplast transit peptide sequence from Arabidopsis thaliana, (iii) a codon-optimized sequence encoding a variant of dicamba monooxygenase (DMO) from Stenotrophomonas maltophilia, and (iv) a 3′ UTR sequence of aluminum-induced Sali3-2 from Medicago truncatula; the second expression cassette comprises in operable linkage (i) a promoter sequence derived from multiple Arabidopsis thaliana promoter sequences, (ii) an intron sequence derived from multiple Arabidopsis thaliana intron sequences. (iii) a codon-optimized coding sequence of the phosphinothricin N-acetyltransferase (PAT) from Streptomyces viridochromogenes, and (iv) a 3′ UTR sequence of a small heat shock protein (Hsp20) from Medicago truncatula; the third expression cassette comprises in operable linkage (i) promoter, leader, intron sequences of a polyubiquitin gene (UBQ10) from Arabidopsis thaliana, (ii) a codon-optimized sequence encoding an alpha-ketoglutarate-dependent non-heme iron dioxygenase variant from Sphingobium herbicidovorans and (iii) a 3′ UTR sequence of a putative protein from Medicago truncatuda; and the fourth expression cassette comprises in operable linkage (I) a promoter sequence derived from multiple Arabidopsis thaliana promoter sequences. (ii) a 5′ UTR leader sequence derived from multiple Arabidopsis thaliana 5′ UTR sequences. (iii) an intron sequence derived from multiple Arabidopsis thaliana intron sequences. (iv) a codon-optimized sequence encoding triketone dioxygenase from Oryza sativa, and (v) a 3′ UTR sequence derived from multiple Zea mays 3′ UTR sequences.

TABLE 2
Transgenic cassettes and elements in soybean event Gm_CSM63714

| Cassette | Element | Description |
|---|---|---|
| 1 | P-At.Ubq3 | Promoter, leader and intron sequences of a ubiquitin |
| | | (UB3) gene from Arabidopsis thaliana. |
| | TS-At.APG6 | APG6 (Albino and Pale Green 6) chloroplast transit |
| | | peptide sequence from Arabidopsis thaliana. |
| | CR-STEma.DMO | Codon-optimized sequence encoding a variant of |
| | | dicamba monooxygenase (DMO) from |
| | | Stenotrophomonas maltophilia. |
| | T-Mt.Sali3-2 | 3′ UTR sequence of aluminum-induced Sali3-2 from |
| | | Medicago truncatula. |
| 2 | P-At.GSP579 | Promoter sequence derived from multiple |
| | | Arabidopsis thaliana promoter sequences. |
| | I-At.GSI102 | Intron sequence derived from multiple |
| | | Arabidopsis thaliana intron sequences. |
| | CR-STRvi.Pat | Codon-optimized coding sequence of the |
| | | phosphinothricin N-acetyltransferase (PAT) from |
| | | Streptomyces viridochromogenes. |
| | T-Mt.Hsp20 | 3′ UTR sequence of a small heat shock protein (Hsp20) |
| | | from Medicago truncatula. |
| 3 | P-At.Ubq10 | Promoter, leader, intron sequences of a polyubiquitin |
| | | gene (UBQ10) from Arabidopsis thaliana. |
| | CR-SPHhe.FT_Tv7 | Codon-optimized sequence encoding an alpha- |
| | | ketoglutarate-dependent non-heme iron dioxygenase |
| | | variant from Sphingobium herbicidovorans. |
| | T-Mt.AC139600v16 | 3′ UTR sequence of a putative protein from |
| | | Medicago truncatula. |
| 4 | P-At.GSP576 | Promoter sequence derived from multiple |
| | | Arabidopsis thaliana promoter sequences. |
| | L-At.GSP576 | 5′ UTR leader sequence derived from multiple |
| | | Arabidopsis thaliana 5′ UTR sequences. |
| | I-At.GSI17 | Intron sequence derived from multiple |
| | | Arabidopsis thaliana intron sequences. |
| | CR-Os.TDO | Codon-optimized sequence encoding triketone |
| | | dioxygenase from Oryza sativa. |
| | T-Zm.GST7 | 3′ UTR sequence derived from multiple Zea mays 3′ |
| | | UTR sequences. |

Soybean plants comprising event Gm_CSM63714 exhibit tolerance to benzoic acid auxins such as dicamba; inhibitors of glutamine synthetase such as glufosinate; phenoxy auxins such as 2,4-D; and β-triketone herbicides (inhibitors of 4-hydroxyphenylpyruvate dioxygenase, or HPPD) such as mesotrione.

As used herein, “MON89788” refers to soybean event MON89788. Soybean seed comprising event MON89788 has been deposited under ATCC Accession No. PTA-6708 and is fully described and characterized in PCT Publication No. WO2006/130436, U.S. Pat. Nos. 7,608,761, 7,632,985, 8,053,184, 8,754,289, 9,017,947, 9,605,272, 9,944,945, 10,273,498, 11,390,881, the entire contents and disclosure of which are incorporated herein by reference in their entirety. Transgenic soybean plants comprising soybean event MON89788 comprise SEQ ID NO:192 (5′ soybean genomic flank sequence+transgenic insert+3′ soybean genomic flank sequence), SEQ ID NO:193 (transgenic insert). SEQ ID NOs:194-195 (5′ junction sequences), and SEQ ID NOs:196-197 (3′ junction sequences). The transgenic insert in soybean plants comprising event MON89788 comprises a CP4 EPSPS expression cassette, comprising in operable linkage (i) a chimeric promoter FMV/Tsfl and related linked elements (referred to as FMV/E1F1α), (ii) an Arabidopsis EPSPS chloroplast transit peptide coding sequence (referred to as CTP2 or TS-AtEPSPS CTP2, (iii) a glyphosate resistant EPSPS coding sequence (referred to as CP4 EPSPS or aroA:CP4, from Agrobacterium tumefaciens strain CP4 codon modified for enhanced expression in plant cells. (iv) a 3′ UTR from pea ribulose 1,5-bisphosphate carboxylase (referred to as E9 3′ or T-Ps.RbcS:E9). Soybean plants comprising event MON89788 exhibit tolerance to glyphosate herbicide.

Any of the soybean plants, plant parts, seeds, cells, progeny or commodity products described herein that comprise soybean event Gm_CSM63717 can further comprise soybean event Gm_CSM63714 and/or MON89788.

Any of the soybean plants, plant parts, seeds, cells, progeny or commodity products described herein can further comprise a recombinant DNA molecule comprising a sequence selected from the group consisting of SEQ ID NO:182; SEQ ID NO:183; SEQ ID NO: 184; SEQ ID NO:185 SEQ ID NO:186; SEQ ID NO:187; SEQ ID NO:188; SEQ ID NO:189; SEQ ID NO:190; SEQ ID NO:191; SEQ ID NO:192; SEQ ID NO:193; SEQ ID NO:194; SEQ ID NO:195; SEQ ID NO:196; SEQ ID NO:197; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:182 or the full length of SEQ ID NO:183 or the full length of SEQ ID NO:192 or the full length of SEQ ID NO:193; and a complete complement of any of the foregoing.

Plants comprising soybean event Gm_CSM63717 and soybean event Gm_CSM63714 and/or soybean event MON89788 can be made by any method known in the art. For example, Plants comprising soybean event Gm_CSM63717 and soybean event Gm_CSM63714 or soybean event MON89788 can be made by crossing a soybean plant comprising soybean event Gm_CSM63717 with a soybean plant comprising either soybean event Gm_CSM63714 or soybean event MON89788 and selecting for progeny plants containing both Gm_CSM63717 and Gm_CSM63714 or selecting for progeny plants containing both Gm_CSM63717 and MON89788. Plants comprising soybean events Gm_CSM63717, Gm_CSM63714 and MON89788 can be made, for example, by crossing a soybean plant comprising event Gm_CSM63717 with a soybean plant comprising both Gm_CSM63714 and MON89788, or by crossing a soybean plant comprising event Gm_CSM63714 with a soybean plant comprising event MON89788 first, selecting for a progeny plant comprising both Gm_CSM63714 and MON89788; and then crossing the progeny plant comprising both Gm_CSM63714 and MON89788 with a plant comprising event Gm_CSM63717, and selecting for progeny plants comprising Gm_CSM63714, MON89788 and Gm_CSM63717. Alternatively, a soybean plant comprising soybean event Gm_CSM63717 can be crossed with a soybean plant comprising event Gm_CSM63714 (or event MON89788) first, selecting for a progeny plant comprising both soybean events Gm_CSM63717 and Gm_CSM63714 (or MON89788), and then crossing the progeny plant comprising both Gm_CSM63717 and Gm_CSM63714 (or MON89788) with a plant comprising event MON89788 (or Gm_CSM63714), and selecting for a progeny plant comprising all three events.

The term “site-specific nuclease” refers to any enzyme that can cleave a nucleotide sequence in a site-specific manner. Site-specific nucleases allow for the precise and/or targeted editing of a specific location in a genome of a plant. Site-specific nucleases include, for example, RNA guided nucleases, zinc-finger nucleases (ZFNs), and transcription activator-like effector nucleases (TALENs).

Some site-specific nucleases, such as zinc finger nucleases (ZFNs) and TALENs, are not RNA-guided and instead rely on their protein structure to determine their target site for causing a DSB (double stranded break) or nick, or they are fused, tethered or attached to a DNA-binding protein domain or motif. The protein structure of the site-specific nuclease (or the fused/attached/tethered DNA binding domain) targets the site-specific nuclease to the target site. ZFNs, and TALENs, may be designed, engineered, and constructed according to known methods to target and bind to a target site.

RNA-guided nucleases are nucleases that form a complex (e.g., a ribonucleoprotein) with a guide RNA, which then guides the complex to a target site within a target sequence. One non-limiting example of guided nucleases are CRISPR nucleases. CRISPR (Clustered Regularly Interspaced Short Palindromic Repeats) nucleases are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells. CRISPR editing systems comprising a CRISPR associated protein (nuclease) and cognate guide RNAs (that can be transcribed from guide DNA polynucleotides) may be used for targeted DNA cleavage or modification. The CRISPR-associated protein can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7.

As further described in Example 2 hereinbelow, soybean event Gm_CSM63717 was integrated into the genome at a site close to the location of the event Gm_CSM63714. In order to accomplish this, bioinformatic analysis was first used to identify genomic target sites for site directed integration (SDI) of transgenes.

As used herein, the term “target site,” “genomic target site,” “target genomic nucleic acid,” or “target soybean genomic nucleic acid,” refers to a polynucleotide sequence that is sufficiently unique in the soybean genome to allow targeted genome modification by a site-specific nuclease. In one aspect, the sequence of the target site is changed from the wildtype sequence, namely the target site is edited. In another aspect, the target site is the site of insertion of a DNA sequence of interest.

The target site can comprise one or more of the criteria selected from the group consisting of: (i) the target site selected is more than 2 kb from a gene (ii) the target site selected is more than 1000 nucleotides (nt) from a small RNA hotspot, and (iii) the target site comprises 700 nucleotides of unique sequence on either side, Target site selection criteria are further described in US Patent Application Publication No. 2020/0024610 and PCT Publication No. WO 2024/129512, the entire contents and disclosure of which are incorporated herein by reference in their entirety.

The target site comprises a sequence that is recognized by a site-specific nuclease. In some embodiments, the target site comprises a sequence that is recognized by a site-specific nuclease resulting in precise or targeted cleavage within the target site. For example, the site-specific nuclease can be selected from the group consisting of an RNA-guided nuclease, a Zinc Finger nuclease, and a TALEN. In some embodiments, the target site comprises a PAM (Protospacer Adjacent Motif) sequence that is recognized by an RNA-guided nuclease (e.g., a CRISPR nuclease system). For example, the target site can comprise a PAM motif that is recognized by a Cas12a/Cpf1 CRISPR nuclease system. The target site can further comprise a sequence that is recognized by and hybridizes to a CRISPR guide RNA. In some embodiments, the target site comprises a sequence that is recognized by and hybridizes to a Cas12a/Cpf1 CRISPR guide RNA.

A DNA sequence of interest can be inserted at a target site using a site-specific nuclease. As used herein, the term “DNA sequence of interest” or “donor sequence” or “donor DNA” refers to a nucleic acid/DNA sequence that has been selected for targeted insertion into a soybean genomic sequence. In one aspect, the soybean genomic sequence is a genomic target site described above. A DNA sequence on interest can be of any length, for example between 2 and 50,000 nucleotides in length (or any integer value therebetween). In some embodiments, the DNA sequence is between about 1,000 and 5,000 nucleotides in length (or any integer value therebetween). In some embodiments, the DNA sequence is between about 5,000 and 10,000 nucleotides in length (or any integer value therebetween). In some embodiments, the DNA sequence is between about 10,000 and 15,000 nucleotides in length (or any integer value therebetween). In some embodiments, the DNA sequence is between about 15,000 and 20,000 nucleotides in length (or any integer value therebetween). In some embodiments, the DNA sequence is between about 20,000 and 25,000 nucleotides in length (or any integer value therebetween). In some embodiments, the DNA sequence is between about 25,000 and 30,000 nucleotides in length (or any integer value therebetween). In some embodiments, the DNA sequence is between about 30,000 and 35,000 nucleotides in length (or any integer value therebetween). In some embodiments, the DNA sequence is between about 35,000 and 40,000 nucleotides in length (or any integer value therebetween). In some embodiments, the DNA sequence is between about 40,000 and 45,000 nucleotides in length (or any integer value therebetween). In some embodiments, the DNA sequence is between about 45,000 and 50,000 nucleotides in length (or any integer value therebetween). A DNA sequence may comprise one or more gene expression cassettes that further comprise actively transcribed and/or translated gene sequences. For example, the DNA sequence of interest can comprise a gene expression cassette comprising a sequence selected from: an herbicide tolerance gene, an insecticidal resistance gene, a nitrogen use efficiency gene, a water use efficiency gene, a nutritional quality gene, a DNA binding gene, a selectable marker gene, a target site for a site-specific nuclease, and any combination thereof. Alternatively, the DNA sequence of interest may comprise a polynucleotide sequence which does not comprise a functional gene expression cassette or an entire gene (e.g., may comprise regulatory sequences such as promoters, enhancers, etc.), or may not contain any identifiable gene expression elements or any actively transcribed gene sequence. In some embodiments, the DNA of interest will have at least one homology arm DNA sequence. The term “homology arm DNA sequence” refers to a polynucleotide sequence that has at least 80%, at least 85%, at least 90%, at least 91%, at least 92%, at least 93%, at least 94%, at least 95%, at least 96%, at least 97%, at least 98%, at least 99% or 100% sequence identity to a target sequence in a plant or plant cell. Further, the DNA sequence can be linear or circular, and can be single-stranded or double-stranded. It can be delivered to the cell as naked nucleic acid, as a complex with one or more delivery agents (e.g., liposomes, poloxamers, T-strand encapsulated with proteins, etc.) or contained in a bacterial or viral delivery vehicle, such as, for example, Agrobacterium tumefaciens or a Gemini Virus, or a nanovirus, respectively.

Once a specific target site is identified, a site-specific nuclease targeting the selected target site can be designed and introduced into the plant, seed, or plant cell. For example, a CRISPR-associated nuclease (e.g., Cas12a/Cpf1) and at least one RNA guide molecule that can hybridize to the target site can be designed and cloned into a plant expression vector and delivered to the plant, seed, or plant cell. If the genome modification is designed to induce a double-strand break (DSB) (i.e., induce a cleavage) with non-homologous end joining (NHEJ) repair for introduction of insertions and deletions (indels), then just the engineered CRISPR nuclease and at least one RNA guide molecule is delivered to the plant, seed or cell. If a DNA sequence of interest is to be incorporated at the target site, then the engineered CRISPR nuclease, at least one RNA guide molecule, and the DNA of interest are co-delivered to the plant, seed, or cell. The DNA of interest may integrate into the target site by NHEJ (Non-homologous End Joining) or by homology-dependent repair (HR). In the latter case, the DNA of interest will have at least one homology arm DNA sequence. An alternative to delivery of the engineered CRISPR nuclease as a DNA expression construct is the delivery of a ribonucleoprotein (RNP) complex of the CRISPR associated nuclease protein in complex with the guide RNA.

Following delivery of the site-specific nuclease to a plant cell, the cells or plants regenerated from the cells are sampled to confirm the presence of the intended site-specific genome modification including insertion of the DNA sequence of interest at or proximal to the target site. Methods of detecting the genome modification are known to one skilled in the art, and include PCR, TaqMan® PCR, droplet digital PCR (ddPCR™, Bio-Rad Laboratories, Hercules, Calif.), sequencing, Sanger sequencing, ABI 3730 DNA fragment analysis (Applied Biosystems, Grand Island, N.Y.), Southern analysis, Northern analysis, phenotypic analysis, or any other technique known to one in the art to detect genome modification.

As further described in Example 2 hereinbelow, twelve target sites within 5 centimorgan (cM) upstream and downstream of event Gm_CSM63714 were identified. The sequences of these target sites arm provided herein as SEQ ID NOs:211-222. Guide RNA spacer sequences corresponding to each of the target site sequences are provided as SEQ ID NOs:223-234.

Soybean plants, plant seeds, plant parts, plant cells, and progeny plants are provided. The plant, seed, plant part, plant cell, or progeny plant comprises a recombinant nucleic acid molecule. The recombinant nucleic acid molecule comprises a target soybean genomic nucleic acid sequence having at least 85% sequence identity, at least 90% sequence identity, or at least 95% sequence identity, to a nucleic acid molecule selected from the group consisting of SEQ ID NOs: 211-222. The recombinant nucleic acid molecule further comprises a DNA sequence of interest. The DNA sequence of interest is inserted into said target soybean genomic nucleic acid sequence. In some embodiments, the plant, seed, plant part, or progeny plant comprises a recombinant nucleic acid molecule comprising a target soybean genomic nucleic acid sequence having a sequence selected from the group consisting of SEQ ID NOs: 211-222.

The DNA sequence of interest can comprise a gene of agronomic interest. For example, the gene of agronomic interest can confer herbicide tolerance in plants, such as a PPO gene for conferring tolerance to PPO herbicides.

In some embodiments, the target soybean genomic nucleic acid sequence is at least 1 kb from the Gm_CSM63714 insertion site. In some embodiments, the target soybean genomic nucleic acid sequence maps to within 3.8 cM of the Gm_CSM63714 insertion site. In some embodiments, the target soybean genomic nucleic acid sequence is more than 2 kb from a gene, is more than 1000 nucleotides from a small RNA hotspot, and comprises 700 nucleotides of unique sequence on either side,

A method of generating a recombinant soybean plant cell is provided. The method comprises: (a) obtaining a soybean plant, seed, or cell, wherein said plant, seed, or cell comprises a target soybean genomic nucleic acid molecule having at least 85% sequence identity, at least 90% sequence identity, or at least 95% sequence identity to a nucleic acid molecule selected from the group consisting of SEQ ID NOs: 211-222; (b) introducing into the soybean plant, seed, or cell a site-specific nuclease that can specifically bind to and cleave the target soybean genomic nucleic acid molecule; (c) introducing a DNA sequence of interest into the soybean plant, seed, or cell; and (d) selecting recombinant soybean plants, seeds or cells comprising the DNA sequence of interest inserted in the target soybean genomic nucleic acid molecule. The site-specific nuclease can be selected from the group consisting of an RNA-guided nuclease, a zinc finger nuclease and a TALEN. For example, the RNA-guided nuclease can be Cas12a. The method can further comprise introducing into the soybean plant, seed, or cell a guide polynucleotide comprising a nucleic acid sequence that is substantially complementary to the target soybean genomic nucleic acid, wherein the guide polynucleotide and the RNA-guided nuclease form a complex that can bind to and cleave the soybean genomic nucleic acid molecule. The guide polynucleotide can comprise a nucleotide sequence having at least at least 85% sequence identity, at least 90% sequence identity, or at least 95% sequence identity to a nucleic acid molecule selected from the group consisting of SEQ ID NOs:223-234 The guide polynucleotide can further comprise SEQ ID NO:207. In some embodiments, the target soybean genomic nucleic acid sequence is at least 1 kb from the Gm_CSM63714 insertion site. In some embodiments, the target soybean genomic nucleic acid sequence maps to within 3.8 cM of the Gm_CSM63714 insertion site. In some embodiments, the target soybean genomic nucleic acid sequence is more than 2 kb from a gene, is more than 1000 nucleotides from a small RNA hotspot, and comprises 700 nucleotides of unique sequence on either side.

A recombinant DNA molecule is provided. The recombinant DNA molecule comprises a DNA sequence having at least 85% sequence identity, at least 90% sequence identity, or at least 95% sequence identity to a nucleic acid molecule selected from the group consisting of SEQ ID NOs:211-222. For example, the nucleic acid molecule can be selected from the group consisting of SEQ ID NOs:211-222. The DNA sequence can be operably linked to a heterologous promoter sequence. The recombinant DNA molecule can further comprise SEQ ID NO:207.

A recombinant RNA molecule is provided. The recombinant RNA molecule comprises an RNA sequence that is at least 85% complementary, at least 90% complementary, or at least 95% complementary, to a nucleic acid molecule selected from the group consisting of SEQ ID NOs: 211-222. In some embodiments, the RNA sequence is 100% complementary to a nucleic acid molecule selected from the group consisting of SEQ ID NOs: 211-222.

The plants described herein can be used to produce progeny or offspring that comprise soybean event Gm_CSM63717. Such progeny may include any plant, seed, and cell and/or regenerable plant part comprising soybean event Gm_CSM63717 inherited or derived from an ancestor or parental soybean plant(s), at least one of which comprises a DNA molecule having or comprising at least one polynucleotide sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, a polynucleotide comprising at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9.

Soybean plants, progeny, and seeds may be homozygous or heterozygous for the event Gm_CSM63717 and the transgenes of event Gm_CSM63717. Progeny may be grown from seeds produced by a soybean plant comprising or containing event Gm_CSM63717 and/or from seeds produced by a plant fertilized with pollen from a soybean plant comprising or containing event Gm_CSM63717 (i.e., fertilized with pollen comprising or containing event Gm_CSM63717). Plants or progeny may also be obtained by tissue culture and regeneration methods from a protoplast, cell, embryo or reproductive or somatic tissue derived from a soybean plant comprising or containing soybean event Gm_CSM63717.

Progeny plants may be self-pollinated (also known as “selfing”) to generate a true breeding line of plants, i.e., plants homozygous for the soybean event Gm_CSM63717 DNA. Alternatively, progeny plants may be outcrossed, i.e., bred with another plant, to produce a varietal or a hybrid seed or plant. The other plant may be transgenic or non-transgenic. A varietal or hybrid seed or plant of the present disclosure may thus be derived by crossing a first parent that lacks the specific and unique DNA of event Gm_CSM63717 with a second parent comprising event Gm_CSM63717, resulting in a hybrid comprising the specific and unique DNA of event Gm_CSM63717. Each parent can be a hybrid or an inbred/variety, so long as the cross or breeding results in a plant or seed of the present disclosure, i.e., a seed having at least one allele comprising the specific and unique DNA of event Gm_CSM63717 and/or at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9.

Sexually crossing one plant with another plant, i.e., cross-pollinating, may be accomplished or facilitated by human intervention, for example: by human hands collecting the pollen of one plant and contacting this pollen with the style or stigma of a second plant; by human hands and/or human actions removing, destroying, or covering the stamen or anthers of a plant (e.g., by manual intervention or by application of a chemical gametocide) so that natural self-pollination is prevented and cross-pollination would have to take place in order for fertilization to occur; by human placement of pollinating insects in a position for “directed pollination” (e.g., by placing beehives in orchards or fields or by caging plants with pollinating insects); by human opening or removing of parts of the flower to allow for placement or contact of foreign pollen on the style or stigma; by selective placement of plants (e.g., intentionally planting plants in pollinating proximity); and/or by application of chemicals to precipitate flowering or to foster receptivity (of the stigma for pollen).

Two different transgenic plants of the same or different genetic backgrounds may thus be crossed to produce inbred or hybrid offspring plants, plant parts and/or seeds that contain two independently segregating transgenes or events wherein at least one of those transgenes or events comprises or is contained within soybean event Gm_CSM63717. For example, transgenic plants comprising soybean event Gm_CSM63717 can be crossed with other transgenic soybean plants to produce a plant having the characteristics of both transgenic parents.

Back-crossing to a parental plant and out-crossing with a non-transgenic plant are also contemplated, as is vegetative propagation. Descriptions of other breeding methods that are commonly used for different traits and crops are known in the art and can be found in one of several references, e.g., Fehr, in Breeding Methods for Cultivar Development, Wilcox J. ed., American Society of Agronomy, Madison WI (1987).

A plant part is provided. As used herein, a “plant part” refers to any part of a plant that is comprised of material directly from or derived from a plant comprising soybean event Gm_CSM63717. Plant parts include but are not limited to microspores, pollen, anthers, ovules, ovaries, flowers, pods, embryos, buds, nodes, stems, leaves, roots, and calli, in whole or part. Plant parts may be viable or nonviable. Plant parts may be regenerable or non-regenerable.

Nonliving or nonregenerable soybean plant materials are provided herein. The nonliving or nonregenerable soybean plant material can comprise any of the recombinant DNA molecules characteristic of soybean event Gm_CSM63717 described herein, or any of the DNA constructs described herein. The nonliving or nonregenerable soybean plant material can comprise soybean event Gm_CSM63717, a representative sample of seed comprising the soybean event soybean event Gm_CSM63717 having been deposited under ATCC Accession No. PTA-127604.

Commodity products that comprise any of the DNA molecules characteristic of soybean event Gm_CSM63717 or any of the DNA constructs described herein are provided. Such commodity products can be produced from plants comprising soybean event Gm_CSM63717. The commodity products contain a detectable amount of DNA comprising a DNA sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide having a sequence that is at least 90%, at least 91%, at least 92%, at least 93%, at least 94%, at least 95%, at least %%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. As used herein, a “commodity product” refers to any composition or product which is comprised of material from plant, seed, cell, or plant part comprising soybean event Gm_CSM63717. Commodity products may be viable or non-living plant material, that is, a material that is not living and derived from a plant, seed, cell, or plant part comprising soybean event Gm_CSM63717. Nonviable commodity products include but are not limited to nonviable seeds, whole or processed seeds, processed plant tissues or plant parts, dehydrated plant tissues or parts, frozen plant tissues or parts, food for human consumption such as soybean oil, soy milk, soy flour, soy grits, soy protein, soy protein concentrate, hydrolyzed vegetable protein, textured soy protein, lecithin, curd, tofu, vegetable soybean (edamame), soy sprouts, soy film (yuba), roasted soybeans, miso, tempeh, soy sauce, or natto; plant parts processed for animal feed such as soy meal; soy fiber, biodiesel; bio-composite building materials such as particleboard, laminated plywood, or lumber products; soy oil-based solvents; soy oil-based industrial lubricants; soy ink; soy candles; soy crayons; soy-based hydraulic fluid; or soy-based foams. Viable commodity products include but are not limited to viable seeds, viable plant parts (such as node, bud, root and leaf) and viable plant cells. A plant comprising event Gm_CSM63717 can thus be used to manufacture any commodity product typically acquired from a soybean plant. Any such commodity product that is derived from the plants comprising event Gm_CSM63717 may contain at least a detectable amount of the specific and unique DNA corresponding to event Gm_CSM63717, and specifically may contain a detectable amount of a polynucleotide having a nucleotide sequence of at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9. Any standard method of detection for polynucleotide molecules may be used, including methods of detection disclosed herein.

Methods for producing such commodity products are also provided. Such methods comprise: (a) obtaining a transgenic soybean plant, plant part, or plant seed comprising soybean event Gm_CSM63717; and (b) producing a commodity product from the transgenic soybean plant, plant part, or plant seed.

A plant tolerant to herbicides may be produced by sexually crossing a plan comprising event Gm_CSM63717 with another plant and thereby producing seed, which is then grown into progeny plants. For example, provided herein is a method of producing a progeny soybean plant comprising event Gm_CSM63717, the method comprising: (a) sexually crossing a first soybean plant that comprises soybean event Gm_CSM63717 with itself or a second soybean plant; (b) collecting one or more seeds produced from the cross; (c) growing one or more seeds to produce one or more progeny plants; and (d) selecting at least a first progeny plant or seed comprising soybean event Gm_CSM63717. Inbred and hybrid soybean plants comprising soybean event Gm_CSM63717 produced by such methods are also provided.

The progeny plants may be analyzed using diagnostic methods to select for progeny plants that comprise event Gm_CSM63717 DNA or for progeny plants tolerant to the PPO herbicides such as flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, and combinations of any thereof. The other plant used may or may not be transgenic. The progeny plant and/or seed produced may be varietal or hybrid seed.

A plant tolerant to PPO herbicides may be produced by selfing a plant comprising event Gm_CSM63717 comprising a polynucleotide having the nucleotide sequence of SEQ ID NOs:1-10, at least 16 consecutive nucleotides of SEQ ID NO:1, at least 16 consecutive nucleotides of SEQ ID NO:2, at least 33 consecutive nucleotides of SEQ ID NO:3, at least 32 consecutive nucleotides of SEQ ID NO:4, at least 53 consecutive nucleotides of SEQ ID NO:5, or at least 52 consecutive nucleotides of SEQ ID NO:6, or a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO:9, and thereby producing seed, which is then grown into progeny plants. These progeny plants may then be analyzed using diagnostic methods to select for progeny plants that comprise event Gm_CSM63717 DNA, or for progeny plants tolerant to the PPO herbicides such as flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, and combinations of any thereof.

Soybean event Gm_CSM63717 contains the PPO expression cassette that provides tolerance to PPO herbicides such as flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, and combinations of any thereof. Soybean plants, progeny, and seeds of event Gm_CSM63717 may contain one or more additional desirable trait(s) such as tolerance to inhibitors of glutamine synthetase such as glufosinate, benzoic acid auxins such as dicamba, phenoxy auxins such as 2,4-D; inhibitors of 4-hydroxyphenylpyruvate dioxygenase or HPPD such as mesotrione, inhibitors of EPSPS such as glyphosate, or combinations of any thereof.

PPO herbicides include diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof. Examples of diphenylethers include, but are not limited to, acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof. Examples of N-phenylphthalimides include, but are not limited to, cinidon-ethyl, flumiclorac, flumiclorac-pentyl, and flumioxazin. Examples of oxadiazoles include, but are not limited to, oxadiargyl and oxadiazon. Examples of oxazolidinediones include, but are not limited to, pentoxazone. Examples of phenylpyrazoles include, but are not limited to, fluazolate, pyraflufen, and pyraflufen-ethyl. Examples of pyrimidinediones or phenyluracils include, but are not limited to, benzfendizone, butafenacil, epyrifencacil, flupropacil, flufenoximacil, saflufenacil, and tiafenacil. Examples of thiadiazoles include, but are not limited to, fluthiacet-methyl and thidiazimin. Examples of triazolinones include, but are not limited to, azafenidin, bencarbazone, carfentrazone, its salts and esters, and sulfentrazone. Examples of benzoxazinone derivatives include, but are not limited to, 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)). Examples of other PPO herbicides include, but are not limited to, chlorphthalim, flufenpyr, flufenpyr-ethyl, flumipropyn, pyraclonil, and profluazol. Further examples of other PPO herbicides include:

1) an herbicidally active compound of the general formula (I) or an agrochemically acceptable salt thereof

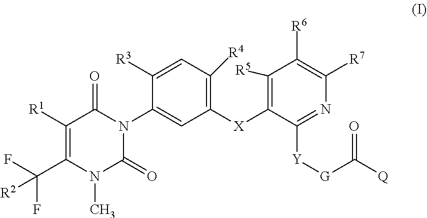

in which:
R¹ is hydrogen,
R² is hydrogen, fluorine, chlorine, bromine, trifluoromethyl, methoxy, ethoxy, prop-1-yloxy, or but-1-yloxy,
R³ is hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R⁵, R⁶ and R⁷ are independently hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, or 2,2,2-trifluoroethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-di-methylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, or 1-ethyl-2-methylpropyl-1-ene,
X and Y are independently O (oxygen) or S (sulfur)
and
Q is one of the following moieties Q-1 to Q-54, Q-56 to Q-57, Q-60 to Q-89, Q-91 to Q-129, Q-131 to Q-139, Q-141 to Q-144, Q-146 to Q-180, Q-182 to Q-185, Q-193 to Q-195, Q-200 to Q-208, Q-210 to Q-370, Q-395 to Q-440:

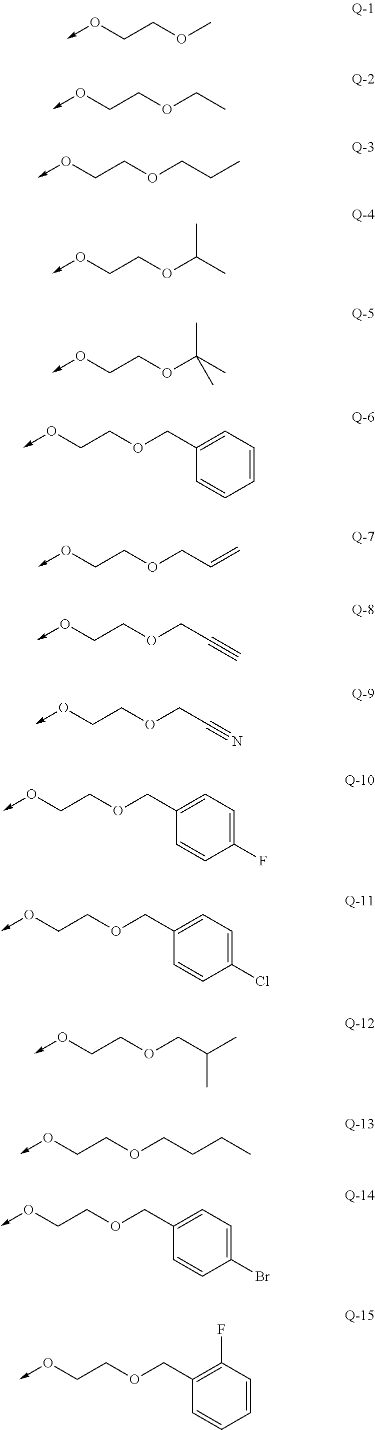

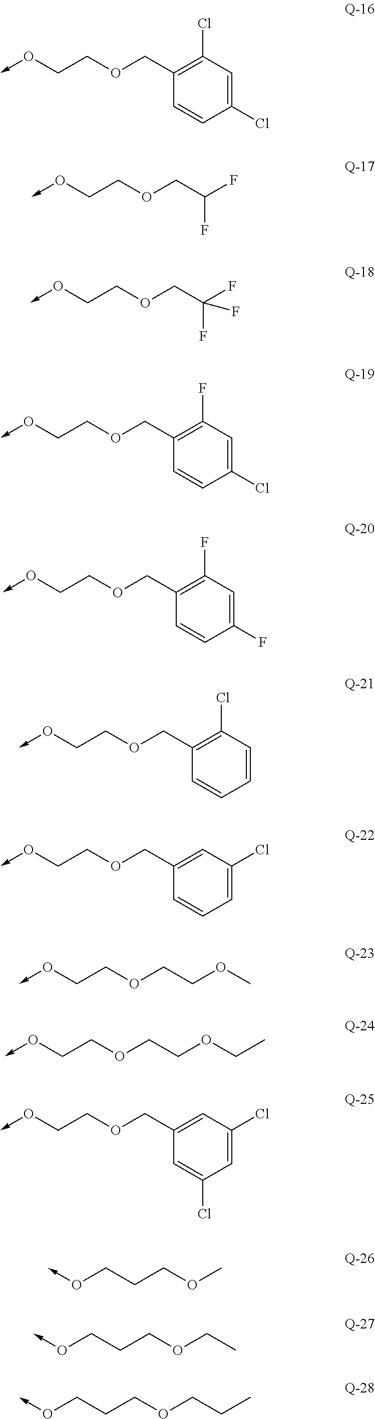

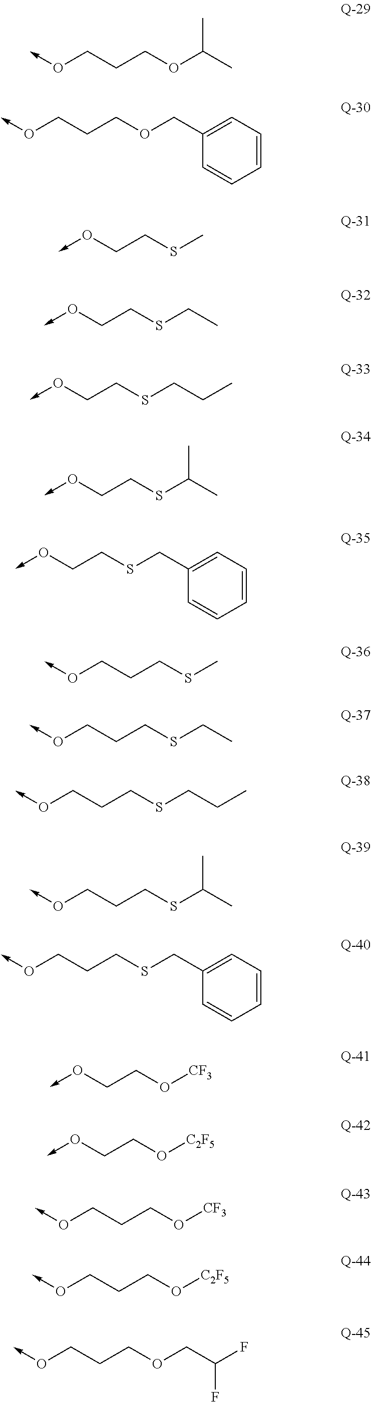

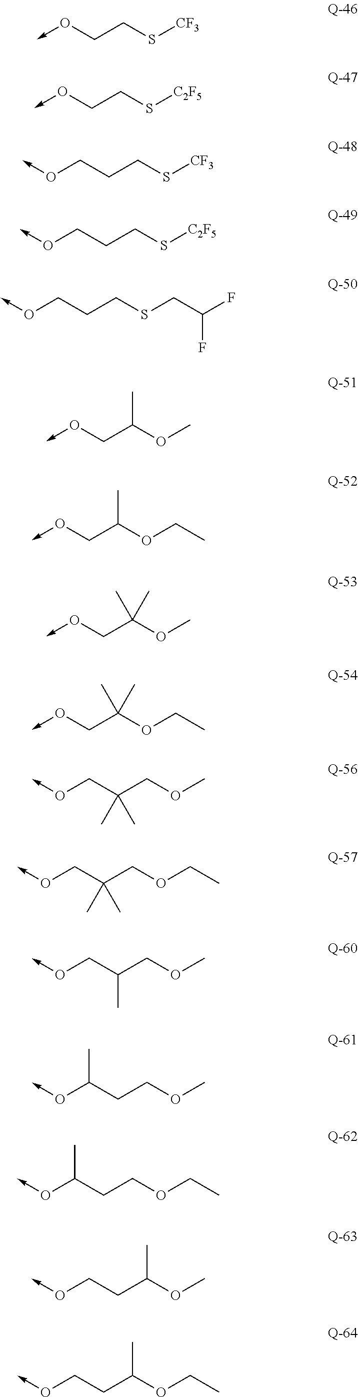

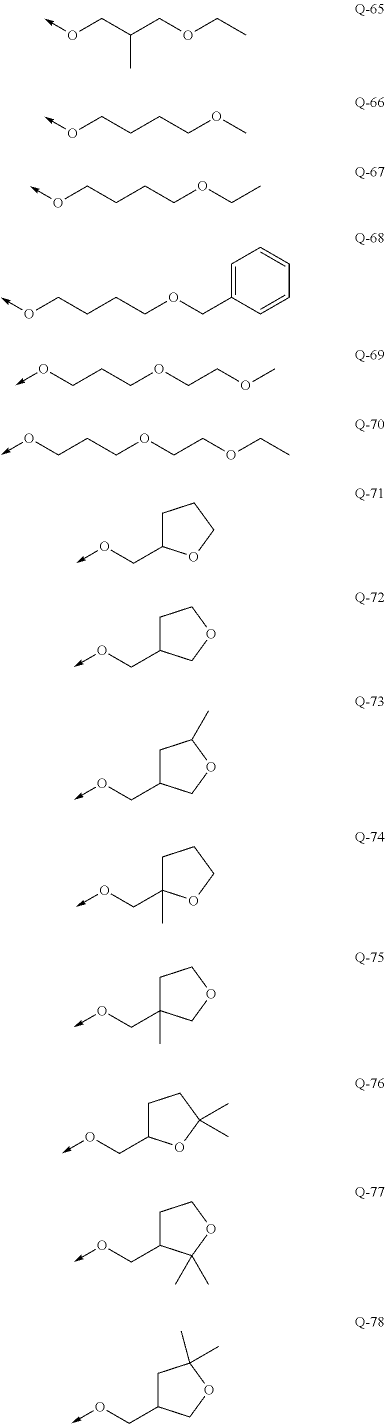

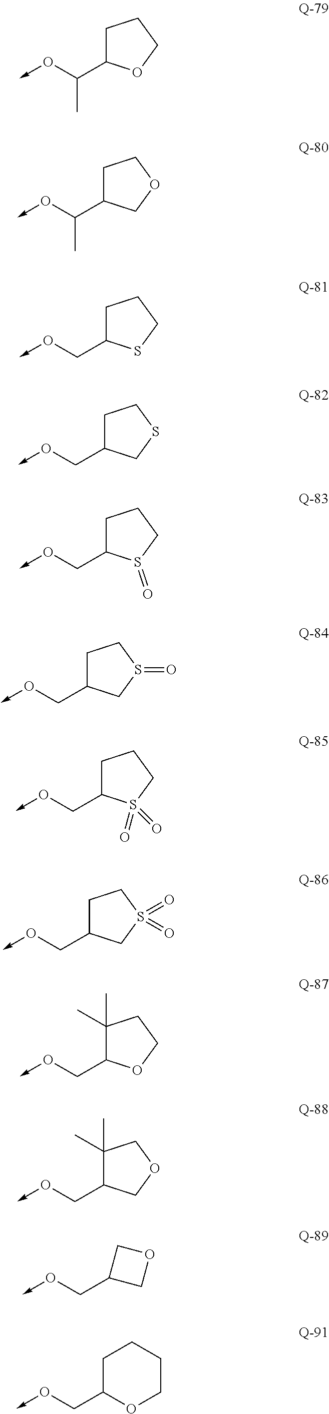

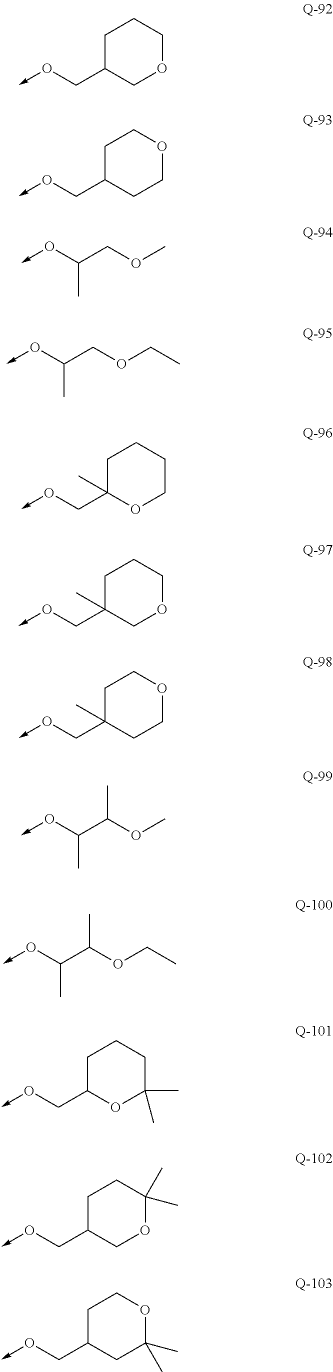

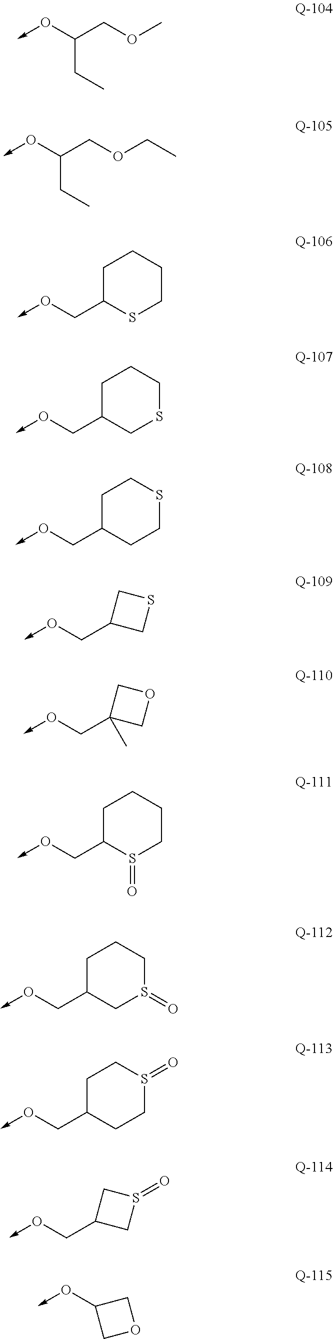

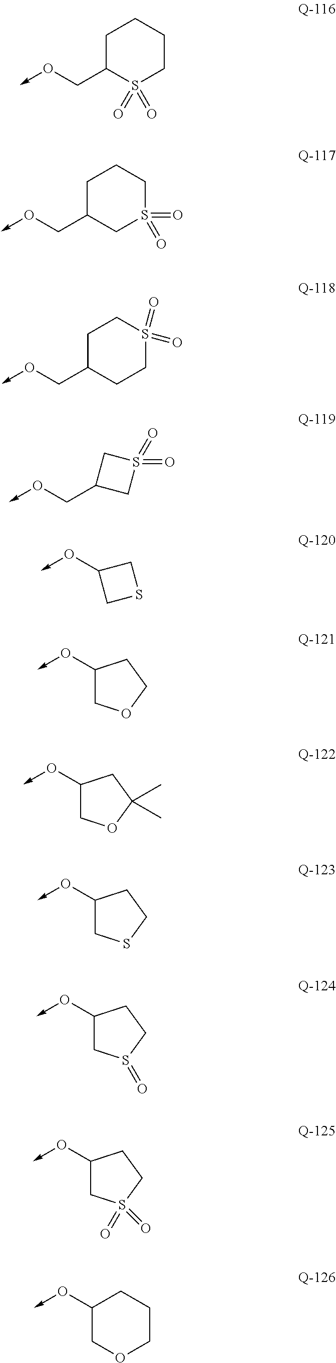

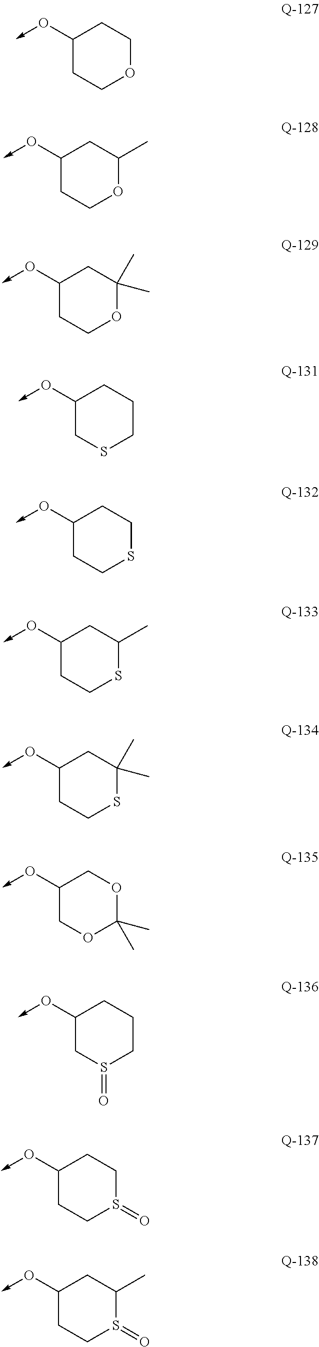

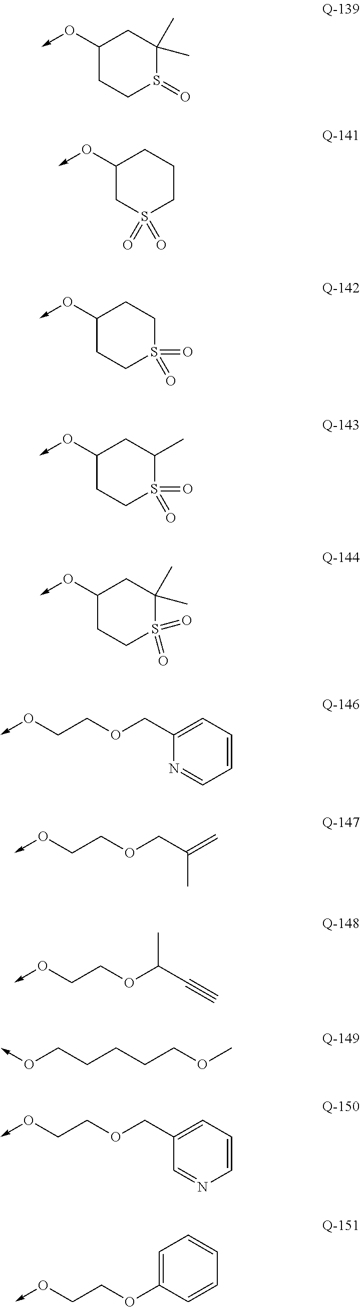

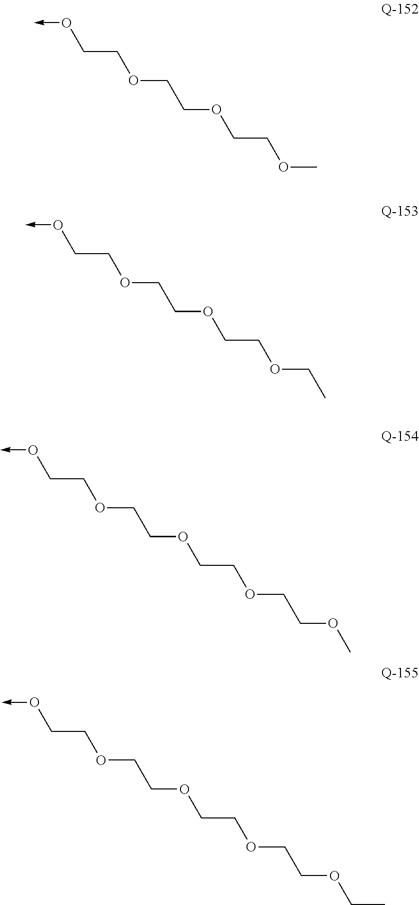

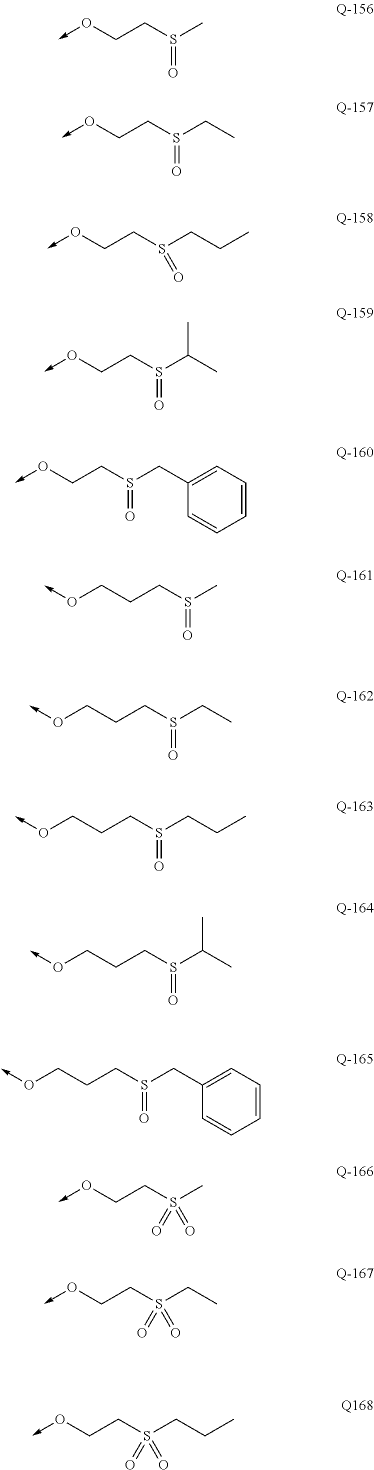

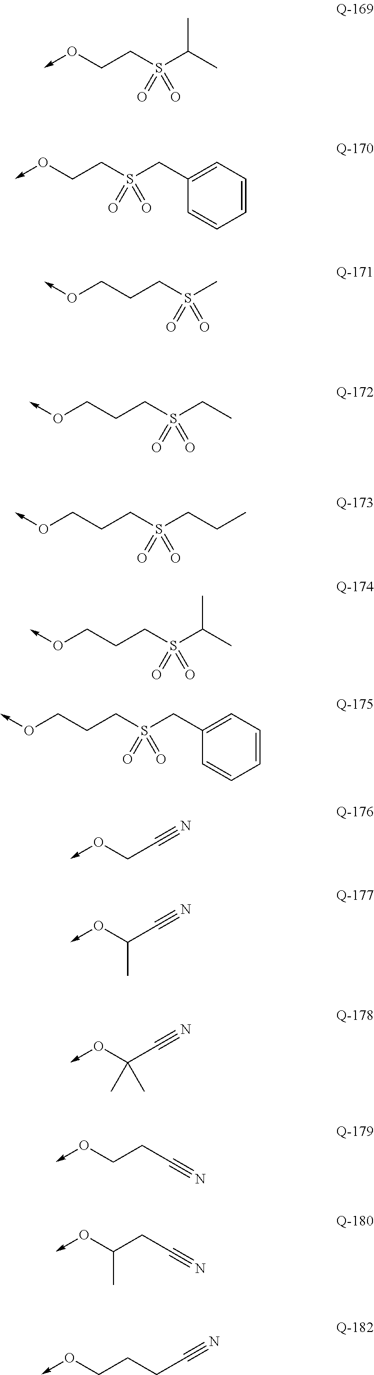

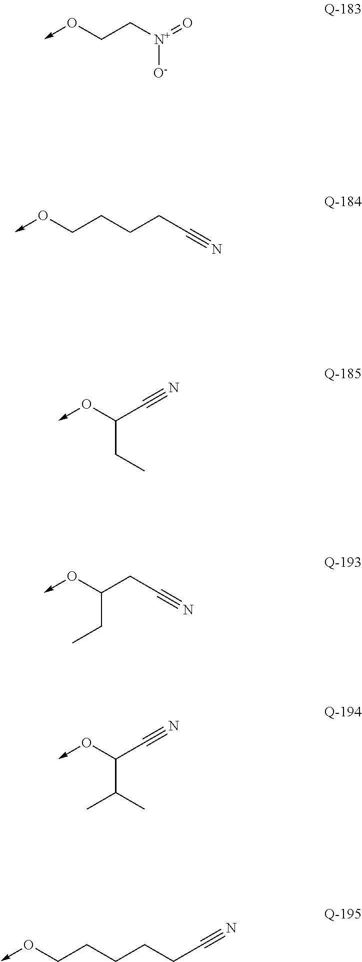

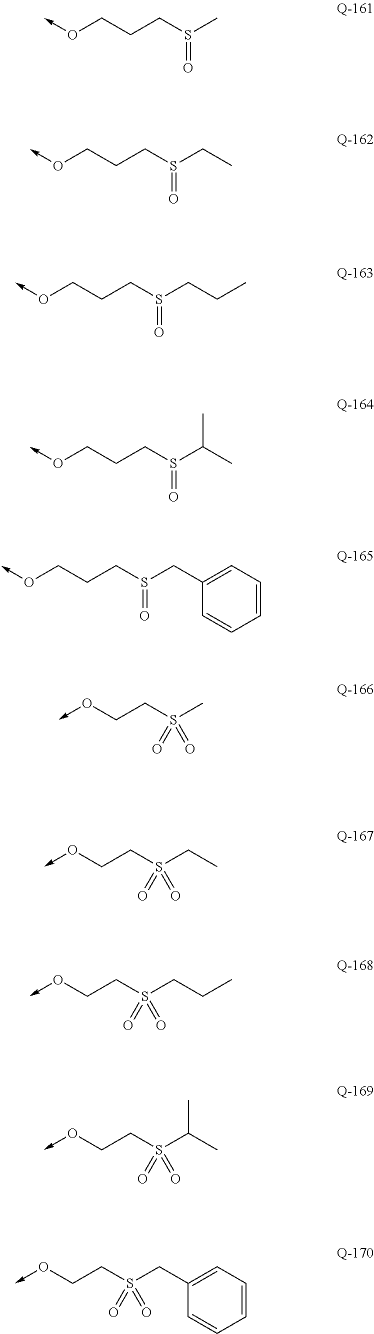

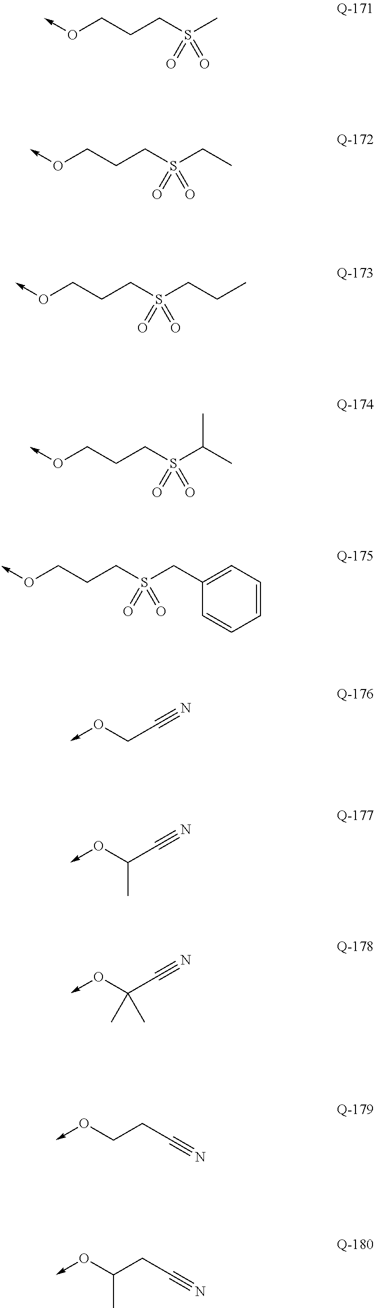

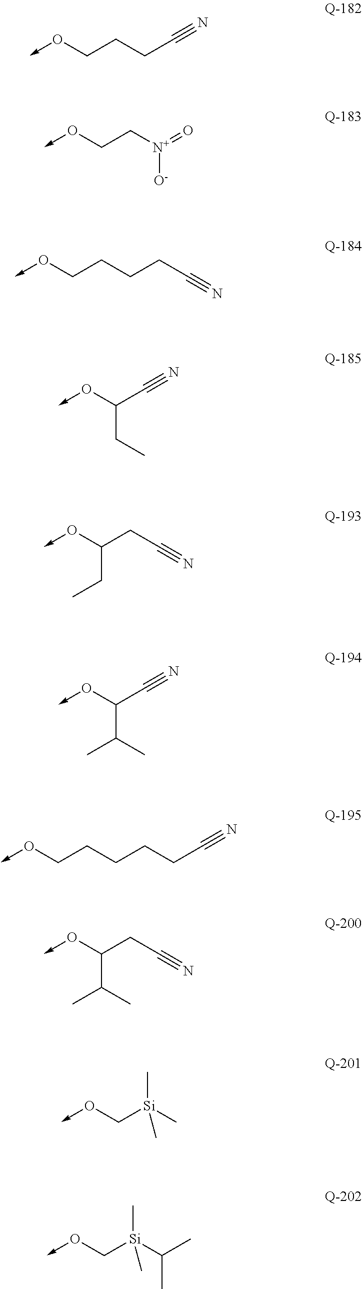

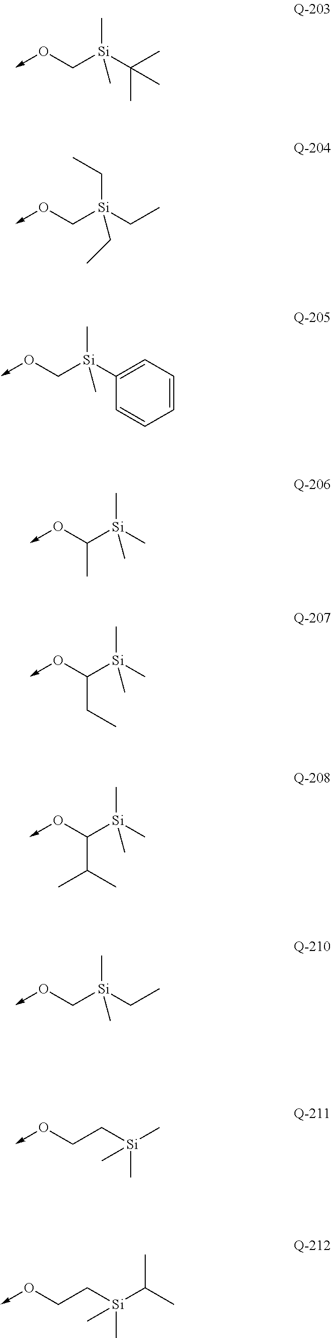

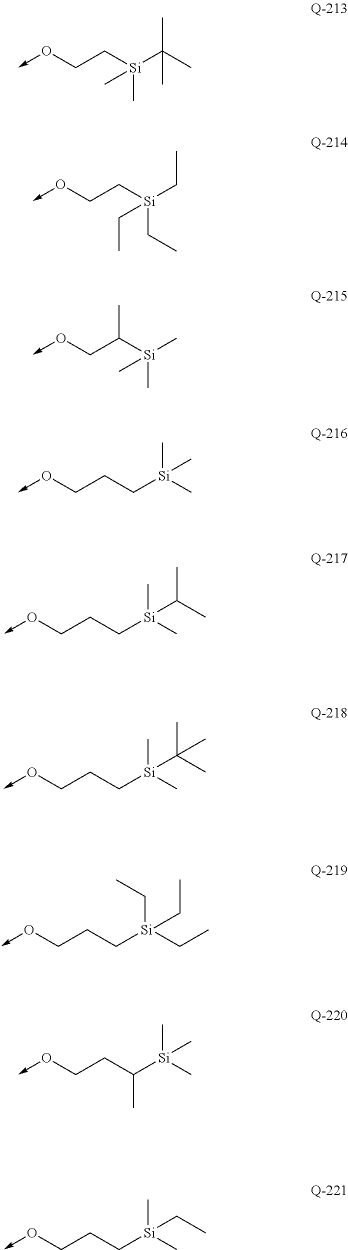

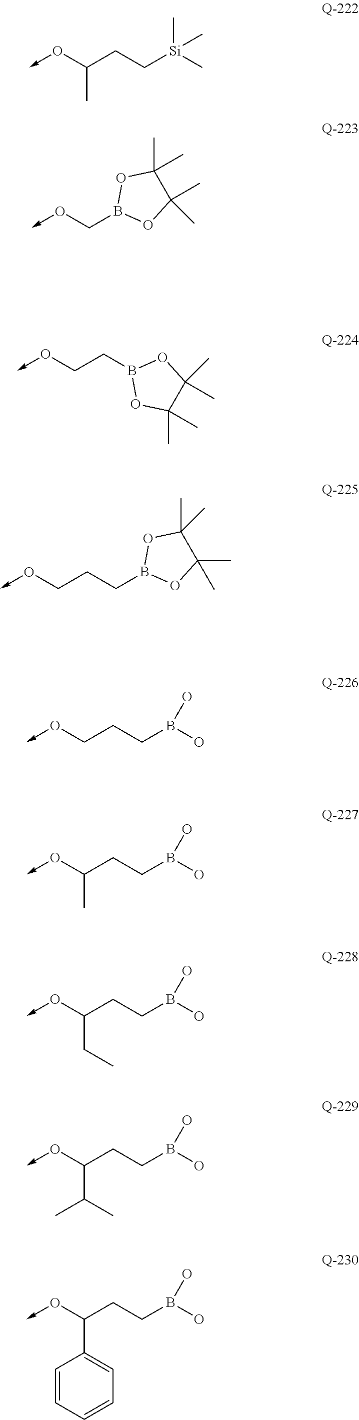

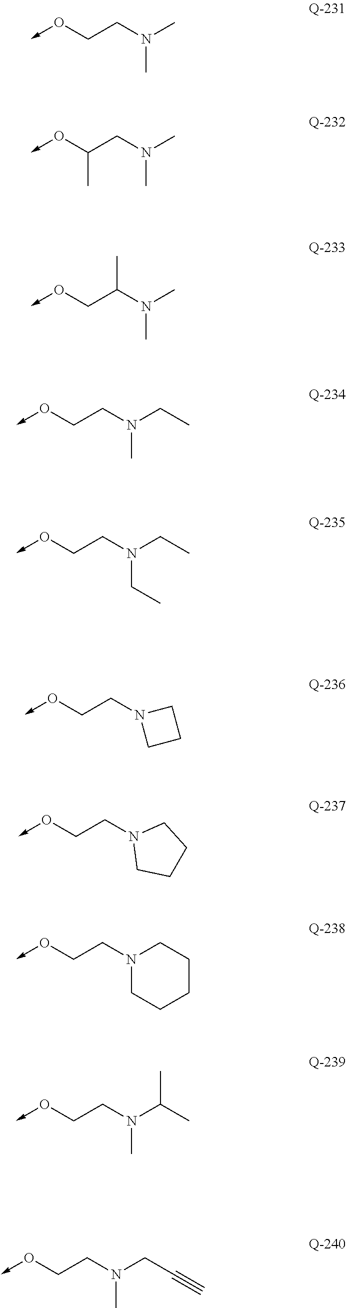

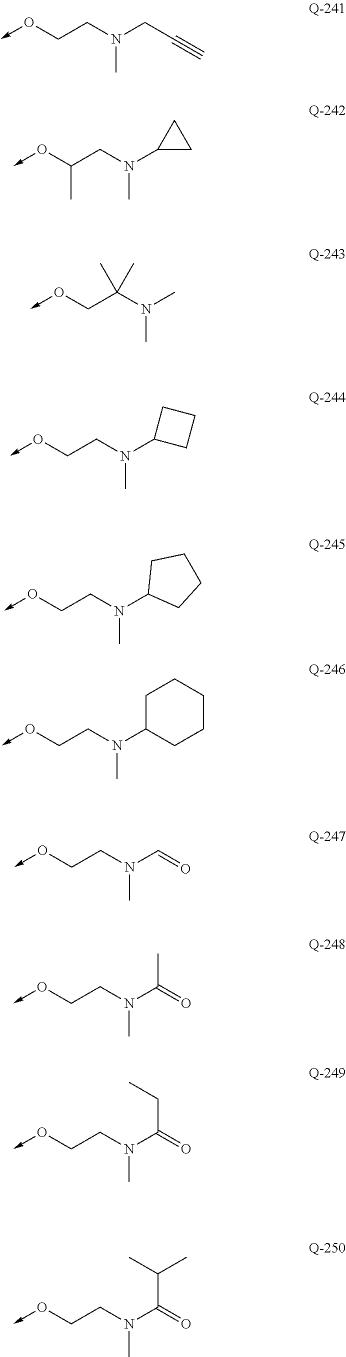

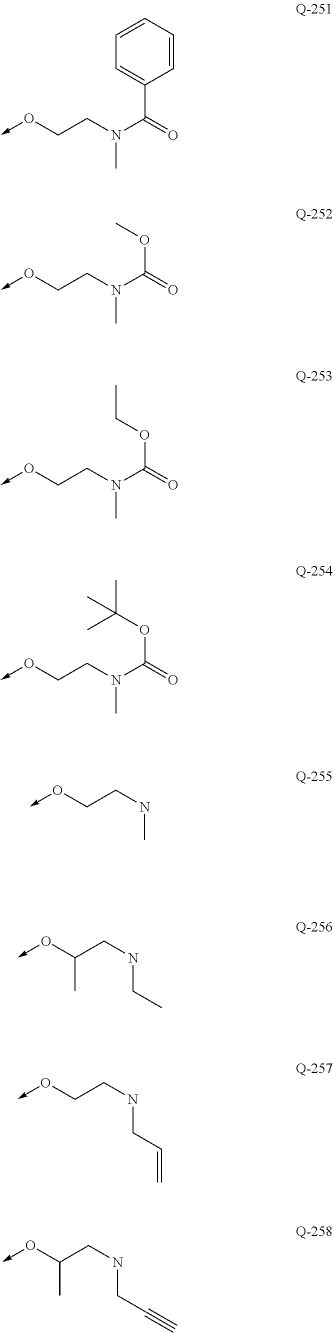

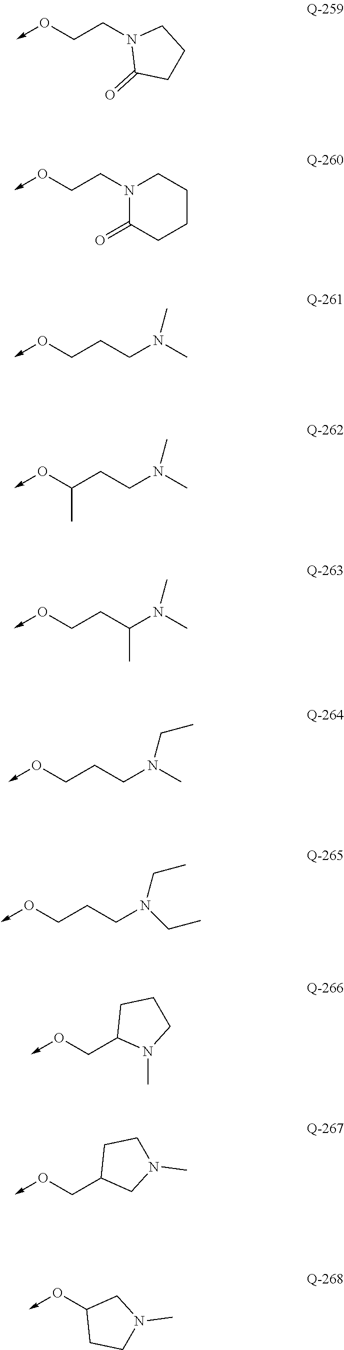

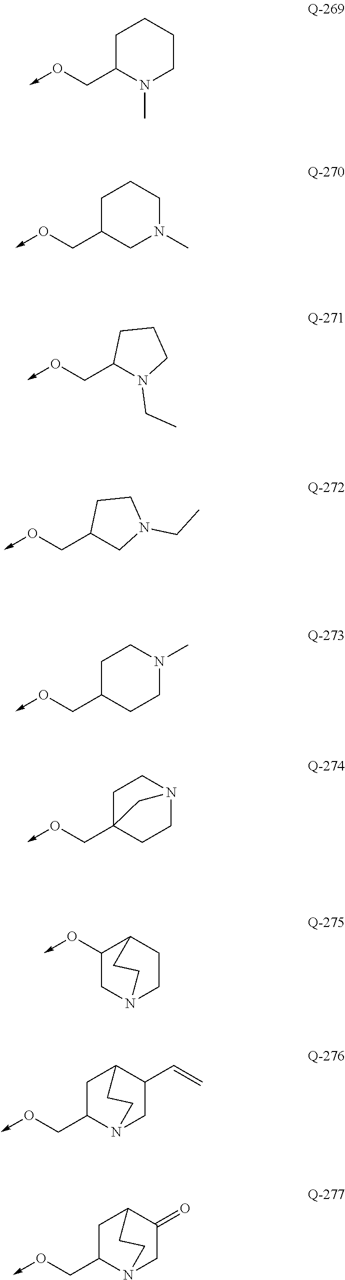

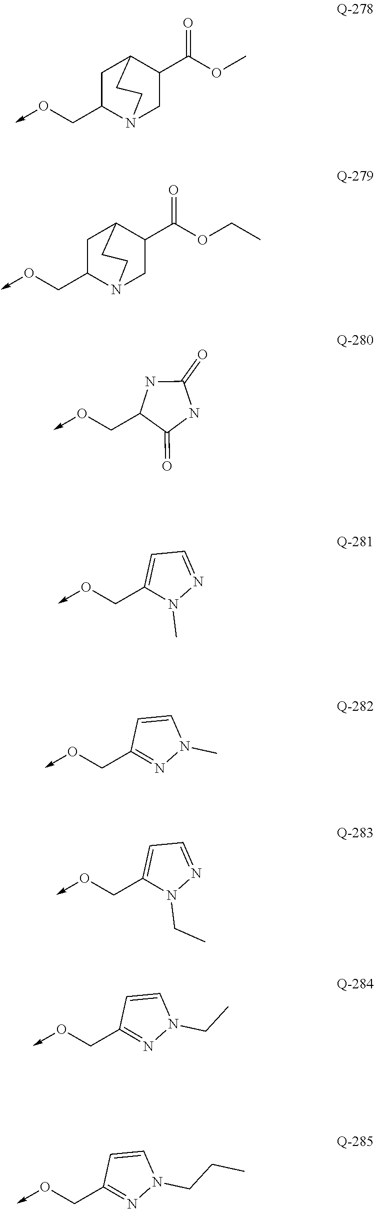

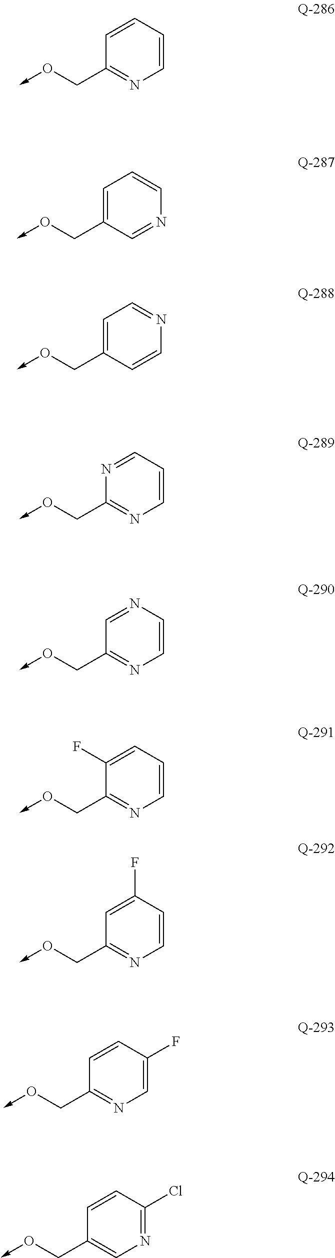

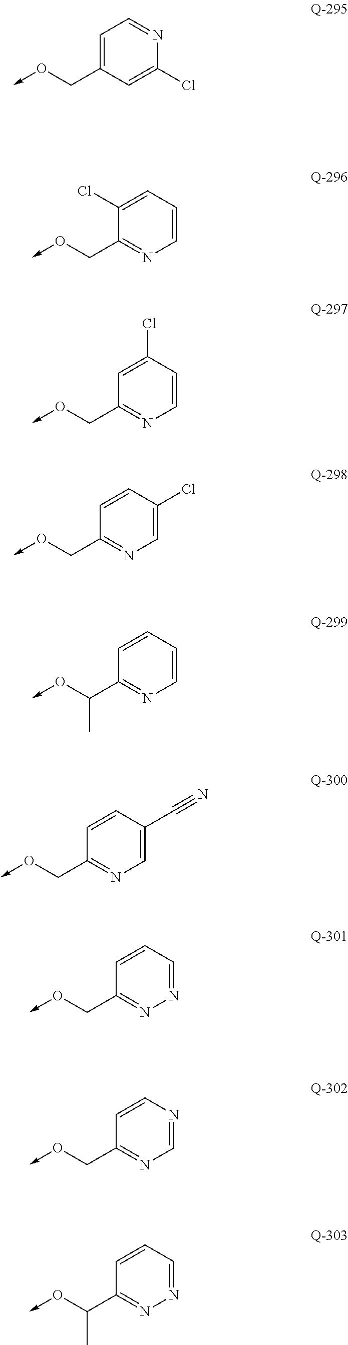

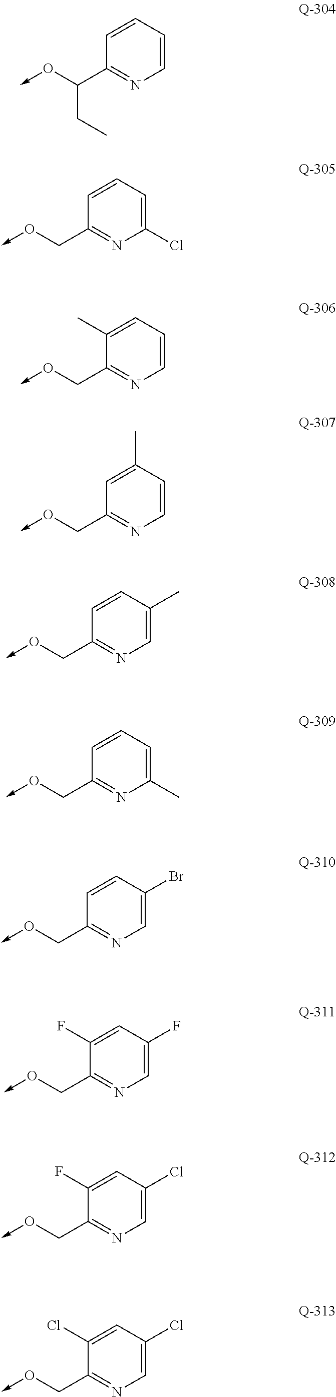

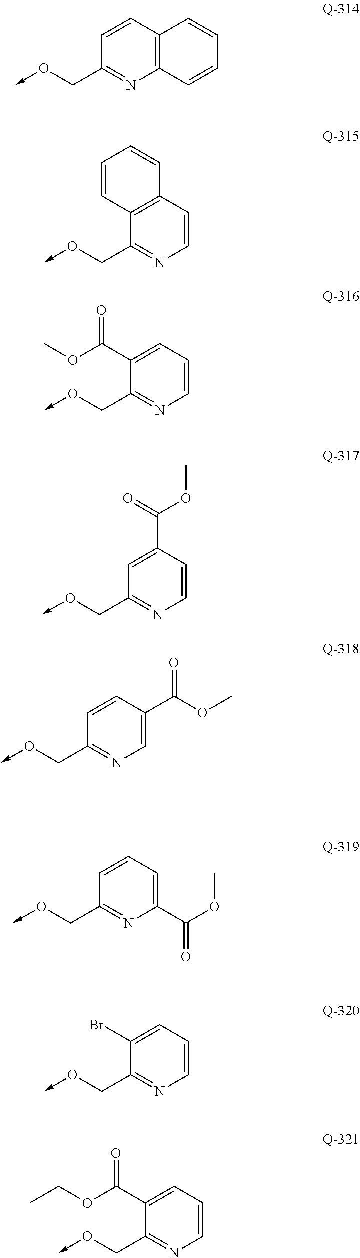

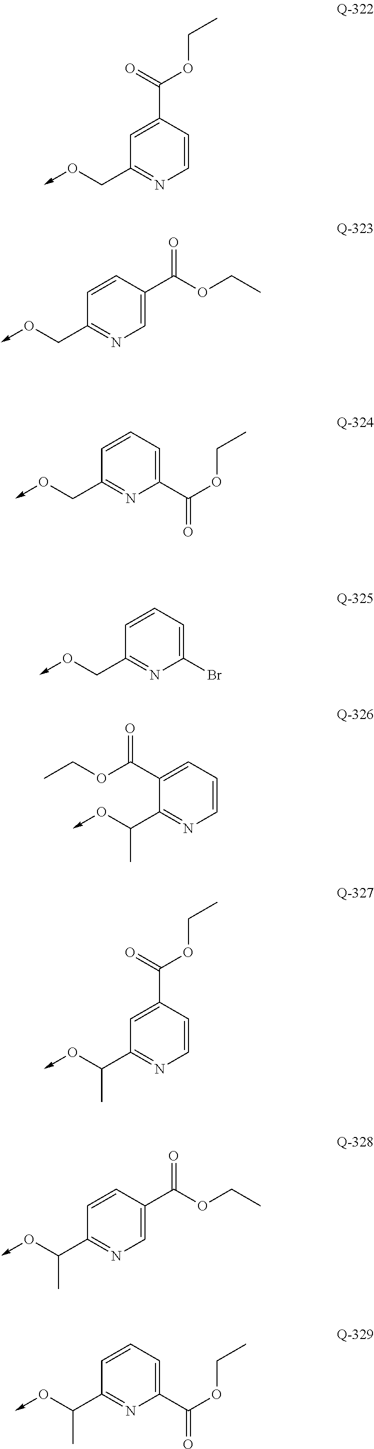

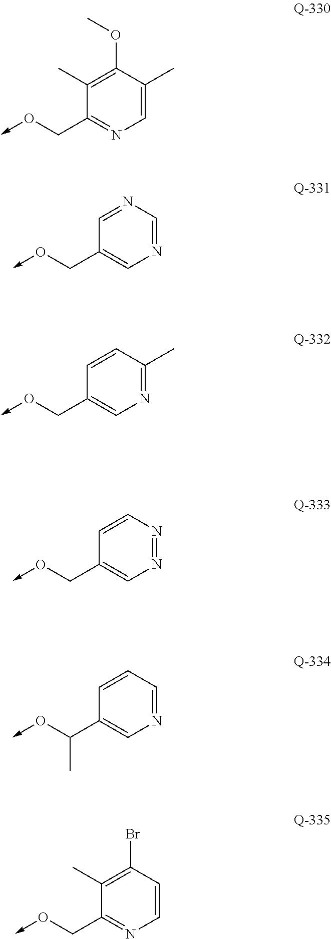

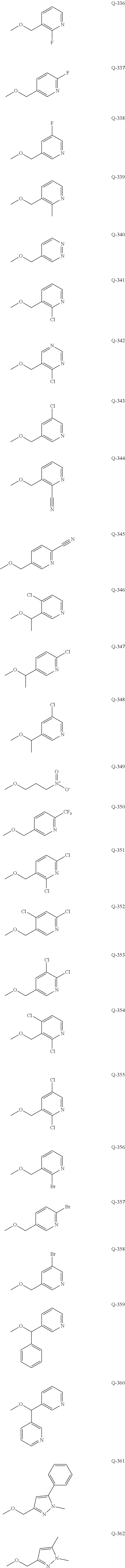

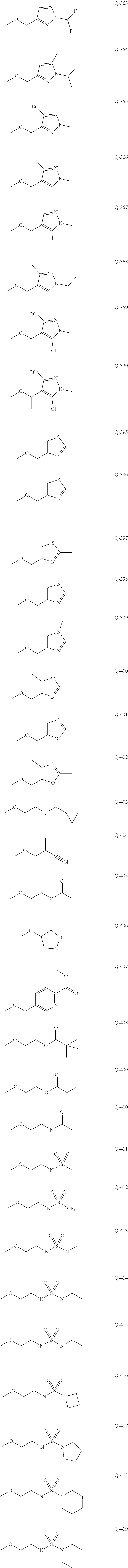

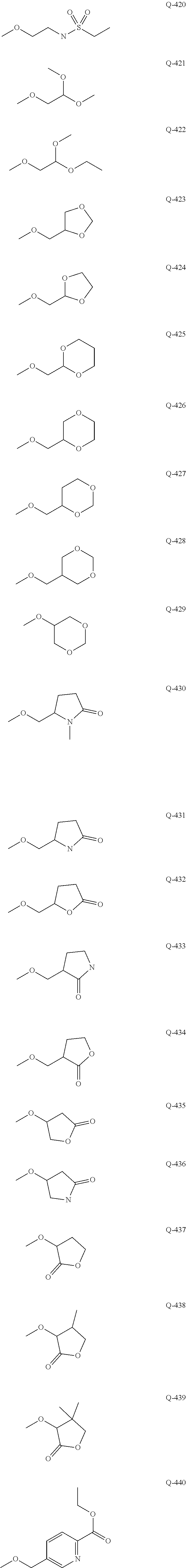

Examples of such herbicidally active compounds within the scope of formula (1) include:

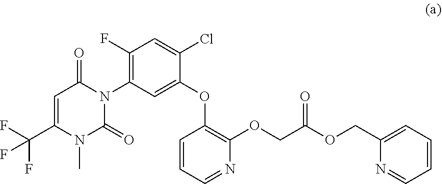 [SMILES: Cn1c(C(F)(F)F)cc(=O)n(-c2cc(Oc3cccnc3OCC(=O)OCc3ccccn3)c(Cl)cc2F)c1=O]

pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

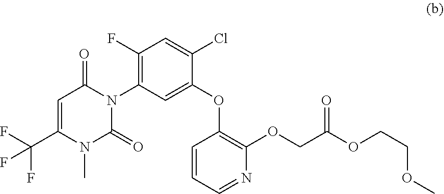 [SMILES: COCCOC(=O)COc1ncccc1Oc1cc(-n2c(=O)cc(C(F)(F)F)n(C)c2=O)c(F)cc1Cl]

2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate;

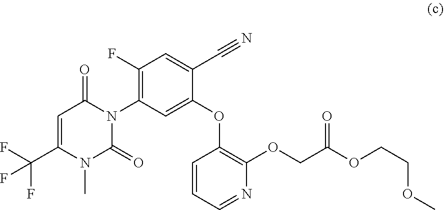 [SMILES: COCCOC(=O)COc1ncccc1Oc1cc(-n2c(=O)cc(C(F)(F)F)n(C)c2=O)c(F)cc1C#N]

2-methoxyethyl[(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate; and

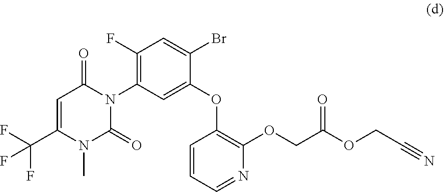 [SMILES: Cn1c(C(F)(F)F)cc(=O)n(-c2cc(Oc3cccnc3OCC(=O)OCC#N)c(Br)cc2F)c1=O]

cyanomethyl[(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate.

2) an herbicidally active compound of the general formula (II) or an agrochemically acceptable salt thereof

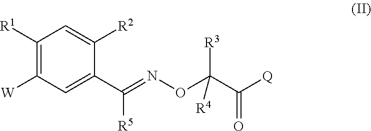

in which:
W represents a group W-1 to W-3

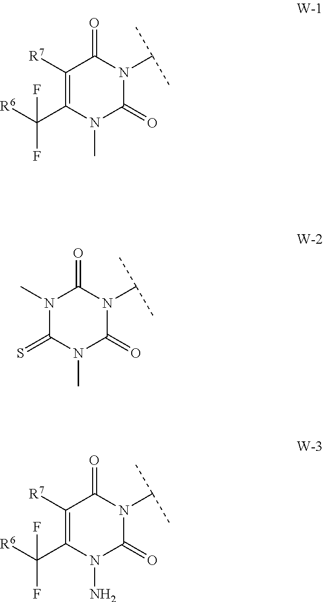

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O—(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl,
or
R³ and R⁴ together with the carbon atom to which they are bonded form a fully saturated or partly saturated 3- to 10-membered carbocyclic ring optionally having further substitution,
R⁵ represents hydrogen, (C₁-C₈)-alkyl, (C₁-C₈)-haloalkyl, R¹³O—(C₁-C₈)-alkyl, (C₂-C₈)-alkenyl, aryl-(C₁-C₈)-alkyl, heteroaryl-(C₁-C₈)-alkyl, heterocyclyl-(C₁-C₈)-alkyl, (C₃-C₈)-cycloalkyl, aryl, heteroaryl, or heterocyclyl,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen or methyl,
Q represents hydroxy or a group Q-1, Q-2

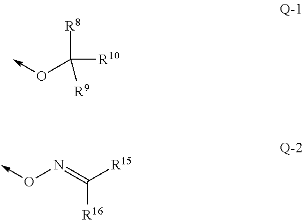

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₁C₈)-haloalkyl, (C₃C₈)-cycloalkyl, (C₃C₈)-cycloalkyl-(C₁C₈)-alkyl, (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O—(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR¹³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl. (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and R¹⁵ and R¹⁶ independently of each other represent (C₁-C₈)-alkyl, (C₃-C₈)-cycloalkyl, aryl, heteroaryl, or heterocyclyl.

Examples of such herbicidally active compounds within the scope of formula (II) include methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (also known as methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate or flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{1[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy} propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, and (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid.

3) an herbicidally active compound of the general formula (LIT) or an agrochemically acceptable salt thereof

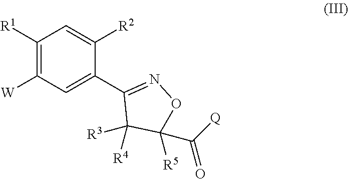

in which:
W represents a group W-1 to W-3

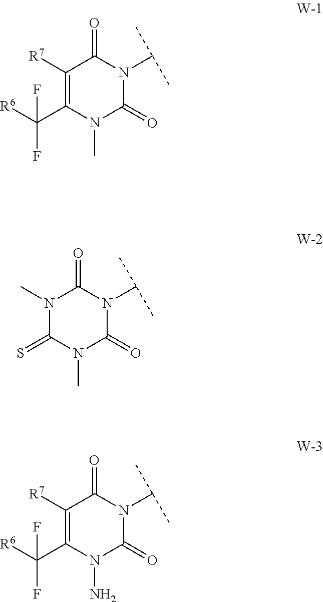

R¹ represents hydrogen, fluorine, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents fluorine, chlorine, bromine, cyano, nitro, C(O)NH₂, C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl,
R⁵ represents hydrogen, (C₁-C₈)-alkyl, (C₁-C₈)-haloalkyl, R¹³O—(C₁-C₈)-alkyl, (C₂-C₅)-alkenyl, aryl-(C₁-C₈)-alkyl, heteroaryl-(C₁-C₈)-alkyl, or heterocyclyl-(C₁-C₈)-alkyl, or
R³ and R⁵ together with the carbon atom to which they are bonded form a fully saturated or partly saturated 3- to 10-membered monocyclic or bicyclic ring optionally interrupted by heteroatoms and optionally having further substitution,
R⁶ represents hydrogen, fluorine, chlorine, bromine, trifluoromethyl, difluoromethyl, methoxy, ethoxy, prop-1-yloxy, or but-1-yloxy,
R⁷ represents hydrogen, methyl,
Q represents hydroxy or a group Q-1, Q-2

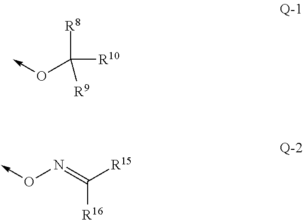

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, or (C₁-C₈)-alkoxy-(C₁-C₈)-alkyl,
R⁹ represents hydrogen, (C₁-C₈)-alkyl,
R¹⁰ represents hydrogen, halogen, cyano, nitro, (C₁-C₈)-alkyl, (C₁-C₈)-aaloalkyl, (C₃-C₈)-cycloalkyl, (C₃-C₈)-cycloalkyl-(C₁-C₈)-alkyl, (C₃-C₈)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹². SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-Dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C—(C₁-C₈)-alkyl, R¹¹R¹²N(O)C—(C₁-C₈)-alkyl, or bis-(C₁-C₈)-alkoxy-(C₁-C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl. (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₈)-cycloalkyl, aryl, heteroaryl, or heterocyclyl.

Examples of the herbicidally active compounds within the scope of formula (III) include ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, and methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate.

4) an herbicidally active compound corresponding to a compound selected from the group consisting of A1, A2, and A3, or an agrochemically acceptable salt thereof, wherein:
A1 corresponds to:

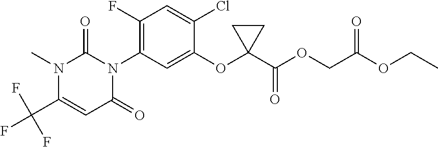 [SMILES: CCOC(=O)COC(=O)C1(Oc2cc(-n3c(=O)cc(C(F)(F)F)n(C)c3=O)c(F)cc2Cl)CC1]

2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
A2 corresponds to:

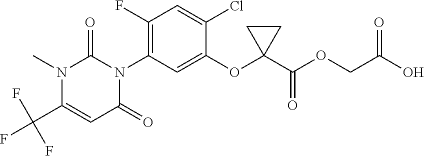 [SMILES: Cn1c(C(F)(F)F)cc(=O)n(-c2cc(OC3(C(=O)OCC(=O)O)CC3)c(Cl)cc2F)c1=O]

{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid; and
A3 corresponds to:

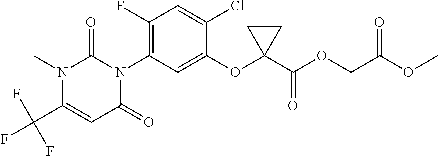 [SMILES: COC(=O)COC(=O)C1(Oc2cc(-n3c(=O)cc(C(F)(F)F)n(C)c3=O)c(F)cc2Cl)CC1]

2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate.

5) an herbicidally active compound of the general formula (IV) or an agrochemically acceptable salt thereof

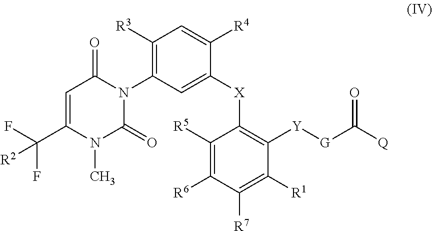

wherein
R¹ is hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy.
R² is hydrogen, fluoro, chloro, bromo, methyl, trifluormethyl, methoxy, ethoxy, prop-1-yloxy, or but-1-yloxy,
R³ is hydrogen, fluoro, chloro, bromo, methoxy, ethoxy, prop-1-yloxy, prop-2-yloxy, but-1-yloxy, but-2-yloxy, 2-methylprop-1-yloxy, or 1,1-dimethyleth-1-yloxy,
R⁴ is fluoro, chloro, bromo, cyano, NO₂, C(O)NH₂, C(S)NH₂, trifluormethyl, difluormethyl, pentafluorethyl, ethinyl, propin-1-yl, 1-butin-1-yl, pentin-1-yl, or hexin-1-yl,
R⁵, R⁶ and R⁷ are independently from each other hydrogen, fluoro, chloro, bromo, iodo, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-di-methylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluormethyl, difluormethyl, pentafluorethyl, 2,2-difluorethyl, 2,2,2-trifluorethyl, methoxy, ethoxy, prop-1-yloxy, prop-2-yloxy, but-1-yloxy, but-2-yloxy, 2-methylprop-1-yloxy, 1,1-dimethyleth-1-yloxy, difluormethoxy, trifluormethoxy, pentafluorethoxy, 2,2-difluorethoxy, or 2,2,2-trifluorethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylen, (1-methyl)ethyl-1-en, (2-methyl)ethyl-1-en, n-butylen, 1-methylpropyl-1-en, 2-methylpropyl-1-en, 3-methylpropyl-1-en, 1,1-dimethylethyl-1-en, 2,2-dimethylethyl-1-en, 1-ethylethyl-1-en, 2-ethylethyl-1-en, 1-(prop-1-yl)ethyl-1-en, 2-(prop-1-yl)ethyl-1-en, 1-(prop-2-yl)ethyl-1-en, 2-(prop-2-yl)ethyl-1-en, 1,1,2-trimethylethyl-1-en, 1,2,2-trimethylethyl-1-en, 1,1,2,2-tetramethylethyl-1-en, n-pentylen, 1-methylbutyl-1-en, 2-methylbutyl-1-en, 3-methylbutyl-1-en, 4-methylbutyl-1-en, 1,1-dimethylpropyl-1-en, 2,2-dimethylpropyl-1-en, 3,3-dimethylpropyl-1-en, 1,2-dimethylpropyl-1-en, 1,3-dimethylpropyl-1-en, 1-ethylpropyl-1-en, n-hexylen, 1-methylpentyl-1-en, 2-methylpentyl-1-en, 3-methylpentyl-1-en, 4-methylpentyl-1-en, 1,1-dimethylbutyl-1-en, 1,2-dimethylbutyl-1-en, 1,3-di-methylbutyl-1-en, 2,2-dimethylbutyl-1-en, 2,3-dimethylbutyl-1-en, 3,3-dimethylbutyl-1-en, 1-ethylbutyl-1-en, 2-ethylbutyl-1-en, 1,1,2-trimethylpropyl-1-en, 1,2,2-trimethylpropyl-1-en, 1-ethyl-1-methylpropyl-1-en, or 1-ethyl-2-methylpropyl-1-en,
X and Y are independently from each other O (oxygen) or S (sulfur)
and
Q is one of the groups Q-1 to Q-25, wherein in the formulae of the following table the arrow stands for a bond of the respective group Q to the carbonyl group in the general formula (I):

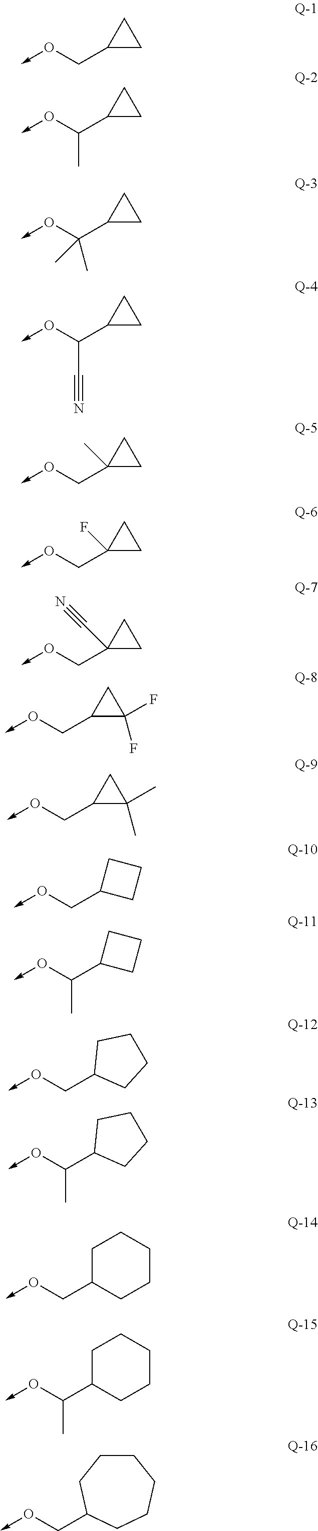

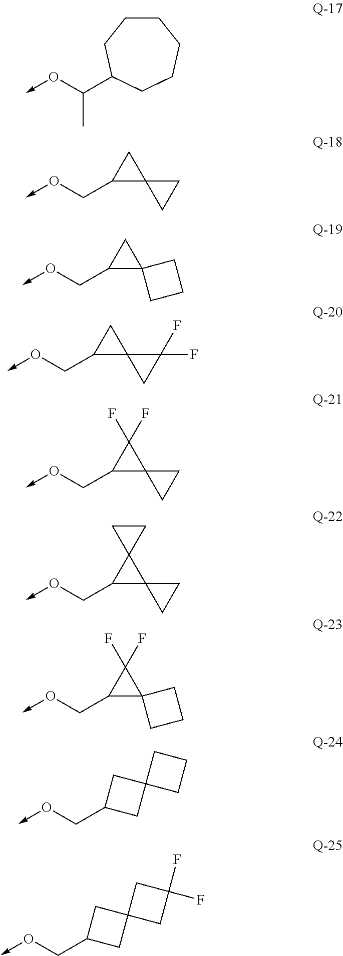

An example of an herbicidally active compound within the scope of formula (IV) is cyclopropylmethyl-(2-{2-chlor-4-fluor-5-[3-methyl-2,6-dioxo-4-(trifluormethyl)-3,6-dihydropyrimidin-1(2H)-yl]phenoxy}phenoxy)acetate, which has the following structure:

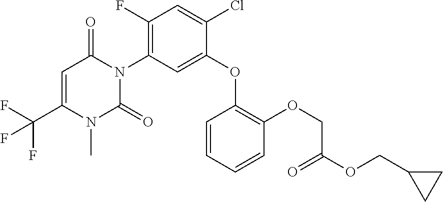 [SMILES: Cn1c(C(F)(F)F)cc(=O)n(-c2cc(Oc3ccccc3OCC(=O)OCC3CC3)c(Cl)cc2F)c1=O]

6) an herbicidally active compound of the general formula (V) or an agrochemically acceptable salt thereof

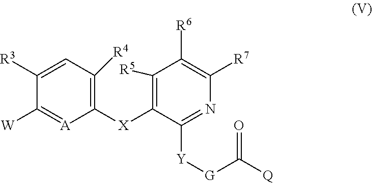

in which:
W represents a group W-1 to W-2

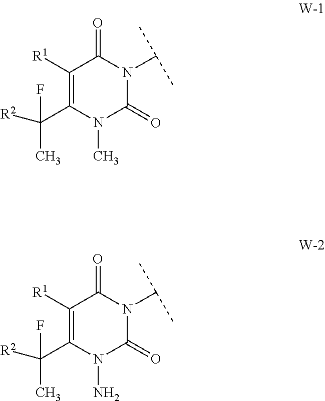

A represents nitrogen or CH;
R¹ represents hydrogen or methyl;
R² represents hydrogen or fluorine;
R³ represents hydrogen, halogen, or (C₁-C₈)-alkoxy;
R⁴ represents halogen, cyano, NO₂, C(O)NH₂, C(S)NH₂, (C₁-C₈)-haloalkyl, or (C₂-C₈)-alkynyl;
R⁵, R⁶, and R⁷ independently of one another represent hydrogen, halogen, cyano, (C₁-C₈)-alkyl, (C₁-C₈)-haloalkyl, (C₁-C₈)-alkoxy, or (C₁-C₈)-haloalkoxy;
G represents a branched or unbranched alkylene group optionally substituted with an (C₁-C₃)-alkoxy group;
Q represents hydroxy or a group Q-1, Q-2;

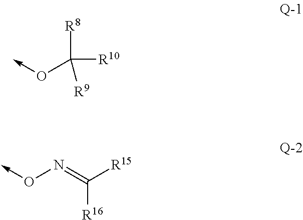

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³. C(O)OR¹³, or (C₁-C₈)-alkoxy-(C₁-C₈)-alkyl;
R⁹ represents hydrogen or (C₁-C₈)-alkyl;
R¹⁹ represents hydrogen, halogen, cyano, NO₂, (C₁-C₈)-alkyl, (C₁-C₈)-haloalkyl, (C₃-C₈)-cycloalkyl, (C₃-C₈)-cycloalkyl-(C₁-C₈)-alkyl, (C₃-C₈)-halocycloalkyl, (C₃-C₈)-halocycloalkyl-(C₁-C₈)-alkyl, (C₂-C₈)-alkenyl, (C₂-C₈)-alkynyl, aryl, aryl-(C₁-C₈)-alkyl, heteroaryl, heteroaryl-(C₁-C₈)-alkyl, heterocyclyl, heterocyclyl-(C₁-C₈)-alkyl, R¹¹R¹²N—(C₁-C₈)-alkyl. R¹³O—(C₁-C₈)-alkyl, cyano-(C₁-C₈)-alkyl, (C₁-C₈)-alkylcarbonyloxy-(C₁-C₅)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, tris-[(C₁-C₈)-alkyl]silyl, bis-hydroxyboryl-(C₁-C₈)-alkyl, bis-[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)OR¹³, C(O)R¹³, C(O)NR¹¹R¹², R¹³O(O)C—(C₁-C₈)-alkyl, R¹¹R¹²N(O)C—(C₁-C₈)-alkyl, or bis-(C₁-C₈)-alkoxy-(C₁-C₈)-alkyl, or
R⁸ and R¹⁰ together with the carbon atom to which they are attached form a fully saturated or partially saturated 3- to 10-membered monocyclic or bicyclic ring optionally interrupted by heteroatoms and optionally having further substitution;
R¹¹ and R¹² are identical or different and independently of one another represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are attached form a fully saturated or partially saturated 3- to 10-membered monocyclic or bicyclic ring optionally interrupted by heteroatoms and optionally having further substitution;
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl. (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₅)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]aminocarbonyl-(C₁-C₈)-alkyl. (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl;
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkyl-amino, aryl-(C₁-C₈)-amino, aryl-(C₁-C₆)-alkyl-amino, aryl-[(C₁-C₈)-alkyl]amino, (C₃-C₈)-cycloalkyl-amino, (C₃-C₈)-cycloalkyl-[(C₁-C₈)-alkyl]amino; N-azetidinyl, N-pyrrolidinyl, N-piperidinyl, or N-morpholinyl;
R¹⁵ and R¹⁶ independently of each other represent (C₁-C₈)-alkyl, (C₃-C₈)-cycloalkyl, aryl, heteroaryl, or heterocyclyl or
R¹⁵ and R¹⁶ together, with the carbon atom to which they are attached, form an unsubstituted (C₃-C₇)-cycloalkyl;
X and Y, independently of one another, represent oxygen or sulfur; and
Z represents nitrogen or CH.

Examples of herbicidally active compounds within the scope of formula (V) include: ethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate; [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetic acid; ethyl (2-{2-chloro-5-[4-(1,1-difluoroethyl)-3-methyl-2,6-dioxo-3,6-dihydropyrimidin-1(2H)-yl]-4-fluorophenoxy}phenoxy)acetate; (2-{2-chloro-5-[4-(1,1-difluoroethyl)-3-methyl-2,6-dioxo-3,6-dihydropyrimidin-1(2H)-yl]-4-fluorophenoxy}phenoxy)acetic acid; ethyl (2-{2-chloro-4-fluoro-5-[4-(1-fluoroethyl)-3-methyl-2,6-dioxo-3,6-dihydropyrimidin-1(2H)-yl]phenoxy}phenoxy)acetate; 2-methoxyethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate; tetrahydrofuran-2-ylmethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate; cyanomethyl [(3-{2-chloro-5-[4-(1,1-difluoroethyl)-3-methyl-2,6-dioxo-3,6-dihydropyrimidin-1(2H)-yl]-4-fluorophenoxy}pyridin-2-yl)oxy]acetate; methyl (2-{2-chloro-5-[4-(1,1-difluoroethyl)-3-methyl-2,6-dioxo-3,6-dihydropyrimidin-1(2H)-yl]-4-fluorophenoxy}phenoxy)(methoxy) acetate; methyl (2-{2-bromo-5-[4-(1,1-difluoroethyl)-3-methyl-2,6-dioxo-3,6-dihydropyrimidin-1(2H)-yl]-4-fluorophenoxy}phenoxy)(methoxy)acetate; [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetic acid; ethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate; 2-methoxyethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate; tetrahydrofuran-2-ylmethyl [(3-{2-bromo-5-[4-(1,1-difluoroethyl)-3-methyl-2,6-dioxo-3,6-dihydropyrimidin-1(2H)-yl]-4-fluorophenoxy}pyridin-2-yl)oxy]acetate; and ethyl 2-[[3-[5-[4-(1,1-difluoroethyl)-3-methyl-2,6-dioxo-pyrimidin-1-yl]-4-fluoro-2-nitro-phenoxy]-2-pyridyl]oxy]acetate.

7) an herbicidally active compound corresponding to a compound selected from the group consisting of B1, B2, B3, B4, B5, and B6, or an agrochemically acceptable salt thereof, wherein:
B1 corresponds to:

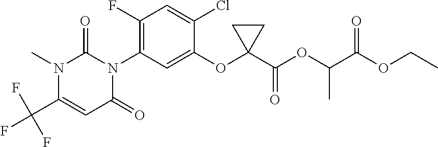 [SMILES: CCOC(=O)C(C)OC(=O)C1(Oc2cc(-n3c(=O)cc(C(F)(F)F)n(C)c3=O)c(F)cc2Cl)CC1]

1-ethoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
B2 corresponds to:

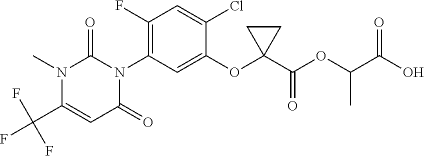 [SMILES: CC(OC(=O)C1(Oc2cc(-n3c(=O)cc(C(F)(F)F)n(C)c3=O)c(F)cc2Cl)CC1)C(=O)O]

2-{[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}propanoic acid;
B3 corresponds to:

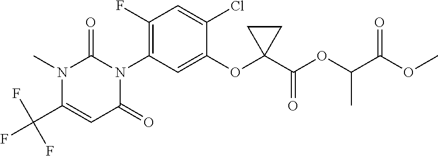 [SMILES: COC(=O)C(C)OC(=O)C1(Oc2cc(-n3c(=O)cc(C(F)(F)F)n(C)c3=O)c(F)cc2Cl)CC1]

1-methoxy-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
B4 corresponds to:

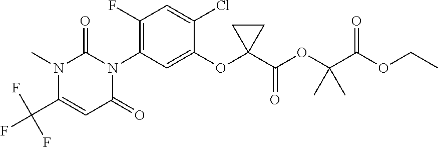 [SMILES: CCOC(=O)C(C)(C)OC(=O)C1(Oc2cc(-n3c(=O)cc(C(F)(F)F)n(C)c3=O)c(F)cc2Cl)CC1]

1-ethoxy-2-methyl-1-oxopropan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
B5 corresponds to:

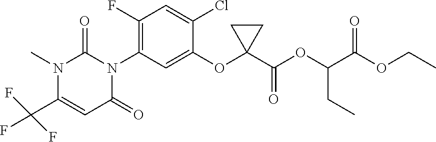 [SMILES: CCOC(=O)C(CC)OC(=O)C1(Oc2cc(-n3c(=O)cc(C(F)(F)F)n(C)c3=O)c(F)cc2Cl)CC1]

1-ethoxy-1-oxobutan-2-yl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
B6 corresponds to:

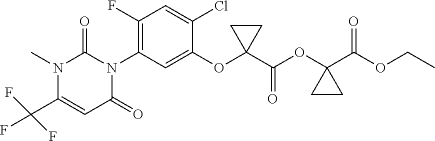 [SMILES: CCOC(=O)C1(OC(=O)C2(Oc3cc(-n4c(=O)cc(C(F)(F)F)n(C)c4=O)c(F)cc3Cl)CC2)CC1]

1-(ethoxycarbonyl)cyclopropyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate.

8) an herbicidally active compound corresponding to a compound selected from the group consisting of C1, C2, C3, C4, C5, C6. C7, C8, C9, C10, C11, and C12 or an agrochemically acceptable salt thereof, wherein:

C1 corresponds to:

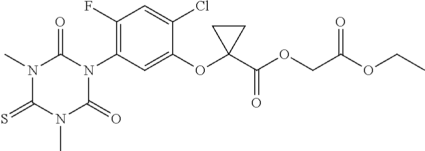 [SMILES: CCOC(=O)COC(=O)C1(Oc2cc(-n3c(=O)n(C)c(=S)n(C)c3=O)c(F)cc2Cl)CC1]

2-ethoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate
C2 corresponds to:

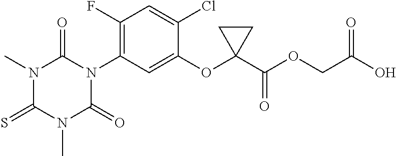 [SMILES: Cn1c(=S)n(C)c(=O)n(-c2cc(OC3(C(=O)OCC(=O)O)CC3)c(Cl)cc2F)c1=O]

[({1-[2-chloro-5-(3,5-dimethyl-2,6-dioxo-4-sulfanylidene-1,3,5-triazinan-1-yl)-4-fluorophenoxy]cyclopropyl}carbonyl)oxy]acetic acid
C3 corresponds to:

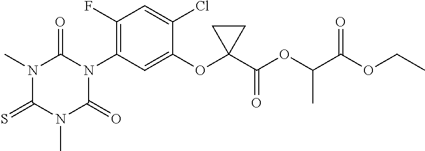 [SMILES: CCOC(=O)C(C)OC(=O)C1(Oc2cc(-n3c(=O)n(C)c(=S)n(C)c3=O)c(F)cc2Cl)CC1]

1-ethoxy-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate
C4 corresponds to:

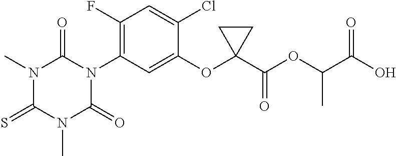 [SMILES: CC(OC(=O)C1(Oc2cc(-n3c(=O)n(C)c(=S)n(C)c3=O)c(F)cc2Cl)CC1)C(=O)O]

2-[({1-[2-chloro-5-(3,5-dimethyl-2,6-dioxo-4-sulfanylidene-1,3,5-triazinan-1-yl)-4-fluorophenoxy]cyclopropyl}carbonyl)oxy]propanoic acid
C5 corresponds to:

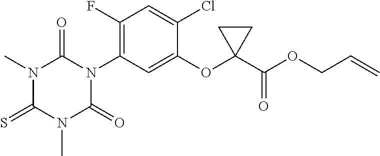 [SMILES: C=CCOC(=O)C1(Oc2cc(-n3c(=O)n(C)c(=S)n(C)c3=O)c(F)cc2Cl)CC1]

allyl 1-[2-chloro-5-(3,5-dimethyl-2,6-dioxo-4-sulfanylidene-1,3,5-triazinan-1-yl)-4-fluorophenoxy]cyclopropanecarboxylate
C6 corresponds to:

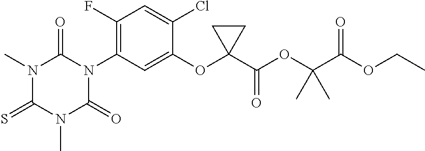 [SMILES: CCOC(=O)C(C)(C)OC(=O)C1(Oc2cc(-n3c(=O)n(C)c(=S)n(C)c3=O)c(F)cc2Cl)CC1]

1-ethoxy-2-methyl-1-oxopropan-2-yl 1-[2-chloro-5-(3,5-dimethyl-2,6-dioxo-4-sulfanylidene-1,3,5-triazinan-1-yl)-4-fluorophenoxy]cyclopropanecarboxylate
C7 corresponds to:

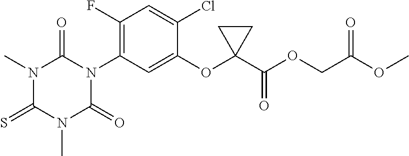

2-methoxy-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate
C8 corresponds to:

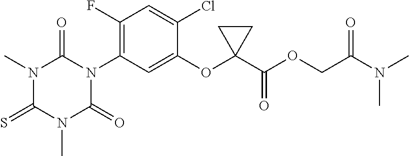

2-(dimethylamino)-2-oxoethyl 1-[2-chloro-5-(3,5-dimethyl-2,6-dioxo-4-sulfanylidene-1,3,5-triazinan-1-yl)-4-fluorophenoxy]cyclopropanecarboxylate
C9 corresponds to:

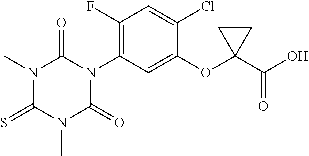 [SMILES: Cn1c(=S)n(C)c(=O)n(-c2cc(OC3(C(=O)O)CC3)c(Cl)cc2F)c1=O]

1-[2-chloro-5-(3,5-dimethyl-2,6-dioxo-4-sulfanylidene-1,3,5-triazinan-1-yl)-4-fluorophenoxy]cyclopropanecarboxylic acid
C10 corresponds to:

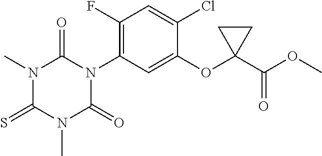

methyl 1-[2-chloro-5-(3,5-dimethyl-2,6-dioxo-4-sulfanylidene-1,3,5-triazinan-1-yl)-4-fluorophenoxy]cyclopropanecarboxylate
C11 corresponds to:

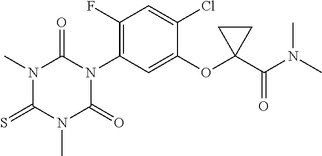

1-[2-chloro-5-(3,5-dimethyl-2,6-dioxo-4-sulfanylidene-1,3,5-triazinan-1-yl)4-fluorophenoxy]-N,N-dimethylcyclopropanecarboxamide
C12 corresponds to:

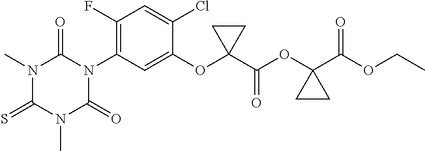 [SMILES: CCOC(=O)C1(OC(=O)C2(Oc3cc(-n4c(=O)n(C)c(=S)n(C)c4=O)c(F)cc3Cl)CC2)CC1]

ethyl 1-({1-[2-chloro-5-(3,5-dimethyl-2,6-dioxo-4-sulfanylidene-1,3,5-triazinan-1-yl)-4-fluorophenoxy]cyclopropyl}carbonyl)cyclopropanecarboxylate.

9) an herbicidally active compound of the general formula (VI) or an agrochemically acceptable salt thereof

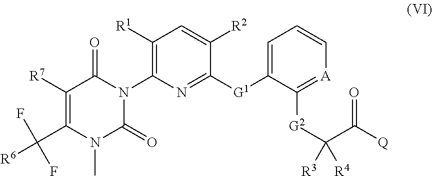

in which
A represents N (nitrogen) or a group C—R⁵,
G¹ and G² independently of each other represent O (oxygen) or S (sulfur),
R¹ represents fluorine, hydrogen, chlorine, bromine, methoxy, ethoxy, prop-1-yloxy, prop-2-yloxy, but-1-yloxy, but-2-yloxy, 2-methylprop-1-yloxy, or 1,1-dimethyleth-1-yloxy,
R² represents chlorine, fluorine, bromine, iodine, cyano, nitro, C(O)NH₂. C(S)NH₂, trifluoromethyl, difluoromethyl, pentafluoroethyl, ethynyl, propyn-1-yl, 1-butyn-1-yl, pentyn-1-yl, or hexyn-1-yl,
R³ and R⁴ independently of each other represent hydrogen, (C₁-C₈)-alkyl, R¹³O—(C₁-C₈)-alkyl, (C₃-C₈)-cycloalkyl, (C₂-C₈)-alkenyl, aryl-(C₁-C₈)-alkyl, heteroaryl-(C₁-C₈)-alkyl, or heterocyclyl-(C₁-C₈)-alkyl, or
R³ and R¹ together with the carbon atom to which they are bonded from a fully saturated or partly saturated 3- to 10-membered carbocyclic ring optionally having further substitution,
R⁵ represents hydrogen, halogen, (C₁-C₈)-alkyl, (C₁-C₈)-haloalkyl, or OR¹³,
R⁶ represents fluorine, hydrogen, chlorine, bromine, trifluoromethyl, difluoromethyl, methoxy, ethoxy, prop-1-yloxy, but-1-yloxy or methyl,
R⁷ represents hydrogen or methyl,
Q represents hydroxy or a group Q-1, Q-2

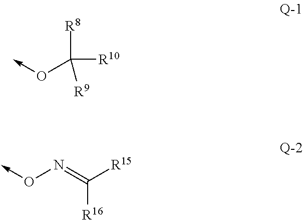

R⁸ represents hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl. C(O)R¹³, C(O)OR¹³, or (C₁-C₈)-alkoxy-(C₁-C₈)-alkyl,
R⁹ represents hydrogen or (C₁-C₈)-alkyl,
R¹⁰ represents hydrogen, halogen, cyano, nitro, (C₁C₈)-alkyl, (C₃C₈)-haloalkyl, (C₃C₈)-cycloalkyl, (C₃C₈)-cycloalkyl-(C₁C₈)-alkyl. (C₃C₈)-halocycloalkyl, (C₃C₈)-halocycloalkyl-(C₁C₈)-alkyl, (C₂C₈)-alkenyl, (C₂C₈)-alkynyl, aryl, aryl-(C₁C₈)-alkyl, heteroaryl, heteroaryl-(C₁C₈)-alkyl, heterocyclyl, heterocyclyl-(C₁C₈)-alkyl, R¹¹R¹²N—(C₁C₈)-alkyl, R¹³O—(C₁C₈)-alkyl, cyano-(C₁C₈)-alkyl, (C₁C₈)-alkylcarbonyloxy-(C₁C₈)-alkyl, (C₃C₈)-cycloalkylcarbonyloxy-(C₁C₈)-alkyl, arylcarbonyloxy-(C₁C₈)-alkyl, heteroarylcarbonyloxy-(C₁C₈)-alkyl, heterocyclylcarbonyloxy-(C₁C₈)-alkyl, OR¹³, NR¹¹R¹², SR¹⁴, S(O)R¹⁴, SO₂R¹⁴, R¹⁴S—(C₁C₈)-alkyl, R¹⁴(O)S—(C₁C₈)-alkyl, R¹⁴O₂S—(C₁C₈)-alkyl, tris-[(C₁C₈)-alkyl]silyl-(C₁C₈)-alkyl, bis-[(C₁C₈)-alkyl](aryl)silyl(C₁C₈)-alkyl, [(C₁C₈)-alkyl]-bis-(aryl)silyl-(C₁C₈)-alkyl, tris-[(C₁C₈)-alkyl]silyl, bis-hydroxyboryl-(C₁C₈)-alkyl, bis-[(C₁C₈)-alkoxy]boryl-(C₁C₈)-alkyl, tetramethyl-1,3,2-Dioxaborolan-2-yl, tetramethyl-1,3,2-dioxaborolan-2-yl-(C₁C₈)-alkyl, nitro-(C₁C₈)-alkyl, C(O)OR¹³, C(O)R¹³, C(O)NR¹¹R¹², R¹³O(O)C—(C₁C₈)-alkyl, R¹¹R¹²N(O)C—(C₁C₈)-alkyl, or bis-(C₁C₈)-alkoxy-(C₁C₈)-alkyl, or
R⁸ and R¹⁰ together with the carbon atom to which they are bonded form a fully saturated or partly saturated 3- to 10-membered monocyclic or bicyclic ring optionally interrupted by heteroatoms and optionally having further substitution,
R¹¹ and R¹² independently of each other represent hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, C(O)R¹³, SO₂R¹⁴, heterocyclyl, (C₁-C₈)-alkoxycarbonyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, or heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₁-C₈)-cycloalkyl-(C₁C₈)-alkyl, (C₄-C₁₀)-cycloalkenyl-(C₁C₈)-alkyl, bis-[(C₁-C₈)-alkyl]aminocarbonyl-(C₁-C₈)-alkyl, (C₁-C₈)-alkyl-aminocarbonyl-(C₁-C₈)-alkyl, aryl-(C₁-C₈)-alkyl-aminocarbonyl-(C₁-C₈)-alkyl, bis-[(C₁-C₈)-alkyl]amino-(C₂-C₆)-alkyl, (C₁-C₈)-alkyl-amino-(C₂-C₆)-alkyl, aryl-(C₁-C₈)-alkyl-amino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris-[(C₁-C₈)-alkyl]silyl-(C₁-C₈)-alkyl, bis-[(C₁-C₈)-alkyl](aryl)silyl(C₁-C₈)-alkyl, [(C₁-C₈)-Alkyl]-bis-(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, or (C₁-C₈)-alkoxycarbonyl,
R¹⁴ represents hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis-[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino, N-azetidinyl, N-pyrrolidinyl, N-piperidinyl, or N-morpholinyl, and
R¹⁵ and R¹⁶ independently of each other represent (C₁-C₈)-alkyl, (C₃-C₈)-cycloalkyl, aryl, heteroaryl, or heterocyclyl.

Examples of herbicidally active compounds within the scope of formula (VT) include:

| Compound | IUPAC name | Structure |
|---|---|---|
| (a) | {[3-([3-chloro-5-fluoro-6-[3-methyl-2,6-dioxo- 4-(trifluoromethyl)-3,6-dihydropyrimidin- 1(2H)-yl]pyridin-2-yl]oxy)pyridin-2- yl]oxy}acetic acid | 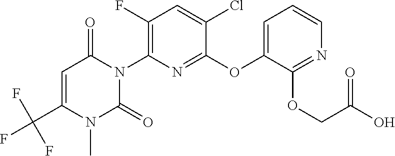 [SMILES: Cn1c(C(F)(F)F)cc(=O)n(-c2nc(Oc3cccnc3OCC(=O)O)c(Cl)cc2F)c1=O] |
| (b) | ethyl {[3-({3-chloro-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetate | 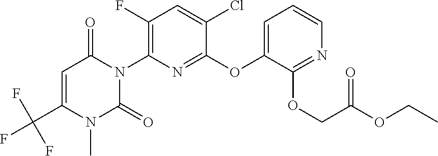 [SMILES: CCOC(=O)COc1ncccc1Oc1nc(-n2c(=O)cc(C(F)(F)F)n(C)c2=O)c(F)cc1Cl] |
| (c) | methyl {[3-({3-chloro-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acerate | 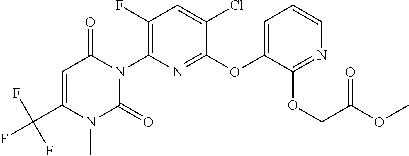 [SMILES: COC(=O)COc1ncccc1Oc1nc(-n2c(=O)cc(C(F)(F)F)n(C)c2=O)c(F)cc1Cl] |
| (d) | pyridazin-3-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 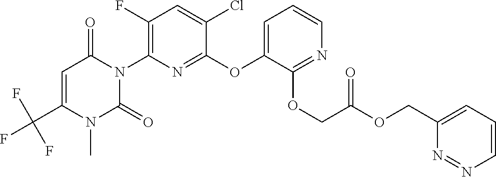 [SMILES: Cn1c(C(F)(F)F)cc(=O)n(-c2nc(Oc3cccnc3OCC(=O)OCc3cccnn3)c(Cl)cc2F)c1=O] |
| (e) | pyridin-2-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 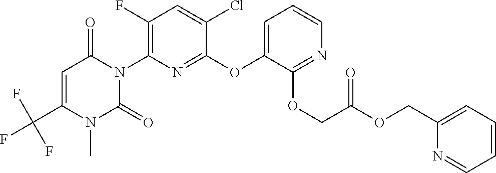 [SMILES: Cn1c(C(F)(F)F)cc(=O)n(-c2nc(Oc3cccnc3OCC(=O)OCc3ccccn3)c(Cl)cc2F)c1=O] |
| (f) | pyridin-4-ylmethyl {[3-({3-chloro-5-fluoro-6- [3-methyl-2,6-dioxo-4-(trifluoromethyl)-3,6- dihydropyrimidin-1(2H)-yl]pyridin-2- yl}oxy)pyridin-2-yl]oxy}acetate | 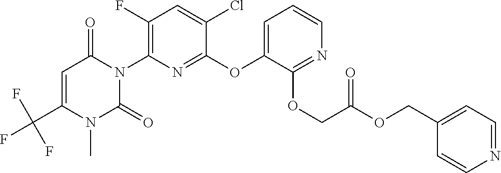 [SMILES: Cn1c(C(F)(F)F)cc(=O)n(-c2nc(Oc3cccnc3OCC(=O)OCc3ccncc3)c(Cl)cc2F)c1=O] |
| (g) | (4-trifluoromethylpyridin-3-yl)methyl {[3-({3- chloro-5-fluoro-6-[3-methyl-2,6-dioxo-4- (trifluoromethyl)-3,6-dihydropyrimidin-1(2H)- yl]pyridin-2-yl}oxy)pyridin-2-yl]oxy}acetate | 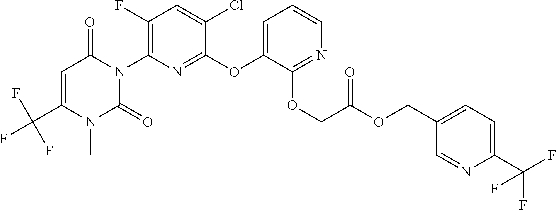 [SMILES: Cn1c(C(F)(F)F)cc(=O)n(-c2nc(Oc3cccnc3OCC(=O)OCc3ccc(C(F)(F)F)nc3)c(Cl)cc2F)c1=O] |
| (h) | ethyl [2-({3-chloro-5-fluoro-6-[3-methyl-2,6- dioxo-4-(trifluoromethyl)-3,6- dihydropyrimidin-1(2H)-yl]pyridin-2- yl}oxy)phenoxy]acetate | 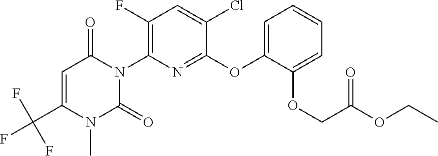 [SMILES: CCOC(=O)COc1ccccc1Oc1nc(-n2c(=O)cc(C(F)(F)F)n(C)c2=O)c(F)cc1Cl] |
| (i) | methyl [2-({3-chloro-5-fluoro-6-[3-methyl-2,6- dioxo-4-(trifluoromethyl)-3,6- dihydropyrimidin-1(2H)-yl]pyridin-2- yl}oxy)phenoxy]acetate | 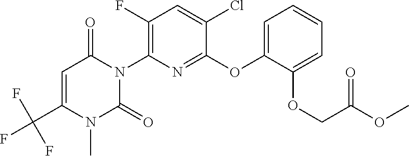 [SMILES: COC(=O)COc1ccccc1Oc1nc(-n2c(=O)cc(C(F)(F)F)n(C)c2=O)c(F)cc1Cl] |
| (j) | [2-({3-chloro-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 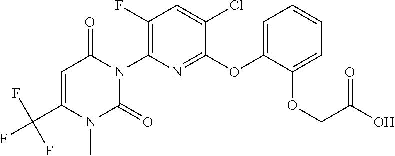 [SMILES: Cn1c(C(F)(F)F)cc(=O)n(-c2nc(Oc3ccccc3OCC(=O)O)c(Cl)cc2F)c1=O] |
| (k) | ethyl {[3-({3-bromo-5-fluoro-6-[3-methyl-2,6- dioxo-4-(trifluoromethyl)-3,6- dihydropyrimidin-1(2H)-yl]pyridin-2- yl}oxy)pyridin-2-yl]oxy}acetate | 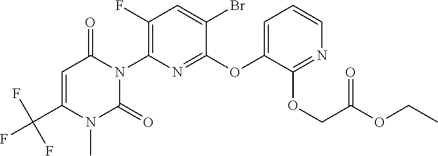 [SMILES: CCOC(=O)COc1ncccc1Oc1nc(-n2c(=O)cc(C(F)(F)F)n(C)c2=O)c(F)cc1Br] |
| (l) | methyl {[3-({3-bromo-5-fluoro-6-[3-methyl- 2,6-dioxo-4-(trifluoromethyl)-3,6- dihydropyrimidin-1(2H)-yl]pyridin-2- yl}oxy)pyridin-2-yl]oxy}acetate | 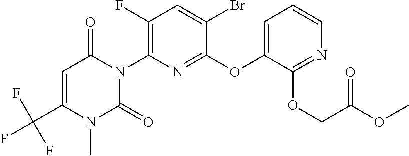 [SMILES: COC(=O)COc1ncccc1Oc1nc(-n2c(=O)cc(C(F)(F)F)n(C)c2=O)c(F)cc1Br] |
| (m) | {[3-({3-chloro-5-fluoro-6-[3-methyl-2,6-dioxo- 4-(trifluoromethyl)-3,6-dihydropyrimidin- 1(2H)-yl]pyridin-2-yl}oxy)pyridin-2- yl]oxy}acetic acid | 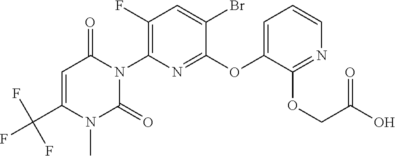 [SMILES: Cn1c(C(F)(F)F)cc(=O)n(-c2nc(Oc3cccnc3OCC(=O)O)c(Br)cc2F)c1=O] |
| (n) | ethyl [2-({3-bromo-5-fluoro-6-[3-methyl-2,6- dioxo-4-(trifluoromethyl)-3,6- dihydropyrimidin-1(2H)-yl]pyridin-2- yl}oxy)phenoxy]acetate | 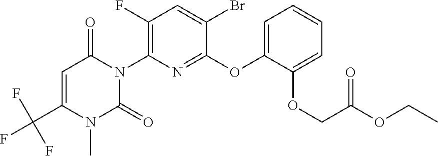 [SMILES: CCOC(=O)COc1ccccc1Oc1nc(-n2c(=O)cc(C(F)(F)F)n(C)c2=O)c(F)cc1Br] |
| (o) | methyl [2-({3-bromo-5-fluoro-6-[3-methyl-2,6- dioxo-4-(trifluoromethyl)-3,6- dihydropyrimidin-1(2H)-yl]pyridin-2- yl}oxy)phenoxy]acetate | 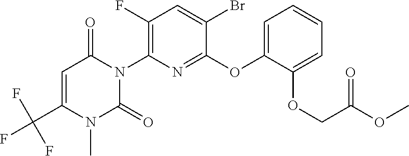 [SMILES: COC(=O)COc1ccccc1Oc1nc(-n2c(=O)cc(C(F)(F)F)n(C)c2=O)c(F)cc1Br] |
| (p) | [2-({3-bromo-5-fluoro-6-[3-methyl-2,6-dioxo- 4-(trifluoromethyl)-3,6-dihydropyrimidin- 1(2H)-yl]pyridin-2-yl}oxy)phenoxy]acetic acid | 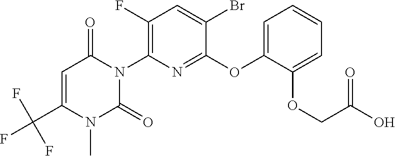 [SMILES: Cn1c(C(F)(F)F)cc(=O)n(-c2nc(Oc3ccccc3OCC(=O)O)c(Br)cc2F)c1=O] |
| (q) | ethyl {[3-({3-chloro-5-fluoro-6-[3-methyl-2,6- dioxo-4-(1,1-difluoroethyl)-3,6- dihydropyrimidin-1(2H)-yl]pyridin-2- yl}oxy)pyridin-2-yl]oxy}acetate | 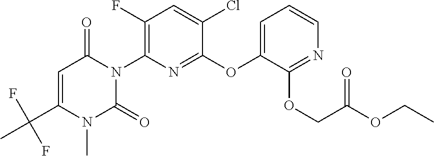 [SMILES: CCOC(=O)COc1ncccc1Oc1nc(-n2c(=O)cc(C(C)(F)F)n(C)c2=O)c(F)cc1Cl] |
| (r) | methyl {[3-({3-chloro-5-fluoro-6-[3-methyl- 2,6-dioxo-4-(1,1-difluoroethyl)-3,6- dihydropyrimidin-1(2H)-yl]pyridin-2- yl}oxy)pyridin-2-yl]oxy}acetate | 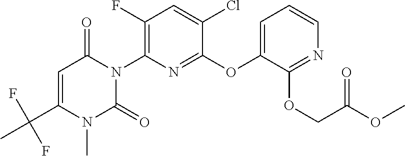 [SMILES: COC(=O)COc1ncccc1Oc1nc(-n2c(=O)cc(C(C)(F)F)n(C)c2=O)c(F)cc1Cl] |
| (s) | {[3-({3-chloro-5-fluoro-6-[3-methyl-2,6-dioxo- 4-(1,1-difluoroethyl)-3,6-dihydropyrimidin- 1(2H)-yl]pyridin-2-yl}oxy)pyridin-2- yl]oxy}acetic acid | 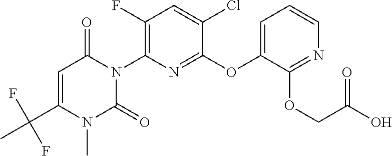 [SMILES: Cn1c(C(C)(F)F)cc(=O)n(-c2nc(Oc3cccnc3OCC(=O)O)c(Cl)cc2F)c1=O] |
| (t) | ethyl [2-({3-chloro-5-fluoro-6-[3-methyl-2,6- dioxo-4-(1,1-difluoroethyl)-3,6- dihydropyrimidin-1(2H)-yl]pyridin-2- yl}oxy)phenoxy]acetate | 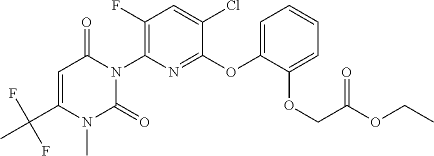 [SMILES: CCOC(=O)COc1ccccc1Oc1nc(-n2c(=O)cc(C(C)(F)F)n(C)c2=O)c(F)cc1Cl] |
| (u) | methyl [2-({3-chloro-5-fluoro-6-[3-methyl-2,6- dioxo-4-(1,1-difluoroetbyl)-3,6- dihydropyrimidin-1(2H)-yl]pyridin-2- yl}oxy)phenoxy]acetate | 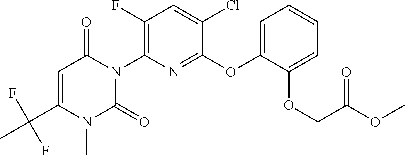 [SMILES: COC(=O)COc1ccccc1Oc1nc(-n2c(=O)cc(C(C)(F)F)n(C)c2=O)c(F)cc1Cl] |
| (v) | [2-({3-chloro-5-fluoro-6-[3-methyl-2,6-dioxo- 4-(1,1-difluoroethyl)-3,6-dihydropyrimidin- 1(2H)-yl]pyridin-2-yl}oxy)phenoxy]acetic acid | 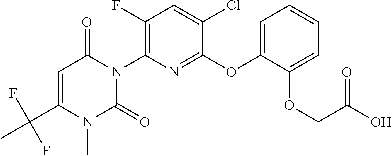 [SMILES: Cn1c(C(C)(F)F)cc(=O)n(-c2nc(Oc3ccccc3OCC(=O)O)c(Cl)cc2F)c1=O] |
| (x) | ethyl {[3-({3-chloro-5-fluoro-6-[3-methyl-2,6- dioxo-4-(trifluoromethyl)-3,6- dihydropyrimidin-1(2H)-yl]pyridin-2- yl}sulfanyl)pyridin-2-yl]oxy}acetate | 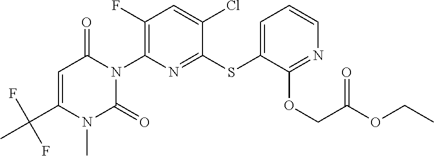 [SMILES: CCOC(=O)COc1ncccc1Sc1nc(-n2c(=O)cc(C(C)(F)F)n(C)c2=O)c(F)cc1Cl] |
| (y) | methyl {[3-({3-chloro-5-fluoro-6-[3-methyl- 2,6-dioxo-4-(trifluoromethyl)-3,6- dihydropyrimidin-1(2H)-yl]pyridin-2- yl}sulfanyl)pyridin-2-yl]oxy}acetate | 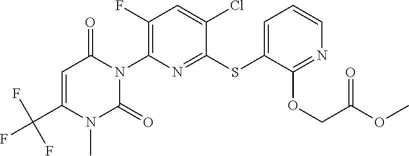 [SMILES: COC(=O)COc1ncccc1Sc1nc(-n2c(=O)cc(C(F)(F)F)n(C)c2=O)c(F)cc1Cl] |

Combinations of PPO herbicides are encompassed within the scope of the present disclosure. For example, the PPO expression cassette in soybean event Gm_CSM63717 provides tolerance to a combination of flumioxazin and pyraflufen-ethyl.

As used herein, synthetic auxins include, but are not limited to, benzoic acid auxins or herbicides, phenoxy acid herbicides, arylpicolinate herbicides, and pyridinyloxy acid herbicides. Examples of a benzoic acid herbicides include, but are not limited to, dicamba (3,6-dichloro-2-methoxybenzoic acid), dicamba salts, dicamba-butotyl, dicamba-diglycolamine salt, dicamba-dimethylammonium, dicamba-diethanolammonium, dicamba-isopropylammonium, dicamba-potassium, dicamba-sodium, and dicamba-trolamine. Examples of phenoxy acid herbicides include, but are not limited to, 2,4-D (2,4-dichlorophenoxyacetic acid), 2,4-D-butotyl, 2,4-D-butyl, 2,4-D-choline, 2,4-D-dimethylammonium, 2,4-D-diolamin, 2,4-D-ethyl, 2,4-D-2-ethylhexyl, 2,4-D-isobutyl, 2,4-D-isoctyl, 2,4-D-isopropyl, 2,4-D-isopropylammonium, 2,4-D-potassium, 2,4-D-sodium, 2,4-D-triisopropanolammonium, 2,4-D-trolamine, clomeprop, dichlorprop, fenoprop, MCPA (2-methyl-4-chlorophenoxyacetic acid), MCPA-butotyl, MCPA-dimethylammonium, MCPA-2-ethylhexyl, MCPA-isopropylammonium, MCPA-potassium, MCPA-sodium, MCPA-thioethyl, 2,4-DB, MCPB (4-(4-chloro-2-methylphenoxy)butanoic acid), MCPB-methyl, MCPB-ethyl-sodium, and mecoprop. Examples of arylpicolinate herbicides include, but are not limited to, halauxifen, halauxifen-methyl, and florpyrauxifen-benzyl. Examples of pyridinyloxy acid herbicides include, but are not limited to, triclopyr, fluroxypyr, aminopyralid, and picloram.

As used herein, inhibitors of glutamine synthetase include, but are not limited to, phosphinothricin, glufosinate, glufosinate salts, glufosinate-ammonium, glufosinate-sodium, glufosinate-P, L-glufosinate-ammonium, L-glufosinate-sodium, and combinations of any thereof.

As used herein, the β-triketone HPPD inhibitor can be selected from the group consisting of mesotrione, benzobicyclon (BBC), tembotrione, sulcotrione, tefuryltrione, and combinations of any thereof.

As used herein, inhibitors of 5-enolpyruvylshikimate-3-phosphate synthase (EPSPS) include, but are not limited to, glyphosate, glyphosate salts, glyphosate-isopropylammonium, glyphosate-ammonium, glyphosate-dimethylammonium, glyphosate-trimesium (=sulfosate), glyphosate-diammonium, glyphosate-potassium, glyphosate-sodium, and combinations of any thereof.

As used herein, “herbicide tolerant” or “herbicide tolerance” or “tolerance” means the ability to be wholly or partially unaffected by the presence or application of one of more herbicide(s), for example to resist the toxic effects of an herbicide when applied. A cell, seed, or plant is “herbicide tolerant” or has “improved tolerance” if it can maintain at least some normal growth or phenotype in the presence of one or more herbicide(s). A trait is an herbicide tolerance trait if its presence can confer improved tolerance to an herbicide upon a cell, plant, or seed as compared to the wildtype or control cell, plant, or seed. Crops comprising an herbicide tolerance trait can continue to grow in the presence of the herbicide and may be minimally affected by the presence of the herbicide. A protein confers “herbicide tolerance” if expression of the protein can confer improved tolerance to an herbicide upon a cell, plant, or seed as compared to the wildtype or control cell, plant, or seed. Examples of herbicide tolerance proteins are protoporphyrinogen oxidase, dicamba monooxygenase, phosphinothricin N-acetyltransferase, the alpha-ketoglutarate-dependent non-heme iron dioxygenase, triketone dioxygenase, and 5-enolpyruvylshikimate-3-phosphate synthase. Herbicide tolerance may be complete or partial insensitivity to a particular herbicide and may be expressed as a percent (%) tolerance or insensitivity to a particular herbicide.

As used herein “herbicide injury” or “injury” refers to injury to a plant because of the application of one or more herbicides. The “injury rate” or “percent injury” refers to the percentage of leaf area of a plant exhibiting damage such as necrosis (brown or dead tissue), chlorosis (yellow tissue or yellow spotting) and malformation (misshapen leaves or plant structures, epinasty or twisting of stem, cupping of leaves) caused by herbicide application based on visual evaluation. It is measured on a scale of 0 to 100, where “0” representing no crop injury and “100” denoting complete crop injury (death).

For soybean plants containing or comprising soybean event Gm_CSM63717, the plant will have decreased injury after application of one or more PPO inhibitors. For example, soybean plants containing or comprising soybean event Gm_CSM63717 will have less than about 5% injury, less than about 10% injury, less than about 15% injury, or less than about 20% injury following application of a PPO herbicide such as flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, or a combination of any thereof, as compared to otherwise identical soybean plants that do not contain soybean event Gm_CSM63717.

As used herein, a “weed” is any undesired plant. A plant may be considered generally undesirable for agriculture or horticulture purposes (for example, Amaranthus species) or may be considered undesirable in a particular situation (for example, a crop plant of one species in a field of a different species, also known as a volunteer plant). Weeds are commonly known in the art and vary by geography, season, growing environment, and time. Lists of weed species are available from agricultural and scientific societies and efforts (such as the Weed Science Society of America, the Canadian Weed Science Society, the Brazilian Weed Science Society, the International Weed Science Society, and the International Survey of Herbicide Resistant Weeds), government agencies (such as the United States Department of Agriculture and the Australia Department of the Environment and Energy), and industry and farmer associations. Major troublesome weeds in soybean production include waterhemp (Amaranthus tuberculatus), ragweed (Ambrosia spp.) such as giant ragweed (Ambrosia trifida), and common ragweed (Ambrosia artemissifolia), common lambsquarters (Chenopodium album), morning glory species (Ipomoea spp.), horseweed (Conyza canadensis), marestail (Erigeron canadensis), palmer amaranth (Amaranthus palmeri), pigweed (Amaranthus spp.), velvetleaf (Abutilon theophrasti Medik.), common cocklebur (Xanthium strumarium), foxtail (Setaria spp.), crabgrass (Digitaria spp.), barnyard grass (Echinochloa crus-galli), Johnsongrass (Sorghum halepense), and thistles (Cirsium spp.) (Heap 2021; Shoup et al, 2016).

Methods for controlling or preventing weed growth in an area are provided. One example of such a method comprises soybean comprising event Gm_CSM63717 in the area and applying an effective amount of a PPO herbicide to control weeds in the area without injury to the soybean or with less than about 10% injury to the soybean. The methods comprise applying one or more PPO herbicides, where seeds or plants comprising soybean event Gm_CSM63717 are planted in the area before, at the time of, or after applying the herbicide and the herbicide application prevents or inhibits weed growth and does not injure the soybean plants comprising event Gm_CSM63717 or has about less than about 5-20% injury. The plant growth area may or may not comprise weed seeds or plants at the time of herbicide application. The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after soybean seed planting), or both. For example, a method for controlling weeds is provided that consists of planting seed comprising soybean event Gm_CSM63717 in an area and applying an herbicidally effective amount over the growing season of one or more PPO herbicides alone or in any combination with another herbicide, for the purpose of controlling weeds in the area with no injury or less than about 5-20% injury to the plants containing soybean event Gm_CSM63717. Such application of herbicide(s) may be pre-planting (any time prior to planting seed comprising soybean event Gm_CSM63717, including for burn-down purposes, that is application to emerging or existing weeds prior to seed plant), pre-emergence (any time after seed comprising soybean event Gm_CSM63717 is planted and before plants comprising soybean event Gm_CSM63717 emerge), or post-emergence (any time after plants comprising soybean event Gm_CSM63717 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Herbicide application in practicing the methods described herein may be at the recommended commercial rate or any fraction or multiple thereof, such as twice the recommended commercial rate. Herbicide rates may be expressed as pounds acid equivalent per acre (lb ae/acre), pounds active ingredient per acre (lb ai/acre) or pounds active ingredient per hectare (lb ai/ha), depending on the herbicide and the formulation. One gram per hectare is equal to 0.000892179 pound per acre. The use of acres or hectare in the herbicide application rates as provided herein is merely instructive; herbicide application rates in the equivalent dosages to any rate provided herein may be used for areas larger or smaller than an acre. The herbicide application can comprise at least one PPO herbicide including, but not limited to flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, another PPO herbicide, or a combination of any thereof. The plant growth area may or may not comprise weed plants at the time of herbicide application.

The effective amount of a PPO herbicide can be about 0.0009 lb/acre to about 1.5 lb/acre over a growing season. Table 3 provides examples of various PPO herbicides and the application rates that can be used for controlling weeds in a soybean crop growing area where Gm_CSM63717 is planted.

TABLE 3
Examples of PPO herbicides and application rates

| | Broad | Intermediate | Labeled |
|---|---|---|---|
| | Rate Range | Rate Range | Rates |
| | (gram/ | (gram/ | (gram/ |
| PPO Herbicide | hectare) | hectare) | hectare) |
| Epyrifencacil (S3100) | 1-200 | 5-100 | 20-40 |
| Flumioxazin | 1-448 | 5-140 | 70-224 |
| Fomesafen | 1-1680 | 5-840 | 280-420 |
| Lactofen | 1-880 | 5-440 | 140-220 |
| Saflufenacil | 1-400 | 5-200 | 22-88 |
| Sulfentrazone | 1-1680 | 5-840 | 112-420 |
| Trifludimoxazin | 1-75 | 10-50 | 25-50 |
| Tiafenacil | 1-75 | 10-50 | 25-50 |
| Acifluorfen | 1-1680 | 5-840 | 280-560 |
| Pyraflufen | 1-50 | 10-25 | 25-50 |
| Oxadiazon | 1-400 | 50-200 | 200-400 |
| Butafenacil | 1-100 | 10-100 | 25-50 |
| pyridin-2-ylmethyl [(3-{2- | 1-200 | 10-100 | 50-100 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| Flufenoximacil | 1-200 | 5-75 | 25-50 |
| 2-methoxyethyl [(3-{2- | 1-200 | 5-75 | 25-50 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}pyridin-2- | | | |
| yl)oxy]acetate | | | |
| cyanomethyl [(3-{2-bromo- | 1-200 | 5-75 | 25-50 |
| 4-fluoro-5-[3-methyl-2,6- | | | |
| dioxo-4-(trifluoromethyl)- | | | |
| 3,6-dihydropyrimidin-1(2H)- | | | |
| yl]phenoxy}pyridin-2- | | | |
| yl)oxy]acetate | | | |
| cyclopropylmethyl (2-{2- | 1-200 | 5-100 | 50-100 |
| chloro-4-fluoro-5-[3- | | | |
| methyl-2,6-dioxo-4- | | | |
| (trifluoromethyl)-3,6- | | | |
| dihydropyrimidin-1(2H)- | | | |
| yl]phenoxy}phenoxy)acetate; | | | |

Methods for controlling or preventing weed growth in an area are provided. One example of such a method comprises planting soybean comprising event Gm_CSM63717, event Gm_CSM63714 and/or event MON89788 in the area and applying one or more herbicides selected from the group consisting of a PPO herbicide, dicamba, glufosinate, 2, 4-D, β-triketone HPPD inhibitor, glyphosate, and combinations of any thereof without injury or with less than 10% injury to the soybean. The methods comprise applying one or more herbicides selected from the group consisting of a PPO herbicide one or more herbicides selected from the group consisting of a PPO herbicide, dicamba, glufosinate, 2, 4-D, β-triketone HPPD inhibitor, glyphosate, and combinations of any thereof, where seeds or plants comprising soybean event Gm_CSM63717, event Gm_CSM63714 and/or event MON89788 are planted in the area before, at the time of, or after applying the one or more herbicides and the herbicide application prevents or inhibits weed growth and does not injure the soybean plants comprising event Gm_CSM63717, event Gm_CSM63714 and/or event MON89788 or has about less than about 5-20% injury. The plant growth area may or may not comprise weed seeds or plants at the time of herbicide application. The herbicide(s) used in the methods described herein can be applied alone, sequentially or in any combinations temporally (for example, as a tank mixture or in sequential applications), spatially (for example, at different times during the growing season including before and after soybean seed planting), or both during the growing season. Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Methods for controlling volunteer soybean comprising soybean event Gm_CSM63717 in an area are provided. The methods comprise applying an herbicidally effective amount of at least one herbicide other than a PPO herbicide, wherein the herbicide application prevents growth of soybean comprising event Gm_CSM63717. Illustrative examples of such herbicides are pyrithiobac, fluometuron, trifloxysulfuron, glyphosate, glufosinate, paraquat, diquat, clopyralid, topramezone, atrazine, (3-(3,4-dichlorophenyl)-1,1-dimethylurea) (DCMU, sold under the tradename Diuron), and combinations of any thereof, wherein the herbicide application prevents growth of volunteer soybean comprising event Gm_CSM63717. For example, to control volunteer soybean comprising event Gm_CSM63717 in a cotton cultivation field, fluometuron and DCMU can be applied preemergence, and trifloxysulfuron and pyrithiobac can be applied postemergence. To control volunteer soybean comprising event Gm_CSM63717 in a corn cultivation field, atrazine can be applied preemergence and post emergence, and topramazone and clopyralid can be applied postemergence

Methods for producing plants and seeds comprising soybean event Gm_CSM63717 are provided. Plants may be bred using any method known in the art. A progeny soybean plant comprising the event Gm_CSM63717 may be produced, for example, by selfing a parent plant or line comprising the event Gn_CSM63717, wherein such parent plant or line is homozygous or hemizygous for the event Gm_CSM63717, or by crossing a first parent plant or line comprising the event Gm_CSM63717, wherein such parent plant or line is homozygous or hemizygous for the event Gm_CSM63717, with a second parent plant or line having a different genotype or germplasm than the first parent line, wherein the second parent plant or line may or may not contain or comprise the event Gm_CSM63717. As described further herein, soybean event Gm_CSM63717 comprises a PPO expression cassette or transgene encoding a protoporphyrinogen oxidase. According to some embodiments, the transgenic soybean plant(s) comprising the event Gm_CSM63717 is/are tolerant to PPO inhibitors, relative to a non-transgenic control plant. Transgenic soybean plants used in these methods may be homozygous or heterozygous for the transgene. Progeny plants produced by these methods may be varietal or hybrid plants; may be grown from seeds produced by soybean event Gm_CSM63717 containing plant and/or from seeds produced by a plant fertilized with pollen from a soybean event Gm_CSM63717 containing plant; and may be homozygous or heterozygous for the transgenes and/or event Gm_CSM63717. Progeny plants may be subsequently self-pollinated to generate a true breeding line of plants, i.e., plants homozygous for the transgene, or alternatively may be out-crossed, e.g., bred with another unrelated plant, to produce a varietal or a hybrid seed or plant.

A method of obtaining a seed of a soybean plant or a soybean plant that is tolerant to PPO herbicides, is provided. The method comprises: (a) obtaining a population of progeny seed or plants grown therefrom, at least one of which comprises soybean event Gm_CSM63717; and (b) identifying at least a first progeny seed or plant grown therefrom that comprises soybean event Gm_CSM63717. Identifying the progeny seed or plant grown therefrom that comprises soybean event Gm_CSM63717 can comprise: (a) growing the progeny seed or plant to produce progeny plants; (b) treating the progeny plants with an effective amount of a PPO herbicide; and (c) selecting a progeny plant that is tolerant to the PPO herbicide. Alternatively or in addition, identifying the progeny seed or plant grown therefrom that comprises soybean event Gm_CSM63717 comprises detecting the presence of soybean event Gm_CSM63717 in a sample derived from the progeny seed or plant grown therefrom. Alternatively or in addition, identifying the progeny seed or plant grown therefrom that comprises soybean event Gm_CSM63717 comprises detecting the presence of the PPO protein encoded by soybean event Gm_CSM63717 in a sample derived from the progeny seed or plant grown therefrom.

As used herein, the terms “line”, “breeding line”, “genotype” or “germplasm” are used interchangeably to refers to a group of plants that show little or no genetic variation between individuals for at least one trait. Such “line”, “breeding line”, “genotype” or “germplasm” can be created by self-pollination for several generations, selection, or vegetative propagation from a single parent using tissue or cell culture techniques. As used herein, the terms “cultivar” and “variety” are synonymous and refer to a line used for commercial production.

The production of double haploids may also be used to produce soybean plants and seeds homozygous for event Gm_CSM63717 DNA in a breeding program. Double haploids are produced by the doubling of a set of chromosomes (1 N) from a heterozygous plant to produce a completely homozygous individual. For example, see Wan, et al., (1989) and U.S. Pat. No. 7,135,615. This can be advantageous because the process omits the generations of selfing needed to obtain a homozygous plant from a heterozygous source. One way of producing haploid and double haploid soybean plant comprising event Gm_CSM63717 is through anther culture of flowers comprising event Gm_CSM63717 (Khan et al., 2010). Other methods such as natural polyembryony, induction with irradiated pollen, crosses with polyploid plants or wild species, unfertilized ovule and microspore culture can also be applied to produce haploid and double haploid soybean plants comprising event Gm_CSM63717.

Seed and progeny plants made by the methods described herein comprise soybean event Gm_CSM63717. Application of one or more herbicide for which soybean event Gm_CSM63717 confers tolerance may be used to select progeny that comprise soybean event Gm_CSM63717. Alternatively, progeny may be analyzed using diagnostic methods to select for plants or seeds comprising soybean event Gm_CSM63717.

Soybean transgenic events are known to one of skill in the art; for example, a list of such traits is provided by the United States Department of Agriculture's (USDA) Animal and Plant Health Inspection Service (APHIS) and can be found on their website at www.aphis.usda.gov. Two or more transgenic events may thus be combined in a progeny seed or plant by crossing two parent plants each comprising one or more transgenic event(s), collecting progeny seed, and selecting for progeny seed or plants that contain the two or more transgenic events; these steps may then be repeated until the desired combination of transgenic events in a progeny is achieved. Back-crossing to a parental plant and out-crossing with a non-transgenic plant are also contemplated, as is vegetative propagation.

Methods of detecting the presence of soybean event Gm_CSM63717 in a sample of DNA derived from a soybean seed, plant, plant part, plant cell, progeny plant, or commodity product are provided. One method comprises: (i) contacting the sample with at least one probe specific to event Gm_CSM63717 DNA under conditions appropriate for DNA sequencing; (ii) performing a DNA sequencing reaction; and (iii) confirming that the nucleotide sequence comprises a nucleotide sequence specific for event Gm_CSM63717, of the transgenic insert comprised therein, such as one selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10.

Another method of detecting the presence of soybean event Gm_CSM63717 in a sample of DNA derived from a soybean seed, plant, plant part, plant cell, progeny plant, or commodity product is provided. The method comprises: (a) contacting the sample with a DNA probe specific for event Gm_CSM63717 DNA; and (b) performing a sequencing reaction to produce a target sequence. The target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,201-4,202 of SEQ ID NO:10.

Another method of detecting the presence of soybean event Gm_CSM63717 in a sample derived from a soybean seed, plant, plant part or plant cell, progeny plant or commodity product comprises: (a) contacting the sample with a primer pair that is capable of producing an amplicon from event Gm_CSM63717 DNA under conditions appropriate for DNA amplification; (b) performing a DNA amplification reaction to produce a DNA amplicon; and (c) detecting the presence of the DNA amplicon. The DNA amplicon comprises a nucleotide sequence specific for event Gm_CSM63717, for example, at least one of (i) a 5′ junction sequence between flanking soybean genomic DNA and the transgenic insert of soybean event Gm_CSM63717, (b) a 3′ junction sequence between flanking soybean genomic DNA and the transgenic insert of soybean event Gm_CSM63717, (c) SEQ ID NO: 9, and (d) a fragment of SEQ ID NO: 9 comprising a sufficient length of contiguous nucleotides of SEQ ID NO: 9 to identify the sequence as a fragment of the transgenic insert of Gm_CSM63717. The presence of the DNA amplicon indicates the presence of soybean event Gm_CSM63717 in the sample. The amplicon should be one that is specific for event Gm_CSM63717, and comprises the junction at nucleotide positions 1000-1001, and/or nucleotide positions 4,201-4,202 of SEQ ID NO:10. Thus, for example, the amplicon can comprise a nucleotide sequence selected from the group consisting of SEQ ID NO:1. SEQ ID NO:2, SEQ ID NO:3, SEQ ID NO:4, SEQ ID NO:5, SEQ ID NO:6, SEQ ID NO:7, SEQ ID NO:8, SEQ ID NO:9, and SEQ ID NO:10; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,201-4,202 of SEQ ID NO:10. Alternatively or in addition, the amplicon can comprise a fragment of SEQ ID NO: 9 comprising a sufficient length of contiguous nucleotides of SEQ ID NO: 9 to identify the sequence as a fragment of the transgenic insert of soybean event Gm_CSM63717. The amplicon can be at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length.

The detection of a nucleotide sequence specific for event Gm_CSM63717 in the amplicon is determinative and/or diagnostic for the presence of the soybean event Gm_CSM63717 specific DNA in the sample. An illustrative primer pair that is capable of producing an amplicon from event Gm_CSM63717 DNA under conditions appropriate for DNA amplification is provided as SEQ ID NO:176 and SEQ ID NO:177. Other primer pairs may be readily designed by one of skill in the art to produce an amplicon diagnostic for soybean event Gm_CSM63717, wherein such a primer pair comprises at least one primer within the genomic region flanking the insert and a second primer within the insert, provided that any primer pair could be designed and used that produces an amplicon comprising a junction sequence and/or all or part of the insert or transgene sequence. Detection of an amplicon could be based on any suitable method, such as sequencing, determining fragment size or migration of the amplicon in a matrix or gel, or a hybridization-based method.

Another method of detecting the presence of soybean event Gm_CSM63717 in a sample derived from a soybean plant, plant part, plant cell, seed, progeny plant, or commodity product comprises: (i) contacting the sample with a DNA probe specific for event Gm_CSM63717 DNA; (ii) subjecting the sample and the DNA probe to stringent hybridization conditions; and (iii) detecting hybridization between the probe and the target DNA in the sample. An example of the sequence of a DNA probe that is specific for event Gm_CSM63717 is provided as SEQ ID NO:178. Other probes may be readily designed by one of skill in the art. Detection of probe hybridization to the DNA in the sample is diagnostic for the presence of soybean event Gm_CSM63717 specific DNA in the sample. Absence of hybridization is alternatively diagnostic of the absence of soybean event Gm_CSM63717 specific DNA in the sample.

Another method of detecting the presence of soybean event Gm_CSM63717 in a sample derived from a soybean plant, plant part, plant cell, seed, progeny plant, or commodity product comprises: (a) contacting the sample with an antibody specific for the PPO (protoporphyrinogen oxidase) protein encoded by soybean event Gm_CSM63717; and (b) detecting binding of the antibody to the protein in the sample. The binding of the antibody indicates the presence of soybean event Gm_CSM63717 in the sample.

Methods for determining the zygosity of the event and transgene with genomic DNA derived from at least one soybean plant, plant part, plant cell or seed comprising soybean event Gm_CSM63717 in a sample are provided. In one such method of determining the zygosity of a soybean plant, plant part, plant seed, or plant cell comprising soybean event Gm_CSM63717, the method comprises: (a) contacting a sample comprising DNA derived from the soybean plant, plant part, plant seed, or plant cell with a first primer set capable of producing a first amplicon diagnostic for the presence of soybean event Gm_CSM63717, and a second primer set capable of producing a second amplicon diagnostic for wildtype soybean genomic DNA not comprising soybean event Gm_CSM63717; (b) performing a nucleic acid amplification reaction; and (c) detecting the first amplicon and the second amplicon, wherein the presence of both amplicons indicates that the plant, plant part, seed or cell is heterozygous for soybean event Gm_CSM63717, and the presence of only the first amplicon indicates that the plant, plant part, seed, or cell is homozygous for soybean event Gm_CSM63717. The presence of only the second amplicon is diagnostic for the absence of event Gm_CSM63717 DNA in the sample. Illustrative sets of primer pairs are SEQ ID NO:176 and SEQ ID NO:177, which produce an amplicon diagnostic for event Gm_CSM63717; and SEQ ID NO:208 and SEQ ID NO:209, which produce an amplicon diagnostic for wildtype soybean genomic DNA not comprising event Gm_CSM63717. A set of probes can also be incorporated into such an amplification method to be used in a real-time PCR format using the primer pair sets described above. An illustrative set of probes are presented as SEQ ID NO:178 (diagnostic for the amplicon for the event Gm_CSM63717) and SEQ ID NO:210 (diagnostic for the amplicon for wildtype soybean genomic DNA not comprising event Gm_CSM63717).

Another method for determining the zygosity of a soybean plant, plant part, plant seed, or plant cell comprising soybean event Gm_CSM63717 comprises (a) contacting a sample comprising DNA derived from the soybean plant, plant part, plant seed, or plant cell with a probe set comprising at least a first probe that specifically hybridizes to soybean event Gm_CSM63717, and at least a second probe that specifically hybridizes to soybean genomic DNA that was disrupted by insertion of the heterologous DNA of soybean event Gm_CSM63717 but does not hybridize to soybean event Gm_CSM63717; and (b) hybridizing the probe set with the sample under stringent hybridization conditions. Detecting hybridization of only the first probe under the hybridization conditions is diagnostic for a soybean plant, plant part, seed or plant cell homozygous for soybean event Gm_CSM63717 and detecting hybridization of both the first probe and the second probe under the hybridization conditions is diagnostic for a soybean plant, plant part, seed, or plant cell heterozygous for soybean event Gm_CSM63717. Detecting hybridization of only the second probe under the hybridization conditions is diagnostic for the absence of event Gm_CSM63717 DNA in the sample. An illustrative probe set that can be used in the method is SEQ ID NO:178 and SEQ ID NO:210.

Yet another method for determining zygosity comprises (i) extracting a sample comprising DNA from at least one soybean plant, plant part, plant cell or seed; (ii) contacting the sample with a first primer pair that is capable of producing a first amplicon diagnostic for event Gm_CSM63717; (iii) contacting the sample with a second primer pair that is capable of producing a second amplicon of an internal standard known to be single-copy and homozygous in the soybean plant; (iv) contacting the sample with a probe set which contains at least a first probe that specifically hybridizes to the first amplicon, and at least a second probe that specifically hybridizes to the second amplicon; (v) performing a DNA amplification reaction using real-time PCR and determining the cycle thresholds (Ct values) of the first and second amplicons; (vi) calculating the difference ( [symbol] Ct) between the Ct value of the first amplicon and the second amplicon; and (vii) determining zygosity, wherein a [symbol] Ct of about zero (0) indicates homozygosity of the event or inserted T-DNA, and a [symbol] Ct of about one (1) indicates heterozygosity of the event or inserted T-DNA. Heterozygous and homozygous events are differentiated by a [symbol] Ct value unit of approximately one (1). Given the normal variability observed in real-time PCR due to multiple factors such as amplification efficiency and ideal annealing temperatures, the range of “about one (1)” is defined as a [symbol] Ct of 0.75 to 1.25, and the range of “about zero (0)” is defined as a [symbol] Ct of −0.25 to 0.25 (or of 0.0 to 0.25 if the [symbol] Ct is measured as an absolute value). Primer pairs and probes for the above method for determining zygosity can amplify and detect amplicons from the transgene or event DNA and the internal DNA standard.

A DNA construct is provided. The DNA construct comprises an expression cassette, wherein the expression cassette comprises, in operable linkage, i) a promoter, a leader sequence, and an intron sequences of ubiquitin 2 from Medicago truncatula, ii) a codon-optimized chloroplast transit peptide coding sequence of APG6 (Albino and Pale Green 6) from Adansonia digitata, iii) a codon-optimized protoporphyrinogen oxidase (PPO) coding sequence from Enterobacter cloacae for conferring tolerance to PPO herbicides, and iv) a synthetic 3′ UTR sequence. For example, the DNA construct can comprise SEQ ID NO: 9.

Expression of the PPO in transgenic plants confers tolerance to PPO herbicides. For example, plants, plant parts, plant cells or seeds containing or comprising soybean event Gm_CSM63717 are tolerant to PPO herbicides flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, and combinations of any thereof.

Any of the DNA constructs or transgenic inserts described herein can further comprise at its 5′ or 3′ end at least 50 contiguous nucleotides of SEQ ID NO:11 or SEQ ID NO:14, or at least 50 contiguous nucleotides of SEQ ID NO:12 or SEQ ID NO:15. Alternatively, any of the DNA constructs or transgenic inserts described herein can further comprise at its 5′ or 3′ end at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14, or at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15.

SEQ ID NOs:11 and 12 are 1,000 nucleotide sequences representing soybean genomic DNA that flanks the transgenic insert at the 5′ and 3′ ends of the insert in soybean event Gm_CSM63717, respectively. SEQ ID NO:11 and SEQ ID NO:12 have been validated by sequencing, as further described in Example 5 hereinbelow. SEQ ID NOs:14 and 15 are 5,000 nucleotide sequences representing soybean genomic DNA that flanks the transgenic insert at the 5′ and 3′ ends of the insert, respectively. Nucleotides 4,001-5,000 of SEQ ID NO:14 are identical to nucleotides 1-1,000 of SEQ ID NO:11. The remaining nucleotides of SEQ ID NO:14 (nucleotides 1-4,000) are based on the genomic sequence of the Williams 82 soybean cultivar (Schmutz et al., 2010). Similarly, nucleotides 1-1000 of SEQ ID NO:15 are identical to nucleotides 1-1,000 of SEQ NO:12. The remaining nucleotides of SEQ ID NO:15 (nucleotides 1,001-5,000) are based on the genomic sequence of the Williams 82 soybean cultivar.

The contiguous nucleotides of SEQ ID NO:11 or SEQ ID NO:14 at the 5′ end of the DNA construct or transgenic insertion may be immediately adjacent to and upstream (on the 5′ end) of the transgenic insertion, or may not be immediately adjacent to, but further upstream (on the 5′ end) and within about 5000 nucleotides, within about 3000 nucleotides, or within about 1000 nucleotides of the transgenic insertion. Likewise, the contiguous nucleotides of SEQ ID NO:12 or SEQ ID NO:15 at the 3′ end of the DNA construct or transgenic insertion may be immediately adjacent to and downstream (on the 3′ end) of the transgenic insertion, or may not be immediately adjacent to but further downstream (on the 3′ end) and within about 5000 nucleotides, within about 3000 nucleotides, or within about 1000 nucleotides of the transgenic insertion. Illustrative examples of sequences comprising 50 contiguous nucleotides of SEQ ID NO:11 are provided in SEQ ID NOs:36-55. Illustrative examples of 50 contiguous nucleotides of SEQ ID NO:12 are provided in SEQ ID NOs:106-125. Illustrative examples of 50 contiguous nucleotides of SEQ ID NO:14 are provided in SEQ ID NOs:56-105. Illustrative examples of 50 contiguous nucleotides of SEQ ID NO:15 are provided in SEQ ID NOs:126-175. However, any sequence comprising at least 50 contiguous nucleotides of SEQ ID NO:11 or SEQ ID NO:14, or at least 50 contiguous nucleotides of SEQ ID NO:12 or SEQ ID NO:15 is within the scope of the present disclosure.

In addition, a DNA construct comprising a PPO expression cassette is provided. The DNA construct further comprises at the 5′ and/or 3′ end of the construct (i) at least 50 contiguous nucleotides of SEQ ID NO:11 or SEQ ID NO:14; and/or (ii) at least 50 contiguous nucleotides of SEQ ID NO:12 or SEQ ID NO:15.

A further DNA construct is provided. The DNA construct comprises a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9. The DNA construct further comprises at the 5′ and/or 3′ end of the construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or 14; and/or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or 15.

For example, any of the DNA constructs can comprise at the 5′ end of said construct one or more nucleotide sequences selected from SEQ ID NOs:36-105. Alternatively or in addition, any of the DNA constructs can comprise at the 3′ end of said construct one or more nucleotide sequences selected from SEQ ID NOs:106-175.

Soybean plants, plant cells, plant parts, and plant seeds comprising any of the DNA constructs described herein are also provided.

Also provided are soybean plants, plant cells, plant parts, and plant seeds comprising a recombinant DNA construct integrated in chromosome 13, wherein the recombinant DNA construct confers tolerance to at least one PPO herbicide. The recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or 14 and at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or 15. The at least 50 contiguous nucleotides of SEQ ID NO:11 or 14 can comprise one or more nucleotide sequences selected from SEQ ID NOs:36-105, and the at least 50 contiguous nucleotides of SEQ ID NO:12 or 15 can comprise one or more nucleotide sequences selected from SEQ ID NOs:106-175.

Methods of improving tolerance to herbicides are provided. The methods comprise: i) inserting a DNA construct comprising a PPO expression cassette, as described herein, into the genome of a soybean cell, ii) generating a soybean plant from the soybean cell; and iii) selecting a soybean plant comprising the DNA construct. The selecting can comprise treating the soybean cell or plant with an effective amount of a PPO herbicide.

Transgenic plants produced by the methods comprise a unique combination of expression elements for optimal expression of the transgene. Furthermore, transgenic plants produced by the methods as described herein acquire tolerance to PPO herbicides. Selecting the regenerated plant comprising the DNA construct may be done using DNA or protein detection methods as described herein. Alternatively or additionally, selecting may comprise treating the transgenic plant or plant cell with an effective amount of at least one PPO herbicide selected from the group consisting of flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, or a combination of any thereof.

Methods of controlling, preventing, or reducing the development of herbicide-tolerant weeds are provided. The methods comprise: a) cultivating in a crop growing environment a soybean plant comprising a DNA construct or transgene of the present disclosure or event Gm_CSM63717 that provides tolerance to PPO herbicides, and b) applying to the crop growing environment at least one PPO herbicide selected from the group consisting of flumioxazin, epyrifenacil, lactofen, acifluorfen, pyraflufen, pyraflufen-ethyl, oxadiazon, butafenacil, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, methyl (2R)-2-{[(E)-({2-chloro-4-fluoro-5-[3-methyl-2,6-dioxo-4-(trifluoromethyl)-3,6-dihydropyrimidin-1(2H)-yl]phenyl}methylidene)amino]oxy}propanoate (flufenoximacil), fomesafen, saflufenacil, sulfentrazone, tiafenacil, and trifludimoxazin, or a combination of any thereof, wherein the soybean plant is tolerant to the at least one PPO herbicide.

Methods of controlling, preventing, or reducing the development of herbicide-tolerant weeds are provided. One such method comprises cultivating in a crop growing environment a soybean plant comprising transgenes that provide tolerance to (i) a PPO herbicide and (ii) herbicides with at least three additional herbicide modes of action, the three additional herbicide modes of action each being different from one another. The at least three additional herbicide modes of action can be selected from the group consisting of inhibition of glutamine synthetase, synthetic auxins such as benzoic acid auxins and phenoxy auxins, inhibition of HPPD, and inhibition of EPSPS.

Another method of controlling, preventing, or reducing the development of herbicide-tolerant weeds is provided. The method comprises: (a) cultivating in a crop growing environment a soybean plant comprising any of the DNA constructs comprising a PPO tolerance gene described herein, and at least three additional transgenes for providing tolerance to herbicides with at least three additional herbicide modes of action, the three additional herbicide modes of action each being different from one another; and (b) applying to the crop growing environment at least one herbicide selected from the group consisting of dicamba, glufosinate, 2,4-D, β-triketone HPPD inhibitor, PPO inhibitor, glyphosate, and any combination thereof, wherein the soybean plant is tolerant to the at least one herbicide.

In any of the methods of controlling, preventing, or reducing the development of herbicide-tolerant weeds, the transgenes that provide tolerance to the herbicides with at least three additional herbicide modes of action can be present at a single genomic location or at more than one genomic locations in the soybean plant. The transgenes that provide tolerance to the herbicides with at least three additional herbicide modes of action can be selected from the group consisting of DMO, PAT, FT_Tv7, TDO, EPSPS, and combinations of any thereof. For example, the soybean plant, plant seed, plant part, or plant cell used in these methods can further comprise soybean event Gm_CSM63714 and/or MON89788.

An illustrative PAT coding sequence and its corresponding amino acid sequence from Streptomyces viridochromogenes are provided as SEQ ID NO:26 and SEQ ID NO:27, respectively. An illustrative DMO coding sequence and its corresponding amino acid sequence from Stenotrophomonas maltophilia are provided as SEQ ID NO:28 and SEQ ID NO:29, respectively. An illustrative FT_Tv7 coding sequence and its corresponding amino acid sequence from Sphingobium herbicidovorans are provided as SEQ ID NO:30 and SEQ ID NO:31, respectively. An illustrative TDO coding sequence and its corresponding amino acid sequence from Oryza sativa are provided as SEQ ID NO:32 and SEQ ID NO:33, respectively. An illustrative EPSPS coding sequence and its corresponding amino acid sequence from Agrobacterium CP4 strain are provided as SEQ ID NO:34 and SEQ ID NO:35, respectively.

The soybean plants, plant seeds, plant parts, or plant cells produced by the preceding methods are tolerant to at least one additional herbicide besides PPO inhibitors in a field, selected from the group consisting of benzoic acid auxins, phenoxy auxins, inhibitors of glutamine synthetase, β-triketone HPPD inhibitors, inhibitors of EPSPS, and combinations of any thereof. An example of benzoic acid auxins includes, but is not limited to, dicamba. An example of the phenoxy auxins includes, but is not limited to, 2,4-D. An example of glutamine synthetase inhibitors includes, but is not limited to, glufosinate. Illustrative examples of β-triketone HPPD inhibitors include, but are not limited to, mesotrione, benzobicyclon (BBC), tembotrione, sulcotrione, and tefuryltrione. An example of the inhibitor of 5-enolpyruvylshikimate-3-phosphate synthase (EPSPS) is glyphosate.

The herbicide(s) used in the methods described herein can be applied alone, sequentially with or in combination with one or more herbicide(s) during the growing season. The herbicide(s) used in the methods described herein can be applied in combination with one or more herbicide(s) temporally (for example, as a tank mixture or in sequential applications), spatially (for example, at different times during the growing season including before and after soybean seed planting), or both. For example, a method for controlling the development of herbicide resistance in weeds is provided that consists of planting seed comprising soybean event Gm_CSM63717 in an area and applying an herbicidally effective amount over the growing season of one or more PPO herbicides alone or in any combination, for the purpose of controlling the development of herbicide resistance in weeds in the area. Such application of herbicide(s) may be pre-planting (any time prior to planting seed comprising soybean event Gm_CSM63717, including for burn-down purposes, that is application to emerging or existing weeds prior to seed plant), pre-emergence (any time after seed comprising soybean event Gm_CSM63717 is planted and before plants comprising soybean event Gm_CSM63717 emerge), or post-emergence (any time after plants comprising soybean event Gm_CSM63717 emerge). Multiple applications of one or more herbicides, or a combination of herbicides together or individually, may be used over a growing season, for example, two applications (such as a pre-planting application and a post-emergence application, or a pre-emergence application and a post-emergence application) or three or more applications (such as a pre-planting application and two post-emergence applications).

Also provided are methods of reducing loci for soybean breeding by site-directed insertion of a transgene that provides tolerance to a PPO herbicide at a genomic location in a soybean plant that is within about 3-8 cM of a locus in the genome of the soybean plant that comprises transgenes for tolerance to at least three additional herbicide modes of action, the three additional herbicide modes of action each being different from one another. The transgenes for tolerance to the at least three additional herbicide modes of action can be selected from the group consisting of PAT, DMO, FT_Tv7, TDO, EPSPS, and combinations of any thereof. These transgenes provide a commercial level of tolerance to at least one herbicide in a field, such as dicamba, glufosinate, 2,4-D, mesotrione, glyphosate, and combinations of any thereof. Illustrative nucleotide and amino acid sequences of the PAT gene are provided as SEQ ID NOs:26-27; illustrative nucleotide and amino acid sequences of the DMO gene are provided as SEQ ID NOs:28-29; illustrative nucleotide and amino acid sequences of the FT_Tv7 gene are provided as SEQ ID NOs:30-31; illustrative nucleotide and amino acid sequences of the TDO gene are provided as SEQ ID NOs:32-33, and illustrative nucleotide and amino acid sequences of the EPSPS gene are provided as SEQ ID NOs:34-35. Crossing of event Gm_CSM63717 with a soybean plant that comprises these transgenes (e.g., with a plant comprising soybean event Gm_CSM63714 and/or with a plant comprising event MON87429) produces progeny plants comprising PPO, PAT, DMO, FT_Tv7, TDO and/or EPSPS, which segregate as a single locus (for progeny plants from a cross between event Gm_CSM63717 and event Gm_CSM63714) or two loci (for progeny plants from a cross between event Gm_CSM63717 and event MON89788, or for progeny plants resulting from three way crosses and comprising event Gm_CSM63717, event Gm_CSM63714 and event MON89788). As used herein, the term “commercial level” in reference to an herbicide refers to the recommended commercial rate (1×) for herbicide application for a specific herbicide. For example, a 1× rate of dicamba is 1 lb/acre pre-emergence and 0.5 lb/acre post-emergence; a 1× rate of 2,4-D is 11b/acre post-emergence; a 1× rate of glufosinate is 0.8 lb/acre; a 1× rate of mesotrione is 0.18 lb/acre pre-emergence, and 0.09 lb/acre post-emergence; a 1× rate of glyphosate is 1.125 lb/acre post-emergence; and a 1× rate of flumioxazin PPO herbicide is 448 g/hectare (0.4 lb/acre) pre-emergence, and 224 g/hectare (0.2 lb/acre) post-emergence. As used herein, “commercial level tolerance” refers to tolerance to one or more herbicides at the recommended commercial rates or higher as a result of transgene expression from one or more expression cassettes in plants comprising event Gm_CSM63717.

Soybean event Gm_CSM63717 with the unique characteristics such as stable integration and expression of the PPO transgene, consistent and superior combinations of efficacy, including herbicide tolerance and agronomic performance, in and across multiple environment conditions in different geographies can be bred or introgressed into elite lines or varieties by conventional breeding methods, and maintained over subsequent generations. Furthermore, since the event was integrated near the transgenic insertion site of a soybean plant comprising the transgenes of PAT, DMO, FT_Tv7 and TDO, the methods of the present disclosure allow for PPO along with PAT, DMO, FT_Tv7 and TDO to segregate as a single locus if bred together, which will allow for rapid trait integration of the multiple transgenes on segregating material, saving time and resources in a breeding program and enabling rapid development of lines, compared to cases where the individual transgenes are inserted into two or more loci, necessitating tedious and laborious multiple crosses over multiple generations to select for plants comprising the multiple genes. The newly introgressed or integrated DNA molecule or polynucleotide of event Gm_CSM63717 comprising SEQ ID NO:9 and/or SEQ ID NO:10 will maintain the expression characteristics of the transgene, and the genomic flanking sequences and chromosomal location, where it will confer tolerance to the PPO herbicides.

### Deposit Information

A deposit of a representative sample of soybean seed comprising event Gm_CSM63717 has been made on Jun. 22, 2023, according to the Budapest Treaty with the American Type Culture Collection (ATCC) Patent Repository having an address at 10801 University Boulevard, Manassas, Virginia, 20110, USA. The ATCC Patent Deposit Designation (accession number) for seeds comprising soybean event Gm_CSM63717 is Accession No. PTA-127604. Access to the deposits will be available during the pendency of the application to the Commissioner of Patents and Trademarks and persons determined by the Commissioner to be entitled thereto upon request. Upon issuance of the patent, all restrictions upon availability to the public will be irrevocably removed. The deposit will be maintained in the depository for a period of thirty (30) years, or five (5) years after the last request, or for the effective life of the patent, whichever is longer, and will be replaced as necessary during that period.

### EXAMPLES

The following examples are included to more fully describe the invention. Summarized are the construction and testing of more than 769 initial proof-of-concept transformation constructs containing various combinations of expression elements and PPO genes/variants, identification, testing and selection of gRNA target sites for site-directed integration of the PPO transgene, leading to the construction and testing of four commercial transformation constructs, the production and testing of over 22,000 unique transformation events, and the analysis of thousands of individual plants over multiple seasons through rigorous molecular characterization, efficacy and agronomic testing both in a controlled environment and in field trials, leading to the creation, identification, and ultimate selection of soybean event Gm_CSM63717 (FIG. 3).

The Examples illustrate certain embodiments of the present disclosure. It should be appreciated by those of skilled in the art that many modifications can be made in the specific examples which are disclosed and still obtain a similar result. Certain agents which are both chemically and physiologically related may be substituted for the agents described herein while achieving the same or similar results. All such substitutions and modifications apparent to those skilled in the art are deemed to be within the scope of the invention.

### Example 1: Testing and Selection of PPO Genes and Expression Elements

This example describes design and testing of different protoporphyrinogen oxidase (PPO) genes and variants, promoters, chloroplast targeting peptides, and 3′ UTRs in many different proof-of-concept constructs to select combinations for commercial transformation to achieve tolerance to PPO herbicides.

Transgene expression and performance in transgenic plants may be influenced by many factors. These include but are not limited to: 1) the expression elements used to drive the expression of the transgene in the expression cassette, and their interactions among themselves and with the transgene; 2) efficient targeting of the transgenic protein to the site of action, such as in the plastids; 3) the relative position and orientation of different expression cassettes when the transgenic insert comprises multiple expression cassettes, each carrying a different transgene conferring a distinct trait or function; 4) the genomic location of the transgenic insertion, also known as positional effect; and 5) faithful integration of the transgene cassette(s) at the intended genomic location on the chromosome for site directed integration. A commercially useful transgenic event requires that the transgene(s) in the transgenic insert expresses in the manner necessary for that trait or function to be successful, e.g., transgene expression and performance across different tissues and developmental stages, in various germplasms, and under different growth conditions.

For these reasons, it is often necessary to create and screen a large number of constructs and transformation events in order to identify combinations of expression elements for constructs (the lead constructs), and then an event (the lead event), which demonstrates optimal expression and performance of the transgenes without phenotypic and agronomic off-types such as yield drag. Prior to such studies, it is not possible to determine whether a particular beneficial event phenotype or performance can be obtained.

In an initial proof-of-concept (POC) and early development stage study, conducted over five years and multiple growing seasons, all microbial genomes curated in Bayer microbial database were annotated with Prodigal (Hyatt et al, 2010; https://github.com/hyattpd/Prodigal). All protein sequences of annotated putative coding genes were scanned with Pfam28.0 (Finn et al, 2014), resulting in a total number of more than 50 million proteins. A total of 2,726 hemG and 15,885 hemY genes were identified and used as input for a ranking algorithm (Xu et al, 2008). The top ranked PPO genes, and chloroplast transit peptides (a.k.a. target signals) mined from more than 30 plant and bacterial genomes for targeting the PPO protein to the plastids were tested. Over 200 different constructs containing 121 PPO genes/variants from different bacteria, 35 different 5′ UTRs, 60 different chloroplast transit peptide sequences, and 20 different 3′UTRs were designed, constructed and assayed first in protoplast expression system. More than 769 constructs were also constructed and transformed into soybean mature seed-derived embryo explants through Agrobacierium-mediated transformation using methods known in the art. Each construct also contained an aadA cassette as a selectable marker. Thousands of transformed plants were regenerated and tested in the greenhouse for protein expression through measurement of protein levels by ELISA, for trait efficacy through herbicide spray treatment, and for agronomic performance. In addition, 18 constructs were designed and constructed to test for site directed integration (SDI) of the PPO cassette (Example 2), and 8 constructs for autoexcision of the selectable marker and the SDI machinery.

Table 4 provides a summary of the proof-of-concept experiments. Thousands of R0 transgenic events were generated from the 769 constructs. These events were subjected to quantitative Taqman PCR assay for transgene copy number. A total of 6075 single copy events were sprayed with PPO herbicides, and less than one-fifth (1085) of these events passed the spray test, showing ≤10% herbicide injury. A total of 658 events from 97 constructs were advanced to R1 nursery for seed increase and field testing for construct selection.

TABLE 4
Summary of proof-of-construct testing

| | |
|---|---|
| # Constructs | 769 |
| # PPO variants | 66 |
| # PPO source organisms | 32 |
| # 5′ UTRs | 35 |
| # Chloroplast transit peptides | 60 |
| # 3′ UTRs | 20 |
| # Events sprayed with PPO herbicides | 6075 |
| # Events passing spray with ≤10% injury | 1085 |
| # Events advanced to field testing | 658 |
| # Constructs advanced to field testing | 97 |

R0 events were generated and screened over a period of time. As they passed the molecular screening and greenhouse spray test, they were advanced to field trials over 11 field trial seasons in North and/or South America. Some events were tested in more than one field trial, while others were included in one trial and then dropped due to lack of efficacy or/and agronomic performance. Table 5 provides a summary of the field trials for the proof-of-concept constructs.

TABLE 5
Summary of field trials on the proof-of-concept constructs

| | | | | | Number of | |
|---|---|---|---|---|---|---|
| Field | | | | | Locations/ | Herbicide |
| Trial | | Number of | Number | Field Trials | Number of | Application |
| Season | Location | Constructs | of Events | Type | Replications | Stage |
| 1 | North | 2 | 8 | Efficacy | 18/2 | V3, R1 |
| | America | 2 | 9 | Agronomics | 4/3 | |
| 2 | South | 11 | 54 | Agronomics | 6/2 | |
| | America | | | | | |
| 3 | North | 24 | 175 | Efficacy | 9/3 | Pre-emergence, |
| | America | 33 | 177 | Agronomics | 18/2 | V3, R1 |
| 4 | South | 34 | 179 | Agronomics | 12/2 | |
| | America | | | | | |
| 5 | North | 41 | 207 | Efficacy | 9/3 | Pre-emergence. |
| | America | 42 | 212 | Agronomics | 18/2 | V3, R1 |
| 6 | South | 21 | 116 | Efficacy | 6/3 | Pre-emergence, |
| | America | 21 | 114 | Agronomics | 12/2 | V3. R1 |
| 7 | North | 18 | 82 | Efficacy | 9/3 | Pre-emergence, |
| | America | 19 | 83 | Agronomics | 18/2 | V3, R1 |
| 8 | South | 8 | 36 | Efficacy | 6/3 | Pre-emergence, |
| | America | 8 | 36 | Agronomics | 12/2 | V3, R1 |
| 9 | North | 15 | 59 | Efficacy | 9/3 | Pre-emergence, |
| | America | 13 | 48 | Agronomics | 17/2 | V3, R1 |
| 10 | South | 13 | 48 | Efficacy | 6/2 | Pre-emergence, |
| | America | 12 | 48 | Agronomics | 12/2 | V3, R1 |
| 11 | North | 8 | 36 | Efficacy | 12/3 | Pre-emergence. |
| | America | 8 | 42 | Agronomics | 20/3 | V3, R1 |

Herbicide treatments were applied using a CO₂ backpack or tractor-mounted sprayer calibrated to deliver 15 gallons per acre (GPA) using air inducted Teejet® TTI nozzles with water as the herbicide carrier. In the first season trials, epyrifenacil was applied at V3 and R1 stages at 40 g/ha. In the third, fifth, sixth, seventh and eighth season trials, flumioxazin was applied at pre-emergence at 210 g/ha, followed by epyrifenacil at V3 and R1 at 60 g/ha. The tenth and eleventh season trials contained two different PPO herbicide treatments. In Treatment 1, flumioxazin was applied at pre-emergence, V3 and R1 at 210 g/ha (in the tenth season trials) or 448 g/ha (in the eleventh season trials). In Treatment 2, epyrifenacil was applied at pre-emergence, V3 and R1 at 60 g/ha. All PPO herbicide applications contained 1% crop oil concentrate (COC). Plots were rated for visual crop injury 14 days after pre-emergence treatment, or 7 days after post emergence treatments, including % injury, chlorosis, necrosis and/or malformation. In addition, emergence, plant height (PHT), moisture (MST), days to flowering (DTF), days of flowering (DOF), harvest weight (HW), maturity (MT), and yield (YLD) data were also collected for both efficacy and agronomics trials. All data were subjected to analysis of variance and means separation at p<0.05.

Based on the results of greenhouse spray test, transgene copy number, and five season field trials, top-performing combinations of expression elements, chloroplast transit peptide and PPO gene were identified, and were used for designing commercial transformation vectors.

### Example 2: Selection of Genomic Target Sites and Guide RNAs

This example describes the bioinformatic analysis and identification of genomic target sites for site directed integration (SDI) of transgenes; screening of LbCas12a (also known as LbCas12a) guide RNAs targeting the genomic sites and identification of unique, high-quality gRNAs to facilitate SDI of transgenes.

In order to identify potential genomic sites for site directed integration of the PPO transgene cassette at a site close to the location of the Gm_CSM63714 event and generate a transgene stack to reduce transgenic loci to facilitate trait introgression and breeding, the Gm_CSM63714 event was first mapped onto the soybean A3555 reference genome assembly. The genome sequence 5 centimorgan (cM) upstream and downstream of the event Gm_CSM63714 insertion site (referred to as the 10 cM window) was identified and the genetic coordinates were linked to physical positions on the A3555 chromosome.

CRISPR Cas12a-based cleavage of DNA sequences requires the use of gRNAs that can target specific sequences (target sites) comprising a Cas12a-recognizable protospacer adjacent motif (PAM) sequence so as to facilitate Cas12a enzymatic activity and DNA cleavage. The 10 cM window was scanned for the presence of the LbCas12a-preferred PAM (TTTV, where “V” is A, C, or G) that were unique in the genome (i.e., that only occur once in the genome). This set was additionally filtered so as to identify gRNA target sites that were more than 2 kb from a coding region, more than 1 kb from a small RNA hotspot, and comprised 700 nucleotides (nt) of unique sequence on either side. A suite of genomic target sites was identified from this analysis and 12 were chosen for testing based on additional factors like GC content, and absence of potential off-targets. Table 6 summarizes the identified target sites that are represented by SEQ ID NOs:211-222. PAM sites are italicized and shown in bold. The corresponding 23 bp gRNA spacer (SP) sequences are also identified and represented by SEQ ID NOs:223-234.

Three Agrobacterium T-DNA vectors were designed to evaluate gRNA and LbCas12a nuclease mediated cleavage at the selected target sites. Each T-DNA vector contained 3 cassettes between the left and right borders on the T-DNA: an aadA selection marker cassette, an LbCas12a Cas12a cassette, and a multiplexed gRNA cassette. The aadA and LbCas12a cassettes were common across the three vectors. The aadA expression cassette contained the aadA gene (also known as Tn7 adenylyltransferase (AAD(3″)) from E. coli under the control of a constitutive promoter, and targeted the aadA protein to the chloroplast for selection of transgenic events resistant to spectinomycin and streptomycin. The LbCas12a cassette comprised a plant codon optimized LbCas12a (SEQ ID NO:236) flanked by the same nuclear localization signal sequence (NLS) at the 5′ and 3′ ends (SEQ ID NO:235) and a 3′UTR sequence (SEQ ID NO:237) derived from a Medicago truncatula gene. The gene cassette comprising NLS-Cas12aLbCas12a-NLS sequence was fused 3′ to an ATGGCG sequence and operably linked to a chimeric promoter sequence (SEQ ID NO:238) comprising an enhancer region from the Banana Streak Virus (Strain Acuminata Vietnam) and promoter with the 5′ UTR region from the Dahlia mosaic virus. The third cassette was unique to each vector and comprised a gRNA cassette array comprising direct repeat sequences (DR) (SEQ ID NO:239) compatible with the Cas12a enzyme and four unique spacers (SP) that when transcribed resulted in four guide RNA units that were complementary to and hybridized with their intended target sites listed in Table 6.

TABLE 6
Cas12a gRNA target sites on Glycine max A3335 chromosome 13 and corresponding
spacer sequences.

| | | Target | | | |
|---|---|---|---|---|---|
| Target | | Site | | | Spacer |
| Site | | SEQ | Spacer | | SEQ |
| Name | gRNA Target Sequence | ID NO | Name | gRNA Spacer | ID NO |
| GmTS1 | TTTGCAAACATCACAT | 211 | SP1 | CAAACATCACAT | 223 |
| | GGCCGGATAAG | | | GGCCGGATAAG | |
| GmTS2 | TTTACTTCCTGAGGAA | 212 | SP2 | CTTCCTGAGGAA | 224 |
| | ATTTGGTTTCA | | | ATTTGGTTTCA | |
| GmTS3 | TTTGGCAAGATCCGCT | 213 | SP3 | GCAAGATCCGCT | 225 |
| | GCCTGCTTGAG | | | GCCTGCTTGAG | |
| GmTS4 | TTTACCTTGATTTGGA | 214 | SP4 | CCTTGATTTGGAT | 226 |
| | TTAACCCGCCA | | | TAACCCGCCA | |
| GmTS5 | TTTACTAAATCGCATA | 215 | SP5 | CTAAATCGCATA | 227 |
| | GCTTGTGTGTT | | | GCTTGTGTGTT | |
| GmTS6 | TTTACATGCTGTCATC | 216 | SP6 | CATGCTGTCATCA | 228 |
| | ATCTGTTCACT | | | TCTGTTCACT | |
| GmTS7 | TTTCCCTATATCTGTTT | 217 | SP7 | CCTATATCTGTTT | 229 |
| | CAGTGATTGC | | | CAGTGATTGC | |
| GmTS8 | TTTGGTGTATACGTGT | 218 | SP8 | GTGTATACGTGT | 223 |
| | GTGAGACAACT | | | GTGAGACAACT | |
| GmTS9 | TTTGCGGAGTAGCAG | 219 | SP9 | CGGAGTAGCAGA | 231 |
| | AGAGGAGATTCG | | | GAGGAGATTCG | |
| GmTS10 | TTTGATCAAAGCTAAA | 220 | SP10 | ATCAAAGCTAAA | 232 |
| | GCATAAGCATC | | | GCATAAGCATC | |
| GmTS11 | TTTCGCTCGCTATATA | 221 | SP11 | GCTCGCTATATAT | 233 |
| | TATGTGTCACA | | | ATGTGTCACA | |
| GmTS12 | TTTGGACATATATGCT | 222 | SP12 | GACATATATGCT | 234 |
| | ATATCATTCTC | | | ATATCATTCTC | |

The guide RNA cassette in vector pG947 comprised a Glycine max Pol III promoter operably linked to a transcribable sequence comprising in order a DR sequence (SEQ ID NO:239), spacer SP10 (SEQ ID NO:232), DR (SEQ ID NO: 239), spacer SP11 (SEQ ID NO:233), DR (SEQ ID NO: 239), spacer SP2 (SEQ ID NO:224), DR (SEQ ID NO: 239), spacer SP12 (SEQ ID NO:234), DR and a poly (T)₇ terminator. The guide RNA cassette in vector pG949 comprised a Glycine max Pol III promoter operably linked to a transcribable sequence comprising in order a DR sequence (SEQ ID NO:239), spacer SP9 (SEQ ID NO:231), DR (SEQ ID NO: 239), spacer SP7 (SEQ ID NO:229), DR (SEQ ID NO: 239), spacer SP1 (SEQ ID NO:223), DR (SEQ ID NO: 239), spacer SP5 (SEQ ID NO:227), DR (SEQ ID NO: 239) and a poly (T)₇ terminator. The guide RNA cassette in vector pG950 comprised a Glycine max Pal III promoter operably linked to a transcribable sequence comprising in order a DR sequence (SEQ II) NO:239), spacer SP3 (SEQ ID NO:225), DR (SEQ ID NO: 239), spacer SP6 (SEQ ID NO:228), DR (SEQ ID NO: 239), spacer SP4 (SEQ ID NO:226), DR (SEQ ID NO: 239), spacer P8 (SEQ ID NO:230), DR and a poly (T)₇ terminator.

The three plant transformation vectors were introduced into soybean seed-derived embryo explants through Agrobacterium-mediated transformation using methods known in the art. Transformed plants were selected on spectinomycin. Genomic DNA was extracted from leaf samples harvested from regenerated plantlets. Each targeted region was amplified by PCR. The amplicons were sequenced by a method known as AmpSeq (Amplicon Sequencing) (see Yang et.al., 2016) to identify modified sequences comprising insertions or deletions (indels) around each target site that are indicative of guide RNA-Cas12a mediated editing.

Table 7 summarizes the results and shows the editing rate detected at each site in stably transformed soybean plants. The editing rate was calculated by taking the number of plants with target site edits, dividing by the number of plants examined, and multiplying by 100. As shown in Table 7, the target site editing rate for the tested gRNAs ranged from 2.4% to 72%.

The top-performing gRNA, targeting GmTS12 (SEQ ID NO:222) and comprising spacer SEQ ID NO:234, had a 72% editing rate and was advanced for site-directed integration of the PPO trait cassette.

TABLE 7
gRNA target site editing rates.

| | | Number of Plants | |
|---|---|---|---|
| gRNA Target | Number of Plants | with Target | Target Editing |
| Site Name | Examined | Site Edits | Rate (%) |
| GmTS1 | 84 | 2 | 2.4 |
| GmTS2 | 82 | 6 | 7.3 |
| GmTS3 | 84 | 7 | 8.3 |
| GmTS4 | 84 | 7 | 8.3 |
| GmTS5 | 84 | 12 | 14.3 |
| GmTS6 | 84 | 15 | 17.9 |
| GmTS7 | 84 | 20 | 23.8 |
| GmTS8 | 84 | 28 | 33.3 |
| GmTS9 | 84 | 31 | 36.9 |
| GmTS10 | 82 | 37 | 45.1 |
| GmTS11 | 82 | 40 | 48.8 |
| GmTS12 | 82 | 59 | 72.0 |

### Example 3: Construct Design, Molecular Analysis, and Event Selection

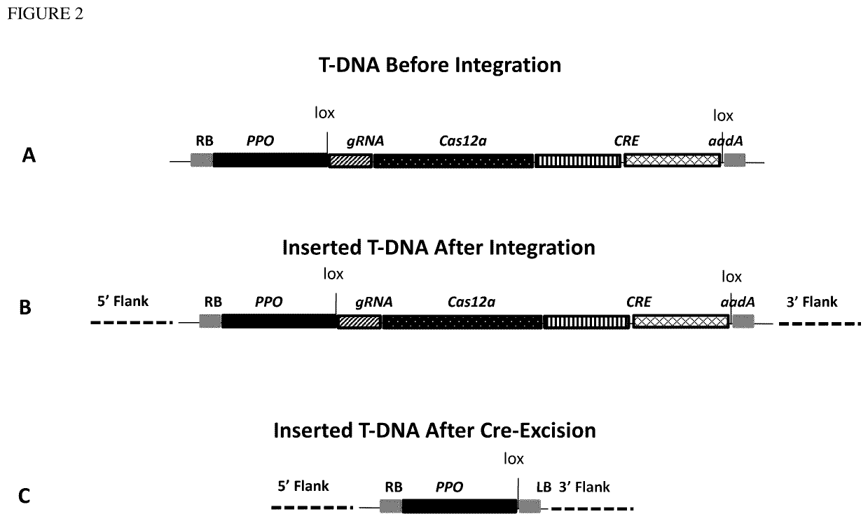

Based on the test results of individual expression cassettes from Example 1, and the results of guide RNA target site testing from Example 2, four commercial transformation constructs were designed and constructed (GmHT5-1, GmHT5-2, GmHT5-3, and GmHT5-4). The constructs each contained five cassettes between the left and right borders on the T-DNA: an aadA cassette, a Cre cassette, a Cas12a cassette, a gRNA cassette, and a PPO cassette. Two lox sites flanked the aadA and the gRNA cassettes. The four constructs contained identical aadA, Cre, and gRNA cassettes, but differed in the Cas12a and PPO cassettes. Three of the constructs contained the same Cas12a cassette, whereas in the fourth construct the Cas12a gene was under the control of different 5′ and 3′ UTRs. Two of the constructs contained identical PPO cassettes, whereas the other two contained different 5′ and 3′ expression elements. Upon transgene insertion and expression of the Cre enzyme, the aadA, Cre, Cas12a and gRNA cassettes were excised out, leading to progenies containing only the PPO cassette (FIG. 2). In addition, two more similar constructs (GmHT5-5 and GmHT5-6) were designed, which contained a different gRNA for targeting the PPO gene to a different genomic location.

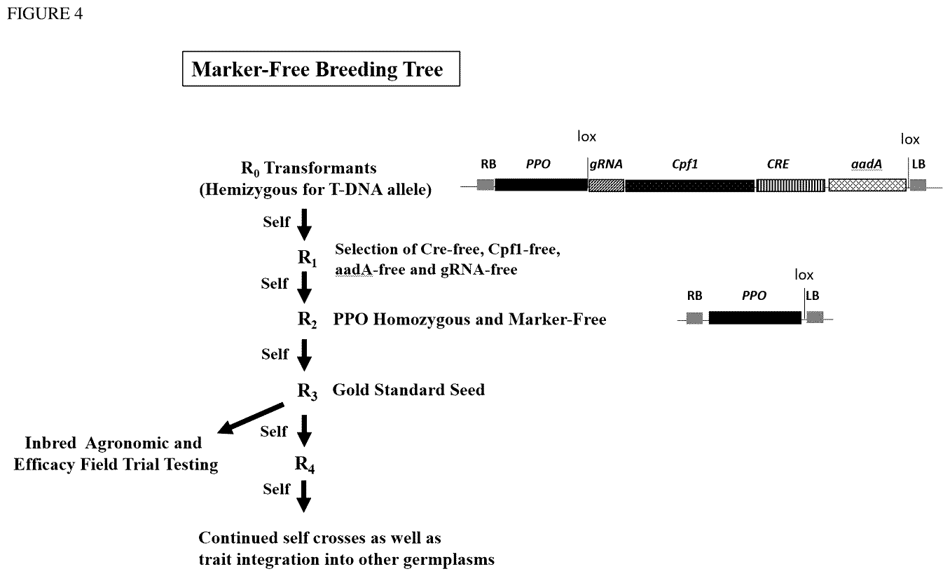

The four (GmHT5-1 to GmHT5-4) different constructs were cloned into plant transformation vectors and introduced into soybean seed-derived embryo explants through Agrobacterium-mediated transformation using methods known in the art. The aadA expression cassette contained the aadA gene (also known as Tn7 adenylyltransferase (AAD(3″))) from E. coli under the control of a constitutive promoter, and targeted the aadA protein to the chloroplast for selection of transgenic events resistant to spectinomycin and streptomycin. The Cre expression cassette encodes the Cre recombinase from Enterobacteria phage P1, under the control of a germline-preferred promoter which was derived from a BURP domain-containing gene, for removal of the aadA. Cre, Cas12a and gRNA cassettes in the progenies. The gRNA (containing the gRNA site GmTS12) in the gRNA cassette was under the control of a Pol III constitutive promoter and targeted the Cas12a enzyme to a genomic location of the soybean genome for site-directed integration of the transgenes. The Cas12a expression cassette encoded a nuclear targeted LbCas12a protein from Lachnospiraceae bacterium ND2006 codon-optimized for soybean expression and under the control of constitutive promoters for site-directed integration of the transgenes. The PPO expression cassette encoded a chloroplast targeted PPO protein from Enterobacter cloacae for conferring tolerance to the PPO herbicides under the control of constitutive promoters. FIG. 2 shows the organization of the T-DNA in these constructs before and after T-DNA insertion into the genome (Panels A and B, respectively), and the inserted T-DNA after excision of the aadA, Cre, Cas12a, and gRNA cassettes following Cre-mediated excision (Panel C). FIG. 4 also shows the organization of the T-DNA in these constructs before insertion into the genome (top right) and the inserted T-DNA after excision of the aadA. Cre. Cas12a, and gRNA cassettes (middle right). A total of 13,142 unique transformation events were produced. R0 plants were subsequently regenerated from the transgenic events. Rooted plants with normal phenotype were transferred to soil for growth and further assessment.

Molecular analyses were conducted concurrently with the controlled environment testing and field trials on events that were advanced. DNA amplification and sequencing were used to determine the composition and intactness of the insert sequence, insert copy number, the presence/absence of Agrobacterium Ti-plasmid backbone sequence, the insertion site properties (e.g., proximity to endogenous genes or repeated regions), and targeted insertion. Specifically, the 13,142 unique R0 events were analyzed for the following desirable molecular characteristics: 1) The PPO transgene was full length, and present either as single copy or two copies at two different loci so that one can be segregated out at R1 generation (in such a case, one of the copies had desirable targeted insertion); 2) The PPO transgene was inserted at the target site defined by the gRNA (targeted insert event); 3) The transgene was not inserted in or near an endogenous gene or a repeated region, and has no SNPs (single nucleotide polymorphisms), to ensure that the sequence in planta matches the sequence in the construct; 4) Removal of the aadA, Cre, Cas12a and gRNA cassettes by auto-excision; 5) The events were free of Agrobacterium Ti plasmid backbone; and 6) The genomic sequence flanking the transgene insert were identified.

As shown in Table 8, from a total of 13,142 unique R0 events generated from the four constructs, 9 unique events met the criteria and were advanced to produce R2 seeds (FIG. 4). The seeds were analyzed to select for events that were homozygous for the PPO transgene, and did not contain Cre, or aadA, Cas12a or the gRNA. As a result, one event from each of constructs GmHT5-1 and GmHT5-2 were selected as the targeted events for further testing. Based on the molecular analyses and field performance of the events as described in Example 4, the lead event was selected from construct GmHT5-1.

In addition, a total of over 10,000 R0 events were generated and tested for constructs GmHT5-5 and GmHT5-6 (data not shown). These constructs/events were deprioritized after selection of GmTS12 as the gRNA target site.

Northern analysis was done to detect and measure mRNA transcript of the PPO transgene in the transgenic events. Protein analysis of plants comprising each event was conducted using techniques known in the art. N-terminal protein sequencing of the PPO proteins purified from transgenic plants containing each event was done to confirm the recombinant protein sequence. Western blot analysis was conducted on protein extracts to confirm the production of the PPO protein from each transgenic event.

TABLE 8
Summary of events selection from the four
commercial transformation constructs

| Construct | GmHT5-1 | GmHT5-2 | GmHT5-3 | GmHT5-4 |
|---|---|---|---|---|
| Total # R0 Event | 5837 | 5730 | 567 | 1008 |
| Targeted Insertion | 7 | 7 | 6 | 2 |
| Flank Identified for | 28 | 45 | 27 | 2 |
| the Targeted Insertion | | | | |
| Molecular Quality | 3 | 5 | 1 | 1 |
| Event | | | | |
| Targeted Insertion and | 3 | 4 | 1 | 1 |
| Auto-Excision in R1, | | | | |
| and Homozygous | | | | |
| Event | | | | |

### Example 4: Field Trials

Field trials were conducted over multiple seasons/years and across many locations in different geographies to evaluate the performance of the constructs/events, and for selecting the best performing construct/event. The field trials comprised efficacy trials for herbicide tolerance, and agronomic trials for yield performance of the events. The performance of many individual plants for each event in each field trial was analyzed as a set. Each event was thus represented by many individual plants. This allowed the performance of each event to be analyzed under many different conditions, in different locations and geographies, and for a variety of properties. Field trials were conducted on homozygous plants to assess trait efficacy for tolerance to commercial rates of PPO herbicides, and agronomic performance.

### First Season Field Trials

The first season efficacy and agronomic field trials were conducted in North America, and included three targeted events and four randomly inserted events from construct GmHT5-1, four each of targeted and randomly inserted events from GmHT5-2, and one targeted event from each of GmHT5-3 and GmHT5-4. The randomly inserted events were included in the trials to serve 1) as controls to gauge whether PPO expression at the sites was within the range observed in the previously generated randomly inserted proof-of-concept events; 2) as potential back-ups in case PPO expression in the targeted events was not in the desirable range. In the agronomy trials, the events were tested at 18 locations with 2 replications per location using a randomized complete block design (RCBD). Data were collected from 16 locations.

In the efficacy trials, the events were tested at 9 locations with 3 replications and three treatment blocks using a group unbalanced block 2 (GUBD2) design. The three treatments consisted of 1) untreated; 2) flumioxazin at 210 g/ha applied at pre-emergence (PRE); and 3) epyrifenacil at 60 g/ha at V3 and R1 stages, respectively. The herbicide formulations also contained 0.5% (v/v) High Surfactant Oil Concentrate (Superb® HC, Winfield Solutions, LLC, P. O. Box 64589, St. Paul, MN 55164-0589). Additional treatments included higher doses of flumioxazin at pre-emergence, and epyrifenacil at V3 and R1 stages to evaluate PPO herbicide tolerance at the construct level. Herbicides were applied using a tractor-mounted or backpack sprayer calibrated to deliver 15 gallons per acre (GPA) with water as the herbicide carrier using Teejet Air Induction (TTI) nozzles.

Plots were two rows by twelve feet long with a three-foot alley with 30-inch row spacing and seeded at 235 seeds per plot. Trial maintenance was designed to optimize grain yield. Data were collected for emergence, vigor, date of first flower, plant height, maturity, and yield. In addition, visual injury data were collected for the efficacy trials approximately seven days after postemergence herbicide applications or 14 days after preemergence applications on a scale of 0 to 100 with “0” being no visible crop injury and “100” being complete crop injury or death.

All data from both agronomic and efficacy trials were subject to analysis of variance and means separated at LSD at alpha 0.05. Statistical analysis was automated using ASReml software for mixed model fitting.

Events were prioritized for further testing based on molecular assessment, yield performance, and crop injury. In the efficacy trials, yield of herbicide treated events was compared to that of the untreated events. In the agronomy trials, broad acre yield (BAY) of events was compared to that of the untransformed (wildtype) control plants.

Results of the efficacy and agronomy trials for the individual events are summarized in Table 9. All events showed excellent crop response in the efficacy trials even though the PPO protein level varied significantly among all events. Events GmHT5-1-5 (random insertion), GmHT5-2-2 (targeted insertion), GmHT5-2-4 (targeted insertion), GmHT5-2-6 (random insertion) had significantly reduced yield following herbicide treatments when compared to their untreated counterparts. Events GmHT5-1-3 (targeted insertion) and GmHT5-3-1 (targeted insertion) showed significantly reduced BAY yield in the agronomics trials when compared to the wildtype controls. In contrast, event Gm-HT5-1-2 (Gm_CSM63717) performed well in both efficacy and agronomics trials.

TABLE 9
Summary of first season efficacy and agronomic trial results

| | | | | | | | | | WT | |
|---|---|---|---|---|---|---|---|---|---|---|
| | | | | | | | | | Control | Event |
| | | | PPO | | | | | | Broad | Broad |
| | | | Protein | Injury | Injury | Injury | Yield | Yield | Acre | Acre |
| Construct | Event | Insertion | (ppm) | (PRE) | (V3) | (R1) | (Untreated) | (Treated) | Yield | Yield |
| GmHT5-1 | GmHT5-1-1 | Targeted | 89.5 | 0.3 | 0.6 | 0.3 | 59.5 | 64.0 | 62.3 | 63.7 |
| GmHT5-1 | GmHT5-1-2 | Targeted | 72.8 | 1.0 | 0.3 | 0.3 | 61.3 | 62.5 | 62.3 | 65.0 |
| GmHT5-1 | GmHT5-1-3 | Targeted | 90.8 | 0.2 | 0.4 | 0.3 | 59.6 | 61.3 | 62.3 | 57.1 |
| GmHT5-1 | GmHT5-1-4 | Random | 99.5 | 0.5 | 0.5 | 0.3 | 62.2 | 61.2 | 61.3 | 63.8 |
| GmHT5-1 | GmHT5-1-5 | Random | 55.8 | 0.5 | 0.6 | 0.4 | 56.9 | 51.7 | 62.3 | 61.9 |
| CmHT5-1 | GmHT5-1-6 | Random | 58.8 | 0.5 | 0.5 | 0.3 | 60.4 | 62.4 | 62.3 | 64.2 |
| GmHT5-1 | GmHT5-1-7 | Random | 63 | 0.7 | 0.4 | 0.3 | 60.3 | 64.4 | 62.3 | 61.6 |
| GmHT5-2 | GmHT5-2-1 | Targeted | 366.7 | 0.1 | 0.4 | 0.3 | 56.7 | 57.0 | 62.3 | 61.1 |
| GmHT5-2 | GmHT5-2-2 | Targeted | 263.3 | 0.5 | 1.0 | 0.3 | 61.4 | 55.3 | 62.3 | 62.2 |
| GmHT5-2 | GmHT5-2-3 | Targeted | 235 | 0.0 | 1.0 | 0.3 | 58.8 | 58.1 | 62.3 | 62.3 |
| GmHT5-2 | GmHT5-2-4 | Targeted | 292.5 | 0.6 | 0.6 | 0.3 | 58.6 | 55.0 | 62.3 | 63.2 |
| GmHT5-2 | GmHT5-2-5 | Random | 157.5 | 0.1 | 0.5 | 0.3 | 59.0 | 58.3 | 61.3 | 63.1 |
| GmHT5-2 | GmHT5-2-6 | Random | 276.7 | 0.7 | 0.5 | 0.3 | 62.2 | 55.9 | 61.3 | 63.7 |
| GmHT5-2 | GmHT5-2-7 | Random | 277.5 | 0.2 | 0.6 | 0.3 | 57.6 | 59.1 | 61.3 | 62,1 |
| GmHT5-2 | GmHT5-2-8 | Random | 252.5 | 0.6 | 0.3 | 0.7 | 59.3 | 56.6 | 62.3 | 63.8 |
| GmHT5-3 | GmHT5-3-1 | Targeted | 45.3 | 0.6 | 0.4 | 0.3 | 47.1 | 47.8 | 62.3 | 49.9 |
| GmHT5-4 | GmHT5-4-1 | Targeted | 40.3 | 0.5 | 0.4 | 0.3 | 59.5 | 63.1 | 62.3 | 62.4 |

### Second Season Feild Trials

The second season field trials were conducted in South America. The trials included three targeted events and four randomly inserted events from construct GmHT5-1, four each of targeted and randomly inserted events from GmHT5-2, and one targeted event from each of GmHT5-3 and GmHT5-4. In the agronomy trials, the events were tested at 12 locations with 2 replications per location using a RCBD design. Data were collected from 10 locations. Test plots were compared to the transformation germplasm control.

In the efficacy trials, the events were tested at 6 locations with 3 replications per location using a group unbalanced block design (GUBD) (data were collected from five locations). The trials consisted of three treatments: 1) untreated; 2) epyrifenacil at 60 g/ha applied at preemergence, V3 and R1 stages, respectively; and 3) flumioxazin at 560 g/ha applied at pre-emergence, followed by 280 g/ha at V3 and R1, respectively. The herbicide formulations also contained 1% (v/v) Crop Oil Concentrate (Prime Oil®, Winfield Solutions, LLC, P. O. Box 64589, St. Paul, MN 55164-0589). Herbicides were applied using a tractor-mounted or backpack sprayer calibrated to deliver 15 gallons per acre (GPA) with water as the herbicide carrier using Teejet Air Induction (TTI) nozzles. Herbicide-treated event plots were compared to the untreated event plots.

Plots were four rows by four meters long with a one-meter alley, 0.52-centimeter row spacing and seeded at 391 seeds/plot. Trial maintenance was designed to optimize grain yield. Data were collected for emergence, vigor, date of first flower, plant height, maturity, and yield. In addition, visual injury data were collected for the efficacy trials approximately seven days after postemergence herbicide applications or 14 days after preemergence applications.

All data from both agronomic and efficacy trials were subject to analysis of variance and means separated at LSD at alpha 0.05. Statistical analysis was automated using ASReml software for mixed model fitting.

The results of the second season efficacy and agronomics trials are shown in Table 10. All events performed well in the efficacy trials. Event GmHT5-2-6 was not tested in the efficacy trial due to seed limitation. Events GmHT5-1-3 and GmHT5-2-1 showed significantly reduced BAY yield in the agronomics trials when compared to the wildtype controls. In contrast, event GmHT5-1-2 (Gm_CSM63717) consistently performed well in both efficacy and agronomics trials.

TABLE 10
Summary of second season efficacy and agronomics trial results

| | | | | | | | | | | | Broad Acre | |
|---|---|---|---|---|---|---|---|---|---|---|---|---|
| | | Flumioxazin | | | Epyrifenacil | | | | | | Yield | |
| | | Injury | | | Injury | | | Yield After Herbicide Treatment | | | WT | |
| Event | Insertion | PRE | V3 | R1 | PRE | V3 | R1 | Untreated | Flumioxazin | Epyrifenacil | Control | Event |
| GmHT5-1-1 | Targeted | 0.8 | 0.9 | 0.8 | 0.4 | 0.5 | 0.4 | 58.6 | 62.8 | 61.1 | 60.9 | 60.4 |
| GmHT5-1-2 | Targeted | 0.5 | 0.4 | 1.2 | 0.3 | 0.3 | 0.3 | 60.0 | 63.1 | 65.5 | 60.9 | 60.3 |
| GmHT5-1-3 | Targeted | 0.7 | 0.3 | 0.1 | 0.5 | 0.5 | 0.5 | 56.0 | 60.6 | 58.1 | 60.9 | 57.4 |
| GmHT5-1-4 | Random | 0.4 | 0.5 | 0.4 | 0.4 | 0.5 | 0.4 | 63.4 | 64.2 | 62.0 | 60.9 | 61.6 |
| GmHT5-1-5 | Random | 0.5 | 0.4 | 0.3 | 0.8 | 0.5 | 0.8 | 63.1 | 65.4 | 66.0 | 60.9 | 59.3 |
| GmHT5-1-6 | Random | 0.7 | 0.3 | 0.3 | 0.4 | 0.4 | 0.4 | 63.9 | 66.7 | 63.9 | 60.9 | 51.5 |
| GmHT5-1-7 | Random | 0.5 | 1 | 0.8 | 0.1 | 0.7 | 0.1 | 58.7 | 64.4 | 66.5 | 60.9 | 61.6 |
| GmHT5-2-1 | Targeted | 0.8 | 0.3 | 0.7 | 0.5 | 0.1 | 0.5 | 59.2 | 63.5 | 59.8 | 60.9 | 55.3 |
| GmHT5-2-2 | Targeted | 0.6 | 0.3 | 0.4 | 0.7 | 0.3 | 0.7 | 61.0 | 61.7 | 62.5 | 60.9 | 62.6 |
| GmHT5-2-3 | Targeted | 0.7 | 0.3 | 1.1 | 0.5 | 0.4 | 0.5 | 61.8 | 64.4 | 58.8 | 60.9 | 62.0 |
| GmHT5-2-4 | Targeted | 0.5 | 0.5 | 1 | 0.5 | 0.1 | 0.5 | 60.6 | 59.8 | 60.1 | 60.9 | 60.4 |
| GmHT5-2-5 | Random | 0.4 | 0.4 | 1.4 | 0.4 | 0.3 | 0.4 | 64.0 | 65.2 | 61.9 | 60.9 | 61.7 |
| GmHT5-2-6 | Random | — | — | — | — | — | — | — | — | — | 60.9 | 59.6 |
| GmHT5-2-7 | Random | 0.3 | 0.3 | 0.2 | 1.9 | 0.3 | 1.9 | 57.0 | 64.0 | 56.1 | 60.9 | 59.4 |
| GmHT5-2-8 | Random | 0.7 | 0.2 | 0.6 | 0.7 | 0.7 | 0.7 | 62.1 | 68.5 | 58.2 | 60.9 | 64.2 |
| GmHT5-3-1 | Targeted | 0.4 | 1.5 | 1.8 | 3.1 | 2.1 | 3.1 | 61.2 | 64.0 | 62.6 | 60.9 | 58.7 |
| GmHT5-4-1 | Targeted | 0.6 | 0.2 | 0.4 | 0.6 | 0.6 | 0.6 | 60.7 | 63.3 | 68.1 | 60.9 | 60.9 |

### Third Season Field Trials

The third season field trials were conducted in North America. The trials included only targeted events: three from construct GmHT5-1, four from construct GmHT5-2, and one from construct GmHT5-4. In the agronomy trials, the events were tested at 20 locations with three replications per location using a RCBD design. Data were collected from 17 locations. Test plots were compared to the transformation germplasm control.

In the efficacy trials, the events were tested at 9 locations with 3 replications per location and three treatment blocks using a GUBD2 design. The treatments included 1) untreated; 2) epyrifenacil at 60 g/ha applied at PRE, V3 and R 1, respectively; and 3) flumioxazin at 448 g/ha applied at applied at PRE, V3 and R1, respectively. The herbicide formulations also contained 1% (v/v) Crop Oil. Samples were also collected at V3, R1 and R5 stages for measurement of PPO protein levels by ELISA.

Plots were two rows by twelve feet long with a three-foot alley with 30-inch row spacing and seeded at 235 seeds per plot. Trial maintenance was designed to optimize grain yield. Data were collected for emergence, vigor, date of first flower, plant height, maturity, and yield. In addition, visual injury data were collected for the efficacy trials approximately seven days after postemergence herbicide applications or fourteen days after preemergence applications.

All data from both agronomic and efficacy trials were subject to analysis of variance and means separated at LSD at alpha 0.05. Statistical analysis was automated using ASReml software for mixed model fitting. Events were prioritized for further testing based on molecular assessment, yield performance, and crop injury. In the efficacy trials, yield of herbicide-treated events was compared to that of the non-treated and each other within a treatment block. In the agronomy trials, yield of events was compared to that of the non-transformed (wildtype) control plots.

The results of the third season efficacy and agronomics trials are shown in Table 11. In the table heading, “Flumi” stands for flumioxazin; “Epy” stands for epyrifenacil, and “WT Ctrl” stands for Wild-type control. All events performed well in the efficacy and agronomics trials, except for event GmHT5-1-3, which showed significantly reduced BAY yield in the agronomics trials when compared to the wildtype controls. Event GmHT5-1-2 (Gm_CSM63717) consistently performed well in both efficacy and agronomics trials.

TABLE 11
Summary of the third season efficacy and agronomics trial results

| | | | | | | | | | | | Yield after | | | | |
|---|---|---|---|---|---|---|---|---|---|---|---|---|---|---|---|
| | | | | | | | | | | | Herbicide | | | Broad Acre | |
| | | PPO Protein | | | Flumioxazin | | | Epyrifenacil | | | Treatment | | | Yield | |
| | | (ppm) | | | Injury | | | Injury | | | Un- | | | WT | |
| Construct | Event | V3 | R1 | R5 | PRE | V3 | R1 | PRE | V3 | R1 | reated | Flum | Epy | Ctrl | Event |
| GmHT5-1 | GmHT5-1-1 | 46 | 116.6 | 76.5 | 0.0 | 0.2 | 0.2 | 0.0 | 0.1 | 0.0 | 58.4 | 58.1 | 60.5 | 57.5 | 56.8 |
| GmHT5-1 | GmHT5-1-2 | 34.9 | 94.4 | 67.0 | 0.0 | 0.0 | 0.0 | 0.0 | 0.3 | 0.0 | 60.0 | 60.3 | 60.7 | 57.5 | 59.1 |
| GmHT5-1 | GmHT5-1-3 | 138.4 | 116.3 | 54.4 | 0.0 | 0.0 | 0.0 | 0.0 | 0.2 | 0.0 | 57.6 | 55.7 | 57.8 | 57.5 | 54.9 |
| GmHT5-2 | GmHT5-2-1 | 235.0 | 297.5 | 86.6 | 0.0 | 0.0 | 0.1 | 0.0 | 0.2 | 0.0 | 58.7 | 56.9 | 56.5 | 57.5 | 57.9 |
| GmHT5-2 | GmHT5-2-2 | 275.0 | 296.3 | 84.5 | 0.0 | 0.0 | 0.0 | 0.0 | 0.1 | 0.0 | 58.5 | 58.4 | 58.4 | 57.5 | 58.5 |
| GmHT5-2 | GmHT5-2-3 | 216.3 | 248.8 | 71.3 | 0.0 | 0.0 | 0.0 | 0.0 | 0.4 | 0.0 | 59.1 | 60.5 | 58.1 | 57.5 | 59.4 |
| GmHT5-2 | GmHT5-2-4 | 220.0 | 236.3 | 127.6 | 0.0 | 0.1 | 0.1 | 0.0 | 0.3 | 0.0 | 59.0 | 59.7 | 57.8 | 57.5 | 58.0 |
| GmHT5-4 | GmHT5-4-1 | 30.0 | 70.6 | 58.0 | 0.0 | 0.1 | 0.0 | 0.0 | 0.3 | 0.0 | 59.5 | 58.7 | 59.6 | 57.5 | 57.8 |

### Fourth Season Field Trials

The fourth season field trials were conducted in South America. The trials included three targeted events from construct GmHT5-1 and four targeted events from GmHT5-2. In the agronomy trials, the events were tested at 12 locations with 3 replications per location using a RCBD design. Data were collected from 9 locations. Test plots were compared to the transformation germplasm control.

In the efficacy trials, the events were tested at 6 locations with 3 replications per location using a GUBD design. The trials consisted of three treatments: 1) untreated; 2) epyrifenacil at 80 g/ha applied at preemergence, V3 and R1 stages, respectively; and 3) flumioxazin at 448 g/ha applied at pre-emergence, followed by at V3 and R1, respectively. The herbicide formulations also contained 1% (v/v) Crop Oil. Herbicides were applied using a tractor-mounted or backpack sprayer calibrated to deliver 15 gallons per acre (GPA) with water as the herbicide carrier using Teejet Air Induction (TTI) nozzles. Herbicide-treated event plots were compared to the untreated event plots.

Plots were four rows by four meters long with a one-meter alley, 0.52-centimeter row spacing and seeded at 391 seeds/plot. Trial maintenance was designed to optimize grain yield. Data were collected for emergence, vigor, date of first flower, plant height, maturity, and yield. In addition, visual injury data were collected for the efficacy trials approximately seven days after postemergence herbicide applications or 14 days after preemergence applications.

All data from both agronomic and efficacy trials were subject to analysis of variance and means separated at LSD at alpha 0.05. Statistical analysis was automated using ASReml software for mixed model fitting.

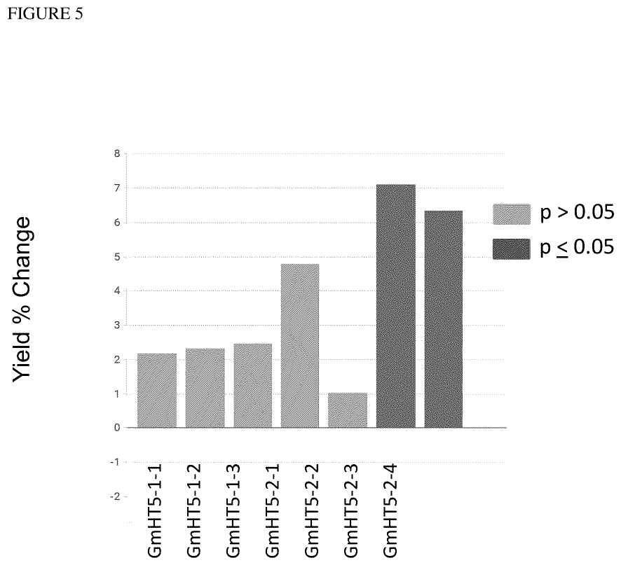

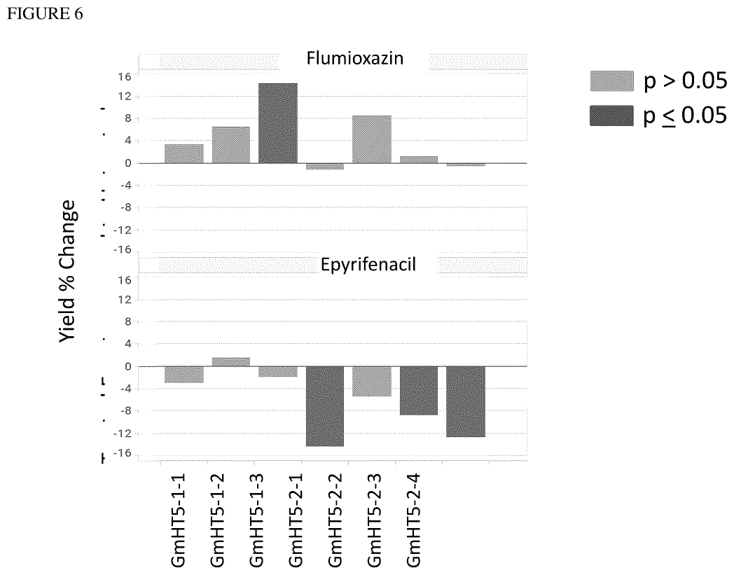

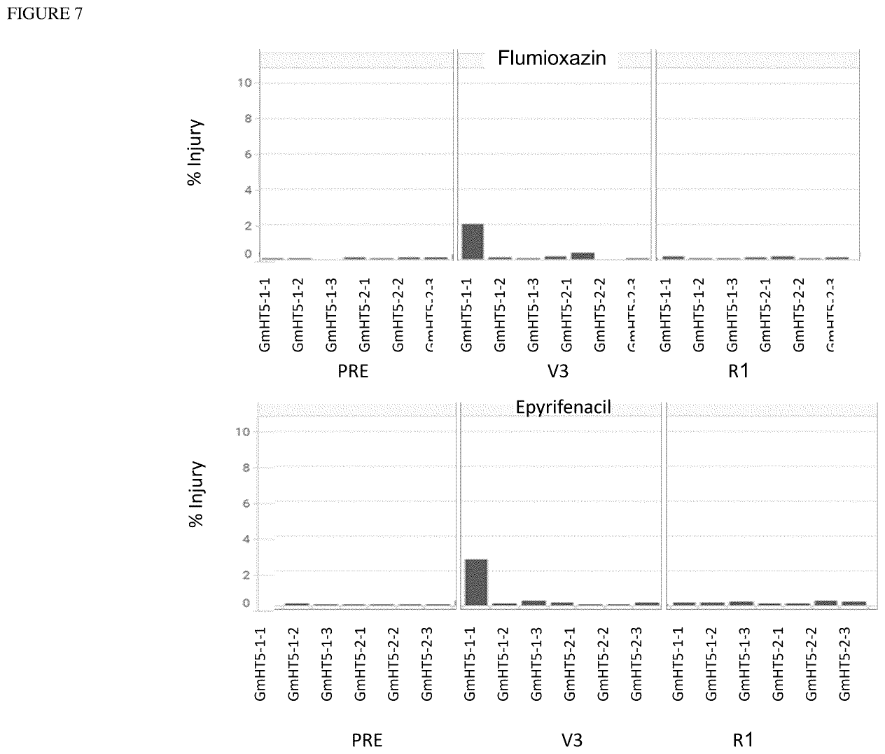

Results from the fourth season field trials showed that for Event GmHT5-1-2 (Gm_CSM63717) no significant difference in yield was observed at p≤0.05 in the agronomics and efficacy trials (FIGS. 5 and 6) when compared to the corresponding controls. In addition, the events performed very well and did not show significant injury after flumioxazin or epyrifenacil applications at PRE, followed by V3 and R1 stages (FIG. 7).

### Meta Analysis

To compare the field trial data and obtain more precise estimate on the performance of the lead transgenic events, a statistical meta-analysis was performed using the aggregate of all plants for the lead events GmHT5-1-1, GmHT5-1-2, GmHT5-1-3, GmHT5-2-1, GmHT5-2-2, GmHT5-2-3, GmHT5-2-4, and GmHT5-4-1 in the multi-season, multi-location field trial data. Table 12 provides the meta-analysis results for yield in the agronomics and efficacy trials. Yield is presented in bushel per acre for the event, or for the untransformed control (wildtype). Based on the results of efficacy and agronomics trials, meta-analysis and molecular assessment. GmHT5-1-2 was selected as the commercial event and was named Gm_CSM63717.

TABLE 12
Meta-analysis of yield from agronomics and efficacy field trials

| | | | | | Broad Acre | |
|---|---|---|---|---|---|---|
| | | | | | Yield | |
| | | Yield after Herbicide Treatment | | | WT | |
| Construct | Event | Untreated | Flumioxazin | Epyrifenacil | Control | Event |
| GmHT5-1 | GmHT5-1-1 | 58.8 | 59.4 | 61.7 | 59.7 | 60.1 |
| GmHT5-1 | GmHT5-1-2 | 60.6 | 61.1 | 62.1 | 59.7 | 61.4 |
| | (Gm_CSM63717) | | | | | |
| GmHT5-1 | GmHT5-1-3 | 57.9 | 57.0 | 59.1 | 59.7 | 56.4 |
| GmHT5-2 | GmHT5-2-1 | 58.2 | 58.6 | 57.3 | 59.7 | 58.4 |
| GmHT5-2 | GmHT5-2-2 | 60.3 | 59.3 | 58.1 | 59.7 | 61.2 |
| GmHT5-2 | GmHT5-2-3 | 59.4 | 61.6 | 58.3 | 59.7 | 61.2 |
| GmHT5-2 | GmHT5-2-4 | 59.1 | 59.8 | 57.0 | 59.7 | 60.5 |
| GmHT5-4 | GmHT5-4-1 | 59.8 | 60.0 | 62.4 | 59.7 | 60.2 |

### Example 5: Tolerance of Event Gm_CSM63717 to Different PPO Herbicides

An efficacy trial was conducted in the controlled environment to evaluate tolerance of event Gm_CSM63717 to various PPO herbicides. The experiment included the lead event (Gm_CSM63717) and a wild-type control in the same transformation germplasm, and consisted of nine individual PPO herbicide treatments, all at 1× rates. These included butafenacil, flumioxazin, fomesafen, lactofen, pyroflufen, saflufenacil, Voraxor (trifludimoxazin and salflufenacil), sulfentrazone, cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate, and an untreated control. The herbicide formulations contained 1% (v/v) Crop Oil Concentrate (COC, Prime Oil, Winfield Solutions, LLC, P. O. Box 64589, St. Paul, MN 55164-0589) for sulfentrazone, fomesafen, lactofen, or 1% (v/v) methylated seed oil (Destiny® HC, Winfield Solutions, LLC, P. O. Box 64589, St. Paul, MN 55164-0589) for flumioxazin, saflufenacil, Voraxor, pyroflufen, butafenacil, and cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate. There were 10 replications per treatment. The plants were grown under standard greenhouse conditions, and were sprayed with each herbicide at V3 stage, and were visually rated for herbicide injury 11 days after herbicide applications.

As shown in Table 13, while the wild-type showed injury across all treatments ranging from about 20%-90% depending on the treatment, whereas the event Gm_CSM63717 showed no injury with any of the treatments.

TABLE 13
Tolerance of Gm_CSM63717 to various PPO herbicides

| | | | Rate | Injury (%) | |
|---|---|---|---|---|---|
| | | Description | (gram | WT | |
| Treatment | Product | (Active Ingredient) | ai/ha) | Control | Gm_CSM63717 |
| 1 | VALOR_SX | flumioxazin (51%) | 140 | 39.5 | 0 |
| 2 | SPARTAN_4F | Sulfentrazone (39.6%) | 240 | 37.5 | 0 |
| 3 | SHARPEN | Saflufenacil (29.74%) | 50 | 85 | 0 |
| 4 | REFLEX | Fomesafen (22.8%) | 420 | 17.5 | 0 |
| 5 | COBRA | Lactofen (24%) | 220 | 30.5 | 0 |
| 6 | VORAXOR | saflufenacil (21.06%) + | 112 | 89 | 0 |
| | | trifludimoxazin (10.67%) | | | |
| 7 | QUICKDOWN | Pyraflufen 24.5% | 100 | 88 | 0 |
| 8 | BPOWER | Butafenacil 100 g/liter | 100 | 77 | 0 |
| 9 | — | cyclopropylmethyl (2-{2-chloro-4- | 100 | 89 | 0 |
| | | fluoro-5-[3-methyl-2,6-dioxo-4- | | | |
| | | (trifluoromethyl)-3,6- | | | |
| | | dihydropyrimidin-1(2H)- | | | |
| | | yl]phenoxy}phenoxy)acetate, | | | |
| | | (20%) | | | |
| 10 | — | Untreated Control | — | 0 | 0 |

### Example 6: Testing of Breeding Stack of Gm_CSM63717 with Event Gm_CSM63714 and Event MON89788

Efficacy trials were conducted in controlled environment to evaluate herbicide tolerance of the breeding triple stack of Gm_CSM63717 (tolerance to PPO inhibitor herbicides), Gm_CSM63714 (tolerance to dicamba, glufosinate, mesotrione and 2,4-D), and MON89788 (tolerant to glyphosate). Plants comprising event Gm_CSM63717 were crossed with plants comprising events Gm_CSM63714 and MON89788 to produce progenies comprising event Gm_CSM63717, event Gm_CSM63714, and event MON89788. Homozygous progenies were identified and used in the trials.

The trials included two entries: the triple stack and the MON89788 control. There were seven treatments and eight replications per treatment. Herbicide treatments consisted of 1× rate each of flumioxazin, dicamba, mesotrione, glufosinate, 2,4-D, glyphosate, and an untreated control. The herbicide formulations also contained 1% (v/v) Crop Oil Concentrate (COC, Prime Oil®, Winfield Solutions, LLC, P. O. Box 64589, St. Paul, MN 55164-0589) for mesotrione, 0.5% (v/v) methylated seed oil (Destiny® HC, Winfield Solutions, LLC, P. O. Box 64589, St. Paul, MN 55164-0589) for fiumioxazin, or 2.5% (v/v) ammonium sulfate (N-Pak® AMS Liquid, Winfield Solutions, LLC, P. O. Box 64589, St. Paul, MN 55164-0589) for glufosinate ammonium. The plants were grown under standard greenhouse conditions, treated with each herbicide at V3 stage, and were visually rated for herbicide injury 14 days after herbicide applications.

The results of the trials are summarized in Table 14. Except for a very minor injury of <5% for 2,4-D, no injury was observed on the triple stack for all herbicides tested. On the other hand, the control MON89788, which is tolerant to glyphosate, sustained significant injuries from the other herbicides, ranging from 50% to 90%.

TABLE 14
Herbicide tolerance of the Gm_CSM63717/Gm_CSM63714/MON89788 triple stack

| | | Description | | | |
|---|---|---|---|---|---|
| | | (Acitve Ingredient | Rate | Injury (%) | |
| | | (ai) or Acid | (gram ae | | Triple |
| Treatment | Product | Equivalent (ae)) | or ai/ha) | MON89788 | Stack |
| 1 | ROUNDUP_POWERMAX_3 | Glyphosate | 1260 | 0 | 0 |
| | | (41.8% ae) | | | |
| 2 | CALLISTO_480SC | Mesotrione | 105 | 51.25 | 0 |
| | | (40% ai) | | | |
| 3 | LIBERTY | Glufosinate | 660 | 90 | 0 |
| | | (24.5% ai) | | | |
| 4 | VALOR_SX | Flumioxazin | 140 | 57.5 | 0 |
| | | (51% ai) | | | |
| 5 | ENLIST_ONE | 2.4-D | 1120 | 80 | 4.75 |
| | | (38% ae) | | | |
| 6 | XTENDIMAX | Dicamba | 560 | 80 | 0 |
| | | (29% ae) | | | |
| 7 | Untreated Control | — | — | 0 | 0 |

### Example 7: Molecular Characterization of Soybean Event Gm_CSM63717

As described above, soybean event Gm_CSM63717 was identified through comprehensive and rigorous molecular characterization and event selection processes carried out from R0 to R5 generations, coupled with field performance testing including trait efficacy and yield. This example describes the extensive molecular characterization upon selection of Gm_CSM63717 as the commercial event, including confirmation of one copy of intact PPO at a targeted locus, absence of Agrobacterium Ti plasmid backbone DNA, aadA, Cas12a, gRNA and Cre sequences; confirmation of the chromosomal location of the PPO transgene, confirmation that the T-DNA did not interrupt any known endogenous gene and did not insert into any repeat regions; and identification of the transgene 5′ and 3′ genomic flanking sequences and the wildtype allele sequence. The transgenic insert of soybean event Gm_CSM63717 contains the elements and sequences described in Table 1.

DNA sequence analysis of the soybean event Gm_CSM63717 was conducted. Whole genome sequencing-based molecular characterization was conducted to confirm that plants of soybean event Gm_CSM63717 contained a single and intact copy of the entire PPO cassette without any transformation vector backbone sequence, or the aadA, Cas12a, gRNA and Cre sequences. The in planta transgenic insert was isolated and sequenced using methods known in the art. Mapping of the sequence reads of the event and the nontransgenic A3555 germplasm against construct GmHT5-1, respectively, confirmed the absence of Agrobacterium Ti plasmid backbone and marker sequences including Cas12a, aadA, gRNA and Cre, and showed that the inserted in planta T-DNA sequence was inserted at the target site on chromosome 13, and perfectly matched the expected T-DNA sequence from the transformation vector GmHT5-1 after the removal of the aadA, Cas12a, Cre and gRNA cassettes.

Amplicons covering transgenic insert and the 5′ or the 3′ flanking sequence were generated by PCR. The transgenic insertion site, and the respective 5′ and 3′ junction sequences, were subsequently determined by amplicon sequencing. The resulting event locus comprised a 1000-nucleotide left flanking genomic sequence, a 3201-nucleotide PPO expression cassette sequence, and a 1000-nucleotide right flanking genomic sequence (see FIG. 1 and Table 1). The WT allele sequence corresponding to the event Gm_CSM63717 transgenic insertion site was also generated from A3555 genomic DNA by PCR and amplicon sequencing. Comparison of the WT allele sequence with that of the event Gm_CSM63717 at the insertion site indicated that the transgenic insertion resulted in an 11-nucleotide genomic sequence deletion in the event. Sequence information for the transgenic insert, the 5′ and 3′ flanking sequences, and the 5′ and 3′ junctions are provided herein as SEQ ID NOs:1-10.

RNA analysis of plants comprising the soybean event Gm_CSM63717 was conducted. Northern hybridization was performed on total RNA and mRNA isolated from immature seeds. The results confirmed RNA transcripts corresponding in size to the PPO mRNA products in soybean event Gm_CSM63717.

Protein analyses of plants comprising soybean event Gm_CSM63717 were also conducted. The N-terminal amino acid sequence of the expressed PPO protein was determined by Edman sequencing and mass spectrometry using immunopurified protein extracts from mature seed and leaf to confirm the authentic N-terminal amino acid sequence. Western blot analysis was conducted on protein extracts from mature seed and leaf of soybean event Gm_CSM63717 to confirm the cleavage of the chloroplast transit peptide and the production of a single expected-sized protein for PPO. In addition, ELISA was used to determine PPO protein level in leaves during different developmental stages (V3, R1 and R5), under different growth conditions (greenhouse, growth chamber, and field), over multiple generations (R0 to R5). The results showed that the PPO protein remained stable across different growth conditions and over multiple generations tested.

### Example 8: Detection of Soybean Event Gm_CSM63717

This example describes methods useful in identifying or detecting the presence of soybean event Gm_CSM63717. Detection of the event in a sample can be achieved using DNA, RNA, or protein detection techniques. Illustrative detection methods and materials are provided below.

### A) Soybean Event Gm_CSM63717 Event-Specific Endpoint Taqman™ Assays

An event-specific endpoint Applied Biosystems™ TaqMan thermal amplification method (Thermo Fisher Scientific) was developed to identify soybean event Gm_CSM63717 in a sample. The DNA primers and probe used in the endpoint assay for this example are shown in Table 15, although it will be appreciated by those of skill in the art that that other primers and probes may also be used.

TABLE 15
Primers and probe for soybean
Gm_CSM63717 event-specific assay

| SEQ ID NO. | Name | Type |
|---|---|---|
| 176 | Primer SQ51702 | Event-specific |
| 177 | Primer SQ52020 | Event-specific |
| 178 | 6FAM ™ probe PB50308 | Event-specific |
| 179 | Primer SQ546 | Internal control |
| 180 | Primer SQ549 | Internal control |
| 181 | VIC ™ Probe PB50207 | Internal control |

TaqMan thermal amplifications use dual-labeled Taqman MGB (Minor Groove Binder) probes incorporating 5′ fluorescent reporter dye 6-FAM™ or VIC™ and a 3′ nonfluorescent quencher. For TaqMan MGB (Minor Groove Binder) probes, the 5′ exonuclease activity of Taq DNA polymerase cleaves the probe from the 5′-end, between the fluorophore and quencher. When hybridized to the target DNA strand, quencher and fluorophore are separated enough to produce a fluorescent signal, thus releasing fluorescence. The pair of primers when used with these reaction methods and the probe produce a DNA amplicon that is diagnostic for soybean event Gm_CSM63717. The controls for this analysis should include a positive control containing soybean event Gm_CSM63717, a negative control from a non-transgenic plant, and a negative control that contains no template DNA. Additionally, a control for the PCR reaction should optimally include internal control primers and an internal control probe specific to a single copy gene in the soybean genome. These assays are optimized for use with the Applied Biosystems GeneAmp® PCR System 9700 (Thermo Fisher Scientific) run at maximum speed, but other equipment may be used.

Examples of PCR reaction components and cycling conditions useful for the event-specific qualitative endpoint TaqMan PCR assay for soybean event Gm_CSM63717 are presented in Table 16 and Table 17. The extracted DNA template was a leaf DNA sample to be analyzed, a negative control (non-transgenic soybean DNA), no template (water) control, or a positive control containing soybean event Gm_CSM63717 DNA.

TABLE 16
Gm_CSM63717 event-specific endpoint TaqMan ™ PCR
reaction components

| | Stock | | |
|---|---|---|---|
| | Concentration | Volume | Final |
| Reagent | (μM) | (μl) | Concentration |
| Reaction Volume | | 5 | |
| 2 × Master Mix | | 2.28 | 1 X |
| Event Specific Primer | 100 | 0.05 | 0.9 μM |
| SQ51702 | | | |
| Event Specific Primer | 100 | 0.05 | 0.9 μM |
| SQ52020 | | | |
| Event Specific 6FAM ™ | 100 | 0.01 | 0.2 μM |
| probe PB50308 | | | |
| Internal Control Primer | 100 | 0.05 | 0.9 μM |
| SQ546 | | | |
| Internal Control Primer | 100 | 0.05 | 0.9 μM |
| SQ549 | | | |
| Internal Control VIC ™ | 100 | 0.01 | 0.2 μM |
| Probe PB50207 | | | |
| Extracted DNA template | | 2.5 | |

TABLE 17
Endpoint TaqMan ™ thermocycler conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

### B) Detection of Soybean Event Gm_CSM63717 Using Antibody

Another example of detection of soybean event Gm_CSM63717 involves the use of antibody specific for the PPO protein encoded by soybean event Gm_CSM63717. For example, a detection kit comprising such antibody can be used. Such a kit may utilize a lateral flow strip comprising reagents activated when the tip of the strip is contacted with an aqueous solution. Illustrative protein sufficient for use in antibody production is the PPO encoded by the sequence provided as SEQ ID NO:9, or any fragment thereof.

A protein detection method can be used to determine whether a sample is from a plant, seed, cell, or plant part comprising soybean event Gm_CSM63717. An antibody specific for the PPO protein encoded by soybean event Gm_CSM63717 is used to detect a protein encoded by soybean event Gm_CSM63717 in a sample. A detection kit comprising an antibody specific for the PPO protein encoded by soybean event Gm_CSM63717 may utilize a lateral flow strip containing reagent activated when the tip of the strip is contacted with an aqueous solution. Samples of soybean tissues may be ground up and protein extracted for analysis using water or an aqueous buffer (for example, phosphate buffered saline containing detergent and bovine serum albumin). Following centrifugation, the aqueous supernatant is analyzed using the ELISA method in a sandwich format on a lateral flow strip containing an absorbent pad. Detection is activated by dipping the tip of the strip into the aqueous solution containing the sample to be tested.

The aqueous solution is carried up the strip by capillary action and solubilizes gold labeled antibody on the strip. The gold-labeled antibody is specific for the PPO protein encoded by soybean event Gm_CSM63717 and will bind to an epitope on the protein in the sample to form an antibody-antigen complex. The gold labeled antibody-antigen complex is then carried up the strip to a nitrocellulose membrane. The membrane comprises a test line of immobilized antibody that binds to a second, separate epitope on the PPO protein encoded by soybean event Gm_CSM63717, causing a visible line to appear across the test strip if the protein encoded by soybean event Gm_CSM63717 is present in the sample.

### C) Detection of Soybean Event Gm_CSM63717 by Southern Analysis

Another method to detect the presence of soybean event Gm_CSM63717 in a plant sample is Southern analysis as generally understood in the art. One of skill in art, based on the present disclosure and description of soybean event Gm_CSM63717, would understand how to design Southern hybridization probe(s) specific for the event and a second Southern hybridization probe specific for a plant which is null for the event (wildtype). With Southern analysis, a signal detected only from the first Southern hybridization probe will be indicative of a plant positive for soybean event Gm_CSM63717; a signal detected only from the second Southern hybridization probe will be indicative that the DNA was extracted from a plant that is null for the event (wildtype).

### Example 9: Zygosity Assays for Soybean Event Gm_CSM63717

This example describes methods useful in determining the zygosity of event Gm_CSM63717. The zygosity assay determines whether a plant comprising soybean event Gm_CSM63717 is heterozygous or homozygous for the event or the wildtype allele. Illustrative detection methods and materials are provided below.

A zygosity assay was developed to determine whether a plant comprising soybean event Gm_CSM63717 is heterozygous or homozygous for the event allele. An amplification reaction assay can be designed using the sequence information provided herein. For example, such a PCR assay would include design of at least three primers: primer-1, primer-2 and primer-3, where primer-1 is specific to soybean genomic DNA on the 5′ flanking DNA of soybean event Gm_CSM63717 (for example, SEQ ID NO:177); primer-2 is specific to soybean event Gm_CSM63717 transgenic insert (for example, SEQ ID NO:176); and primer-3 is specific to the wildtype allele (for example, SEQ ID NO:208). Alternatively, primer-3 (SEQ ID NO:208) and primer-4 (SEQ ID NO:209) are both specific to the wildtype allele. When used as a primer pair in an amplification reaction, primer-1 with primer-2 will produce a PCR amplicon specific for soybean event Gm_CSM63717. When used as a primer pair in an amplification reaction, primer-1 with primer-3, or primer-3 with primer-4 will produce a PCR amplicon specific for wildtype allele. In a PCR reaction performed on soybean event Gm_CSM63717, the respective PCR amplicons generated from primer-1+primer-2 and those generated from primer-1+primer-3 or primer-4+primer-3 will differ in sequence and size of the amplicon. When the three or four primers are included in a PCR reaction with DNA extracted from a plant homozygous for soybean event Gm_CSM63717, only the primer-1+primer-2 amplicon (specific for the soybean event Gm_CSM63717) will be generated. When the three or four primers are included in a PCR reaction with DNA extracted from a plant heterozygous for soybean event Gm_CSM63717, both the primer-1+primer-2 amplicon (specific for the soybean event Gm_CSM63717 insert) and the primer-1+primer-3 and/or primer-4+primer-3 amplicon (specific for the wildtype allele or absence of the soybean event Gm_CSM63717 insert) will be generated. When the three or four primers are mixed together in a PCR reaction with DNA extracted from a plant that is null for soybean event Gm_CSM63717 (i.e., wildtype), only the primer-1+primer-3 and/or primer-4+primer-3 amplicon (specific for the wildtype allele) will be generated. The amplicons produced using the PCR reaction may be identified or distinguished using any method known in the art.

Another zygosity assay for soybean event Gm_CSM63717 is a Taqman™ thermal amplification method. Two fluorescently labeled probes are included in addition to the primers as described in the proceeding section. Probe-1, containing a fluorescent label (for example, the 6-FAM™-labeled SEQ ID NO:178), is specific for soybean event Gm_CSM63717; whereas Probe-2, containing a different fluorescent label (for example, the VIC™-labeled SEQ ID NO:210), is specific for a wildtype soybean plant that is null for soybean event Gm_CSM63717.

When the three (SEQ ID NO:176, SEQ ID NO:177, and SEQ ID NO:208) or four primers (SEQ ID NO:176, SEQ ID NO:177, SEQ ID NO:208, and SEQ ID NO:209) and two probes (SEQ ID NO:178 and SEQ ID NO:210) are mixed together in a PCR reaction with DNA extracted from a plant homozygous for soybean event Gm_CSM63717, a fluorescent signal from the 6FAM™-labeled probe PB50308 (SEQ ID NO:178) is released, which is indicative of and diagnostic for a plant homozygous for soybean event Gm_CSM63717. When the three or four primers and two probes are mixed together in a PCR reaction with DNA extracted from a plant heterozygous for soybean event Gm_CSM63717, two distinct fluorescent signals are generated, one from the 6FAM™-labeled probe PB50308 (SEQ ID NO:178), and one from the VIC™-labeled probe PB50725 (SEQ ID NO:210). When the three or four primers and the two probes are mixed together in a PCR reaction with DNA extracted from a wildtype plant which is null for soybean event Gm_CSM63717, a fluorescent signal from the VIC™-labeled probe PB50725 (SEQ ID NO:210) is generated.

Examples of PCR reaction components and cycling conditions useful for TaqMan™ PCR zygosity assay for soybean event Gm_CSM63717 are presented in Table 18 (for an assay where four primers and two probes are used), and Table 19. The extracted DNA template was a DNA sample to be analyzed, a negative control (non-transgenic soybean DNA), no template (water) control, or a positive control containing soybean event Gm_CSM63717 DNA.

TABLE 18
Gm_CSM63717 zygosity TaqMan ™ PCR
reaction components (four primers)

| | Stock | | Final |
|---|---|---|---|
| | Concen- | Volume | Concen- |
| Reagent | tration (μM) | (μl) | tration |
| Reaction Volume | | 5 | |
| 2 × Master Mix | | 2.3 | 1 X |
| Event Specific Primer | 100 | 0.045 | 0.9 μM |
| SQ51702 (SEQ ID NO: 176) | | | |
| Event Specific Primer | 100 | 0.045 | 0.9 μM |
| SQ52020 (SEQ ID NO: 177) | | | |
| Event Specific 6FAM ™ probe | 100 | 0.01 | 0.2 μM |
| PB50308 (SEQ ID NO: 178) | | | |
| Wildtype allele primer | 100 | 0.045 | 0.9 μM |
| SQ52185 (SEQ ID NO: 208) | | | |
| Wildtype allele primer | 100 | 0.045 | 0.9 μM |
| SQ52186 (SEQ ID NO: 209) | | | |
| Wildtype allele VIC ™ probe | 100 | 0.01 | 0.2 μM |
| PB50725 (SEQ ID NO: 210) | | | |
| Extracted DNA template | | 2.5 | |

TABLE 19
Zygosity TagMan ™ thermocycler conditions

| Step No. | Cycle No. | Settings |
|---|---|---|
| 1 | 1 | 95° C., 20 seconds |
| 2 | 35 | 95° C., 3 seconds |
| | | 60° C., 20 seconds |

Another method to detect the presence and zygosity of soybean event Gm_CSM63717 in a plant sample is Southern blot analysis. One of skill in art would understand how to design a first Southern hybridization probe(s) specific for soybean event Gm_CSM63717 and a second Southern hybridization probe specific for a soybean plant which is null for the soybean event Gm_CMS63717 (wildtype). With Southern blot analysis, a signal detected only from the first Southern hybridization probe is indicative a plant homozygous for soybean event Gm_CSM63717; signals detected from both the first and the second hybridization probes is indicative of a plant heterozygous for soybean event Gm_CSM63717; and a signal detected only from the second Southern hybridization probe indicates that the DNA was extracted from a plant that is null for soybean event Gm_CSM63717 (wildtype).

### Example 10: Modification of Soybean Event Gm_CSM63717 with Genome Editing Techniques Using a Single Guide RNA

This example describes how one may alter or excise all or a part of the transgenic insertion present in soybean event Gm_CSM63717, as well as flanking genomic DNA segments, such as by making one or more insertions, deletions, substitutions, or transpositions using genomic editing techniques. For example, such alterations can be made using Clustered Regularly Interspersed Short Palindromic Repeats (CRISPR) editing systems comprising a single guide RNA by genome editing methods. Sequences useful in excision of the event Gm_CSM63717 transgenic insertion or the expression cassette within SEQ ID NO:9 or SEQ ID NO:10 can be introduced through genome editing using a variety of methods. In one embodiment, Clustered Regularly Interspersed Short Palindromic Repeats (CRISPR) editing systems comprising a CRISPR associated protein and cognate guide RNAs may be used for targeted excision. The CRISPR-associated protein is an RNA-guided nuclease and can be selected from a Type I CRISPR-associated protein, a Type II CRISPR-associated protein, a Type III CRISPR-associated protein, a Type IV CRISPR-associated protein, a Type V CRISPR-associated protein, or a Type VI CRISPR-associated protein, such as, but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and one or more guide RNAs (gRNAs) can be introduced into a plant cell corresponding to soybean event Gm_CSM63717 to target a specific sequence within the transgene insertion locus. In one embodiment, the CRISPR nuclease system cleaves at two identical guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination of any thereof, at the genomic target site. One or more guide RNA hybridization sequences can be inserted within the event Gm_CSM63717 transgene insertion locus which can subsequently allow for the excision all or a portion of the transgene insertion from event Gm_CSM63717 or the expression cassette within SEQ ID NO:9 or SEQ ID NO:10.

Sequences corresponding to the 5′ and 3′ flanking genomic sequences and the transgenic insert of event Gm_CSM63717 (SEQ ID NOs:11, 12, and 9, respectively), the 5′ and 3′ junction regions (SEQ ID NOs:1-6) and the transgenic insertion (SEQ ID NO:9) were scanned for potential originator guide RNA recognition sites (OgRRS). As used herein, the term “originator guide RNA recognition site” or “OgRRS” refers to an endogenous DNA polynucleotide in the flanking genomic sequence or the integrated transgenic polynucleotide comprising a protospacer adjacent motif (PAM) site operably linked to a guide RNA hybridization site (i.e., protospacer sequence). In some embodiments, an OgRRS can be located in the flanking 5′ or 3′ genomic sequence (i.e., in non-transgenic DNA of a junction polynucleotide). In some embodiments, an OgRRS can be located in the 5′ or 3′ junction region (i.e., in both transgenic DNA and non-transgenic DNA of a junction polynucleotide or spanning transgenic and non-transgenic DNA in a DNA junction polynucleotide). In some embodiments, an OgRRS can be located in the transgenic insert. The OgRRS can be determined based upon the specific CRISPR editing system chosen. For example, Cas9 recognizes a G-rich protospacer-adjacent motif (PAM) that is 3′ to its guide RNA hybridization site whereas Cas12a systems recognize a T-rich protospacer-adjacent motif (PAM) that is 5′ to its guide RNA hybridization site.

The OgRRS sequence is then used to define a cognate guide RNA recognition site (CgRRS) which is inserted into the transgenic insertion locus of event Gm_CSM63717 using a CRISPR editing system. As used herein, the term “cognate guide RNA recognition site” or “CgRRS” refers to a DNA polynucleotide comprising a PAM site operably linked to a guide RNA hybridization site (i.e., protospacer sequence), where the CgRRS is absent from event Gm_CSM63717 comprising the original transgenic locus that is unmodified and where the CgRRS and its corresponding OgRRS can hybridize to a single gRNA. A CgRRS can be located in the flanking 5′ or 3′ genomic sequence (i.e., in non-transgenic DNA of a junction polynucleotide), in the 5′ or 3′ junction region (i.e., in both transgenic DNA and non-transgenic DNA of a junction polynucleotide or spanning transgenic and non-transgenic DNA in a DNA junction polynucleotide), or in the transgenic insert. A CgRRS comprises the same gRNA target sequence as the corresponding OgRRS. The CgRRS is inserted in a region within the transgenic insertion locus of event Gm_CSM63717 that is on the opposite side of the transgenic insertion, relative to the OgRRS in a manner that will permit the excision of a fragment of DNA corresponding to either the entire transgenic insertion of event Gm_CSM63717, or a fragment within the transgene insert of event Gm_CSM63717 such as an expression cassette or genetic element within the transgene cassette, using a single gRNA. For example, if the OgRRS is located within the 3′ flanking genomic sequence or the 3′ junction region, then the CgRRS can be inserted within the 5′ flanking genomic sequence, or the 5′ junction region, or within the transgene insert such as between genetic elements within the expression cassette. Insertion of the CgRRS on the opposite side of the transgenic insertion or within the transgenic insert, relative to the OgRRS allows for excision of the transgenic insertion or specific expression element(s) or expression cassette to be excised using a single gRNA. An OgRRS located in the expression cassette of event Gm_CSM63717 can be used to design a CgRRS that can be inserted in either the 5′ or 3′-flanking genomic sequence to permit excision of one or more expression elements or the expression cassette using a single gRNA.

Table 20 shows OgRRS sequences located within the 5′ and 3′-flanking genomic sequences, spanning the 5′ and 3′ junction sequences, and in the transgenic insertion of event Gm_CSM63717 that can be used in a CRISPR editing system employing Cas12a, a Type V CRISPR-associated protein. The analysis was performed for four Cas12a endonucleases. FnCas12a (SEQ ID NO:202) refers to Francisella novicida U112 Cas12a (also known as FnCas12a) and requires the PAM sequence of 5′-TTN, where N is A, C, G or T (Zetsche et al., 2015). LbCas12a (SEQ ID NO:199) refers to the Cas12a from Lachnospiraceae bacterium ND2006 (also known as LbCas12a) and requires the PAM sequence of 5′-TTTV, where V is A, C, or G. LbCas12a-TYC and LbCas12a-TAT refer to engineered variants of Lachnospiraceae bacterium ND2006 Cas12a (Gao et al., 2017). The LbCas12a-TYC variant (SEQ ID NO:200) contains the G532R/K595R mutations and recognizes 5′-TYCV PAM; whereas the LbCas12a-TAT variant (SEQ ID NO:201) contains the G532R/K538V/Y542R mutations and recognizes 5′-TATV PAM, where Y is C or T, and V is A, C or G. The PAM sequence, the coordinates of the gRNA hybridization site (also known as OgRRS) relative to SEQ ID NO:10, and the corresponding Cas12a endonuclease are shown under the headings of “PAM”, “Cas12a Nuclease”, and “Start . . . End of gRNA Hybridization Site in SEQ ID NO:10”, respectively. “Strand of SEQ ID NO: 10” indicates whether the identified gRNA hybridization site along with its PAM sequence is on the forward strand (+) or the complementary strand (−). “Target Site” refers to the location of the gRNA hybridization site in the soybean event CSM63717 locus.

TABLE 20
OgRRS sequences within event Gm_CSM63717

| | | | Start..End of | | |
|---|---|---|---|---|---|
| Name of gRNA | | | gRNA Hybridization | Strand of | |
| Hybridization | | Cas12a | Site in | SEQ ID | |
| Sit e(OgRRS) | PAM | Nuclease | SEQ ID NO: 10 | NO: 10 | Target Site |
| 5F-1 | TTTG | LbCas12a | 24:46 | + | 5′ Flanking genomic DNA |
| 5F-2 | TTTG | LbCas12a | 22:44 | − | 5′ Flanking genomic DNA |
| 5F-3 | TTTA | LbCas12a | 52:74 | − | 5′ Flanking genomic DNA |
| 5F-4 | ATTG | FnCas12a | 101:123 | + | 5′ Flanking genomic DNA |
| 5F-5 | CTTA | FnCas12a | 111:133 | + | 5′ Flanking genomic DNA |
| 5F-6 | TATC | LbCas12a_TATV | 113:135 | + | 5′ Flanking genomic DNA |
| 5F-7 | TCCA | LbCas12a_TYC | 115:137 | + | 5′ Flanking genomic DNA |
| 5F-8 | GTTG | FnCas12a | 137:159 | + | 5′ Flanking genomic DNA |
| 5F-9 | GTTG | FnCas12a | 150:172 | + | 5′ Flanking genomic DNA |
| 5F-10 | CTTC | FnCas12a | 137:159 | − | 5′ Flanking genomic DNA |
| 5F-11 | TTTA | LbCas12a | 160:182 | + | 5′ Flanking genomic DNA |
| 5F-12 | GTTG | FnCas12a | 167:189 | + | 5′ Flanking genomic DNA |
| 5F-13 | ATTG | FnCas12a | 178:200 | + | 5′ Flanking genomic DNA |
| 5F-14 | GTTA | FnCas12a | 187:209 | + | 5′ Flanking genomic DNA |
| 5F-15 | TATC | LbCas12a_TATV | 166:188 | − | 5′ Flanking genomic DNA |
| 5F-16 | GTTG | FnCas12a | 197:219 | + | 5′ Flanking genomic DNA |
| 5F-17 | TTTG | LbCas12a | 179:201 | − | 5′ Flanking genomic DNA |
| 5F-18 | TCCA | LbCas12a_TYC | 204:226 | + | 5′ Flanking genomic DNA |
| 5F-19 | ATTA | FnCas12a | 215:237 | + | 5′ Flanking genomic DNA |
| 5F-20 | ATTG | FnCas12a | 196:218 | − | 5′ Flanking genomic DNA |
| 5F-21 | TATG | LbCas12a_TATV | 228:250 | + | 5′ Flanking genomic DNA |
| 5F-22 | GTTG | FnCas12a | 235:257 | + | 5′ Flanking genomic DNA |
| 5F-23 | GTTG | FnCas12a | 238:260 | + | 5′ Flanking genomic DNA |
| 5F-24 | GTTG | FnCas12a | 244:266 | + | 5′ Flanking genomic DNA |
| 5F-25 | ATTG | FnCas12a | 254;276 | + | 5′ Flanking genomic DNA |
| 5F-26 | GTTG | FnCas12a | 257:279 | + | 5′ Flanking genomic DNA |
| 5F-27 | GTTC | FnCas12a | 260:282 | + | 5′ Flanking genomic DNA |
| 5F-28 | CTTA | FnCas12a | 263:285 | + | 5′ Flanking genomic DNA |
| 5F-29 | TTCG | LbCas12a_TYC | 242:264 | − | 5′ Flanking genomic DNA |
| 5F-30 | CTTC | FnCas12a | 243:265 | − | 5′ Flanking genomic DNA |
| 5F-31 | ATTC | FnCas12a | 266:288 | − | 5′ Flanking genomic DNA |
| 5F-32 | TTCG | LbCas12a_TYC | 267:289 | + | 5′ Flanking genomic DNA |
| 5F-33 | ATTG | FnCas12a | 275:297 | + | 5′ Flanking genomic DNA |
| 5F-34 | GTTG | FnCas12a | 285:307 | + | 5′ Flanking genomic DNA |
| 5F-35 | TTTG | LbCas12a | 267:289 | − | 5′ Flanking genomic DNA |
| 5F-36 | GTTG | FnCas12a | 303:325 | + | 5′ Flanking genomic DNA |
| 5F-37 | GTTG | FnCas12a | 285:307 | − | 5′ Flanking genomic DNA |
| 5F-38 | TCCA | LbCas12a_TYC | 310:332 | + | 5′ Flanking genomic DNA |
| 5F-39 | GTTA | FnCas12a | 289:311 | − | 5′ Flanking genomic DNA |
| 5F-40 | ATTG | FnCas12a | 320:342 | + | 5′ Flanking genomic DNA |
| 5F-41 | TTTG | LbCas12a | 298:320 | − | 5′ Flanking genomic DNA |
| 5F-42 | GTTG | FnCas12a | 307:329 | − | 5′ Flanking genomic DNA |
| 5F-43 | TCCA | LbCas12a_TYC | 332:354 | + | 5′ Flanking genomic DNA |
| 5F-44 | ATTA | FnCas12a | 314:336 | − | 5′ Flanking genomic DNA |
| 5F-45 | ATTG | FnCas12a | 343:365 | + | 5′ Flanking genomic DNA |
| 5F-46 | TTTG | LbCas12a | 352:374 | + | 5′ Flanking genomic DNA |
| 5F-47 | TTTG | LbCas12a | 331:353 | − | 5′ Flanking genomic DNA |
| 5F-48 | GTTC | FnCas12a | 355:377 | + | 5′ Flanking genomic DNA |
| 5F-49 | TTCA | LbCas12a_TYC | 356:378 | + | 5′ Flanking genomic DNA |
| 5F-50 | TTTG | LbCas12a | 341:363 | − | 5′ Flanking genomic DNA |
| 5F-51 | GTTG | FnCas12a | 346:368 | − | 5′ Flanking genomic DNA |
| 5F-52 | TTTG | LbCas12a | 349:371 | − | 5′ Flanking genomic DNA |
| 5F-53 | GTTG | FnCas12a | 354:376 | − | 5′ Flanking genomic DNA |
| 5F-54 | TTTA | LbCas12a | 360:382 | − | 5′ Flanking genomic DNA |
| 5F-55 | CTTC | FnCas12a | 415:437 | + | 5′ Flanking genomic DNA |
| 5F-56 | CTTG | FnCas12a | 418:440 | + | 5′ Flanking genomic DNA |
| 5F-57 | GTTG | FnCas12a | 423:445 | + | 5′ Flanking genomic DNA |
| 5F-58 | TATC | LbCas12a_TATV | 405:427 | − | 5′ Flanking genomic DNA |
| 5F-59 | GTTG | FnCas12a | 412:434 | − | 5′ Flanking genomic DNA |
| 5F-60 | GTTG | FnCas12a | 420:442 | − | 5′ Flanking genomic DNA |
| 5F-61 | GTTG | FnCas12a | 425:447 | − | 5′ Flanking genomic DNA |
| 5F-62 | GTTG | FnCas12a | 428:450 | − | 5′ Flanking genomic DNA |
| 5F-63 | ATTG | FnCas12a | 438:460 | − | 5′ Flanking genomic DNA |
| 5F-64 | ATTG | FnCas12a | 445:467 | − | 5′ Flanking genomic DNA |
| 5F-65 | TTCA | LbCas12a_TYC | 448:470 | − | 5′ Flanking genomic DNA |
| 5F-66 | TTTC | LbCas12a | 449:471 | − | 5′ Flanking genomic DNA |
| 5F-67 | ATTA | FnCas12a | 484:506 | + | 5′ Flanking genomic DNA |
| 5F-68 | TTCA | LbCas12a_TYC | 481:503 | − | 5′ Flanking genomic DNA |
| 5F-69 | CTTC | FnCas12a | 482:504 | − | 5′ Flanking genomic DNA |
| 5F-70 | TTTC | LbCas12a | 485:507 | − | 5′ Flanking genomic DNA |
| 5F-71 | TTCA | LbCas12a_TYC | 505:527 | − | 5′ Flanking genomic DNA |
| 5F-72 | TTTC | LbCas12a | 506:528 | − | 5′ Flanking genomic DNA |
| 5F-73 | ATTA | FnCas12a | 562:584 | + | 5′ Flanking genomic DNA |
| 5F-74 | CTTA | FnCas12a | 581:603 | + | 5′ Flanking genomic DNA |
| 5F-75 | ATTC | FnCas12a | 584:606 | + | 5′ Flanking genomic DNA |
| 5F-76 | TTCA | LbCas12a_TYC | 585:607 | + | 5′ Flanking genomic DNA |
| 5F-77 | TCCA | LbCas12a_TYC | 565:587 | − | 5′ Flanking genomic DNA |
| 5F-78 | TTTA | LbCas12a | 600:622 | + | 5′ Flanking genomic DNA |
| 5F-79 | TTCA | LbCas12a_TYC | 581:603 | − | 5′ Flanking genomic DNA |
| 5F-80 | GTTC | FnCas12a | 582:604 | − | 5′ Flanking genomic DNA |
| 5F-81 | GTTG | FnCas12a | 588:610 | − | 5′ Flanking genomic DNA |
| 5F-82 | CTTC | FnCas12a | 612:634 | + | 5′ Flanking genomic DNA |
| 5F-83 | TTCA | LbCas12a_TYC | 613:635 | + | 5′ Flanking genomic DNA |
| 5F-84 | TTTG | LbCas12a | 593:615 | − | 5′ Flanking genomic DNA |
| 5F-85 | GTTG | FnCas12a | 603:625 | − | 5′ Flanking genomic DNA |
| 5F-86 | CTTC | FnCas12a | 627:649 | + | 5′ Flanking genomic DNA |
| 5F-87 | TTCA | LbCas12a_TYC | 628:630 | + | 5′ Flanking genomic DNA |
| 5F-88 | ATTG | FnCas12a | 622:644 | − | 5′ Flanking genomic DNA |
| 5F-89 | GTTA | FnCas12a | 631:653 | − | 5′ Flanking genomic DNA |
| 5F-90 | CTTA | FnCas12a | 690:712 | + | 5′ Flanking genomic DNA |
| 5F-91 | CTTA | FnCas12a | 672:594 | − | 5′ Flanking genomic DNA |
| 5F-92 | TTTA | LbCas12a | 597:719 | + | 5′ Flanking genomic DNA |
| 5F-93 | ATTG | FnCas12a | 705:727 | + | 5′ Flanking genomic DNA |
| 5F-94 | TTTA | LbCas12a | 686:708 | − | 5′ Flanking genomic DNA |
| 5F-95 | CTTA | FnCas12a | 711:733 | + | 5′ Flanking genomic DNA |
| 5F-96 | TTTA | LbCas12a | 591:713 | − | 5′ Flanking genomic DNA |
| 5F-97 | ATTA | FnCas12a | 716:738 | + | 5′ Flanking genomic DNA |
| 5F-98 | CTTC | FnCas12a | 595:717 | − | 5′ Flanking genomic DNA |
| 5F-99 | ATTG | FnCas12a | 705:727 | − | 5′ Flanking genomic DNA |
| 5F-100 | TTTG | LbCas12a | 717:739 | − | 5′ Flanking genomic DNA |
| 5F-101 | TTTC | LbCas12a | 749:771 | + | 5′ Flanking genomic DNA |
| 5F-102 | TTCA | LbCas12a_TYC | 750:772 | + | 5′ Flanking genomic DNA |
| 5F-103 | GTTA | FnCas12a | 728:750 | − | 5′ Flanking genomic DNA |
| 5F-104 | TTCG | LbCas12a_TYC | 731:753 | − | 5′ Flanking genomic DNA |
| 5F-105 | ATTA | FnCas12a | 753:775 | + | 5′ Flanking genomic DNA |
| 5F-106 | TTTC | LbCas12a | 732:754 | − | 5′ Flanking genomic DNA |
| 5F-107 | TTTG | LbCas12a | 738:760 | − | 5′ Flanking genomic DNA |
| 5F-108 | TATC | LbCas12a_TATV | 746:768 | − | 5′ Flanking genomic DNA |
| 5F-109 | ATTG | FnCas12a | 768.790 | + | 5′ Flanking genomic DNA |
| 5F-110 | CTTA | FnCas12a | 748:770 | − | 5′ Flanking genomic DNA |
| 5F-111 | TTTC | LbCas12a | 751:773 | − | 5′ Flanking genomic DNA |
| 5F-112 | ATTG | FnCas12a | 762.784 | − | 5′ Flanking genomic DNA |
| 5F-113 | GTTC | FnCas12a | 786:808 | + | 5′ Flanking genomic DNA |
| 5F-114 | TTCA | LbCas12a_TYC | 787:809 | + | 5′ Flanking genomic DNA |
| 5F-115 | ATTA | FnCas12a | 768:790 | − | 5′ Flanking genomic DNA |
| 5F-116 | ATTG | FnCas12a | 791.813 | + | 5′ Flanking genomic DNA |
| 5F-117 | TCCA | LbCas12a_TYC | 771:793 | − | 5′ Flanking genomic DNA |
| 5F-118 | TTTG | LbCas12a | 779:801 | − | 5′ Flanking genomic DNA |
| 5F-119 | GTTG | FnCas12a | 802:824 | + | 5′ Flanking genomic DNA |
| 5F-120 | ATTA | FnCas12a | 785:807 | − | 5′ Flanking genomic DNA |
| 5F-121 | CTTA | FnCas12a | 788:810 | − | 5′ Flanking genomic DNA |
| 5F-122 | TTTA | LbCas12a | 820:842 | + | 5′ Flanking genomic DNA |
| 5F-123 | ATTA | FnCas12a | 823:845 | + | 5′ Flanking genomic DNA |
| 5F-124 | TCCC | LbCas12a_TYC | 802:824 | − | 5′ Flanking genomic DNA |
| 5F-125 | TATC | LbCas12a_TATV | 804:826 | − | 5′ Flanking genomic DNA |
| 5F-126 | ATTA | FnCas12a | 806:828 | − | 5′ Flanking genomic DNA |
| 5F-127 | TCCA | LbCas12a_TYC | 813:835 | − | 5′ Flanking genomic DNA |
| 5F-128 | TTTA | LbCas12a | 836:858 | + | 5′ Flanking genomic DNA |
| 5F-129 | TATG | LbCas12a_TATV | 838:860 | + | 5′ Flanking genomic DNA |
| 5F-130 | TATA | LbCas12a_TATV | 828:850 | − | 5′ Flanking genomic DNA |
| 5F-131 | ATTA | FnCas12a | 830:852 | − | 5′ Flanking genomic DNA |
| 5F-132 | CTTA | FnCas12a | 853:875 | + | 5′ Flanking genomic DNA |
| 5F-133 | TATA | LbCas12a_TATV | 855:877 | + | 5′ Flanking genomic DNA |
| 5F-134 | CTTA | FnCas12a | 833:855 | − | 5′ Flanking genomic DNA |
| 5F-135 | TCCC | LbCas12a_TYC | 843:865 | − | 5′ Flanking genomic DNA |
| 5F-136 | TATA | LbCas12a_TATV | 853:875 | − | 5′ Flanking genomic DNA |
| 5F-137 | ATTA | FnCas12a | 855:877 | − | 5′ Flanking genomic DNA |
| 5F-138 | TATA | LbCas12a_TATV | 880:902 | + | 5′ Flanking genomic DNA |
| 5F-139 | TATG | LbCas12a_TATV | 885:907 | + | 5′ Flanking genomic DNA |
| 5P-140 | TTTA | LbCas12a | 865:887 | − | 5′ Flanking genomic DNA |
| 5F-141 | ATTA | FnCas12a | 890:912 | + | 5′ Flanking genomic DNA |
| 5F-142 | TTCA | LbCas12a_TYC | 869:891 | − | 5′ Flanking genomic DNA |
| 5F-143 | TTTC | LbCas12a | 870:892 | − | 5′ Flanking genomic DNA |
| 5F-144 | TTCC | LbCas12a_TYC | 874:896 | − | 5′ Flanking genomic DNA |
| 5F-145 | ATTA | FnCas12a | 886:908 | − | 5′ Flanking genomic DNA |
| 5F-146 | TTTA | LbCas12a | 916:938 | + | 5′ Flanking genomic DNA |
| 5F-147 | ATTC | FnCas12a | 919:941 | + | 5′ Flanking genomic DNA |
| 5F-148 | CTTA | FnCas12a | 922:944 | + | 5′ Flanking genomic DNA |
| 5F-149 | ATTA | FnCas12a | 903:925 | − | 5′ Flanking genomic DNA |
| 5F-150 | TTCA | LbCas12a_TYC | 908:930 | − | 5′ Flanking genomic DNA |
| 5F-151 | TATG | LbCas12a_TATV | 933.955 | + | 5′ Flanking genomic DNA |
| 5F-152 | ATTA | FnCas12a | 943:965 | + | 5′ Flanking genomic DNA |
| 5F-153 | TATC | LbCas12a_TATV | 924:946 | − | 5′ Flanking genomic DNA |
| 5F-154 | TATG | LbCas12a_TATV | 948:970 | + | 5′ Flanking genomic DNA |
| 5F-155 | TATA | LbCas12a TATV | 926:948 | − | 5′ Flanking genomic DNA |
| 5F-156 | TATA | LbCas12a_TATY | 953:975 | + | 5′ Flanking genomic DNA |
| 5F-157 | ATTA | FnCas12a | 945:967 | − | 5′ Flanking genomic DNA |
| 5F-158 | TTCA | LbCas12a_TYC | 952:974 | − | 5′ Flanking genomic DNA |
| 5F-159 | TTTC | LbCas12a | 953:975 | − | 5′ Flanking genomic DNA |
| 5J-1 | TTTG | LbCas12a | 993:1015 | + | S' junction |
| 5J-2 | TTTG | LbCas12a | 997:1019 | − | S' junction |
| TI-1 | GTTA | FnCas12a | 1029:1051 | + | Transgenic insert |
| TI-2 | TTCG | LbCas12a_TYC | 1010:1032 | − | Transgenic insert |
| TI-3 | ATTC | FnCas12a | 1011:1033 | − | Transgenic insert |
| TI-4 | TTCA | LbCas12a_TYC | 1019:1041 | − | Transgenic insert |
| TI-5 | TTTC | LbCas12a | 1020:1042 | − | Transgenic insert |
| TI-6 | TTTG | LbCas12a | 1044:1066 | + | Transgenic insert |
| TI-7 | TTTC | LbCas12a | 1027:1049 | − | Transgenic insert |
| TI-8 | ATTA | FnCas12a | 1050:1072 | + | Transgenic insert |
| TI-9 | ATTG | FnCas12a | 1036:1058 | − | Transgenic insert |
| TI-10 | ATTG | FnCas12a | 1065:1087 | + | Transgenic insert |
| TI-11 | TTTG | LbCas12a | 1054:1076 | − | Transgenic insert |
| TI-12 | ATTC | FnCas12a | 1084:1106 | + | Transgenic insert |
| TI-13 | TTCG | LbCas12a_TYC | 1085:1107 | + | Transgenic insert |
| TI-14 | CTTA | FnCas12a | 1091:1113 | + | Transgenic insert |
| TI-15 | ATTG | FnCas12a | 1074:1096 | − | Transgenic insert |
| TI-16 | ATTA | FnCas12a | 1124:1146 | − | Transgenic insert |
| TI-17 | ATTA | FnCas12a | 1149:1171 | + | Transgenic insert |
| TI-18 | TCCA | LbCas12a_TYC | 1157:1179 | + | Transgenic insert |
| TI-19 | CTTG | FnCas12a | 1162:1184 | + | Transgenic insert |
| TI-20 | GTTG | FnCas12a | 1153:1175 | − | Transgenic insert |
| TI-21 | CTTG | FnCas12a | 1172:1194 | − | Transgenic insert |
| TI-22 | TTTG | LbCas12a | 1183:1205 | − | Transgenic insert |
| TI-23 | CTTA | FnCas12a | 1224:1246 | + | Transgenic insert |
| TI-24 | TATC | LbCas12a_TATV | 1212:1234 | − | Transgenic insert |
| TI-25 | CTTA | FnCas12a | 1214:1236 | − | Transgenic insert |
| TI-26 | TTTC | LbCas12a | 1217:1239 | − | Transgenic insert |
| TI-27 | ATTG | FnCas12a | 1224:1246 | − | Transgenic insert |
| TI-28 | TTTG | LbCas12a | 1254.1276 | + | Transgenic insert |
| TI-29 | GTTA | FnCas12a | 1238:1260 | − | Transgenic insert |
| TI-30 | GTTA | FnCas12a | 1263:1285 | + | Transgenic insert |
| TI-31 | GTTG | FnCas12a | 1245:1267 | − | Transgenic insert |
| TI-32 | TCCA | LbCas12a_TYC | 1270:1292 | + | Transgenic insert |
| TI-33 | TCCC | LbCas12a_TYC | 1259:1281 | − | Transgenic insert |
| TI-34 | TTTC | LbCas12a | 1281:1303 | + | Transgenic insert |
| TI-35 | TTCA | LbCas12a_TYC | 1282:1304 | + | Transgenic insert |
| TI-36 | CTTG | FnCas12a | 1292:1314 | + | Transgenic insert |
| TI-37 | CTTA | FnCas12a | 1275:1297 | − | Transgenic insert |
| TI-38 | CTTA | FnCas12a | 1300:1322 | + | Transgenic insert |
| TI-39 | TTTG | LbCas12a | 1320:1342 | + | Transgenic insert |
| TI-40 | GTTG | FnCas12a | 1323:1345 | + | Transgenic insert |
| TI-41 | TTTG | LbCas12a | 1322:1344 | − | Transgenic insert |
| TI-42 | GTTG | FnCas12a | 1332:1354 | − | Transgenic insert |
| TI-43 | TTTG | LbCas12a | 1336:1358 | − | Transgenic insert |
| TI-44 | TTTA | LbCas12a | 1386:1408 | + | Transgenic insert |
| TI-45 | ATTA | FnCas12a | 1389:1411 | + | Transgenic insert |
| TI-46 | TATA | LbCas12a_TATV | 1391:1413 | + | Transgenic insert |
| TI-47 | TATA | LbCas12a_TATV | 1393:14:5 | + | Transgenic insert |
| TI-48 | ATTC | FnCas12a | 1406:1428 | + | Transgenic insert |
| TI-49 | TTCA | LbCas12a_TYC | 1407:1429 | + | Transgenic insert |
| TI-50 | TTTG | LbCas12a | 1385:1407 | − | Transgenic insere |
| TI-51 | TTTG | LbCas12a | 1389:1411 | − | Transgenic insert |
| TI-52 | TTTC | LbCas12a | 1420:1442 | + | Transgenic insert |
| TI-53 | CTTC | FnCas12a | 1423:1445 | + | Transgenic insert |
| TI-54 | TCCC | LbCas12a_TYC | 1402:1424 | − | Transgenic insert |
| TI-55 | TTCC | LbCas12a_TYC | 1403:1425 | − | Transgenic insert |
| TI-56 | TTTC | LbCas12a | 1404:1426 | − | Transgenic insert |
| TI-57 | TATA | LbCas12a_TATV | 1409:1431 | − | Transgenic insert |
| TI-58 | GTTA | FnCas12a | 1411:1433 | − | Transgenic insert |
| TI-59 | TATA | LbCas12a_TATV | 1436:1458 | + | Transgenic insert |
| TI-60 | TTTG | LbCas12a | 1415:1437 | − | Transgenic insert |
| TI-61 | CTTG | FnCas12a | 1423:1445 | − | Transgenic insert |
| TI-62 | TTTC | LbCas12a | 1447:1469 | + | Transgenic insert |
| TI-63 | TTCA | LbCas12a_TYC | 1448:1470 | + | Transgenic insert |
| TI-64 | CTTA | FnCas12a | 1461:1483 | + | Transgenic insert |
| TI-65 | ATTG | FnCas12a | 1464:1486 | + | Transgenic insert |
| TI-66 | TTTG | LbCas12a | 1446:1468 | − | Transgenic insert |
| TI-67 | TCCC | LbCas12a_TYC | 1470:1492 | + | Transgenic insert |
| TI-68 | CTTG | FnCas12a | 1454:1476 | − | Transgenic insert |
| TI-69 | TTTG | LbCas12a | 1461:1483 | − | Transgenic insert |
| TI-70 | TTTG | LbCas12a | 1466:1488 | − | Transgenic insert |
| TI-71 | TTTG | LbCas12a | 1471:1493 | − | Transgenic insert |
| TI-72 | GTTG | FnCas12a | 1476;1498 | − | Transgenic insert |
| TI-73 | TTTG | LbCas12a | 1479:1501 | − | Transgenic insert |
| TI-74 | TATC | LbCas12a_TATV | 1495:1517 | − | Transgenic insert |
| TI-75 | TATC | LbCas12a_TATV | 1524:1546 | + | Transgenic insert |
| TI-76 | TCCA | LbCas12a_TYC | 1528:1550 | + | Transgenic insert |
| TI-77 | TTTG | LbCas12a | 1513:1535 | − | Transgenic insert |
| TI-78 | TTTA | LbCas12a | 1518:1540 | − | Transgenic insert |
| TI-79 | ATTA | FnCas12a | 1528:1550 | − | Transgenic insert |
| TI-80 | ATTA | FnCas12a | 1553:1575 | + | Transgenic insert |
| TI-81 | ATTC | FnCas12a | 1560:1582 | + | Transgenic insert |
| TI-82 | TTCA | LbCas12a_TYC | 1561:1583 | + | Transgenic insert |
| TI-83 | TATA | LbCas12a_TATV | 1567:1589 | + | Transgenic insert |
| TI-84 | ATTA | FnCas12a | 1570:1592 | + | Transgenic insert |
| TI-85 | TTTA | LbCas12a | 1577:1599 | + | Transgenic insert |
| TI-86 | TATC | LbCas12a_TATV | 1567:1589 | − | Transgenic insert |
| TI-87 | ATTG | FnCas12a | 1571:1593 | − | Transgenic insert |
| TI-88 | ATTC | FnCas12a | 1600:1622 | + | Transgenic insert |
| TI-89 | CTTG | FnCas12a | 1603:1625 | + | Transgenic insert |
| TI-90 | TTCG | LbCas12a_TYC | 1586:1608 | − | Transgenic insert |
| TI-91 | CTTC | FnCas12a | 1587:1609 | − | Transgenic insert |
| TI-92 | TTTG | LbCas12a | 1591:16:3 | − | Transgenic insert |
| TI-93 | ATTA | FnCas12a | 1621:1643 | + | Transgenic insert |
| TI-94 | TTTC | LbCas12a | 1600:1622 | − | Transgenic insert |
| TI-95 | GTTG | FnCas12a | 1611:1633 | − | Transgenic insert |
| TI-96 | GTTC | FnCas12a | 1635:1657 | + | Transgenic insert |
| TI-97 | TTCA | LbCas12a_TYC | 1636:1658 | + | Transgenic insert |
| TI-98 | TTTG | LbCas12a | 1614:1636 | − | Transgenic insert |
| TI-99 | CTTG | FnCas12a | 1619:1641 | − | Transgenic insert |
| TI-100 | TCCC | LbCas12a_TYC | 1651:1673 | + | Transgenic insert |
| | TTTG | LbCas12a | 1655:1677 | + | Transgenic insert |
| TI-102 | TTTG | LbCas12a | 1646:1668 | − | Transgenic insert |
| TI-103 | TTTC | LbCas12a | 1657:1679 | − | Transgenic insert |
| TI-104 | TATA | LbCas12a_TATV | 1694:1716 | + | Transgenic insert |
| TI-105 | CTTC | FnCas12a | 1703:1725 | − | Transgenic insert |
| TI-106 | TCCA | LbCas12a_TYC | 1726:1748 | + | Transgenic insert |
| TI-107 | ATTA | FnCas12a | 1735:1757 | + | Transgenic insert |
| TI-108 | CTTC | FnCas12a | 1758:1780 | + | Transgenic insert |
| TI-109 | CTTG | FnCas12a | 1761:1783 | + | Transgenic insert |
| TI-110 | TATC | LbCas12a_TATV | 1741:1763 | − | Transgenic insert |
| TI-111 | TTTG | LbCas12a | 1751:1773 | − | Transgenic insert |
| TI-112 | TTTG | LbCas12a | 1774:1796 | + | Transgenic insert |
| TI-113 | TTCG | LbCas12a_TYC | 1763:1785 | − | Transgenic insert |
| TI-114 | TCCC | LbCas12a_TYC | 1786:1808 | + | Transgenic insert |
| TI-115 | GTTC | FnCas12a | 1764:1786 | − | Transgenic insert |
| TI-116 | GTTA | FnCas12a | 1772:1794 | − | Transgenic insert |
| TI-117 | CTTC | FnCas12a | 1794:1816 | + | Transgenic insert |
| TI-118 | CTTA | FnCas12a | 1797:1819 | + | Transgenic insert |
| TI-119 | ATTG | FnCas12a | 1783:1805 | − | Transgenic insert |
| TI-120 | TCCC | LbCas12aTYC | 1807:1829 | + | Transgenic insert |
| TI-121 | TCCG | LbCas12a_TYC | 1813:1835 | + | Transgenic insert |
| TI-122 | TCCC | LbCas12a_TYC | 1859:1881 | + | Transgenic insert |
| TI-123 | ATTA | FnCas12a | 1864:1886 | + | Transgenic insert |
| TI-124 | ATTA | FnCas12a | 1867:1889 | + | Transgenic insert |
| TI-125 | TCCA | LbCas12aTYC | 1846:1868 | − | Transgenic insert |
| TI-126 | CTTG | FnCas12a | 1884:1906 | + | Transgenic insert |
| TI-127 | TCCA | LbCas12a_TYC | 1892:1914 | + | Transgenic insert |
| TI-128 | TATC | LbCas12a_TATV | 1900:1922 | + | Transgenic insert |
| TI-129 | TTCG | LbCas12a_TYC | 1885:1907 | − | Transgenic insert |
| TI-130 | CTTC | FnCas12a | 1885:1908 | − | Transgenic insert |
| TI-131 | TTCG | LbCas12a_TYC | 1898:1920 | − | Transgenic insert |
| TI-132 | GTTC | FnCas12a | 1899:1921 | − | Transgenic insert |
| TI-133 | GTTG | FnCas12a | 1911:1933 | − | Transgenic insert |
| TI-134 | TTTC | LbCas12a | 1972:1994 | + | Transgenic insert |
| TI-135 | TTCG | LbCas12a_TYC | 1973:1995 | + | Transgenic insert |
| TI-136 | TATG | LbCas12a_TATV | 1952:1974 | − | Transgenic insert |
| TI-137 | CTTA | FnCas12a | 1954:1976 | − | Transgenic insert |
| TI-138 | TTTG | LbCas12a | 1987:2009 | + | Transgenic insert |
| TI-139 | TCCC | LbCas12a_TYC | 1968:1990 | − | Transgenic Insert |
| TI-140 | CTTC | FnCas12a | 2004:2026 | + | Transgenic insert |
| TI-141 | CTTC | FnCas12a | 2013:2035 | + | Transgenic insert |
| TI-142 | TTCC | LbCas12a_TYC | 2014:2036 | + | Transgenic insert |
| TI-143 | CTTG | FnCas12a | 2017:2039 | + | Transgenic insert |
| TI-144 | TTTA | LbCas12a | 2029:2051 | + | Transgenic insert |
| TI-145 | CTTC | FnCas12a | 2010:2032 | − | Transgenic insert |
| TI-146 | GTTC | FnCas12a | 2013:2035 | − | Transgenic insert |
| TI-147 | CTTG | FnCas12a | 2047:2069 | + | Transgenic insert |
| TI-148 | GTTG | FnCas12a | 2055:2077 | + | Transgenic insert |
| TI-149 | GTTC | FnCas12a | 2058:2080 | + | Transgenic insert |
| TI-150 | TTCA | LbCas12a_TYC | 2059:2081 | + | Transgenic insert |
| TI-151 | TCCA | LbCas12a_TYC | 2068:2090 | + | Transgenic insert |
| TI-152 | TTTC | LbCas12A | 2074:2096 | + | Transgenic insert |
| TI-153 | TTCA | LbCas12a_TYC | 2057:2079 | − | Transgenic insert |
| TI-154 | CTTG | FnCas12a | 2079:2101 | + | Transgenic insert |
| TI-155 | GTTC | FnCas12a | 2058:2080 | − | Transgenic insert |
| TI-156 | CTTG | FnCas12a | 2088:2110 | + | Transgenic insert |
| TI-157 | CTTG | FnCas12a | 2066:2088 | − | Transgenic insert |
| TI-158 | TTCA | LbCas12a_TYC | 2078:2100 | − | Transgenic insert |
| TI-159 | CTTC | FnCas12a | 2079:2101 | − | Transgenic insert |
| TI-160 | GTTG | FnCas12a | 2103:2125 | + | Transgenic insert |
| TI-161 | GTTA | FnCas12a | 2089:2111 | − | Transgenic insert |
| TI-162 | TTCG | LbCas12a_TYC | 2092:2114 | − | Transgenic insert |
| TI-163 | ATTC | FnCas12a | 2093:2115 | − | Transgenic insert |
| TI-164 | ATTA | FnCas12a | 2134:2156 | + | Transgenic insert |
| TI-165 | ATTG | FnCas12a | 2150:2172 | + | Transgenic insert |
| TI-166 | GTTC | FnCas12a | 2160:2182 | + | Transgenic insert |
| TI-167 | TTCA | LbCas12a_TYC | 2161:2183 | + | Transgenic insert |
| TI-168 | TCCC | LbCas12a_TYC | 2170:2192 | + | Transgenic insert |
| TI-169 | ATTC | FnCas12a | 2198:2220 | + | Transgenic insert |
| TI-170 | TTCG | LbCas12a_TYC | 2199:2221 | + | Transgenic insert |
| TI-171 | CTTC | FnCas12a | 2205:2227 | + | Transgenic insert |
| TI-172 | CTTC | FnCas12a | 2211:2233 | + | Transgenic insert |
| TI-173 | TTCA | LbCas12a_TYC | 2212:2234 | + | Transgenic insert |
| TI-174 | TTCA | LbCas12a_TYC | 2205:2227 | − | Transgenic insert |
| TI-175 | CTTC | FnCas12a | 2206:2228 | − | Transgenic insert |
| TI-176 | TATG | LbCas12a_TATV | 2230:2252 | + | Transgenic insert |
| TI-177 | ATTG | FnCas12a | 2210:2232 | − | Transgenic insert |
| TI-178 | ATTG | FnCas12a | 2239:2261 | + | Transgenic insert |
| TI-179 | TCCC | LbCas12a_TYC | 2257:2279 | + | Transgenic insert |
| TI-180 | CTTG | FnCas12a | 2250:2272 | − | Transgenic insert |
| TI-181 | CTTC | FnCas12a | 2283:2305 | + | Transgenic insert |
| TI-182 | TTCG | LbCas12a_TYC | 2284:2306 | + | Transgenic insert |
| TI-183 | TTCG | LbCas12a_TYC | 2279.2301 | − | Transgenic insert |
| TI-184 | CTTC | FnCas12a | 2280:2302 | − | Transgenic insert |
| TI-185 | TCCG | LbCas12a_TYC | 2312:2334 | + | Transgenic insert |
| TI-186 | TCCA | LbCas12a_TYC | 2291:2313 | − | Transgenic insert |
| TI-187 | GTTC | FnCas12a | 2304:2326 | − | Transgenic insert |
| TI-188 | CTTC | FnCas12a | 2334:2356 | + | Transgenic insert |
| TI-189 | TTTG | LbCas12a | 2312:2334 | − | Transgenic insert |
| TI-190 | TCCG | LbCas12a_TYC | 2346:2368 | + | Transgenic insert |
| TI-191 | GTTC | FnCas12a | 2349:2371 | + | Transgenic insert |
| TI-192 | CTTC | FnCas12a | 2352:2374 | + | Transgenic insert |
| TI-193 | CTTC | FnCas12a | 2355:2377 | + | Transgenic insert |
| TI-194 | TTCA | LbCas12a_TYC | 2356:2378 | + | Transgenic insert |
| TI-195 | TCCA | LbCas12a_TYC | 2335:2357 | − | Transgenic insert |
| TI-196 | TTCA | LbCas12a_TYC | 2345:2367 | − | Transgenic insert |
| TI-197 | CTTC | FnCas12a | 2346:2368 | − | Transgenic insert |
| TI-198 | CTTG | FnCas12a | 2370:2392 | + | Transgenic insert |
| TI-199 | TTTG | LbCas12a | 2355:2377 | − | Transgenic insert |
| TI-200 | CTTC | FnCas12a | 2379:2401 | + | Transgenic insert |
| TI-201 | TTCA | LbCas12a_TYC | 2380:2402 | + | Transgenic insert |
| TI-202 | ATTG | FnCas12a | 2388:2410 | + | Transgenic insert |
| TI-203 | CTTA | FnCas12a | 2394:2416 | + | Transgenic insert |
| TI-204 | TCCC | LbCas12a_TYC | 2374:2396 | − | Transgenic insert |
| TI-205 | TTCC | LbCas12a_TYC | 2375:2397 | − | Transgenic insert |
| TI-206 | GTTC | FnCas12a | 2376:2398 | − | Transgenic insert |
| TI-207 | TTCG | LbCas12a_TYC | 2385:2407 | − | Transgenic insert |
| TI-208 | TTTC | LbCas12a | 2386:2408 | − | Transgenic insert |
| TI-209 | TCCG | LbCas12a_TYC | 2402:2424 | − | Transgenic insert |
| TI-210 | ATTC | FnCas12a | 2406:2428 | − | Transgenic insert |
| TI-211 | TATC | LbCas12a_TATV | 2411:2433 | − | Transgenic insert |
| TI-212 | TCCA | LbCas12a_TYC | 2423:2445 | − | Transgenic insert |
| TI-213 | CTTG | FnCas12a | 2445:2467 | + | Transgenic insert |
| TI-214 | GTTG | FnCas12a | 2448:2470 | + | Transgenic insert |
| TI-215 | GTTG | FnCas12a | 2445:2467 | − | Transgenic insert |
| TI-216 | CTTC | FnCas12a | 2460:2482 | − | Transgenic insert |
| TI-217 | TCCG | LbCas12a_TYC | 2471:2493 | − | Transgenic insert |
| TI-218 | TTCG | LbCas12a_TYC | 2475:2497 | − | Transgenic insert |
| TI-219 | CTTC | FnCas12a | 2476:2498 | − | Transgenic insert |
| TI-220 | TATA | LbCas12a_TATV | 2513:2535 | − | Transgenic insert |
| TI-221 | ATTA | FnCas12a | 2515:2537 | − | Transgenic insert |
| TI-222 | TTTG | LbCas12a | 2520:2542 | − | Transgenic insert |
| TI-223 | TTTG | LbCas12a | 2525:2347 | − | Transgenic insert |
| TI-224 | TTTC | LbCas12a | 2529:2551 | − | Transgenic insert |
| TI-225 | TTTC | LbCas12a | 2583:2605 | + | Transgenic insert |
| TI-226 | TTCA | LbCas12a_TYC | 2584:2606 | + | Transgenic insert |
| TI-227 | ATTG | FnCas12a | 2587:2609 | + | Transgenic insert |
| TI-228 | TTTA | LbCas12a | 2579:260: | − | Transgenic insert |
| TI-229 | GTTA | FnCas12a | 2604:2626 | + | Transgenic insert |
| TI-230 | ATTC | FnCas12a | 2584:2606 | − | Transgenic insert |
| TI-231 | TATG | LbCas12a_TATV | 2614:2636 | + | Transgenic insert |
| TI-232 | TTCA | LbCas12a_TYC | 2596:2618 | − | Transgenic insert |
| TI-233 | GTTA | FnCas12a | 2619:2641 | + | Transgenic insert |
| TI-234 | TTTC | LbCas12a | 2597:2619 | − | Transgenic insert |
| TI-235 | TATG | LbCas12a_TATV | 2621:2643 | + | Transgenic insert |
| TI-236 | TCCA | LbCas12a_TYC | 2603:2625 | − | Transgenic insert |
| TI-237 | TTCC | LbCas12a_TYC | 2604:2626 | − | Transgenic insert |
| TI-238 | CTTC | FnCas12a | 2605:2627 | − | Transgenic insert |
| TI-239 | ATTG | FnCas12a | 2620:2642 | − | Transgenic insert |
| TI-240 | TATA | LbCas12a_TATV | 2624:2646 | − | Transgenic insert |
| TI-241 | ATTA | FnCas12a | 2626:2648 | − | Transgenic insert |
| TI-242 | ATTA | FnCas12a | 2649:2671 | + | Transgenic insert |
| TI-243 | TATA | LbCas12a_TATV | 2651:2673 | + | Transgenic insert |
| TI-244 | ATTG | FnCas12a | 2630:2652 | − | Transgenic insert |
| TI-245 | ATTG | FnCas12a | 2635:2657 | − | Transgenic insert |
| TI-246 | GTTG | FnCas12a | 2642:2664 | − | Transgenic insert |
| TI-247 | TTTG | LbCas12a | 2652:2674 | − | Transgenic insert |
| TI-248 | CTTG | FnCas12a | 2687:2709 | + | Transgenic insert |
| TI-249 | ATTC | FnCas12a | 2699:2721 | + | Transgenic insert |
| TI-250 | TCCC | LbCas12a_TYC | 2703:2725 | + | Transgenic insert |
| TI-251 | ATTG | FnCas12a | 2683:2705 | − | Transgenic insert |
| TI-252 | ATTG | FnCas12a | 2687:2709 | − | Transgenic insert |
| TI-253 | TTTA | LbCas12a | 2699:2721 | − | Transgenic insert |
| TI-254 | TATC | LbCas12a_TATV | 2733:2755 | + | Transgenic insert |
| TI-255 | TCCA | LbCas12a_TYC | 2735:2757 | + | Transgenic insert |
| TI-256 | TTTA | LbCas12a | 2726:2748 | − | Transgenic insert |
| TI-257 | TTTA | LbCas12a | 2803:2825 | + | Transgenic insert |
| TI-258 | TATA | LbCas12a_TATV | 2805:2827 | + | Transgenic insert |
| TI-259 | TATG | LbCas12a_TATV | 2807:2829 | + | Transgenic insert |
| TI-260 | ATTG | FnCas12a | 2796:2818 | − | Transgenic insert |
| TI-261 | TTTA | LbCas12a | 2801:2823 | − | Transgenic insert |
| TI-262 | TTCA | LbCas12a_TYC | 2814:2836 | − | Transgenic insert |
| TI-263 | TTTC | LbCas12a | 2815.2837 | − | Transgenic insert |
| TI-254 | ATTA | FnCas12a | 2854:2876 | + | Transgenic insert |
| TI-265 | TTTA | LbCas12a | 2248:2870 | − | Transgenic insert |
| TI-265 | CTTG | FnCas12a | 2854:2876 | − | Transgenic insert |
| TI-267 | TTTA | LbCas12a | 2858:2880 | − | Transgenic insert |
| TI-268 | ATTG | FnCas12a | 2867:2889 | − | Transgenic insert |
| TI-269 | GTTC | FnCas12a | 2891.2913 | + | Transgenic insert |
| TI-270 | TTCA | LbCas12a_TYC | 2892.2914 | + | Transgenic insert |
| TI-271 | CTTC | FnCas12a | 2874:2896 | − | Transgenic insert |
| TI-272 | ATTC | FnCas12a | 2896.2918 | + | Transgenic insert |
| TI-273 | TTCA | LbCas12a_TYC | 2897:2919 | + | Transgenic insert |
| TI-274 | ATTG | FnCas12a | 2883:2905 | − | Transgenic insert |
| TI-275 | TTTC | LbCas12a | 2907:2929 | + | Transgenic insert |
| TI-276 | TTCA | LbCas12a_TYC | 2908:2930 | + | Transgenic insert |
| TI-277 | TTCA | LbCas12a_TYC | 2891:2913 | − | Transgenic insert |
| TI-278 | TTTC | LbCas12a | 2892:2914 | − | Transgenic insert |
| TI-279 | TTTC | LbCas12a | 2896:2918 | − | Transgenic insert |
| TI-280 | TTTG | LbCas12a | 2904:2926 | − | Transgenic insert |
| TI-281 | ATTA | FnCas12a | 2912:2934 | − | Transgenic insert |
| TI-282 | TTCA | LbCas12a_TYC | 2916:2938 | − | Transgenic insert |
| TI-283 | ATTC | FnCas12a | 2917:2939 | − | Transgenic insert |
| TI-284 | TITA | LbCas12a | 2922:2944 | − | Transgenic insert |
| TI-285 | TTTA | LbCas12a | 2947:2969 | + | Transgenic insert |
| T1-286 | TCCG | LbCas12a_TYC | 2931:2953 | − | Transgenic insert |
| TI-287 | TTTG | LbCas12a | 2953:2975 | + | Transgenic insert |
| TI-288 | TTTG | LbCas12a | 2936:2958 | − | Transgenic insert |
| TI-289 | TTTA | LbCas12a | 2953:2975 | − | Transgenic insert |
| TI-290 | ATTA | FnCas12a | 2978:3000 | + | Transgenic insert |
| TI-291 | TATC | LbCas12a_TATV | 2958:2980 | − | Transgenic insert |
| TI-292 | ATTA | FnCas12a | 2967:2989 | − | Transgenic insert |
| TI-293 | ATTA | FnCas12a | 2970:2992 | − | Transgenic insert |
| TI-294 | ATTA | FnCas12a | 2973:2995 | − | Transgenic insert |
| TI-295 | TTTA | LbCas12a | 2976:2998 | − | Transgenic insert |
| TI-296 | TATA | LbCas12a_TATV | 3004:3026 | − | Transgenic insert |
| TI-297 | ATTA | FnCas12a | 3029:3051 | + | Transgenic insert |
| TI-298 | TATA | LbCas12a_TATV | 3031:3033 | + | Transgenic insert |
| TI-299 | TTCA | LbCas12a_TYC | 3011:3033 | − | Transgenic insert |
| TI-300 | TTTC | LbCas12a | 3012:3034 | − | Transgenic insert |
| TI-301 | CTTG | FnCas12a | 3036:3058 | + | Transgenic insert |
| TI-302 | TCCG | LbCas12a_TYC | 3016:3038 | − | Transgenic insert |
| TI-303 | TTCC | LbCas12a TYC | 3017:3039 | − | Transgenic insert |
| TI-304 | ATTC | FnCas12a | 3018:3040 | − | Transgenic insert |
| TI-305 | ATTG | FnCas12a | 3047:3069 | + | Transgenic insert |
| TI-306 | TTCA | LbCas12a_TYC | 3035:3057 | − | Transgenic insert |
| TI-307 | ATTC | FnCas12a | 3036:3058 | − | Transgenic inseit |
| TI-308 | TTTG | LbCas12a | 3060:3082 | + | Transgenic insert |
| TI-309 | TTCA | LbCas12a_TYC | 3039:3061 | − | Transgenic insert |
| TI-310 | ATTC | FnCas12a | 3040:3062 | − | Transgenic insert |
| TI-311 | TTTG | LbCas12a | 3052:3074 | − | Transgenic insert |
| TI-312 | TTTC | LbCas12a | 3075:3097 | + | Transgenic insert |
| TI-313 | TTCC | LbCas12a_TYC | 3076:3098 | + | Transgenic insert |
| TI-314 | TCCA | LbCas12a_TYC | 3077:3099 | + | Transgenic insert |
| TI-315 | TTCC | LbCas12a_TYC | 3056:3078 | − | Transgenic insert |
| TI-316 | GTTC | FnCas12a | 3057:3079 | − | Transgenic insert |
| TI-317 | TTTG | LbCas12a | 3092:3114 | + | Transgenic insert |
| TI-318 | TCCC | LbCas12a_TYC | 3077:3099 | − | Transgenic insert |
| TI-319 | TTCC | LbCas12a_TYC | 3078:3100 | − | Transgenic insert |
| TI-320 | CTTC | FnCas12a | 3079:3101 | − | Transgenic insert |
| TI-321 | TTTC | LbCas12a | 3082:3104 | − | Transgenic insert |
| TI-322 | ATTC | FnCas12a | 3087:3109 | − | Transgenic insert |
| TI-323 | TTCA | LbCas12a_TYC | 3090:3112 | − | Transgenic insert |
| TI-324 | CTTC | FnCas12a | 3091:3113 | − | Transgenic insert |
| TI-325 | CTTC | FnCas12a | 3094:3116 | − | Transgenic insert |
| TI-326 | TTTA | LbCas12a | 3125:3147 | + | Transgenic insert |
| TI-327 | TTTA | LbCas12a | 3130:3152 | + | Transgenic insert |
| TI-328 | TATA | LbCas12a_TATV | 3120:3142 | − | Transgenic insert |
| TI-329 | TTTG | LbCas12a | 3125:3147 | − | Transgenic insert |
| TI-330 | ATTG | FnCas12a | 3161:3183 | + | Transgenic insert |
| TI-331 | GTTA | FnCas12a | 3168:3190 | + | Transgenic insert |
| TI-332 | TATA | LbCas12a_TATV | 3170:3192 | + | Transgenic insert |
| TI-333 | ATTA | FnCas12a | 3173:3195 | + | Transgenic insert |
| TI-334 | GTTG | FnCas12a | 3153:3175 | − | Transgenic Insert |
| TI-335 | ATTG | FnCas12a | 3168:3190 | − | Transgenic insert |
| TI-336 | CTTC | FnCas12a | 3191:3213 | + | Transgenic insert |
| TI-337 | TTCC | LbCas12a_TYC | 3192:3214 | + | Transgenic insert |
| TI-338 | TCCA | LbCas12a_TYC | 3193:3215 | + | Transgenic insert |
| TI-339 | TTTA | LbCas12a | 3172:3194 | − | Transgenic insert |
| TI-340 | ATTA | FnCas12a | 3197:3219 | + | Transgenic insert |
| TI-341 | TCCA | LbCas12a_TYC | 3186:3208 | − | Transgenic insert |
| TI-342 | TTTG | LbCas12a | 3190:3212 | − | Transgenic insert |
| TI-343 | CTTG | FnCas12a | 3227:3249 | + | Transgenic insert |
| TI-344 | GTTA | FnCas12a | 3230:3252 | + | Transgenic insert |
| TI-345 | GTTA | FnCas12a | 3209:3231 | − | Transgenic insert |
| TI-346 | TATC | LbCas12a_TATV | 3220:3242 | − | Transgenic insert |
| TI-347 | TTTG | LbCas12a | 3242:3264 | + | Transgenic insert |
| TI-348 | GTTG | FnCas12a | 3227:3249 | − | Transgenic insert |
| TI-349 | TATA | LbCas12a_TATV | 3237:3259 | − | Transgenic insert |
| TI-350 | TTTG | LbCas12a | 3259:3281 | + | Transgenic insert |
| TI-351 | CTTA | FnCas12a | 3239:3261 | − | Transgenic insert |
| TI-352 | TATA | LbCas12a_TATV | 3264:3286 | + | Transgenic insert |
| TI-353 | TCCA | LbCas12a_TYC | 3274:3296 | + | Transgenic insert |
| TI-354 | TATA | LbCas12a_TATV | 3258:3280 | − | Transgenic insert |
| TI-355 | ATTA | FnCas12a | 3283:3305 | + | Transgenic insert |
| TI-356 | TATA | LbCas12a_TATV | 3285:3307 | + | Transgenic insert |
| TI-357 | TATG | LbCas12a_TATV | 3287:3309 | + | Transgenic insert |
| TI-358 | CTTG | FnCas12a | 3295:3317 | + | Transgenic insert |
| TI-359 | ATTG | FnCas12a | 3285:3307 | − | Transgenic insert |
| TI-360 | ATTC | FnCas12a | 3314:3336 | + | Transgenic insert |
| TI-361 | GTTA | FnCas12a | 3294:3316 | − | Transgenic insert |
| TI-362 | CTTC | FnCas12a | 3317:3339 | + | Transgenic insert |
| TI-363 | CTTA | FnCas12a | 3298:3320 | − | Transgenic insert |
| TI-364 | TATC | LbCas12a_TATV | 3311:3333 | − | Transgenic insert |
| TI-365 | ATTG | FnCas12a | 3333:3355 | + | Transgenic insert |
| TI-366 | ATTG | FnCas12a | 3327:3349 | − | Transgenic insert |
| TI-367 | ATTG | FnCas12a | 3332:3354 | − | Transgenic insert |
| TI-368 | TTCA | LbCas12a_TYC | 3345:3367 | − | Transgenic insert |
| TI-369 | ATTC | FnCas12a | 3346:3368 | − | Transgenic insert |
| TI-370 | ATTA | FnCas12a | 3396:3418 | + | Transgenic insert |
| TI-371 | TATC | LbCas12a_TATV | 3397:3419 | − | Transgenic insert |
| TI-372 | CTTA | FnCas12a | 3419:3441 | + | Transgenic insert |
| TI-373 | TATG | LbCas12a_TATV | 3421:3443 | + | Transgenic insert |
| TI-374 | GTTA | FnCas12a | 3404:3426 | − | Transgenic insert |
| TI-375 | ATTG | FnCas12a | 3407:3429 | − | Transgenic insert |
| TI-376 | TATA | LbCas12a_TATV | 3415:3437 | − | Transgenic insert |
| TI-377 | ATTA | FnCas12a | 3417:3439 | − | Transgenic insert |
| TI-378 | TTTA | LbCas12a | 3420:3442 | − | Transgenic insert |
| TI-379 | ATTA | FnCas12a | 3469:3491 | + | Transgenic insert |
| TI-380 | ATTA | FnCas12a | 3489:3511 | + | Transgenic insert |
| TI-381 | TTTA | LbCas12a | 3521:3343 | − | Transgenic insert |
| TI-382 | TATA | LbCas12a_TATV | 3569:3591 | + | Transgenic insert |
| TI-383 | ATTA | FnCas12a | 3579:3601 | + | Transgenic insert |
| TI-384 | TTTC | LbCas12a | 3586:3608 | + | Transgenic insert |
| TI-385 | TTCA | LbCas12a_TYC | 3587:3609 | + | Transgenic insert |
| TI-386 | TATG | LbCas12a_TATV | 3591:3613 | + | Transgenic insert |
| TI-387 | TATC | LbCas12a_TATV | 3572:3594 | − | Transgenic insert |
| TI-388 | ATTG | FnCas12a | 3602:3624 | + | Transgenic insert |
| TI-389 | TTTC | LbCas12a | 3582:3604 | − | Transgenic insert |
| TI-390 | TTTA | LbCas12a | 3592:3614 | − | Transgenic insert |
| TI-391 | GTTC | FnCas12a | 3597:3619 | − | Transgenic insert |
| TI-392 | GTTA | FnCas12a | 3602:3624 | − | Transgenic insert |
| TI-393 | ATTA | FnCas12a | 3608:3630 | − | Transgenic insert |
| TI-394 | TTTA | LbCas12a | 3639:3661 | + | Transgenic insert |
| TI-395 | TTTA | LbCas12a | 3643:3665 | + | Transgenic insert |
| TI-396 | GTTG | FnCas12a | 3649:3671 | + | Transgenic insert |
| TI-397 | GTTA | FnCas12a | 3628:3650 | − | Transgenic insert |
| TI-398 | TTCA | LbCas12a_TYC | 3632:3654 | − | Transgenic insert |
| TI-399 | ATTC | FnCas12a | 3633:3655 | − | Transgenic insert |
| TI-400 | GTTC | FnCas12a | 3679:3701 | + | Transgenic insert |
| TI-401 | CTTC | FnCas12a | 3682:3704 | + | Transgenic insert |
| TI-402 | TTCC | LbCas12a_TYC | 3683:3705 | + | Transgenic insert |
| TI-403 | CTTG | FnCas12a | 3661:3683 | − | Transgenic insert |
| TI-404 | TCCA | LbCas12a_TYC | 3684:3706 | + | Transgenic insert |
| TI-405 | TTTC | LbCas12a | 3664:3686 | − | Transgenic insert |
| TI-406 | TATA | LbCas12a_TATV | 3668:3690 | − | Transgenic insert |
| TI-407 | ATTA | FnCas12a | 3670:3692 | − | Transgenic insert |
| TI-408 | TATA | LbCas12a_TATV | 3695:3717 | + | Transgenic insert |
| TI-409 | CTTA | FnCas12a | 3676:3698 | − | Transgenic insert |
| TI-410 | TTTA | LbCas12a | 3701:3723 | + | Transgenic insert |
| TI-411 | TTTC | LbCas12a | 3709:3731 | + | Transgenic insert |
| TI-412 | TTCA | LbCas12a_TYC | 3710:3732 | + | Transgenic insert |
| TI-413 | ATTC | FnCas12a | 3691:3713 | − | Transgenic insert |
| TI-414 | ATTG | FnCas12a | 3695:3717 | − | Transgenic insert |
| TI-415 | TATA | LbCas12a_TATV | 3699:3721 | − | Transgenic insert |
| TI-416 | ATTA | FnCas12a | 3724:3746 | + | Transgenic insert |
| TI-417 | TATA | LbCas12a_TATV | 3726:3748 | + | Transgenic insert |
| TI-418 | TATG | LbCas12a_TATV | 3709:3731 | − | Transgenic insert |
| TI-419 | TTTA | LbCas12a | 3715:3737 | − | Transgenic insert |
| TI-420 | TATC | LbCas12a_TATV | 3738:3760 | + | Transgenic insert |
| TI-421 | TTCA | LbCas12a_TYC | 3721:3743 | − | Transgenic insert |
| TI-422 | ATTC | FnCas12a | 3722:3744 | − | Transgenic insert |
| TI-423 | ATTG | FnCas12a | 3746:3768 | + | Transgenic insert |
| TI-424 | TATG | LbCas12a_TATV | 3726:3748 | − | Transgenic insert |
| TI-425 | TATA | LbCas12a_TATV | 3728:3750 | − | Transgenic insert |
| TI-426 | TATA | LbCas12a_TATV | 3736:3758 | − | Transgenic insert |
| TI-427 | TTCA | LbCas12a_TYC | 3741:3763 | − | Transgenic insert |
| TI-428 | ATTC | FnCas12a | 3742:3764 | − | Transgenic Insert |
| TI-429 | TTTC | LbCas12a | 3810:3832 | + | Transgenic insert |
| TI-430 | TATC | LbCas12a_TATV | 3823:3845 | + | Transgenic insert |
| TI-431 | TATA | LbCas12a_TATV | 3827:3849 | + | Transgenic insert |
| TI-432 | TATC | LbCas12a_TATV | 3834:3856 | + | Transgenic insert |
| TI-433 | TTTC | LbCas12a | 3814:3836 | − | Transgenic insert |
| TI-434 | CTTA | FnCas12a | 3837:3859 | + | Transgenic insert |
| TI-435 | TATC | LbCas12a_TATV | 3845:3867 | + | Transgenic insert |
| TI-436 | CTTG | FnCas12a | 3849:3871 | + | Transgenic insert |
| TI-437 | TATA | LbCas12a_TATV | 3828:3850 | − | Transgenic insert |
| TI-438 | TATA | LbCas12a_TATV | 3855:3877 | + | Transgenic insert |
| TI-439 | TATG | LbCas12a_TATV | 3857:3879 | + | Transgenic insert |
| TI-440 | TATG | LbCas12a_TATV | 3844:3866 | − | Transgenic insert |
| TI-441 | TATC | LbCas12a_TATV | 3866:3888 | + | Transgenic insert |
| TI-442 | CTTA | FnCas12a | 3846:3868 | − | Transgenic insert |
| TI-443 | TATC | LbCas12a_TATV | 3855:3877 | − | Transgenic insert |
| TI-444 | TTTA | LbCas12a | 3857:3879 | − | Transgenic insert |
| TI-445 | TTTA | LbCas12a | 3869:3891 | − | Transgenic insert |
| TI-446 | ATTA | FnCas12a | 3901:3923 | + | Transgenic insert |
| TI-447 | ATTA | FnCas12a | 3910:3932 | + | Transgenic insert |
| TI-448 | TATG | LbCas12a_TATV | 3915:3937 | + | Transgenic insert |
| TI-449 | CTTC | FnCas12a | 3922:3944 | + | Transgenic insert |
| TI-450 | TTCG | LbCas12a_TYC | 3923:3945 | + | Transgenic insert |
| TI-451 | TATC | LbCas12a_TATV | 3913:3935 | − | Transgenic insert |
| TI-452 | TATC | LbCas12a_TATV | 3935:3957 | + | Transgenic insert |
| TI-453 | TCCG | LbCas12a_TYC | 3937:3959 | + | Transgenic insert |
| TI-454 | TATG | LbCas12a_TATV | 3942:3964 | + | Transgenic insert |
| TI-455 | TTCA | LbCas12a_TYC | 3934:3956 | − | Transgenic insert |
| TI-456 | CTTC | FnCas12a | 3935:3957 | − | Transgenic insert |
| TI-457 | ATTG | FnCas12a | 3959:3981 | + | Transgenic insert |
| TI-458 | TATA | LbCas12a_TATV | 3939:3961 | − | Transgenic jasert |
| TI-459 | TATA | LbCas12a_TATV | 3966:3988 | + | Transgenic insert |
| TI-460 | TATG | LbCas12a_TATV | 3950:3972 | − | Transgenic insert |
| TI-461 | TTTC | LbCas12a | 3974:3996 | + | Transgenic insert |
| TI-462 | TTCA | LbCas12a_TYC | 3975:3997 | + | Transgenic insert |
| TI-463 | TATC | LbCas12a_TATV | 3957:3979 | − | Transgenic insert |
| TI-464 | TTTC | LbCas12a | 3994:4016 | + | Transgenic insert |
| TI-465 | TTCA | LbCas12a_TYC | 3995:4017 | + | Transgenic insert |
| TI-466 | TTTG | LbCas12a | 4003:4025 | + | Transgenic insert |
| TI-467 | ATTA | FnCas12a | 3982:4004 | − | Transgenic insert |
| TI-468 | TTTG | LbCas12a | 3991:4013 | − | Transgenic insert |
| TI-469 | TATG | LbCas12a_TATV | 3996:4018 | − | Transgenic insert |
| TI-470 | CTTA | FnCas12a | 3998:4020 | − | Transgenic insert |
| TI-471 | TTTA | LbCas12a | 4002:4024 | − | Transgenic insert |
| TI-472 | ATTG | FnCas12a | 4038:4060 | + | Transgenic insert |
| TI-473 | TATG | LbCas12a_TATV | 4018:4040 | − | Transgenic insert |
| TI-474 | TTTA | LbCas12a | 4020:4042 | − | Transgenic insert |
| TI-475 | TTTA | LbCas12a | 4064:4086 | + | Transgenic insert |
| TI-476 | ATTA | FnCas12a | 4116:4138 | + | Transgenic insert |
| TI-477 | ATTA | FnCas12a | 4119.4141 | + | Transgenic insert |
| TI 478 | TATA | LbCas12a_TATV | 4127:4149 | + | Transgenic insert |
| TI-479 | TATA | LbCas12a_TATV | 4110:4132 | − | Transgenic insert |
| TI-480 | CTTC | FnCas12a | 4132:4154 | + | Transgenic insert |
| TI-481 | TTCG | LbCas12a_TYC | 4133:4155 | + | Transgenic insert |
| T1-482 | TATG | LbCas12a_TATV | 4115:4137 | − | Transgenic insert |
| T1-483 | TATA | LbCas12a_TATV | 4137:4159 | + | Transgenic insert |
| TI-484 | TATA | LbCas12a_TATV | 4122:4144 | − | Transgenic insert |
| TI-485 | ATTA | FnCas12a | 4147:4169 | + | Transgenic insert |
| TI-486 | TTCG | LbCas12a_TYC | 4126:4148 | − | Transgenic insert |
| T1-487 | TATA | LbCas12a_TATV | 4149:4171 | + | Transgenic insert |
| TI-488 | CTTC | FnCas12a | 4127:4149 | − | Transgenic insert |
| T1-489 | GTTA | FnCas12a | 4157:4179 | + | Transgenic insert |
| TI-490 | TATC | LbCas12a_TATV | 4159:4181 | + | Transgenic insert |
| T1-491 | TTCA | LbCas12a_TYC | 4:60:4182 | − | Transgenic insert |
| T1-492 | CTTC | FnCas12a | 4161:4183 | − | Transgenic insert |
| T1-493 | CTTA | FnCas12a | 4174:4196 | − | Transgenic insett |
| TI-494 | TATG | LbCas12a_TATV | 4178:4200 | − | Transgenic insert |
| 3J-1 | TATA | LbCas12a_TATV | 4180:4202 | − | 3′ Junction |
| 3J-2 | TTTG | LbCas12a | 4188:4210 | − | 3′ Junction |
| 3J-3 | TTTC | LbCas12a | 4192:4214 | + | 3′ Junction |
| 3J-4 | GTTA | FnCas12a | 4198:4220 | − | 3′ Junction |
| 3F-1 | TATA | LbCas12a_TATV | 4207:4229 | − | 3′ Flanking genomic DNA |
| 3F-2 | TATG | LbCas12a_TATV | 4209:4231 | + | 3′ Flanking genomic DNA |
| 3F-3 | TCCA | LbCas12a_TYC | 4213:4235 | + | 3′ Flanking genomic DNA |
| 3F-4 | CTTA | FnCas12a | 4202:4224 | − | 3′ Flanking genomic DNA |
| 3F-5 | TTTC | LbCas12a | 4234:4256 | + | 3′ Flanking genomic DNA |
| 3F-6 | TTCA | LbCas12a_TYC | 4235:4257 | + | 3′ Flanking genomic DNA |
| 3F-7 | GTTA | FnCas12a | 4218:4240 | − | 3′ Flanking genomic DNA |
| 3F-8 | TTTA | LbCas12a | 4243:4265 | + | 3′ Flanking genomic DNA |
| 3F-9 | TTTG | LbCas12a | 4226:4248 | − | 3′ Flanking genomic DNA |
| 3F-10 | TTTC | LbCas12a | 4250:4272 | + | 3′ Flanking genomic DNA |
| 3F-11 | TTCA | LbCas12a_TYC | 4251:4273 | + | 3′ Flanking genomic DNA |
| 3F-12 | TTCA | LbCas12a_TYC | 4230:4252 | − | 3′ Flanking genomic DNA |
| 3F-13 | GTTC | FnCas12a | 4231:4253 | − | 3′ Flanking genomic DNA |
| 3F-14 | ATTA | FnCas12a | 4236:4258 | − | 3′ Flanking genomic DNA |
| 3F-15 | GTTG | FnCas12a | 4244:4266 | − | 3′ Flanking genomic DNA |
| 3F-16 | TTTC | LbCas12a | 4268:4290 | + | 3′ Flanking genomic DNA |
| 3F-17 | TTCA | LbCas12a_TYC | 4269:4291 | + | 3′ Flanking genomic DNA |
| 3F-18 | TTTA | LbCas12a | 4275:4297 | + | 3′ Flanking genomic DNA |
| 3F-19 | ATTA | FnCas12a | 4255:4277 | − | 3′ Flanking genomic DNA |
| 3F-20 | ATTC | FnCas12a | 4278:4300 | + | 3′ Flanking genomic DNA |
| 3F-21 | TTTC | LbCas12a | 4288:4310 | + | 3′ Flanking genomic DNA |
| 3F-22 | TATA | LbCas12a_TATV | 4268:4290 | − | 3′ Flanking genomic DNA |
| 3F-23 | CTTC | FnCas12a | 4291:4313 | − | 3′ Flanking genomic DNA |
| 3F-24 | ATTA | FnCas12a | 4270:4292 | − | 3′ Flanking genomic DNA |
| 3F-25 | TATA | LbCas12a_TATV | 4295:4317 | + | 3′ Flanking genomic DNA |
| 3F-26 | TTCC | LbCas12a_TYC | 4275:4297 | − | 3′ Flanking genomic DNA |
| 3F-27 | TTTC | LbCas12a | 4276:4298 | − | 3′ Flanking genomic DNA |
| 3F-28 | ATTG | FnCas12a | 4310:4332 | + | 3′ Flanking genomic DNA |
| 3F-29 | TTCA | LbCas12a_TYC | 4289:4311 | − | 3′ Flanking genomic DNA |
| 3F-30 | CTTC | FnCas12a | 4290:4312 | − | 3′ Flanking genomic DNA |
| 3F-31 | CTTG | FnCas12a | 4314:4336 | + | 3′ Flanking genomic DNA |
| 3F-32 | TTTC | LbCas12a | 4293:4315 | − | 3′ Flanking genomic DNA |
| 3F-33 | GTTC | FnCas12a | 4297:4319 | − | 3′ Flanking genomic DNA |
| 3F-34 | TTCA | LbCas12a_TYC | 4306:4328 | − | 3′ Flanking genomic DNA |
| 3F-35 | TTTC | LbCas12a | 4307:4329 | − | 3′ Flanking genomic DNA |
| 3F-36 | GTTG | FnCas12a | 4331:4353 | + | 3′ Flanking genomic DNA |
| 3F-37 | TTTG | LbCas12a | 4312:4334 | − | 3′ Flanking genomic DNA |
| 3F-38 | TTTC | LbCas12a | 4343:4365 | + | 3′ Flanking genomic DNA |
| 3F-39 | TTCC | LbCas12a_TYC | 4344:4366 | + | 3′ Flanking genomic DNA |
| 3F-40 | TTCC | LbCas12a_TYC | 4324:4346 | − | 3′ Flanking genomic DNA |
| 3F-41 | CTTC | FnCas12a | 4325:4347 | − | 3′ Flanking genomic DNA |
| 3F-42 | TTTA | LbCas12a | 4329:4351 | − | 3′ Flanking genomic DNA |
| 3F-43 | CTTG | FnCas12a | 4339:4361 | − | 3′ Flanking genomic DNA |
| 3F-44 | TTTG | LbCas12a | 4361:4383 | + | 3′ Flanking genomic DNA |
| 3F-45 | CTTC | FnCas12a | 4394:4416 | + | 3′ Flanking genomic DNA |
| 3F-46 | TTCA | LbCas12a_TYC | 4395:4417 | + | 3′ Flanking genomic DNA |
| 3F-47 | CTTG | FnCas12a | 4382:4404 | − | 3′ Flanking genomic DNA |
| 3F-48 | TATC | LbCas12a_TATV | 4395:4417 | − | 3′ Flanking genomic DNA |
| 3F-49 | ATTA | FnCas12a | 4418:4440 | + | 3′ Flanking genomic DNA |
| 3F-50 | TATC | LbCas12a_TATV | 4399:4421 | − | 3′ Flanking genomic DNA |
| 3F-51 | TATA | LbCas12a_TATV | 4401:4423 | − | 3′ Flanking genomic DNA |
| 3F-52 | TTCA | LbCas12_TYC | 4420:4442 | − | 3′ Flanking genomic DNA |
| 3F-53 | TTTC | LbCas12a | 4421:4443 | − | 3′ Flanking genomic DNA |
| 3F-54 | TTTC | LbCas12a | 4480:4502 | + | 3′ Flanking genomic DNA |
| 3F-55 | TTCC | LbCas12a_TYC | 4481:4503 | + | 3′ Flanking genomic DNA |
| 3F-56 | TCCC | LbCas12a_TYC | 4482:4504 | + | 3′ Flanking genomic DNA |
| 3F-57 | CTTC | FnCas12a | 4486:4508 | + | 3′ Flanking genomic DNA |
| 3F-58 | TTCA | LbCas12a_TYC | 4487:4509 | + | 3′ Flanking genomic DNA |
| 3F-59 | GTTG | FnCas12a | 4501:4523 | + | 3′ Flanking genomic DNA |
| 3F-60 | CTTG | FnCas12a | 4508:4530 | + | 3′ Flanking genomic DNA |
| 3F-61 | TATG | LbCas12a_TATV | 4490:4512 | − | 3′ Flanking genomic DNA |
| 3F-62 | TATA | LbCas12a_TATV | 4492:4514 | − | 3′ Flanking genomic DNA |
| 3F-63 | TATA | LbCas12a_TATV | 4494:4316 | − | 3′ Flanking genomic DNA |
| 3F-64 | TATA | LbCas12a_TATV | $496:4518 | − | 3′ Flanking genomic DNA |
| 3F-65 | TATA | LbCas12a_TATV | 4519:4541 | + | 3′ Flanking genomic DNA |
| 3F-66 | TATA | LbCas12a_TATV | 4521:4543 | + | 3′ Flanking genomic DNA |
| 3F-67 | TATA | LbCas12a_TATV | 4523:4543 | + | 3′ Flanking genomic DNA |
| 3F-68 | TTCG | LbCas12a_TYC | 4505:4527 | − | 3′ Flanking genomic DNA |
| 3F-69 | TTTC | LbCas12a | 4506:4528 | − | 3′ Flanking genomic DNA |
| 3F-70 | TTTG | LbCas12a | 4511:4533 | − | 3′ Flanking genomic DNA |
| 3F-71 | ATTA | FnCas12a | 4522:4544 | − | 3′ Flanking genomic DNA |
| 3F-72 | TTTC | LbCas12a | 4585:4607 | + | 3′ Flanking genomic DNA |
| 3F-73 | CTTA | FnCas12a | 4588:4610 | + | 3′ Flanking genomic DNA |
| 3F-74 | TTTA | LbCas12a | 4586:4608 | − | 3′ Flanking genomic DNA |
| 3F-75 | CTTA | FnCas12a | 4591:4613 | − | 3′ Flanking genomic DNA |
| 3F-76 | ATTA | FnCas12a | 4659:4681 | + | 3′ Flanking genomic DNA |
| 3F-77 | TTTC | LbCas12a | 4667:4689 | + | 3′ Flanking genomic DNA |
| 3F-78 | TTCA | LbCas12a_TYC | 4668:4690 | + | 3′ Flanking genomic DNA |
| 3F-79 | TTTG | LbCas12a | 4674:4696 | + | 3′ Flanking genomic DNA |
| 3F-80 | TTTA | LbCas12a | 4659:4681 | − | 3′ Flanking genomic DNA |
| 3F-81 | TATG | LbCas12a_TATV | 4664:4686 | − | 3′ Flanking genomic DNA |
| 3F-82 | CTTA | FnCas12a | 4666:4688 | − | 3′ Flanking genomic DNA |
| 3F-83 | TTTG | LbCas12a | 4680:4702 | − | 3′ Flanking genomic DNA |
| 3F-84 | TATA | LbCas12a_TATV | 4736:4758 | − | 3′ Flanking genomic DNA |
| 3F-85 | TATA | LbCas12a_TATV | 4745:4767 | − | 3′ Flanking genomic DNA |
| 3F-86 | TTTA | LbCas12a | 4809:4831 | − | 3′ Flanking genomic DNA |
| 3F-87 | TATA | LbCas12a_TATV | 4811:4833 | + | 3′ Flanking genomic DNA |
| 3F-88 | CTTC | FnCas12a | 4822:4844 | + | 3′ Flanking genomic DNA |
| 3F-89 | TTCG | LbCas12a_TYC | 4823:4845 | + | 3′ Flanking genomic DNA |
| 3F-90 | TTCG | LbCas12a_TYC | 4806:4828 | + | 3′ Flanking genomic DNA |
| 3F-91 | TTTC | LbCas12a | 4807:4829 | − | 3′ Flanking genomic DNA |
| 3F-92 | GTTG | FnCas12a | 4818:4840 | − | 3′ Flanking genomic DNA |
| 3F-93 | TTTA | LbCas12a | 4841:4863 | − | 3′ Flanking genomic DNA |
| 3F-94 | TATG | LbCas12a_TATV | 4821:4843 | + | 3′ Flanking genomic DNA |
| 3F-95 | ATTA | FnCas12a | 4832:4854 | − | 3′ Flanking genomic DNA |
| 3F-96 | TTCG | LbCas12a_TYC | 4839:4861 | − | 3′ Flanking genomic DNA |
| 3F-97 | CTTC | FnCas12a | 4840:4862 | − | 3′ Flanking genomic DNA |
| 3F-98 | TTTC | LbCas12a | 4879:4901 | − | 3′ Flanking genomic DNA |
| 3F-99 | TTTA | LbCas12a | 4875:4897 | + | 3′ Flanking genomic DNA |
| 3F-100 | TTTA | LbCas12a | 4909:4931 | − | 3′ Flanking genomic DNA |
| 3F-101 | TTTA | LbCas12a | 4934:4956 | − | 3′ Flanking genomic DNA |
| 3F-102 | TATA | LbCas12a_TATV | 4917:4939 | + | 3′ Flanking genomic DNA |
| 3F-103 | ATTA | FnCas12a | 4939:4961 | − | 3′ Flanking genomic DNA |
| 3F-104 | TATA | LbCas12a_TATV | 4919:4941 | + | 3′ Flanking genomic DNA |
| 3F-105 | ATTA | FnCas12a | 4942:4964 | − | 3′ Flanking genomic DNA |
| 3F-106 | TATA | LbCas12a_TATV | 4944:4966 | + | 3′ Flanking genomic DNA |
| 3F-107 | TATA | LbCas12a_TATV | 4946:4968 | + | 3′ Flanking genomic DNA |
| 3F-108 | TATA | LbCas12a_TATV | 4948:4970 | + | 3′ Flanking genomic DNA |
| 3F-109 | TATA | LbCas12a_TATV | 4950:4972 | + | 3′ Flanking genomic DNA |
| 3F-110 | TTTA | LbCas12a | 4956:4978 | + | 3′ Flanking genomic DNA |
| 3F-111 | GTTA | FnCas12a | 4960:4982 | + | 3′ Flanking genomic DNA |
| 3F-112 | ATTA | FnCas12a | 4944:4966 | − | 3′ Flanking genomic DNA |
| 3F-113 | TTCA | LbCas12a_TYC | 4954:4976 | − | 3′ Flanking genomic DNA |
| 3F-114 | TTTC | LbCas12a | 4955:4977 | − | 3′ Flanking genomic DNA |
| 3F-115 | TATG | LbCas12a_TATV | 4967:4989 | − | 3′ Flanking genomic DNA |
| 3F-116 | TTTC | LbCas12a | 4989:5011 | + | 3′ Flanking genomic DNA |
| 3F-117 | CTTA | FnCas12a | 4969:4991 | − | 3′ Flanking genomic DNA |
| 3F-118 | TATG | LbCas12a_TATV | 4974:4996 | − | 3′ Flanking genomic DNA |
| 3F-119 | TTTA | LbCas12a | 4976:4998 | − | 3′ Flanking genomic DNA |
| 3F-120 | ATTA | FnCas12a | 4984:5006 | − | 3′ Flanking genomic DNA |
| 3F-121 | TATG | LbCas12a_TATV | 4992:5014 | − | 3′ Flanking genomic DNA |
| 3F-122 | TTTC | LbCas12a | 5032:5054 | + | 3′ Flanking genomic DNA |
| 3F-123 | TTTA | LbCas12a | 5038:5060 | + | 3′ Flanking genomic DNA |
| 3F-124 | TATA | LbCas12a_TATV | 5040:5062 | + | 3′ Flanking genomic DNA |
| 3F-123 | TATC | LbCas12a_TATV | 5042:5064 | + | 3′ Flanking genomic DNA |
| 3F-126 | TTTA | LbCas12a | 5072:5094 | + | 3′ Flanking genomic DNA |
| 3F-127 | TTTA | LbCas12a | 5078:5100 | + | 3′ Flanking genomic DNA |
| 3F-128 | TTTC | LbCas12a | 5094:5116 | + | 3′ Flanking genomic DNA |
| 3F-129 | CTTG | FnCas12a | 5073:5095 | − | 3′ Flanking genomic DNA |
| 3F-130 | TTTC | LbCas12a | 5076:5098 | − | 3′ Flanking genomic DNA |
| 3F-131 | ATTA | FnCas12a | 5080:5102 | − | 3′ Flanking genomic DNA |
| 3F-132 | CTTC | FnCas12a | 5111:5133 | + | 3′ Flanking genomic DNA |
| 3F-133 | TATC | LbCas12a_TATV | 5091:5113 | − | 3′ Flanking genomic DNA |
| 3F-134 | CTTC | FnCas12a | 5114:5136 | + | 3′ Flanking genomic DNA |
| 3F-135 | TTCG | LbCas12a_TYC | 5115:5137 | + | 3′ Flanking genomic DNA |
| 3F-136 | TCCA | LbCas12a_TYC | 5096:5118 | − | 3′ Flanking genomic DNA |
| 3F-137 | TTCC | LbCas12a_TYC | 5097:5119 | − | 3′ Flanking genomic DNA |
| 3F-138 | GTTC | FnCas12a | 5098:5120 | − | 3′ Flanking genomic DNA |
| 3F-139 | ATTG | FnCas12a | 5121:5143 | + | 3′ Flanking genomic DNA |
| 3F-140 | ATTG | FnCas12a | 5107:5129 | − | 3′ Flanking genomic DNA |
| 3F-141 | TTCA | LbCas12a_TYC | 5115:5137 | − | 3′ Flanking genomic DNA |
| 3F-142 | TTTC | LbCas12a | 5116:5138 | − | 3′ Flanking genomic DNA |
| 3F-143 | ATTA | FnCas12a | 5121:5143 | − | 3′ Flanking genomic DNA |
| 3F-144 | TTTC | LbCas12a | 5168:5190 | + | 3′ Flanking genomic DNA |
| 3F-145 | TTCA | LbCas12a_TYC | 5169:5191 | + | 3′ Flanking genomic DNA |
| 3F-146 | TTCA | LbCas12a_TYC | 5163:5185 | − | 3′ Flanking genomic DNA |
| 3F-147 | TTTC | LbCas12a | 5164:5186 | − | 3′ Flanking genomic DNA |

gRNAs may include a G leader sequence to facilitate transcription start from a pol III promoter positioned 5′ to a gRNA repeat, resulting in GAATTTCTACTAAGTGTAGAT (SEQ ID NO:203) for LbCas12a, or GTAATTTCTACTGTTGTAGAT (SEQ ID NO:204) for FnCas12a and an OgRRS sequence (as shown in the fourth column of Table 20). For Agrobacterium-based plant expression vectors, the cassette further comprises a poly-T transcript termination region (TTTTTTT). These gRNAs can be used to target the Cas12a nuclease to cut within both the OgRRS and CgRRS sequences. Illustrative examples of such gRNAs for FnCas12a expressed in stable plants are shown in Table 21, wherein the gRNA repeat of TAATTTCTACTGTTGTAGAT is underlined, and the poly-T transcript termination sequence of TTTTTTT is shown in italic font.

TABLE 21
Illustrative examples of gRNAs useful in targeting FnCas12a nuclease

| gRNA | Sequence |
|---|---|
| gRNA_5F-134 | GTAATTTCTACTGTTGTAGATTTATAAGGTGACCTCCTCCATAATTTTTTT |
| (SEQ ID NO: 205) | |
| gRNA_3F-15 | GTAATTTCTACTGTTGTAGATAAAGATTAGGTTCATTTGAAAAGTTTTTTT |
| (SEQ ID NO: 206) | |

Any of the OgRRS sequences presented in Table 20 can be used alternatively as a site to insert a CgRRS that is designed using a different OgRRS. For example, a CgRRS can be inserted into a flanking sequence to allow for the excision of the entire transgenic insertion of event Gm_CSM63717. To illustrate this approach, OgRRS 3F-15 is selected as the OgRRS that can be used to design a corresponding CgRRS 3F-15 comprising DNA fragment, and OgRRS 5F-134 is selected as the target site into which the CgRRS 3F-15 comprising DNA fragment is inserted. Using a Cas12a editing system such as with the FnCas12a endonuclease, the OgRRS 5F-134 site is targeted using the gRNA, gRNA_5F-134 presented in Table 21 to cut within the OgRRS 5F-134 site. The CgRRS 3F-15 comprising DNA fragment that comprises the OgRRS 3F-15 target site is then inserted within the cut site that was introduced into the OgRRS 5F-134 sequence. After selection of a transgenic event comprising the introduced CgRRS 3F-15 site, the event can be bred into another germplasm. When desired, the transgenic insert of Gm_CSM63717 can be excised from the plant using a Cas12a editing system and the gRNA, gRNA_3F-15 as presented in Table 21.

The CgRRS can be introduced into the transgenic insertion locus through multiple methods using a CRISPR system. For example, a CRISPR system can be used for targeting 5′ insertion of a blunt-end double-stranded DNA fragment into a genomic target site of interest such as an OgRRS that is not the OgRRS that has been selected for the design of the CgRRS. The CRISPR-mediated endonuclease activity can introduce a double stand break (DSB) in the selected genomic target site and DNA repair, such as microhomology-driven nonhomologous end-joining DNA repair, resulting in insertion of the blunt-end double-stranded DNA fragment into the DSB. Blunt-end double-stranded DNA fragments can be designed with 1-10 bp of microhomology, on both the 5′ and 3′ ends of the DNA fragment, that correspond to the 5′ and 3′-flanking sequence at the cut site of the protospacer in the genomic target site.

The CRISPR system can be introduced into event Gm_CSM63717 by several methods, including but not limited to Agrobacterium-mediated transformation, polyethylene glycol-mediated transformation, biolistic transformation, liposome-mediated transfection, viral transduction, the use of one or more delivery particles, microinjection, or electroporation. One or more expression cassettes encoding the gRNA and/or CRISPR associated protein components of a Type I, Type II, Type III, Type IV, Type V, or Type VI CRISPR-Cas system is transiently introduced into a cell. The CRISPR-associated protein and guide RNA can be synthesized and assembled in vitro to form a ribonucleoprotein complex (RNP). The ribonucleoprotein along with a DNA fragment encoding the CgRRS can be provided to the plant via polyethylene glycol-mediated transformation, biolistic transformation, liposome-mediated transfection, the use of one or more delivery particles, microinjection, or electroporation. The introduced one or more gRNAs, or expression cassettes encoding the gRNA and/or CRISPR associated protein, along with a DNA fragment comprising the CgRRS is provided in sufficient quantity to modify the cell but does not persist after a contemplated period of time has passed or after one or more cell divisions. In such embodiments, no further steps are needed to remove or segregate the one or more expression cassettes encoding the gRNA and/or CRISPR associated protein from the modified cell. Double-stranded DNA fragments can also be transiently introduced into a cell along with one or more expression cassettes encoding the gRNA and/or CRISPR associated protein. The introduced double-stranded DNA fragments are provided in sufficient quantity to modify the cell but do not persist after a contemplated period of time has passed or after one or more cell divisions.

Alternatively, an expression construct comprising one or more expression cassettes for the expression of one or more gRNAs, and an expression construct encoding a Type I, Type II, Type III, Type IV, Type V, or Type VI CRISPR associated protein is stably transformed into event Gm_CSM63717 to modify the plant cell in the targeted region of the transgene insertion locus, to introduce the CgRRS within the desired target locus.

### Example 11: Modification of Soybean Event Gm_CSM63717 with Genome Editing Techniques Using Two Guide RNAs

This example describes how one may alter or excise all or a part of the transgenic insertion present in soybean event Gm_CSM63717, as well as flanking genomic DNA segments, such as by making one or more insertions, deletions, substitutions, or transpositions using genomic editing techniques. Excision of all or a portion of the event Gm_CSM63717 transgenic insertion or expression element(s) or cassette within SEQ ID NO:9 or SEQ ID NO:10 can be performed through genome editing using a variety of methods. In one embodiment, Clustered Regularly Interspersed Short Palindromic Repeats (CRISPR) editing systems comprising a CRISPR associated protein and two cognate guide RNAs may be used for targeted excision. The CRISPR-associated protein is an RNA guided nuclease and can be selected from a Type I CRISPR-associated protein, a Type II CRISPR-associated protein, a Type III CRISPR-associated protein, a Type IV CRISPR-associated protein, Type V CRISPR-associated protein, or a Type VI CRISPR-associated protein, such as but not limited to, Cas1, Cas1B, Cas2, Cas3, Cas4, Cas5, Cas6, Cas7, Cas8, Cas9 (also known as Csn1 and Csx12), Cas10, Cas12a (also known as Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, CasX, CasY, and Mad7. The CRISPR-associated protein and two guide RNAs (gRNA) can be introduced into a plant cell comprising the soybean event Gm_CSM63717 to target a specific sequence within the transgene insertion locus. In one embodiment, the CRISPR nuclease system cleaves at two distinct guide RNA hybridization sites thereby permitting the excision of the intervening sequence. Following DNA cleavage, the genomic sequence can be repaired via a double strand break repair pathway, which may include, for example, non-homologous end-joining (NHEJ), microhomology-mediated end joining (MMEJ), homologous recombination, synthesis-dependent strand annealing (SDSA), single-strand annealing (SSA), or a combination thereof, at the genomic target site.

Sequences corresponding to the 5′ and 3′ flanking genomic sequences, and the transgenic insert of event Gm_CSM63717 (SEQ ID NOs:11, 12, and 9, respectively) and the 5′ and 3′ junction regions (SEQ ID NOs:1-6) were scanned for potential guide RNA recognition sites which comprise a protospacer adjacent motif (PAM) site that will be recognized by a Cas12a endonuclease, operably linked to a guide RNA hybridization site, and the results are shown in Table 20 above. The identified gRNA recognition sites are located within the 5′ or 3′ flanking genomic sequence, within the 5′ or 3′ junction regions, or within the transgenic insertion.

Two functional guide RNAs (gRNAs) for an RNA guided nuclease system are created to target the event Gm_CSM63717 transgenic insertion locus in a manner that will permit the excision of a fragment of DNA corresponding to either the entire transgenic insertion of event Gm_CSM63717, or a fragment within the transgenic insertion of event Gm_CSM63717 such as the expression cassette or a genetic element within the transgene cassette. Illustrative examples are described below using a FnCas12a editing system (see Table 21 above), in which the gRNAs may include a G leader sequence to facilitate transcription start and a gRNA repeat GTAATTTCTACTGITGTAGAT (SEQ ID NO:204, gRNA repeat underlined), an OgRRS sequence (as shown in the fourth column of Table 20) and a TTTTTTT (poly-T transcript termination region, italic in Table 21 above) to target the FnCas12a nuclease to the gRNA recognition sites. Similar methods can be used to excise either the entire transgene insertion or a fragment within the transgene insertion such as the expression cassette by selecting gRNAs targeted to the specific regions. Alternatively, the LbCas12a editing systems can be used, in which the gRNAs include a G leader sequence to facilitate transcription start and a gRNA repeat GAATTTCTACTAAGTGTAGAT (SEQ ID NO:203, gRNA repeat underlined), an OgRRS sequence (as shown in the third column of Table 21 above), and a TITITIT (poly-T transcript termination region).

To excise the entire transgenic insertion of event Gm_CSM63717, the first gRNA targets an area in the 5′ flanking genomic sequence such as 5F-134 (Table 21), and the second gRNA targets a region in the 3′ flanking genomic sequence such as 3F-15 (Table 21). A transfer DNA (T-DNA) construct suitable for use in Agrobacterium-mediated transformation is used. The T-DNA construct comprises several expression cassettes between a left border (LB) sequence and a right border (RB) sequence. The first expression cassette comprises a promoter that is operable in a plant cell operably linked to a polynucleotide encoding a Cas12a RNA guided nuclease. A second expression cassette comprises a promoter that is operable in a plant cell operably linked to a selection marker gene, such as aadA for conferring resistance to spectinomycin and/or streptomycin. The construct also comprises expression cassettes comprising Polymerase I or Polymerase II promoters operable in a plant cell operably linked to polynucleotides encoding the two gRNAs, gRNA_5F-134 and gRNA_3F-15 (Table 21).

Following Agrobacterium-mediated transformation of soybean comprising event Gm_CSM63717, and upon expression of the integrated polynucleotides, the gRNAs guide the nuclease to each of the two target sites at the transgenic insertion locus, where the nuclease creates a double-stranded break at each target site, resulting in deletion of the region between the target sites, and non-homologous end-joining repair mechanisms joins the flanking regions. Suitable methods known in the art (e.g., PCR, DNA hybridization (Southern) blots, sequencing) are used to identify plants comprising a complete deletion.

### REFERENCES

X. Chen, L. Levine and P.-Y. Kwok (1999). Fluorescence polarization in homogeneous nucleic acid analysis. Genome Res. 9:492498.
S. Cheng, C. Fockler, W. M. Barnes and R. Higuchi (1994). Effective amplification of long targets from cloned inserts and human genomic DNA. Proc. Natl. Acad. Sci. USA 91:5695-5699.
C. D. Day, E. Lee, J. Kobayashi, L. D. Holappa, H. Albert and D. W. Ow (2000). Transgene integration into the same chromosome location can produce alleles that express at a predictable level, or alleles that are differentially silenced. Genes Dev. 14 (22):2869-2880.
F. B. Dean. S. Hosono, L. Fang, X. Wu, A. F. Faruqi, P. Bray-Ward, Z. Sun, Q. Zong, Y. Du, J. Du, M. Driscoll, W. Song, S. F. Kingsmore, M. Egholm and R. S. Lasken (2002). Proc. Natl. Acad Sci. USA 99:5261-5266.
A. D. Ellington and J. W. Szostak, J. (1990). In vitro selection of RNA molecules that bind specific ligands. Nature 346:818-822.
R. D. Finn, A. Bateman, J. Clements, P. Coggill, R. Y. Eberhardt, S. R. Eddy, A. Heger, K. Ketherington, L. Holm, J. Mistry, E. L. L. Sonnhmmer, J. Tate and M. Punta (2014). Pfam: the protein families database. Nucleic Acids Research 42 (D1):D222-D230.
A. Fire and S. Q. Xu (1995). Rolling replication of short DNA circles. Proc. Natl. Acad Sci. USA 92:4641-4645.
L. Gao, D. B. T. Cox, W. X. Yan, J. C. Manteiga, M. W. Schneider, T. Yamano, H. Nishimasu, O. Nureki, N. Crosetto and F. Zhang (2017). Engineered Cpf1 variants with altered PAM specificities. Nature Biotechnology 35(8):789-792.
J. C. Guatelli, K. M. Whitefield, D. Y. Kwon, KJ. Barringer, D. D. Richman and T. R. Gingeras (1990). Isothermal, in vitro amplification of nucleic acids by a multienzyme reaction modeled after retroviral replication. Proc. Natl. Acad. Sci. USA 87:1874-1878.
I. M. Heap (2021). International Survey of Herbicide Resistant Weeds. http://www.weedscience.org/Home.aspx.
D. Hyatt, G.-L. Chen, P. F. LoCascio, M. L. Land, F. W. Larimer and L. J. Hauser (2010). Prodigal: prokaryotic gene recognition and translation initiation site identification. BMC Bioinfonmatics 11, 119.
S. Khan, A. Latif, S. Q, Ahmad, F. Ahmad and M. Ghaffar (2010). Double haploid technique: in soybean and other species. International Journal of Applied Agricultural Research 5:649-655.
C. T. Larue, J. E. Ream, X. Zhou, F. Moshiri, A. Howe, M. Goley, O. C. Sparks, S. T. Voss, E. Hall, C. Ellis, J. Weihe, Q. Qi, D. Ribeiro, X. Wei, S. Guo, A. G. Evdokimov, M. J. Varagona and J. K. Roberts (2019). Microbial HemG-type protoporphyrinogen IX oxidase enzymes for biotechnology applications in plant herbicide tolerance traits. Pest Management Sci. 76 (3):1031-1038.
L. Lermontova, E. Kruse, H. P. Mock, and B. Grimm (1997). Cloning and characterization of plastidal and mitochondrial isoform of tobacco protoporphyrinogen IX oxidase. Proc. Natl. Acad. Sci. USA 94:8895-8900.
Q. Li, Y. Huo, Y. Guo, X. Zheng, W. Sun and Z. Hao (2017) Generation and applications of a DNA aptamer against gremlin-1. Molecules 22:706
D. Liu, S. L. Daubendiek, M. Zillman, K. Ryan and E. T. Kool (1996). Rolling circle DNA synthesis: small circular oligonucleotides as efficient templates for DNA polymerases. J. Am. Chem. Soc. 118:1587-1594.
P. M. Lizardi, X. Huang, Z. Zhu, P. Bray-Ward, D. C. Thomas and D. C. Ward (1998). Mutation detection and single-molecule counting using isothermal rolling-circle amplification. Nature Genetics 19:225-232.
T. T. Nikiforov, R. B. Rendie, P. Goelet, Y.-H. Rogers, M. L. Kotewicz, S. Anderson, G. L. Trainor and M. R. Knapp (1994). Genetic bit analysis: a solid phase method for typing single nucleotide polymorphisms. Nucleic Acids Res. 22:4167-4175.
T. Notomi, H. Okayama, H. Masubuchi, T. Yonekawa, K. Watanabe, N. Amino, T. Hase (2000). Loop-mediated isothermal amplification of DNA”. Nucleic Acids Res. 28 (12): 63e-63.
D. T. Patterson (1995). Effects of environmental stress on weed/crop interactions. Weed Science 43:483-490.
M. D. Pearson, L. Nguyen, Y. Zhao, W. L. McKenna, T. J. Morin and W. B. Dunbar (2019). Past and accurate quantification of insertion-site specific transgene levels from raw seed samples using solid-state nanopore technology. PloS One 14 (12): e0226719.
D. L. Robertson and J. G. Joyce (1990). Selection in vitro of an RNA enzyme that specifically cleaves single-stranded DNA. Nature 344:467-468.
S. S. Sekhon, H. J. Um, W. R. Shin, S. H. Lee, J. Min, J. Y. Ahn and Y. H. Kim. (2017). Aptabody-aptatope interactions in aptablotting assays. Nanoscale 9:7464-7475.
D. Shoup, S. Duncan and IA. Ciampitti (2016). Soybean Production Handbook. Kansas State University Agricultural Experiment Station and Cooperative Extension Service.
N. Soltani, J. A. Dille, I. C. Burke, W. J. Everman, M. J. VanGessel, V. M. Davis, and P. H. Sikkema (2017). Perspectives on potential soybean yield losses from weeds in North America. Weed Technology 31 (1): 148-154.
M. Svobodova, V. Skouridou, M. Jauset-Rubio, I. Viéitez, A. Fernández-Villar, J. J. C. Alvargonzalez, E. Poveda, C. B. Bofill, T. Sans, A. Bashammakh, A. O. Alyoubi and C. K. O'Sullivan (2021). Aptamer sandwich assay for detection of SARS-CoV-2-spike protein antigen. ACS Omega 6:35657-35666.
A. D. Taylor, L. Mataseje, C. J. Urfano, L. Schmidt, K. S. Antonation, M. R. Mulvey and C. R. Corbett (2018). Evaluation of Oxford Nanopore's MinION Sequencing Device for Microbial Whole Genome Sequencing Applications. Scientific Reports 8:10931.
C. Tuerk and L. Gold (1990). Systematic evolution of ligands by exponential enrichment: RNA ligands to bacteriophage T4 DNA polymerase. Science 249:505-510.
S. Tyagi and F. R. Kramer (1996). Molecular beacons: probes that fluoresce upon hybridization. Nature Biotechnology 14:303-308.
A. Verkest, S. Bourout, J. Debaveye, K. Reynaert, B. Saey, I. Van den Brande and K. D'Halluin (2019). Impact of differential DNA methylation on transgene expression in cotton (Gossypium hirsutum L.) events generated by targeted sequence insertion. Plant Biotechnology J. 17:1236-1247.
M. Vincent, Y. Xu and H. Kong (2004). Helicase-dependent isothermal DNA amplification. EMBO Reports 5(8):795-800.
Y. Wan, J. F. Petolino and J. M. Widholm (1989). Efficient Production of Doubled Haploid Plants Through Colchicine Treatment of Anther-Derived Maize Callus. Theor. Appl. Genet. 77:889-892.
T. Wang, C. Chen, L. M. Larcher, R. A. Banero and R. N. Veedu (2019). Three decades of nucleic acid aptamer technologies: Lessons learned, progress and opportunities on aptamer development. Biotechnol. Adv. 37:28-50.
Y. Wang, Y. Zhao, A. Bollas, Y. Wang and K. F. Au (2021). Nanopore sequencing technology, bioinformatics and applications. Nature Biotechnology 39:1348-1365.
Y. Wang, Z. Li and H. Yu (2020). Aptamer-baased Western blot for selective protein recognition. Frontiers in Chem. 8:1-9.
M. Winge (2000). Pyrosequencing-a new approach to DNA analysis. Innovation in Pharma. Tech. 00:18-24.
Y. Xu, X. Zhou and W. Zhang (2008) MicroRNA prediction with a novel ranking algorithm based on random walk. Bioinformatics 24 (13):i50-458.
S. Yang, J. Fresnedo-Ramirez, M. Wang, L. Cote, P. Schweitzer, P. Barba, E. M. Takacs, M. Clark, J. Luby. D. C. Manns, G. Sacks, A. K. Mansfield, J. Londo, A. Fennell, D. Gadoury, B. Reisch, L. Cadle-Davidson, and Q. Sun (2016). A next-generation marker genotyping platform (AmpSeq) in heterozygous crops: a case study for marker-assisted selection in grapevine. Hort. Res. 3:16002.
B. Zetsche, J. S. Gootenberg, 0.0. Abudayyeh, I. M. Slaymaker, K. S. Makarova, P. Essletzbichler, S. E. Volz, J. Joung, J. v. d. Oost, A. Regev, E. V. Koonin and F. Zhang (2015). Cpf1 is a single RNA-guided endonuclease of a class 2 CRISPR-Cas system. Cell 163(3):759-771.

1. A recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing.

2. The recombinant DNA molecule of claim 1, wherein:
a) the recombinant DNA molecule is derived from a soybean plant, seed, plant part, plant cell, progeny plant, or commodity product comprising soybean event Gm_CSM63717, a representative sample of seed comprising the event having been deposited as ATCC Accession No. PTA-127604;
b) the recombinant DNA molecule is comprised in a soybean plant, seed, plant part, plant cell, or progeny plant comprising soybean event Gm_CSM63717, or a commodity product produced therefrom, a representative sample of seed comprising the event having been deposited as ATCC Accession No. PTA-127604;
c) the recombinant DNA molecule is formed by the insertion of a heterologous nucleic acid molecule into the genomic DNA of a soybean plant or soybean cell; or
d) the recombinant DNA molecule comprises an amplicon diagnostic for the presence of soybean event Gm_CSM63717.

3. A soybean plant, plant part, plant seed, or plant cell that comprises soybean event Gm_CSM63717, a representative sample of seed comprising the event having been deposited as ATCC Accession No. PTA-127604

4-5. (canceled)

6. A DNA molecule comprising:
a) a polynucleotide segment of sufficient length to function as a DNA probe that hybridizes specifically under stringent hybridization conditions with soybean event Gm_CSM63717 DNA in a sample obtained from the soybean plant, plant part, plant seed, or plant cell of claim 3 or a progeny plant thereof or a commodity product produced therefrom, wherein detecting hybridization of the DNA molecule under the stringent hybridization conditions is diagnostic for the presence of soybean event Gm_CSM63717 in the sample; or
b) a polynucleotide segment of sufficient length to function as a DNA probe specific for detecting in a sample at least one of:
a 5′ junction sequence between flanking soybean genomic DNA and the transgenic insert of soybean event Gm_CSM63717;
a 3′ junction sequence between the transgenic insert of soybean event Gm_CSM63717 and flanking soybean genomic DNA;
SEQ ID NO:9; and
a fragment of SEQ ID NO:9 comprising a sufficient length of contiguous nucleotides of SEQ ID NO:9 to identify the sequence as a fragment of the transgenic insert of Gm_CSM63717.

7. (canceled)

8. The DNA molecule of claim 6, wherein:
a) the DNA molecule comprises SEQ ID NO:178;
b) the DNA molecule comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; and a complement of any of the foregoing; or
c) the sample is derived from a soybean plant, seed, plant part, plant cell, progeny plant, or commodity product.

9-10. (canceled)

11. A pair of DNA molecules comprising a first DNA molecule and a second DNA molecule, wherein the first and the second DNA molecules are different from one another, and each comprise a fragment of SEQ ID NO:10 or a complement thereof and function as DNA primers when used together in an amplification reaction with DNA obtained from the soybean plant, plant part, plant seed, or plant cell of claim 3 or a progeny plant thereof or a commodity product produced therefrom to produce an amplicon diagnostic for soybean event Gm_CSM63717 in a sample.

12. The pair of DNA molecules of claim 11, wherein:
a) the first and the second DNA molecules comprise SEQ ID NO:176 and SEQ ID NO:177; or
b) the amplicon comprises a nucleotide sequence selected from the group consisting of:
SEQ ID NO:1;
SEQ ID NO:2;
SEQ ID NO:3;
SEQ ID NO:4;
SEQ ID NO:5;
SEQ ID NO:6;
SEQ ID NO:7;
SEQ ID NO:8;
SEQ ID NO:9;
SEQ ID NO:10; and
a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, wherein the fragment is at least 10 nucleotides in length and comprises nucleotides 1,000-1,001 or 4,201-4,202 of SEQ ID NO:10.

13. (canceled)

14. A method of detecting the presence of soybean event Gm_CSM63717 in a sample derived from a soybean seed, plant, plant part, plant cell, progeny plant, or commodity product, the method comprising:
a) contacting the sample with the DNA molecule that functions as a DNA probe of claim 6;
b) subjecting the sample and the DNA molecule that functions as a probe to stringent hybridization conditions; and
c) detecting the hybridization of the DNA molecule that functions as a probe to a DNA molecule in the sample,
wherein the hybridization of the DNA molecule that functions as a probe to the DNA molecule in the sample is diagnostic for the presence of soybean event Gm_CSM63717 in the sample.

15. A method of detecting the presence of soybean event Gm_CSM63717 in a sample derived from a soybean seed, plant, plant part or plant cell, progeny plant or commodity product, the method comprising:
a) contacting the sample with the pair of DNA molecules of claim 11;
b) performing an amplification reaction sufficient to produce a DNA amplicon; and
c) detecting the presence of the DNA amplicon;
wherein the DNA amplicon comprises at least one of:
a 5′ junction sequence between flanking soybean genomic DNA and the transgenic insert of soybean event Gm_CSM63717,
a 3′ junction sequence between flanking soybean genomic DNA and the transgenic insert of soybean event Gm_CSM63717, SEQ ID NO: 9, and
a fragment of SEQ ID NO: 9 comprising a sufficient length of contiguous nucleotides of SEQ ID NO: 9 to identify the sequence as a fragment of the transgenic insert of Gm_CSM63717; and
wherein the presence of the DNA amplicon indicates the presence of soybean event Gm_CSM63717 in the sample.

16. The method of claim 15, wherein;
a) the DNA amplicon is at least 10 nucleotides in length, at least 11 nucleotides in length, at least 12 nucleotides in length, at least 13 nucleotides in length, at least 14 nucleotides in length, at least 15 nucleotides in length, at least 16 nucleotides in length, at least 17 nucleotides in length, at least 18 nucleotides in length, at least 19 nucleotides in length, at least 20 nucleotides in length, at least 25 nucleotides in length, at least 30 nucleotides in length, at least 35 nucleotides in length, at least 40 nucleotides in length, at least 45 nucleotides in length, at least 50 nucleotides in length, at least 60 nucleotides in length, at least 70 nucleotides in length, at least 80 nucleotides in length, at least 90 nucleotides in length, or at least 100 nucleotides in length; or
b) the DNA amplicon comprises a nucleotide sequence selected from the group consisting of SEQ ID NO:10; SEQ ID NO:9; SEQ ID NO:8; SEQ ID NO:7; SEQ ID NO:6; SEQ ID NO:5; SEQ ID NO:4; SEQ ID NO:3; SEQ ID NO:2; SEQ ID NO:1; and a fragment of any of SEQ ID NO:10, SEQ ID NO:8, SEQ ID NO:7, SEQ ID NO:6, SEQ ID NO:5, SEQ ID NO:4, SEQ ID NO:3, SEQ ID NO:2, and SEQ ID NO:1 that is at least 10 nucleotides in length and comprises nucleotides 1,000-1,001 or 4,201-4,202 of SEQ ID NO:10.

17. (canceled)

18. A method of detecting the presence of soybean event Gm_CSM63717 in a sample of DNA derived from a soybean seed, plant, plant part, plant cell, progeny plant or commodity product, the method comprising:
a) contacting the sample with the DNA molecule that functions as a probe of claim 6; and
b) performing a sequencing reaction to produce a target sequence,
wherein the target sequence comprises a nucleotide sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a complete complement of any thereof; and a fragment of any of SEQ ID NO:1, SEQ ID NO:2, SEQ ID NO:3, SEQ ID NO:4, SEQ ID NO:5, SEQ ID NO:6, SEQ ID NO:7, SEQ ID NO:8, and SEQ ID NO:10 that is at least 10 nucleotides long and comprises nucleotides 1,000-1,001 or 4,201-4,202 of SEQ ID NO:10.

19. A method of detecting the presence of soybean event Gm_CSM63717 in a sample derived from the soybean plant, plant part, plant seed, or plant cell of claim 3, or a progeny plant thereof or a commodity product produced therefrom, the method comprising:
a) contacting the sample with an antibody specific for the PPO (protoporphyrinogen oxidase) protein encoded by soybean event Gm_CSM63717; and
b) detecting binding of the antibody to the protein in the sample,
wherein the binding of the antibody indicates the presence of soybean event Gm_CSM63717 in the sample.

20. A DNA detection kit for detecting the presence of soybean event Gm_CSM63717 in a sample, wherein the kit comprises:
a) a pair of DNA molecules comprising a first DNA molecule and a second DNA molecule, wherein the first and the second DNA molecules are different from one another, and each comprise a fragment of SEQ ID NO:10 or a complement thereof and function as DNA primers when used together in an amplification reaction with DNA comprising soybean event Gm_CSM63717 to produce an amplicon diagnostic for soybean event Gm_CSM63717 in a sample;
b) the DNA molecule that functions as a DNA probe of claim 6; or
c) an antibody specific for the PPO protein encoded by soybean event Gm_CSM63717; wherein detecting binding of the antibody to the protein encoded by soybean event Gm_CSM63717 in a sample is diagnostic for the presence of soybean event Gm_CSM63717 in the sample.

21. (canceled)

22. A method of determining the zygosity of the soybean plant, plant part, plant seed, or plant cell of claim 3 or a progeny plant thereof or a commodity product produced therefrom, the method comprising:
a) contacting a sample comprising DNA derived from the soybean plant, plant part, plant seed, or plant cell with a first primer set capable of producing a first amplicon diagnostic for the presence of soybean event Gm_CSM63717, and a second primer set capable of producing a second amplicon diagnostic for wildtype soybean genomic DNA not comprising soybean event Gm_CSM63717;
b) performing a nucleic acid amplification reaction; and
c) detecting the first amplicon and the second amplicon, wherein the presence of both amplicons indicates that the plant, plant part, seed or cell is heterozygous for soybean event Gm_CSM63717, and the presence of only the first amplicon indicates that the plant, plant part, seed, or cell is homozygous for soybean event Gm_CSM63717.

23. The method of claim 22, wherein the first primer set comprises SEQ ID NO:176 and SEQ ID NO:177, and the second primer set comprises SEQ ID NO:208 and SEQ ID NO:209.

24. A method of determining the zygosity of h soybean plant, plant part, plant seed, or plant cell of claim 3 or a progeny plant thereof or a commodity product produced therefrom, the method comprising:
a) contacting a sample comprising DNA derived from the soybean plant, plant part, plant seed, or plant cell with a probe set comprising at least a first probe that specifically hybridizes to soybean event Gm_CSM63717, and at least a second probe that specifically hybridizes to soybean genomic DNA that was disrupted by insertion of the heterologous DNA of soybean event Gm_CSM63717 but does not hybridize to soybean event Gm_CSM63717; and
b) hybridizing the probe set with the sample under stringent hybridization conditions, wherein detecting hybridization of only the first probe under the hybridization conditions is diagnostic for a soybean plant, plant part, seed or plant cell homozygous for soybean event Gm_CSM63717, and wherein detecting hybridization of both the first probe and the second probe under the hybridization conditions is diagnostic for a soybean plant, plant part, seed, or plant cell heterozygous for soybean event Gm_CSM63717.

25. The method of claim 24, wherein the probe set comprises SEQ ID NO:178 and SEQ ID NO:210.

26. A DNA construct comprising:
a)_an expression cassette, wherein the expression cassette comprises in operable linkage i) a ubiquitin 2 (UBQ) promoter, a leader sequence, and an intron sequence from Medicago truncatula, ii) a codon optimized chloroplast transit peptide coding sequence of APG6 (Albino and Pale Green 6) from Adansonia digitata, iii) a codon-optimized protoporphyrinogen oxidase coding sequence from Enterobacter cloacae, and iv) a synthetic 3′ UTR sequence; or
b) a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% identical to the full length of SEQ ID NO: 9; and wherein the DNA construct comprises at the 5′ or 3′ end of said construct (i) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

27. The DNA construct of claim 26, wherein:
a) the DNA construct comprises SEQ ID NO:9;
b) the DNA construct comprises at the 5′ or 3′ end of said construct:
at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEO ID NO:14; or
at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:12 or SEQ ID NO:15;
c) the DNA construct comprises at the 5′ end of said construct one or more nucleotide sequences selected from SEQ ID NOs:36-105; or
d) the construct comprises at the 3′ end of said construct one or more nucleotide sequences selected from SEQ ID NOs:106-175.

28-31. (canceled)

32. A method for controlling or preventing weed growth in an area, the method comprising planting the soybean plant or plant seed of claim 3 in the area and applying an effective amount of a PPO herbicide to control weeds in the area without injury to the soybean plant or plant seed or with less than about 10% injury to the soybean.

33. A method for controlling volunteer soybean comprising the soybean plant or plant seed of claim 3 in an area, the method comprising applying an herbicidally effective amount of at least one herbicide other than a PPO herbicide, wherein the herbicide application prevents growth of soybean comprising soybean event.

34. The method of claim 33, wherein the herbicide other than a PPO herbicide is selected from the group consisting of atrazine, topramezone, clopyralid, pyrithiobac, fluometuron, (3-(3,4-dichlorophenyl)-1,1-dimethylurea) (DCMU), trifloxysulfuron, paraquat, diquat, glyphosate, glufosinate, and combinations of any thereof.

35. A method of obtaining a seed of a soybean plant or a soybean plant that is tolerant to PPO herbicides, the method comprising:
a) obtaining a population of progeny seed of the soybean plant of claim 3 or plants grown therefrom, at least one of which comprises soybean event Gm_CSM63717; and
b) identifying at least a first progeny seed or plant grown therefrom that comprises soybean event Gm_CSM63717.

36. The method of claim 35, wherein:
a) identifying the progeny seed or plant grown therefrom that comprises soybean event Gm_CSM63717 comprises:
i) growing the progeny seed or plant to produce progeny plants;
ii) treating the progeny plants with an effective amount of a PPO herbicide; and
iii) selecting a progeny plant that is tolerant to the PPO herbicide;
b) identifying the progeny seed or plant grown therefrom that comprises soybean event Gm_CSM63717 comprises detecting the presence of soybean event Gm_CSM63717 in a sample derived from the progeny seed or plant grown therefrom: or
c) identifying the progeny seed or plant grown therefrom that comprises soybean event Gm_CSM63717 comprises detecting the presence of the PPO protein encoded by soybean event Gm_CSM63717 in a sample derived from the progeny seed or plant grown therefrom.

37-38. (canceled)

39. A method of improving tolerance to PPO herbicides in a soybean plant comprising:
a) inserting the DNA construct of claim 26 into the genome of a soybean cell;
b) generating a soybean plant from the soybean cell; and
c) selecting a soybean plant comprising the DNA construct.

40. The method of claim 39, wherein the selecting comprises treating the soybean cell or plant with an effective amount of a PPO herbicide.

41. A soybean plant, plant seed, plant part, or plant cell comprising:
a) a recombinant DNA molecule comprising a sequence selected from the group consisting of SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; SEQ ID NO:10; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing; or
b) the DNA construct of claim 26.

42. The soybean plant, plant seed, plant part, or plant cell of claim 41, wherein:
a) the plant, plant seed, plant part, or plant cell expresses a PPO herbicide tolerance gene;
b) the plant, plant seed, plant part, or plant cell is tolerant to one or more PPO herbicides:
c) the plant, plant seed, plant part, or plant cell further comprises at least one additional transgene for tolerance to at least one additional herbicide:
d) the plant, plant seed, plant part, or plant cell comprises soybean event Gm_CSM63717, a representative sample of seed comprising the event having been deposited under ATCC Accession No. PTA-127604:
e) the plant, plant seed, plant part, or plant cell is further defined as a progeny plant of any generation of a soybean plant comprising soybean event Gm_CSM63717, or a soybean plant part, plant seed, or plant cell derived therefrom;
f) the plant part comprises a microspore, pollen, an anther, an ovule, an ovary, a pod, a flower, an embryo, a stem, a bud, a node, a leaf, a root, or a callus.

43-48. (canceled)

49. The soybean plant part of claim 3, wherein the plant part comprises a microspore, pollen, an anther, an ovule, an ovary, a pod, a flower, an embryo, a stem, a bud, a node, a leaf, a root, or a callus.

50. A soybean plant, plant seed, plant part, or plant cell obtained by the method of claim 35.

51. A soybean plant, plant cell, plant part, or plant seed comprising a recombinant DNA construct integrated in chromosome 13, wherein the recombinant DNA construct confers tolerance to at least one PPO herbicide, and wherein the recombinant DNA construct is integrated in a position of said chromosome flanked by at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO:11 or SEQ ID NO:14; or (ii) at least 10, at least 11, at least 12, at least 13, at least 14, at least 15, at least 16, at least 17, at least 18, at least 19, at least 20, at least 21, at least 22, at least 23, at least 24, at least 25, at least 30, at least 35, at least 40, at least 45, at least 50, at least 60, at least 70, at least 80, at least 90, at least 100, at least 150, at least 200, at least 250, at least 300, at least 400, at least 500, at least 1,000, at least 1,500, or at least 2,000 contiguous nucleotides of SEQ ID NO: 12 or SEQ ID NO:15.

52. The soybean plant, plant cell, plant part, or plant seed of claim 51, wherein:
a) the at least 50 contiguous nucleotides of SEQ ID NO:11 or SEQ ID NO:14 comprise one or more nucleotide sequences selected from SEQ ID NOs:36-105; or
b) the at least 50 contiguous nucleotides of SEQ ID NO:12 or SEQ ID NO:15 comprise one or more nucleotide sequences selected from SEQ ID NOs:106-175.

53. (canceled)

54. The soybean plant, plant seed, plant part, or plant cell of claim 42, wherein:
a) the PPO herbicide is selected from the group consisting of diphenylethers, N-phenylphthalimides, oxadiazoles, oxazolidinediones, phenylpyrazoles, pyrimidinediones, thiadiazoles, triazolinones, benzoxazinone derivatives, other PPO herbicides, and combinations of any thereof;
b) the at least one additional transgene is selected from the group consisting of FT_Tv7, dicamba monooxygenase (DMO), phosphinothricin N-acetyltransferase (PAT), triketone dioxygenase (TDO), 5-enolpyruvylshikimate-3-phosphate synthase (EPSPS), and combinations of any thereof;
c) the at least one additional transgene provides tolerance to a herbicide having a mode of action selected from the group consisting of inhibitors of glutamine synthetase, inhibitors of EPSPS, β-triketone HPPD inhibitors, synthetic auxins, and combinations of any thereof;
d) the soybean plant, plant seed, plant part, or plant cell further comprises soybean event MON89788 or soybean event Gm_CSM63714;
e) the soybean plant, plant seed, plant part, or plant cell further comprises a recombinant DNA molecule comprising a sequence selected from the group consisting of SEQ ID NO:182; SEQ ID NO:183; SEQ ID NO:184; SEQ ID NO:185; SEQ ID NO:186; SEQ ID NO:187; SEQ ID NO:188; SEQ ID NO:189; SEQ ID NO:190; SEQ ID NO:191; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:182 or the full length of SEQ ID NO: 183; and a complete complement of any of the foregoing; or
f) the soybean plant, plant seed, plant part, or plant cell further comprises a recombinant DNA molecule comprising a sequence selected from the group consisting of SEQ ID NO:192, SEQ ID NO:193, SEQ ID NO:194, SEQ ID NO:195, SEQ ID NO:196, SEQ ID NO:197; a polynucleotide having a sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:192 or the full length of SEQ ID NO:193; and a complete complement of any of the foregoing.

55. The soybean plant, plant seed, plant part, or plant cell of claim 54, wherein:
a) the diphenylether is selected from the group consisting of acifluorfen, bifenox, ethoxyfen, fluorodifen, fluoronitrofen, furyloxyfen, halosafen, chlomethoxyfen, chlornitrofen, ethoxyfen-ethyl, fluoroglycofen, lactofen, nitrofen, oxyfluorfen, fomesafen, a salt of any thereof, and an ester of any thereof; the N-phenylphthalimide is selected from the group consisting of cinidon-ethyl, flumiclorac, flumiclorac-pentyl, and flumioxazin; the oxadiazole is selected from the group consisting of oxadiargyl and oxadiazon; the oxazolidinedione is pentoxazone; the phenylpyrazole is selected from the group consisting of fluazolate, pyraflufen, and pyraflufen-ethyl; the pyrimidinedione is selected from the group consisting of benzfendizone, butafenacil, epyrifencacil (S-3100), flupropacil, flufenoximacil, saflufenacil, and tiafenacil; the thiadiazole is selected from the group consisting of fluthiacet-methyl and thidiazimin; the triazolinone is selected from the group consisting of azafenidin, bencarbazone, carfentrazone, its salts and esters, and sulfentrazone; the benzoxazinone derivative is 1,5-dimethyl-6-thioxo-3-(2,2,7-trifluoro-3,4-dihydro-3-oxo-4-prop-2-ynyl-2H-1,4-benzoxazin-6-yl)-1,3,5-triazinane-2,4-dione (trifludimoxazin)); the other PPO herbicide is selected from the group consisting of chlorphthalim, flufenpyr, flufenpyr-ethyl, flumipropyn, pyraclonil, profluazol, pyridin-2-ylmethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-chloro-4-fluoro-5-[3-methyl-2,6-dioxo-4-(trifluoromethyl)-3,6-dihydropyrimidin-1(2H)-yl]phenoxy}pyridin-2-yl)oxy]acetate, 2-methoxyethyl [(3-{2-cyano-4-fluoro-5-[3-methyl-2,6-dioxo-4-(trifluoromethyl)-3,6-dihydropyrimidin-1(2H)-yl]phenoxy}pyridin-2-yl)oxy]acetate, cyanomethyl [(3-{2-bromo-4-fluoro-5-[3-methyl-2,6-dioxo-4-(trifluoromethyl)-3,6-dihydropyrimidin-1(2H)-yl]phenoxy}pyridin-2-yl)oxy]acetate; cyclopropylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate; methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate (flufenoximacil), methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, methyl 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(Z)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, ethyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}propanoic acid, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}-2-methylpropanoate, methyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy} butanoate, 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2R)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, (2S)-2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoic acid, ethyl 2-{[(E)-{2-chloro-4-fluoro-5-[3-methyl-2,6-dioxo-4-(trifluoromethyl)-3,6-dihydropyrimidin-1(2H)-yl]benzylidene}amino]oxy}butanoate, methyl 2-({(E)-[2-chloro-5-(3,5-dimethyl-2,6-dioxo-4-sulfanylidene-1,3,5-triazinan-1-yl)-4-fluorobenzylidene]amino}oxy)propanoate methyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, 2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoic acid, ethyl 2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2R)-2-({(E)-[2-chloro-5-(3,5-dimethyl-2,6-dioxo-4-sulfanylidene-1,3,5-triazinan-1-yl)-4-fluorobenzylidene]amino}oxy)propanoate, ethyl (2S)-2-({(E)-[2-chloro-5-(3,5-dimethyl-2,6-dioxo-4-sulfanylidene-1,3,5-triazinan-1-yl)-4-fluorobenzylidene]amino}oxy)propanoate, methyl 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, methyl (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoate, 2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2R)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, (2S)-2-{[(E)-{5-[3-amino-2,6-dioxo-4-(trifluoromethyl)-3,6-dihydropyrimidin-1(2H)-yl]-2-chloro-4-fluorobenzylidene}amino]oxy}propanoic acid, ethyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, methyl 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, 3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylic acid, ethyl (5S)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl (5R)-3-{2-chloro-4-fluoro-5-[3-methyl-2,6-dioxo-4-(trifluoromethyl)-3,6-dihydropyrimidin-1(2H)-yl]phenyl}-5-methyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-propyl-4,5-dihydro-1,2-oxazole-5-carboxylate, ethyl 3-{2-chloro-4-fluoro-5-[3-methyl-2,6-dioxo-4-(trifluoromethyl)-3,6-dihydropyrimidin-1(2H)-yl]phenyl}-5-ethyl-4,5-dihydro-1,2-oxazole-5-carboxylate, 3-[4-chloro-2-fluoro-5-(5-{[(isopropylideneamino)oxy]carbonyl}-5-methyl-4,5-dihydro-1,2-oxazol-3-yl)phenyl]-1-methyl-6-(trifluoromethyl)pyrimidine-2,4(1H,3H)-dione, ethyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, methyl 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylate, 3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5R)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, (5S)-3-[2-chloro-5-(3,5-dimethyl-2,6-dioxo-4-sulfanylidene-1,3,5-triazinan-1-yl)-4-fluorophenyl]-5-methyl-4,5-dihydro-1,2-oxazole-5-carboxylic acid, 3-[4-chloro-2-fluoro-5-(5-{[(isopropylideneamino)oxy]carbonyl}-5-methyl-4,5-dihydro-1,2-oxazol-3-yl)phenyl]-1,5-dimethyl-6-sulfanylidene-1,3,5-triazinane-2,4-dione, ethyl 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylate, 3-{5-[3-amino-2,6-dioxo-4-(trifluoromethyl)-3,6-dihydropyrimidin-1(2H)-yl]-2-chloro-4-fluorophenyl}-5-methyl-4,5-dihydro-1,2-oxazole-5-carboxylic acid, methyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, ethyl 3-{2-chloro-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, methyl 3-{2-bromo-4-fluoro-5-[3-methyl-2,6-dioxo-4-(trifluoromethyl)-3,6-dihydropyrimidin-1(2H)-yl]phenyl}-3a,4,5,6-tetrahydro-6aH-cyclopenta[d][1,2]oxazole-6a-carboxylate, 2-ethoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate, {[(1-{2-chloro-4-fluoro-5-[3-methyl-2,6-dioxo-4-(trifluoromethyl)-3,6-dihydropyrimidin-1(2H)-yl]phenoxy}cyclopropyl)carbonyl]oxy}acetic acid, and 2-methoxy-2-oxoethyl 1-{2-chloro-4-fluoro-5-[3-methyl-2,6-dioxo-4-(trifluoromethyl)-3,6-dihydropyrimidin-1(2H)-yl]phenoxy}cyclopropanecarboxylate;
b) the FT_Tv7 transgene comprises a polynucleotide sequence encoding a protein having the amino acid sequence of SEQ ID NO:31; the DMO transgene comprises a polynucleotide sequence encoding a protein having the amino acid sequence of SEQ ID NO:29; the PAT transgene comprises a polynucleotide sequence encoding a protein having the amino acid sequence of SEQ ID NO:27; the TDO transgene comprises a polynucleotide sequence encoding a protein having the amino acid sequence of SEQ ID NO: 33, and the EPSPS transgene comprises a polynucleotide sequence encoding a protein having the amino acid sequence of SEQ ID NO:35; or
c) the synthetic auxin is selected from the group consisting of dicamba, 2,4-D, dichlorprop, mecoprop, 2,4,5-T (2,4,5-trichlorophenoxyacetic acid), and combinations of any thereof; the inhibitor of glutamine synthetase comprises glufosinate; the β-triketone HPPD inhibitor is selected from the group consisting of mesotrione, benzobicyclon (BBC), tembotrione, sulcotrione, tefuryltrione, and combinations of any thereof, and the inhibitor of 5-enolpyruvylshikimate-3-phosphate synthase (EPSPS) comprises glyphosate.

56. The method of claim 32, where in the effective amount of PPO herbicide is about 0.0009 lb/acre to about 1.5 lb/acre over a growing season.

57. A method of producing a progeny soybean plant comprising soybean event Gm_CSM63717 comprising:
a) sexually crossing the soybean plant of claim 3 with itself or a second soybean plant;
b) collecting one or more seeds produced from the cross;
c) growing one or more seeds to produce one or more progeny plants; and
d) selecting at least a first progeny plant or seed comprising soybean event Gm_CSM63717.

58. An inbred or hybrid soybean plant or seed comprising soybean event Gm_CSM63717 produced by the method of claim 57.

59. A nonliving or nonregenerable soybean plant material or a commodity product comprising:
a) a recombinant DNA molecule comprising a nucleotide sequence selected from the group consisting of SEQ ID NO:10; SEQ ID NO:1; SEQ ID NO:2; SEQ ID NO:3; SEQ ID NO:4; SEQ ID NO:5; SEQ ID NO:6; SEQ ID NO:7; SEQ ID NO:8; SEQ ID NO:9; a polynucleotide having a nucleotide sequence that is at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to the full length of SEQ ID NO:10 or the full length of SEQ ID NO: 9; and a complete complement of any of the foregoing;
b) the DNA construct of claim 26; or
c) soybean event Gm_CSM63717, a representative sample of seed comprising the soybean event soybean event Gm_CSM63717 having been deposited under ATCC Accession No. PTA-127604.

60-61. (canceled)

62. The commodity product of claim 59, wherein:
a) the commodity product is produced from a transgenic soybean plant, plant part, plant seed, or plant cell comprising soybean event Gm_CSM63717; or
b) the commodity product comprises whole or processed seeds; viable or nonviable seeds; viable plant parts (such as roots, nodes, buds or leaves); viable plant cells; processed plant parts; processed plant tissues; dehydrated plant tissues; dehydrated plant parts; frozen plant tissues; frozen plant parts; food for human consumption such as soybean oil, soy milk, soy flour, soy grits, soy protein, soy protein concentrate, hydrolyzed vegetable protein, textured soy protein, lecithin, curd, tofu, vegetable soybean (edamame), soy sprouts, soy film (yuba), roasted soybeans, miso, tempeh, soy sauce, or natto; plant parts processed for animal feed such as soy meal; soy fiber; biodiesel; bio-composite building materials such as particleboard, laminated plywood, or lumber products; soy oil-based solvents; soy oil-based industrial lubricants; soy ink; soy candles; soy crayons; soy-based hydraulic fluid; or soy-based foams.

63. (canceled)

64. A method of producing a commodity product, the method comprising:
a) obtaining the soybean plant, plant part, or plant seed of claim 3; and
b) producing a commodity product from the transgenic soybean plant, plant part, or plant seed.

65. A method of controlling, preventing, or reducing the development of herbicide-tolerant weeds comprising cultivating in a crop growing environment a soybean plant comprising transgenes that provide tolerance to (i) a PPO herbicide and (ii) herbicides with at least three additional herbicide modes of action, the three additional herbicide modes of action each being different from one another.

66. A method for controlling, preventing, or reducing the development of herbicide-tolerant weeds comprising:
a) cultivating in a crop growing environment a soybean plant comprising the DNA construct of claim 26 or event Gm_CSM63717, and at least three additional transgenes for providing tolerance to herbicides with at least three additional herbicide modes of action, the three additional herbicide modes of action each being different from one another; and
b) applying to the crop growing environment at least one herbicide selected from the group consisting of dicamba, glufosinate, 2,4-D, a PPO inhibitor, a β-triketone HPPD inhibitor, glyphosate, and any combination thereof, wherein the soybean plant is tolerant to the at least one herbicide.

67. The method of claim 66, wherein the transgenes that provide tolerance to the herbicides with the at least three additional herbicide modes of action are present at a single genomic location in the soybean plant.

68. A method of reducing loci for soybean breeding by site-directed insertion of a transgene that provides tolerance to a PPO herbicide at a genomic location in a soybean plant that is within about 1-6 cM of a locus in the genome of the soybean plant that comprises transgenes for tolerance to at least three additional herbicide modes of action, the three additional herbicide modes of action each being different from one another.

69-75. (canceled)

76. A method for controlling or preventing weed growth in an area, comprising planting the soybean plant or plant seed of claim 3, wherein said soybean plant or plant seed further further comprises event Gm_CSM63714 or event MON89788 in the area and applying one or more herbicides selected from the group consisting of a PPO herbicide, dicamba, glufosinate, 2, 4-D, β-triketone HPPD inhibitor, glyphosate, and combinations of any thereof to control weeds in the area without injury or with less than 10% injury to the soybean.
